(19) United States
(12) Patent Application Publication
(10) Pub. No.: US 2022/0323594 A1
(43) Pub. Date: Oct. 13, 2022

(54) LIPIDATED CATIONIC PEPTIDE-PEG COMPOSITIONS FOR NUCLEIC ACID DELIVERY

(71) Applicant: NUTCRACKER THERAPEUTICS, INC., Emeryville, CA (US)

(72) Inventor: Colin James McKinlay, Fremont, CA (US)

(21) Appl. No.: 17/615,857

(22) PCT Filed: Aug. 7, 2020
(86) PCT No.: PCT/US2020/045508

§ 371 Date: Dec. 2, 2021

Related U.S. Application Data
(60) Provisional application No. 62/907,470, filed on Sep. 27, 2019.
(60) Provisional application No. 62/885,022, filed on Aug. 9, 2019.

(51) Int. Cl. A61K 47/64; A61K 47/54; A61K 47/60; C12N 15/88; A61K 48/00; C07K 7/06

(52) U.S. Cl. CPC A61K 47/6455; A61K 47/543; A61K 47/60; C12N 15/88; A61K 48/0041; C07K 7/06

(57) ABSTRACT

The present disclosure relates to complexes and compositions of cationic compounds combined with low mass percentages of PEGylated compounds, such as PEGylated lipids, for the delivery of nucleic acids and other polyanionic cargoes to cells, methods for preparing complexes and compositions comprising one or more cationic compounds and a low mass percentage of PEGylated compounds with polyanionic compounds, and methods for delivering the polyanionic compounds to cells.

### CROSS REFERENCE TO RELATED APPLICATIONS

This application claims priority to and the benefit of U.S. Provisional Application No. 62/885,022, filed on Aug. 9, 2019, and U.S. Provisional Application No. 62/907,470, filed on Sep. 27, 2019, the disclosures of which are incorporated herein by reference in their entireties.

### FIELD

The present disclosure relates to complexes and compositions of cationic compounds combined with low mass percentages of PEGylated compounds, such as PEGylated lipids or PEGylated diacylglycerol derivatives, for the delivery of nucleic acids and other polyanionic cargoes. More specifically, the present disclosure relates to tertiary amino lipidated and/or PEGylated peptoids, and complexes and compositions thereof combined with small amounts of PEGylated lipids for nucleic acid delivery. The present disclosure also provides methods for preparing the complexes and compositions thereof comprising one or more cationic compounds and a low mass percentage of PEGylated compounds with polyanionic compounds, such as nucleic acids, and methods for delivering polyanionic compounds to cells.

### BACKGROUND

The ability to deliver nucleic acids consistently and effectively into cells is a critical requirement for the practicable implementation of gene therapy, siRNA-, miRNA-, antisense-, or mRNA-based therapy in a clinical setting. A variety of cationic compounds have been explored as delivery vehicles, which can encapsulate polyanionic nucleic acids and stabilize the nucleic acids against nucleases in vivo. Lipitoid 1 (shown in FIG. 1) is an example of a cationic peptoid-phospholipid conjugate construct that has been widely evaluated as a standalone cationic delivery vehicle. However, complexes of lipitoids with nucleic acids may still suffer from further complications in vivo, including, for example, particle instability and aggregation.

Multicomponent lipid mixtures, such as lipid nanoparticle formulations, have been a large area of focus for achieving the requisite pharmacokinetic properties to support nucleic acid delivery in vivo, including improved formulation (particle) stability. Lipid nanoparticles incorporate cationic compounds with a number of compatible lipid co-components (e.g., shielding lipid, structural lipid, and phospholipid) that provide charge insulation and confer structural and chemical stability to the delivery vehicle-nucleic acid complex under physiological conditions.

Yet despite the promise of lipid nanoparticle formulations to achieve the requisite properties for in vivo nucleic acid transfer, optimization of lipid formulations is often difficult due to the interplay of many variables, including identifying compatible co-components for the nucleic acid to be delivered and carefully tuning the relative amounts of each component to achieve the desired pharmacokinetic properties. Moreover, the majority of lipid-based formulations typically involve no fewer than four distinct components, and the bulk of existing formulations is made up largely of the delivery components rather than the nucleic acid material itself. Consequently, the active therapeutic agent constitutes only a small fraction of the overall formulation by weight.

Thus, there remains a need for formulations for gene therapy that achieve effective, efficient delivery of a higher mass percentage of active nucleic acid material without compromising the pharmacokinetic properties required for in vivo applications.

### BRIEF SUMMARY

The present disclosure provides complexes and compositions comprising cationic compounds, such as lipitoids or tertiary amino lipidated and/or PEGylated cationic peptide compounds, in combination with PEGylated compounds, such as PEG lipids, as delivery systems for polyanionic compounds. The complexes and compositions described herein utilize a minimal quantity of PEG compound(s) by weight basis relative to the content of the cationic compounds and polyanionic compounds in order to stabilize against particle aggregation, thus providing an easily optimizable, stable formulation having a high mass percentage of active polyanionic material.

In one aspect, the present disclosure includes a composition, comprising complexes of:

(i) one or more polyanionic compounds,
(ii) one or more PEG compounds; and
(iii) one or more cationic compounds, wherein the one or more cationic compounds are selected from the group consisting of:

(a) one or more tertiary amino lipidated and/or PEGylated cationic peptide compounds of formula (I) or salts thereof:

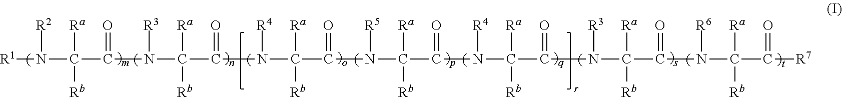

wherein:
m is 0;
n is an integer from 0 to 5;
s is an integer from 0 to 5;
t is 0;

wherein at least one of n and s is nonzero;
r is an integer from 1 to 20;
each o is independently an integer 0, 1, 2, 3 or 4;
each q is independently an integer 0, 1, 2, 3 or 4;
each p is independently an integer 1 or 2;
R¹ is —H, alkyl, alkylaryl, —COR^{1a} or a lipid moiety, wherein R^{1a} is —H, —OH, alkyl, aryl, alkylaryl, —O-alkyl, or —O-alkylaryl;
each R² is independently an ethylene glycol moiety of the formula —CH₂CH₂O(CH₂CH₂O)ᵤCH₃, and wherein each u is independently an integer from 1 to 200;
each R³ is independently a lipid moiety;
each R⁴ is independently a neutral spacer moiety or a lipid moiety;
each R⁵ is independently a cationic moiety;
each R⁶ is independently an ethylene glycol moiety of the formula —CH₂CH₂O(CH₂CH₂O)ᵥCH₃, and wherein each v is independently an integer from 1 to 200;
R⁷ is —H, alkyl, acyl, —OH, —OR^{7a}, —NH₂, —NHR^{7a}, or a lipid moiety, wherein R^{7a} is alkyl, acyl, or a lipid moiety; and
each R^{a} and R^{b} are independently —H, C₁-C₄-alkyl, or a side chain moiety found on a naturally- or non-naturally-occurring amino acid;

(b) one or more lipitoids of formula (II):

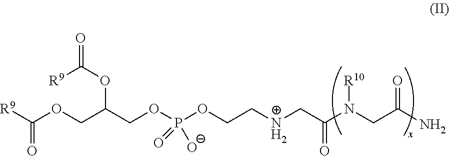

wherein:
x is a integer from 1 to 100;
each R⁹ is independently a lipid moiety; and
each R¹⁰ is independently a cationic or neutral spacer moiety;

(c) one or more lipid-like compounds of formula (III):

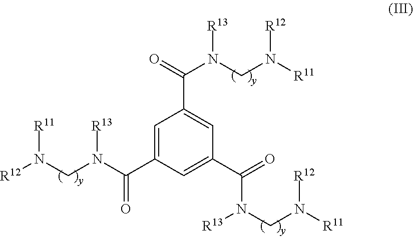

wherein:
each R¹¹ is independently substituted or unsubstituted alkyl;
each R¹² is independently substituted or unsubstituted alkyl;
each R¹³ is independently hydrogen or substituted or unsubstituted alkyl; and each y is independently an integer from 1 to 8;
   or

(d) any combination thereof;

and wherein the one or more cationic compounds, the one or more PEG compounds and the one or more polyanionic compounds have a combined mass percentage of at least 90% w/w of the complexes.

In some embodiments of this aspect, the one or more cationic compounds are one or more tertiary amino lipidated and/or PEGylated cationic peptide compounds. In certain embodiments, the one or more tertiary amino lipidated and/or PEGylated cationic peptide compounds comprises a block of N-lipidated amino acid residues, wherein either n or s is at least 2. In certain embodiments, the one or more tertiary amino lipidated and/or PEGylated cationic peptide compounds comprises a block of N-lipidated amino acid residues, wherein either n or s is 4. In other embodiments of the present aspect, each R³ is independently C₄-C₂₂-alkyl or C₄-C₂₂-alkenyl, and wherein the C₄-C₂₂-alkenyl is optionally mono- or poly-unsaturated. In certain embodiments, which may be combined with one or more of the preceding embodiments, each R³ is independently C₈-C₁₂ alkyl. In still yet other embodiments, which may be combined with one or more of the preceding embodiments, each R³ is independently selected from the group consisting of 2-ethylhex-1-yl, caproyl, oleyl, stearyl, linoleyl, myristyl, and lauryl. In some embodiments, which may be combined with one or more of the preceding embodiments, the one or more tertiary amino lipidated and/or PEGylated cationic peptide compounds comprises a block of N-lipidated amino acid residues, comprising a set of mixed N-lipidated amino acid residues having one of two different lipid moieties R^{3a} and R^{3b}. In still other embodiments, which may be combined with one or more of the preceding embodiments, at least one lipid moiety R³ (or R^{3a} or R^{3b}) is a branched aliphatic moiety. In certain embodiments, which may be combined with one or more of the preceding embodiments, at least one lipid moiety R³ (or R^{3a} or R^{3b}) is 2-ethylhex-1-yl.

In some embodiments of the present aspect, at least one of the one or more tertiary amino lipidated and/or PEGylated cationic peptide compounds comprises a cationic domain comprising at least two cationic amino acid residues having cationic moieties R⁵. In other embodiments, which may be combined with one or more of any of the previous embodiments, each R⁵ is independently aminoalkyl, alkylaminoalkyl, aminoalkylaminoakyl, guanidinoalkyl, N-heterocyclylalkyl or N-heteroaryl. In certain embodiments, which may be combined with any of the foregoing embodiments, each R⁵ is independently selected from the group consisting of:

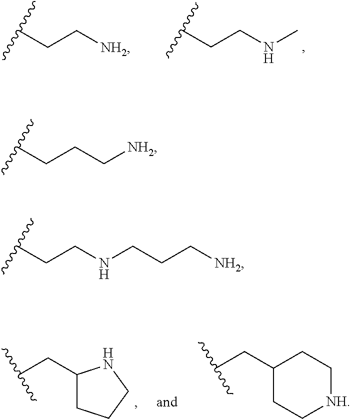

In other embodiments, each R⁵ is a —(C₁-C₄alkylene)-NR^{5a}R^{5b}, wherein R^{5a} and R^{5b} are independently H, C₁-C₃ alkyl, or C₆-C₁₀ aryl, or R^{5a} and R^{5b} are taken together with the nitrogen atom to which they are attached to form a 4-7 membered N-heterocycloalkyl ring, wherein the N-heterocycloalkyl ring may optionally include one additional heteroatom selected from the group consisting of N and O. In still other embodiments, each R⁵ is

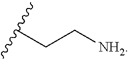 [SMILES: *CCN]

In some embodiments, each R⁵ is independently

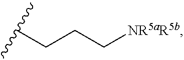

wherein R^{5a} and R^{5b} are independently H, C₁-C₃ alkyl, or C₆-C₁₀ aryl, or R^{5a} and R^{5b} are taken together with the nitrogen atom to which they are attached to form a 4-7 membered N-heterocycloalkyl ring, wherein the N-heterocycloalkyl ring may optionally include one additional heteroatom selected from the group consisting of N and O. In certain embodiments, each R⁵ is

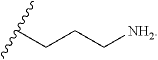 [SMILES: *CCCN]

In yet other embodiments of the present aspect, each neutral spacer moiety R⁴ is independently a C₁-C₅ straight chain alkyl, a C₁-C₄-alkyl substituted by cycloalkyl, heterocyclylalkyl, alkylaryl, arylalkyl, alkylheteroaryl, heteroarylalkyl, alkoxy, alkoxyalkyl, or hydroxyalkyl, wherein each cycloalkyl, heterocyclylalkyl, alkylaryl, arylalkyl, alkylheteroaryl, heteroarylalkyl, alkoxy, alkoxyalkyl, or hydroxyalkyl is optionally substituted with one or more substituents —OH, halo, or alkoxy. In some embodiments, which may be combined with one or more of any of the preceding embodiments, each neutral spacer moiety R⁴ is independently selected from the group consisting of: —CH₃,

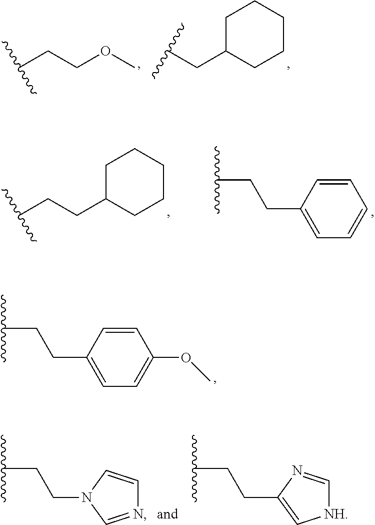

In certain embodiments, each neutral spacer moiety R⁴ is

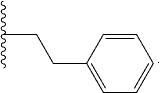 [SMILES: *CCc1ccccc1]

In yet further embodiments, which may be combined with one or more of any of the previous embodiments, at least one of the one or more tertiary amino lipidated and/or PEGylated cationic peptide compounds comprises a cationic domain comprising at least two amino acid residues having R⁵ cationic moieties and wherein each of the at least two cationic amino acid residues within the cationic domain are separated by at least one amino acid residue having a neutral spacer or lipid moiety R⁴. In some embodiments of the present aspect, which may be combined with one or more of any of the foregoing embodiments, at least one of the one or more tertiary amino lipidated and/or PEGylated cationic peptide compounds comprises at least one trimer subunit —R^{cation}—R^{neutral}—R^{neutra}, and wherein R^{cation} is an amino acid residue comprising a cationic moiety R⁵ and each R^{neutral} is an amino acid residue comprising a neutral spacer moiety R⁴. In other embodiments of the present aspect, at least one of the one or more tertiary amino lipidated and/or PEGylated cationic peptide compounds comprises at least one trimer subunit —R^{cation}—R^{neutral}—R^{lipid}, and wherein R^{cation} is an amino acid residue comprising a cationic moiety R⁵, each R^{neutral} is an amino acid residue comprising a neutral spacer moiety R⁴ and each R^{lipid} is a lipid moiety R⁴. In certain embodiments of any of the foregoing embodiments, each cationic moiety R⁵ is

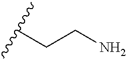 [SMILES: *CCN]

and each neutral spacer moiety R⁴ is

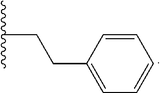 [SMILES: *CCc1ccccc1]

In other embodiments of this aspect, which may be combined with any one or more of the previous embodiments, R^{a} and R^{b} are independently selected from the group consisting of —H and —CH₃. In certain other embodiments of this aspect, which may be combined with any one or more of the previous embodiments, R^{a} and R^{b} are —H.

In still other embodiments of the present aspect, which may be combined with one or more of any of the previous embodiments, the one or more cationic peptide compounds and one or more PEG compounds are present at a mass ratio of peptide compound(s)-to-PEG compound(s) between 90:10 and 99:1. In yet further embodiments of the present aspect, which may be combined with one or more of any of the preceding embodiments, the one or more cationic peptide compounds and the one or more PEG compounds are present at a mass ratio of peptide compounds-to-PEG compound(s) between 95:5 and 98:2. In certain embodiments of the present aspect, which may be combined with one or more of any of the preceding embodiments, the one or more PEG compounds comprises DMG-PEG2000. In still yet other embodiments of this aspect, which may be combined with one or more of any of the preceding embodiments, the one or more PEG compounds comprises one or more tertiary amino lipidated and/or PEGylated cationic peptide compounds of formula (I) or salts thereof:

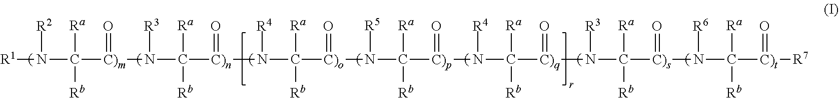

wherein:
m is an integer from 0 to 15;
n is an integer from 0 to 5;
s is an integer from 0 to 5;
t is an integer from 0 to 15;
   wherein at least one of m and t is nonzero;
r is an integer from 1 to 20;
each o is independently an integer 0, 1, 2, 3 or 4;
each q is independently an integer 0, 1, 2, 3 or 4;
each p is independently an integer 1 or 2;
R¹ is —H, alkyl, alkylaryl, —COR^{1a} or a lipid moiety,
wherein R^{1a} is —H, —OH, alkyl, aryl, alkylaryl, —O-alkyl, or —O-alkylaryl;
each R² is independently an ethylene glycol moiety of the formula —CH₂CH₂O(CH₂CH₂O)ᵤCH₃, and wherein each u is independently an integer from 1 to 200;
each R³ is independently a lipid moiety;
each R⁴ is independently a neutral spacer moiety or a lipid moiety;
each R⁵ is independently a cationic moiety;
each R⁶ is independently an ethylene glycol moiety of the formula —CH₂CH₂O(CH₂CH₂O)ᵥCH₃, and wherein each v is independently an integer from 1 to 200;
R⁷ is —H, alkyl, acyl, —OH, —OR^{7a}, —NH₂, —NHR^{7a}, or a lipid moiety,
wherein R^{7a} is alkyl, acyl, or a lipid moiety; and
each R^{a} and R^{b} are independently —H, C₁-C₄-alkyl, or a side chain moiety found on a naturally- or non-naturally-occurring amino acid.

In yet other embodiments of the present aspect wherein the one or more PEG compounds comprises one or more tertiary amino lipidated and/or PEGylated cationic peptide compounds, when m is an integer from 0 to 3, each u is independently an integer from 30 to 50; and when m is an integer from 4 to 15, each u is independently an integer from 1 to 10. In still yet other embodiments wherein the one or more PEG compounds comprises one or more tertiary amino lipidated and/or PEGylated cationic peptide compounds, which may be combined with one or more of any of the previous embodiments, the composition comprises one or more tertiary amino lipidated and/or PEGylated cationic peptide compounds wherein when t is an integer from 0 to 3, each v is independently an integer from 30 to 50; when t is an integer from 4 to 15, each v is independently an integer from 1 to 10. In certain embodiments, which may be combined with any of the preceding embodiments, m is 1, and u is an integer from 40 to 45; or t is 1, and v is an integer from 40 to 45.

In yet other embodiments of the present aspect, which may be combined with one or more of any of the preceding embodiments, the one or more polyanionic compounds comprises a nucleic acid. In still yet another embodiment of the present aspect, which may be combined with one or more of any of the preceding embodiments, the one or more polyanionic compounds comprises a nucleic acid and wherein the mass ratio of the one or more cationic peptide compounds to the nucleic acid is between 0.5:1 and 20:1. In some embodiments of the present aspect, which may be combined with one or more of any of the previous embodiments, the one or more polyanionic compounds comprises a nucleic acid and wherein the nucleic acid is an mRNA encoding a polypeptide. In certain embodiments of the present aspect, which may be combined with one or more of any of the preceding embodiments, the one or more polyanionic compounds comprises a nucleic acid and wherein the nucleic acid is an mRNA encoding a protein.

In still further embodiments of the present aspect, which may be combined with one or more of any of the previous embodiments, the complexes further comprise one or more second agents. In yet other embodiments, which may be combined with any of the preceding embodiments, the one or more second agents are selected from the group consisting of polyethylene glycol, a targeting element, and a combination thereof. In still yet another embodiment, which may be combined with any of the preceding embodiments, the complexes further comprise one or more small molecule active agents or drug substances

In yet another aspect of the present disclosure, provided herein are delivering a polyanionic compound to a cell comprising contacting the cell with the composition of the preceding aspect in any and all of its various embodiments. In certain embodiments of this aspect, the contacting is by endocytosis. In other embodiments of this aspect, which may be combined with the preceding aspect, the cell is contacted in vivo or in vitro. In certain embodiments of this aspect that include a nucleic acid encoding a polypeptide, which may be combined with either or both of the preceding embodiments, the cell expresses the polypeptide after being contacted with the composition. In still further embodiments that include a nucleic acid encoding a protein, which may be combined with one or more of the preceding embodiments, the cell expresses the protein after being contacted with the composition.

An additional aspect of this disclosure provides methods of preparing the compositions of the preceding aspect relating to compositions and any and all of its various embodiments, by contacting one or more tertiary amino lipidated and/or PEGylated cationic peptide compounds with one or more PEG compounds and one or more polyanionic compounds. In an embodiment of this aspect, the compositions are formed by contacting a solution comprising the one or more tertiary amino lipidated and/or PEGylated cationic peptide compounds and one or more PEG compounds with a solution comprising the one or more polyanionic compounds.

In still another aspect of the present disclosure, provided herein is a method of delivering a polyanionic compound to lungs of a subject comprising administering to the subject a composition of the preceding aspect, wherein the one or more cationic compounds are one or more tertiary amino lipidated and/or PEGylated cationic peptide compounds of formula (I), wherein the mass ratio of the one or more tertiary amino lipidated and/or PEGylated cationic peptide compounds of formula (I) to the polyanionic compound is between 1:1 and 10:1. In some embodiments of the present aspect, the one or more tertiary amino lipidated and/or PEGylated cationic peptide compounds of formula (I) comprise compound 8, compound 24, compound 28, compound 73, compound 79, compound 93, compound 98, or compound 112, or any combinations thereof. In some embodiments of the present aspect, the one or more cationic peptide compounds and one or more PEG compounds are present at a mass ratio of peptide compound(s)-to-PEG compound(s) between 90:10 and 99:1. In other embodiments, which may be combined with the preceding embodiment, the administration is intravenous (IV) (e.g. bolus injection or intravenous infusion), subcutaneous (SC), intramuscular (IM), intrathecal, or intratumoral injection. In other embodiments, which may be combined with any of the preceding embodiments, the polyanionic compound comprises the nucleic acid which is the mRNA encoding a polypeptide and the cell expresses the polypeptide after being contacted with the composition. In certain embodiments, which may be combined with any of the preceding embodiments, the polyanionic compound comprises the nucleic acid which is the mRNA encoding a protein and the cell expresses the protein after being contacted with the composition.

In another aspect, provided herein is a method of delivering a polyanionic compound to lungs and/or spleen of a subject comprising administering to the subject the composition of claim 1, wherein the one or more cationic compounds are one or more tertiary amino lipidated and/or PEGylated cationic peptide compounds of formula (I), wherein the mass ratio of the one or more tertiary amino lipidated and/or PEGylated cationic peptide compounds of formula (I) to the polyanionic compound is between 1:1 and 10:1. In some embodiments of the present aspect, the one or more tertiary amino lipidated and/or PEGylated cationic peptide compounds of formula (I) comprise compound 8, compound 19, compound 24, compound 28, compound 73, compound 79, compound 93, compound 98, or compound 112, or any combinations thereof. In some embodiments of the present aspect, the one or more cationic peptide compounds and one or more PEG compounds are present at a mass ratio of peptide compound(s)-to-PEG compound(s) between 90:10 and 99:1. In other embodiments, which may be combined with the preceding embodiment, the administration is intravenous (IV) (e.g. bolus injection or intravenous infusion), subcutaneous (SC), intramuscular (IM), intrathecal, or intratumoral injection. In other embodiments, which may be combined with any of the preceding embodiments, the polyanionic compound comprises the nucleic acid which is the mRNA encoding a polypeptide and the cell expresses the polypeptide after being contacted with the composition. In certain embodiments, which may be combined with any of the preceding embodiments, the polyanionic compound comprises the nucleic acid which is the mRNA encoding a protein and the cell expresses the protein after being contacted with the composition.

### DESCRIPTION OF THE FIGURES

The patent or application file contains at least one drawing executed in color. Copies of this patent or patent application publication with color drawings will be provided by the office upon request and payment of the necessary fee.

FIG. 1 shows an exemplary secondary amine phospholipidated cationic peptide “Lipitoid 1”.

FIGS. 2A-2F show the tertiary amino lipidated cationic peptoids generally. FIG. 2A shows the generalized structure for the tertiary amino lipidated peptoids. FIG. 2B shows an exemplary tertiary amino lipidated cationic peptoid. FIG. 2C shows exemplary lipid monomers. FIG. 2D shows exemplary cationic monomers. FIG. 2E shows exemplary spacer monomers and a polyethylene glycol monomer. FIG. 2F shows exemplary tertiary amino lipidated cationic peptoid compound 8.

FIGS. 3A-3E show an exemplary process for preparing tertiary amino lipidated and/or PEGylated cationic peptide compounds via sequential addition of amino acid residues in the solid-phase. FIG. 3A shows the generalized acylation reaction of a resin-bound secondary amine to acylating agent bromoacetic acid. FIG. 3B shows the reaction of the resulting acylation product with a suitable amine via nucleophilic displacement to produce the corresponding N-substituted amino acid residue. FIG. 3C and FIG. 3D show successive iterations of the acylation and nucleophilic displacement reactions to produce the desired resin-bound cationic peptide compound. FIG. 3E shows cleavage of the cationic peptide compound from the solid resin support to provide the free peptide.

FIGS. 4A and 4B show the variation in particle size stability over time and transfection efficiency in formulations of exemplary amino lipidated peptoid compound 8 formulated with Fluc mRNA alone (no PEG compound) or with DMG-PEG2000 incorporate at either 2% w/w or 5% w/w. FIG. 4A shows a graph of the number average particle size observed for each of the three formulations immediately following formulation (30 min post-formulation) and 8 hours after formulation. FIG. 4B shows a bar chart of the mean bioluminescence for HeLa cells treated with the three formulations as recorded 8 hours after treatment.

FIGS. 5A-5D show the in vivo whole body distribution of a formulation of amino lipidated peptoid compound 28 with Fluc mRNA and 2% w/w DMG-PEG2000 as a function of storage duration at 4° C. prior to injection. FIGS. 5A-5C show the whole body luminescence after 1 hour (FIG. 5A), 3 days (FIG. 5B) and 7 days (FIG. 5C) of storage. FIG. 5D shows a plot of the total photon flux observed for each of the time points.

FIGS. 6A-6E show the bioluminescence (RLU) recorded in HeLa cells 24 hours after treatment with a formulation of amino lipidated peptoid and Fluc mRNA with and without PEG incorporation (DMG-PEG2000). FIG. 6A depicts the observed bioluminescence for cells treated with formulations of amino lipidated peptoid compounds (1-18) and Fluc mRNA alone (no DMG-PEG2000). FIGS. 6B and 6C depict the observed bioluminescence for cells treated with formulations of amino lipidated peptoid compounds (1-36) and Fluc mRNA with 2% w/w DMG-PEG2000 added. FIGS. 6D and 6E depict the observed mean bioluminescence for HeLa cells treated with formulations of amino lipidated peptoid compounds 1-72 and Fluc mRNA at a peptoid:mRNA ratio of 5:1 with 2% w/w DMG-PEG2000 added.

FIGS. 7A-7C show the in vivo whole body distribution and organ-specific distribution of formulations of amino lipidated peptoids with Fluc mRNA and 2% w/w DMG-PEG2000 as represented by bioluminescence intensity and total photon flux. FIG. 7A shows the whole body distribution of the formulations in Balb/c mice. FIG. 7B shows the corresponding organ-specific biodistribution (in lungs, kidney, liver, spleen, inguinal lymph nodes and mesenteric lymph nodes) of the formulations. FIG. 7C depicts a bar chart quantifying the organ-specific biodistribution of the formulations as a function of total photon flux.

FIGS. 8A-8C depict total photon flux observed in Balb/c mice treated with subcutaneous and intramuscular injections of formulations of amino lipidated peptoids with 2% w/w DMG-PEG2000 and Fluc mRNA. FIG. 8A shows a plot of total photon flux for formulations of amino lipidated peptoid compound 28 with 2% w/w DMG-PEG 2000 and Fluc mRNA at different peptoid:mRNA ratios (1:1, 2:1, 5:1, 7.5:1 and 10:1) administered to the Balb/c mice either subcutaneously (SC) or intramuscularly (IM). FIGS. 8B and 8C shows a plot of the total photon fluxes observed in mice treated with formulations of amino lipidated peptoid compounds 1-72 with 2% DMG-PEG 2000 and Fluc mRNA at a peptoid:mRNA ratio of 5:1.

FIGS. 9A-9C depict normalized total photon fluxes (cps) in an in vitro evaluation of transfection efficiency of Fluc mRNA for various cell types (HeLa in FIG. 9A; HepG2 in FIG. 9B; and JAWSII in FIG. 9C) as provided in different formulations of amino lipidated peptoids (Compounds 24 and 79) in combination with commercially available PEG lipids (DMG-PEG2000; 18:0 PEG1000PE; 14:0 PEG2000 PE; 14:0 PEG5000 PE), linear amino PEGylated peptoids (Compounds 48, 56, 64, and 72), and branched amino PEGylated peptoids (Compounds 106, 107, 108, and 109).

### DETAILED DESCRIPTION

The following description sets forth exemplary methods, parameters and the like. It should be recognized, however, that such description is not intended as a limitation on the scope of the present disclosure but is instead provided as a description of exemplary embodiments.

The present disclosure relates to complexes and compositions of cationic compounds, such as tertiary amino lipidated and/or PEGylated cationic peptide compounds, with low mass percentages of PEGylated compounds, such as PEGylated lipids, for the delivery of nucleic acids and other polyanionic cargoes. More specifically, the present disclosure relates to tertiary amino lipidated and/or PEGylated peptoids, and complexes and compositions thereof having low concentrations of PEGylated lipids for nucleic acid delivery. The present disclosure also provides methods for preparing complexes and compositions comprising one or more of the tertiary amino lipidated and/or PEGylated cationic peptides and a low mass percentage of PEGylated compounds with polyanionic compounds, such as nucleic acids, and methods for delivering polyanionic compounds to cells. In one aspect, provided herein are complexes and compositions comprising one or more polyanionic compounds, one or more PEG compounds, and one or more cationic compounds.

It should be recognized that the one or more cationic compounds may form a complex with the one or more polyanionic compounds within the composition due to their favorable charge-charge interactions. It should be further recognized that the one or more PEGylated compounds may further combine with the charge complex of the cationic compound-polyanionic compound to form a complex. The compositions provided herein should be understood to contain all the components present in the complexes comprising the polyanionic compounds with the cationic compounds and PEGylated compounds, in addition to any further components which do not form a complex with the cationic compound and polyanionic compound.

Surprisingly, it was observed that the addition of PEGylated compound(s) (such as PEG lipid DMG-PEG2000) to complexes and compositions comprising cationic delivery vehicle compounds and polyanionic compounds could provide improved particle stability over time without requiring the further incorporation of structural lipids and phospholipids used as co-components for standard lipid nanoparticle formulations. Moreover, it was also observed that the long-term stabilization effect could be achieved by incorporating only a small mass percentage of PEGylated compounds relative to the overall complex, thus providing stable, readily optimizable formulations able to support delivery of a much larger weight fraction of active polyanionic material in the complex to cells. The complexes comprising the small mass percentage of PEGylated compounds and, thus also, the compositions comprising the complexes, were shown to have comparable transfection efficiencies for their polyanionic cargo as complexes of the cationic compounds and the polyanionic compounds alone but with significantly improved formulation stability.

“Alkenyl” as used herein refers to an unsaturated linear or branched univalent hydrocarbon chain or combination thereof, having at least one site of olefinic unsaturation (i.e., having at least one moiety of the formula C═C) and having the number of carbon atoms designated (i.e., C₂-C₁₀ means two to ten carbon atoms). The alkenyl group may be in “cis” or “trans” configurations, or alternatively in “E” or “Z” configurations. Particular alkenyl groups are those having 2 to 20 carbon atoms (a “C₂-C₂₀ alkenyl”), having 2 to 8 carbon atoms (a “C₂-C₈ alkenyl”), having 2 to 6 carbon atoms (a “C₂-C₆ alkenyl”), or having 2 to 4 carbon atoms (a “C2-C4 alkenyl”). Examples of alkenyl include, but are not limited to, groups such as ethenyl (or vinyl), prop-1-enyl, prop-2-enyl (or allyl), 2-methylprop-1-enyl, but-1-enyl, but-2-enyl, but-3-enyl, buta-1,3-dienyl, 2-methylbuta-1,3-dienyl, homologs and isomers thereof, and the like.

The term “alkyl” refers to and includes saturated linear and branched univalent hydrocarbon structures and combination thereof, having the number of carbon atoms designated (i.e., C₁-C₁₀ means one to ten carbons). Particular alkyl groups are those having 1 to 20 carbon atoms (a “C₁-C₂₀ alkyl”). More particular alkyl groups are those having 1 to 8 carbon atoms (a “C₁-C₈ alkyl”), 3 to 8 carbon atoms (a “C₃-C₈ alkyl”), 1 to 6 carbon atoms (a “C₁-C₆ alkyl”), 1 to 5 carbon atoms (a “C₁-C₅ alkyl”), or 1 to 4 carbon atoms (a “C₁-C₄ alkyl”). Examples of alkyl include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like.

“Alkylene” as used herein refers to the same residues as alkyl, but having bivalency. Particular alkylene groups are those having 1 to 6 carbon atoms (a “C1-C6 alkylene”), 1 to 5 carbon atoms (a “C1-C5 alkylene”), 1 to 4 carbon atoms (a “C1-C4 alkylene”) or 1 to 3 carbon atoms (a “C₁-C₃ alkylene”). Examples of alkylene include, but are not limited to, groups such as methylene (—CH₂—), ethylene (—CH₂CH₂—), propylene (—CH₂CH₂CH₂—), butylene (—CH₂CH₂CH₂CH₂—), and the like.

“Alkynyl” as used herein refers to an unsaturated linear or branched univalent hydrocarbon chain or combination thereof, having at least one site of acetylenic unsaturation (i.e., having at least one moiety of the formula C≡C) and having the number of carbon atoms designated (i.e., C₂-C₁₀ means two to ten carbon atoms). Particular alkynyl groups are those having 2 to 20 carbon atoms (a “C₂-C₂₀ alkynyl”), having 2 to 8 carbon atoms (a “C₂-C₈ alkynyl”), having 2 to 6 carbon atoms (a “C₂-C₆ alkynyl”), or having 2 to 4 carbon atoms (a “C₂-C₄ alkynyl”). Examples of alkynyl include, but are not limited to, groups such as ethynyl (or acetylenyl), prop-1-ynyl, prop-2-ynyl (or propargyl), but-1-ynyl, but-2-ynyl, but-3-ynyl, homologs and isomers thereof, and the like.

The term “aryl” refers to and includes polyunsaturated aromatic hydrocarbon groups. Aryl may contain additional fused rings (e.g., from 1 to 3 rings), including additionally fused aryl, heteroaryl, cycloalkyl, and/or heterocyclyl rings. In one variation, the aryl group contains from 6 to 14 annular carbon atoms. Examples of aryl groups include, but are not limited to, phenyl, naphthyl, biphenyl, and the like.

“Carbonyl” refers to the group C═O.

“Complex” as used herein includes any chemical association between two or more molecules, which may be mediated by ionic interactions, hydrogen bonding, van der Waals interactions, metal-ligand coordination, other chemical forces, and combinations of one or more of the foregoing. The complexes may form higher order structures including, for example, polyplexes, coacervate complexes, nanocomplexes, nanoparticles, and microparticles.

The term “cycloalkyl” refers to and includes cyclic univalent hydrocarbon structures, which may be fully saturated, mono- or polyunsaturated, but which are non-aromatic, having the number of carbon atoms designated (e.g., C₁-C₁₀ means one to ten carbons). Cycloalkyl can consist of one ring, such as cyclohexyl, or multiple rings, such as adamantly, but excludes aryl groups. A cycloalkyl comprising more than one ring may be fused, spiro or bridged, or combinations thereof. A preferred cycloalkyl is a cyclic hydrocarbon having from 3 to 13 annular carbon atoms. A more preferred cycloalkyl is a cyclic hydrocarbon having from 3 to 8 annular carbon atoms (a “C₃-C₈ cycloalkyl”). Examples of cycloalkyl include, but are not limited to, cyclopropyl, cyclobutyl, cyclopentyl, cyclohexyl, 1-cyclohexenyl, 3-cyclohexenyl, cycloheptyl, norbornyl, and the like.

“Halo” or “halogen” refers to elements of the Group 17 series having atomic number 9 to 85. Preferred halo groups include fluoro, chloro, bromo and iodo. Where a residue is substituted with more than one halogen, it may be referred to by using a prefix corresponding to the number of halogen moieties attached, e.g., dihaloaryl, dihaloalkyl, trihaloaryl etc. refer to aryl and alkyl substituted with two (“di”) or three (“tri”) halo groups, which may be but are not necessarily the same halo; thus 4-chloro-3-fluorophenyl is within the scope of dihaloaryl. An alkyl group in which each hydrogen is replaced with a halo group is referred to as a “perhaloalkyl.” A preferred perhaloalkyl group is trifluoroalkyl (—CF₃). Similarly, “perhaloalkoxy” refers to an alkoxy group in which a halogen takes the place of each H in the hydrocarbon making up the alkyl moiety of the alkoxy group. An example of a perhaloalkoxy group is trifluoromethoxy (—OCF₃).

The term “heteroaryl” refers to and includes unsaturated aromatic cyclic groups having from 1 to 10 annular carbon atoms and at least one annular heteroatom, including but not limited to heteroatoms such as nitrogen, oxygen and sulfur, wherein the nitrogen and sulfur atoms are optionally oxidized, and the nitrogen atom(s) are optionally quaternized. A heteroaryl group can be attached to the remainder of the molecule at an annular carbon or at an annular heteroatom. Heteroaryl may contain additional fused rings (e.g., from 1 to 3 rings), including additionally fused aryl, heteroaryl, cycloalkyl, and/or heterocyclyl rings. Examples of heteroaryl groups include, but are not limited to, pyridyl, pyrimidyl, thiophenyl, furanyl, thiazolyl, and the like.

The term “heterocycle” or “heterocyclyl” refers to a saturated or an unsaturated non-aromatic group having from 1 to 10 annular carbon atoms and from 1 to 4 annular heteroatoms, such as nitrogen, sulfur or oxygen, and the like, wherein the nitrogen and sulfur atoms are optionally oxidized, and the nitrogen atom(s) are optionally quaternized. A heterocyclyl group may have a single ring or multiple condensed rings, but excludes heteroaryl groups. A heterocycle comprising more than one ring may be fused, spiro or bridged, or any combination thereof. In fused ring systems, one or more of the fused rings can be aryl or heteroaryl. Examples of heterocyclyl groups include, but are not limited to, tetrahydropyranyl, dihydropyranyl, piperidinyl, piperazinyl, pyrrolidinyl, thiazolinyl, thiazolidinyl, tetrahydrofuranyl, tetrahydrothiophenyl, 2,3-dihydrobenzo[b]thiophen-2-yl, 4-amino-2-oxopyrimidin-1(2H)-yl, and the like.

“Oxo” refers to the moiety ═O.

“Thiocarbonyl” refers to the group C═S.

“Optionally substituted” unless otherwise specified means that a group may be unsubstituted or substituted by one or more (e.g., 1, 2, 3, 4 or 5) of the substituents listed for that group in which the substituents may be the same of different. In one embodiment, an optionally substituted group has one substituent. In another embodiment, an optionally substituted group has two substituents. In another embodiment, an optionally substituted group has three substituents. In another embodiment, an optionally substituted group has four substituents. In some embodiments, an optionally substituted group has 1 to 2, 2 to 5, 3 to 5, 2 to 3, 2 to 4, 3 to 4, 1 to 3, 1 to 4 or 1 to 5 substituents.

The term “substituted” refers to the replacement of one or more hydrogen atoms of a moiety with a monovalent or divalent radical. “Optionally substituted” indicates that the moiety may be substituted or unsubstituted. Suitable substituent groups include, for example, hydroxyl, nitro, amino (e.g., —NH₂ or dialkyl amino), imino, cyano, halo (such as F, Cl, Br, I), haloalkyl (such as —CCl₃ or —CF₃), thio, sulfonyl, thioamido, amidino, imidino, oxo, oxamidino, methoxamidino, imidino, guanidino, sulfonamido, carboxyl, formyl, alkyl, alkoxy, alkoxy-alkyl, alkylcarbonyl, alkylcarbonyloxy (—OCOR), aminocarbonyl, arylcarbonyl, aralkylcarbonyl, carbonylamino, heteroarylcarbonyl, heteroaralkyl-carbonyl, alkylthio, aminoalkyl, cyanoalkyl, carbamoyl (—NHCOOR— or —OCONHR—), urea (—NHCONHR—), aryl and the like, where R is any suitable group, e.g., alkyl or alkylene. In some embodiments, the optionally substituted moiety is optionally substituted only with select radicals, as described. In some embodiments, the above groups (e.g., alkyl groups) are optionally substituted with, for example, alkyl (e.g., methyl or ethyl), haloalkyl (e.g., —CCl₃, —CH₂CHCl₂ or —CF₃), cycloalkyl (e.g., —C₃H5, —C₄H7, —C₅H₉), amino (e.g., —NH₂ or dialkyl amino), alkoxy (e.g., methoxy), heterocycloalkyl (e.g., as morpholine, piperazine, piperidine, azetidine), hydroxyl, and/or heteroaryl (e.g., oxazolyl). In some embodiments, a substituent group is itself optionally substituted. In some embodiments, a substituent group is not itself substituted. The group substituted onto the substitution group can be, for example, carboxyl, halo, nitro, amino, cyano, hydroxyl, alkyl, alkenyl, alkynyl, alkoxy, aminocarbonyl, —SR, thioamido, —SO₃H, —SO₂R or cycloalkyl, where R is any suitable group, e.g., a hydrogen or alkyl.

### Cationic Compounds as Delivery Vehicles

As detailed above, the complexes and compositions provided herein comprise one or more polyanionic compounds, one or more PEGylated compounds, and one or more cationic compounds. By virtue of their positive charge, the cationic compounds in the complexes and composition can counterbalance the negative charge on the polyanionic cargoes and form charge neutral complexes (also referred to as cationic compound-polyanionic compound complexes), thus facilitating the transport of the anionic material through the lipophilic cell membrane into the target cell.

The cationic compounds as described herein have a net zero charge or a net positive charge. In some embodiments wherein the complex or composition comprises one or more cationic compounds, the one or more cationic compounds independently have a net zero charge or a net positive charge. In certain embodiments, the one or more cationic compounds independently have a net positive charge of at least +1. In other embodiments, the one or more cationic compounds independently have a net positive charge of at least +2. In yet other embodiments, one or more cationic compounds independently have a net positive charge of less than or equal to +4 or less than or equal to +3. It should be recognized that the net charge present on the one or more cationic compounds may vary depending upon environmental conditions. For example, in some embodiments, the one or more cationic compounds independently have a stable, net positive charge at physiologically relevant pH ranges. For example, physiological pH is at least about 5.5 and typically at least about 6.0. More typically, physiological pH is at least about 6.5. Usually, physiological pH is less than about 8.5 and typically less than about 8.0. More typically, physiological pH is less than about 7.5.

It should be recognized that there are a variety of cationic compounds that may serve as delivery vehicles for nucleic acids and are suitable for the complexes and compositions described herein. The cationic compounds in the complexes and compositions described herein may include discrete small molecules as well as larger polymeric constructs. Suitable cationic compounds may include but are not limited to cationic lipids, cationic lipid-peptide conjugates (e.g., lipitoids), cationic peptides, cationic polymers, and lipid-like (lipophilic) cationic compounds. In some embodiments, the complexes and compositions of the present disclosure comprise one or more cationic compounds, wherein the one or more cationic compounds are selected from the group consisting of cationic lipids, cationic lipid-peptide conjugates (e.g., lipitoids), cationic peptides, cationic polymers and lipid-like (lipophilic) cationic compounds, and any combinations thereof.

Tertiary Amino Lipidated and/or PEGylated Cationic Peptide Compounds

In some embodiments, the complexes and compositions of the present disclosure comprise one or more tertiary amino lipidated and/or PEGylated cationic peptide compounds. The tertiary amino lipidated and/or PEGylated cationic peptide compounds are peptide chains comprising N-substituted amino acid residues. The tertiary amino lipidated and/or PEGylated cationic peptide compounds of the present disclosure comprise an oligopeptide backbone, wherein the oligopeptide backbone comprises repeating subunits of N-substituted cationic amino acid residues optionally interleaved with N-substituted neutral (“spacer”) and/or lipid amino acid residues. The oligopeptide backbone is further capped at the N- and/or C-terminus by amino acid residues that are N-substituted with lipid moieties (“N-lipidated”) and/or N-substituted with oligoethylene glycol and/or polyethylene glycol (“N-PEGylated”).

In one aspect, provided herein is a tertiary amino lipidated and/or PEGylated cationic peptide compound of formula (I) or a salt thereof:

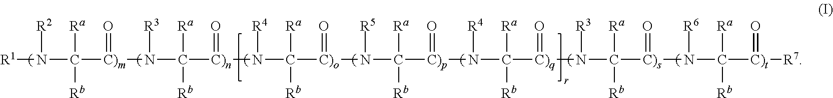

In some embodiments, the tertiary amino lipidated and/or PEGylated cationic peptide compound may be characterized by a total number of amino acid residues present in the peptide compound, wherein each amino acid residue is represented by the general structure —(NR—CR^{a}R^{b}—C(O))—. In some embodiments, the total number of amino acid residues is between 2 and 40 amino acid residues, between 2 and 30 amino acid residues, between 3 and 25 amino acid residues, between 5 and 20 amino acid residues, or between 7 and 15 amino acid residues. In certain embodiments, the tertiary amino lipidated and/or PEGylated cationic peptide compound comprises between 5 and 20 amino acid residues. In certain embodiments, the total number of amino acid residues is the sum of m, n, s, t, and [r×(o+p+q)].

In other embodiments, the tertiary amino lipidated and/or PEGylated cationic peptide compound has a net zero charge or a net positive charge. In certain embodiments wherein the tertiary amino lipidated and/or PEGylated cationic peptide compound is a tertiary amino lipidated cationic peptide compound, the tertiary amino lipidated cationic peptide compound has a net positive charge of at least +1. In other embodiments wherein the tertiary amino lipidated and/or PEGylated cationic peptide compound is a tertiary amino PEGylated cationic peptide compound or a tertiary amino lipidated and PEGylated cationic peptide compound, the cationic peptide compound has a net zero charge (i.e., is charge neutral) or a net positive charge. In some embodiments wherein the tertiary amino lipidated and/or PEGylated cationic peptide compound is a tertiary amino PEGylated cationic peptide compound or a tertiary amino lipidated and PEGylated cationic peptide compound, the cationic peptide compound has a net positive charge of +1. In certain embodiments, the tertiary amino lipidated and/or PEGylated cationic peptide compound has a net positive charge of (r×p)+.

In certain embodiments, the amino acid residues of the cationic peptide compounds described herein are N-substituted variants of naturally-occurring amino acids or non-naturally occurring amino acids, wherein the carbon side chains are represented by R^{a} and R^{b}. The amino acid residues may be present in either D- or L-configurations. In addition, it should be recognized that the substitution of the N-position of amino acid residues may restrict free rotation of the amide bond. As such, the tertiary amino lipidated and/or PEGylated cationic peptide compounds of the present disclosure may also exist in various rotational isomeric conformations (rotamers).

In some embodiments, each R^{a} and each R^{b} are independently a side chain moiety found on naturally- or non-naturally-occurring amino acids. As used herein, the term “naturally-occurring amino acid” refers to Gly, Ala, Val, Leu, Ile, Ser, Thr, Cys, Met, Asp, Glu, Asn, Gln, Lys, Arg, Phe, Tyr, His, or Trp. The term “non-naturally-occurring amino acid” refers to amino acids typically not found in nature, including, for example, D-isomers of naturally-occurring amino acids, 2-aminoadipic acid, 2-aminobutyric acid, norvaline, norleucine, and ornithine. In certain embodiments, each R^{a} and R^{b} are independently —H, —CH₃, or

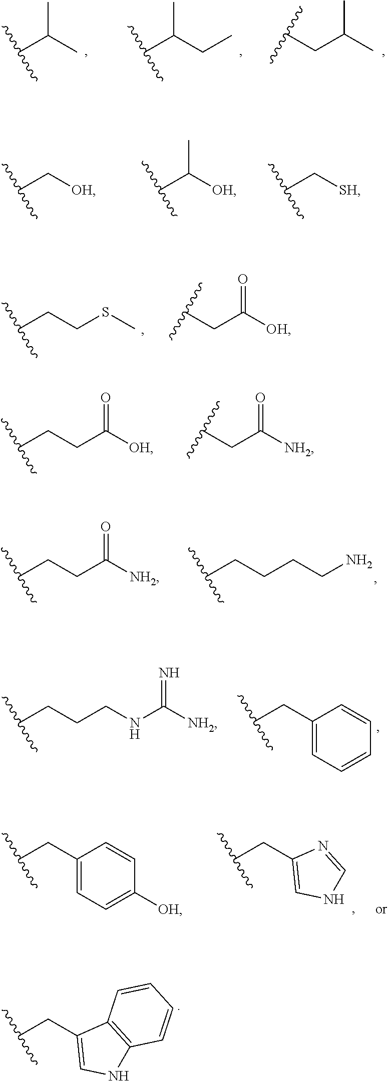

In other embodiments, each R^{a} and each R^{b} are independently —H or C₁-C₄-alkyl. In certain embodiments, each R^{a} and each R^{b} are independently —H or CH₃. In other embodiments, R^{a} and R^{b} are —H. In embodiments wherein R^{a} and R^{b} are —H, the tertiary amino lipidated and/or PEGylated cationic peptide compounds may also be referred to as N-lipidated and/or PEGylated polyglycine compounds or N-lipidated and/or PEGylated peptoid compounds. In certain embodiments, each R^{a} and each R^{b} is independently —H, C1-C₄-alkyl, or a side chain moiety found on a naturally- or non-naturally-occurring amino acid.

### Oligopeptide Backbone, or Repeating Subunits, and Structural Motifs

As described herein, the tertiary amino lipidated and/or PEGylated cationic peptide compounds may be useful for complexation with polyanionic compounds, such as nucleic acids, and for the delivery of such polyanionic compounds into cells. The tertiary amino lipidated and/or PEGylated cationic peptide compounds of the present disclosure comprise an oligopeptide backbone of repeating subunits of N-substituted cationic amino acid residues optionally interleaved with N-substituted neutral spacer amino acid residues and/or N-lipidated amino acid residues, as shown in the fragment of formula (I) below:

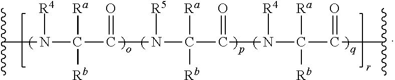

The cationic amino acid residues in the repeating subunits of the oligopeptide backbone confer positive charge to the compounds of the present disclosure, which allows for favorable electrostatic interaction with and charge neutralization of polyanionic species like nucleic acids. The interleaving of neutral or lipidated amino acid residues in between the cationic residues allows for greater control over the spatial distribution of positive charge throughout the tertiary amino lipidated and/or PEGylated cationic peptide compounds, which enables improved complexation of the cationic peptide compounds to polyanionic species having specific lengths, charge distributions and/or conformations.

In the oligopeptide backbone, r represents the number of repeating subunits of cationic and neutral and/or lipidated amino acid residues in the tertiary amino lipidated and/or PEGylated cationic peptide compound. In some embodiments, r is an integer from 1 to 25. In certain embodiments, r is an integer from 1 to 20. In other embodiments, r is an integer from 1 to 15. In some embodiments, r is an integer from 1 to 5. In certain embodiments, r is an integer from 2 to 4.

It should be recognized that each subunit r may or may not strictly be identical to the other subunits within the entire tertiary amino lipidated and/or PEGylated cationic peptide compound. For example, within a tertiary amino lipidated and/or PEGylated cationic peptide compound, each subunit r may comprise cationic amino acid residues, and optionally also neutral and/or lipidated amino acid residues, that are independently chosen with respect to the cationic and neutral and/or lipidated amino acid residues present in other subunits.

### Cationic Moieties

Each repeating subunit of the tertiary amino lipidated and/or PEGylated cationic peptide compounds of the present disclosure comprises at least one cationic amino acid residue. The cationic amino acid residues provide the positive charge that enables the peptide compounds described herein to form electrostatic complexes with nucleic acids or other polyanionic compounds, by interaction with negative charges on the nucleic acids or polyanionic compounds. Complexation of nucleic acids partially or fully shields the negative charge of the nucleic acid and facilitates transport through the lipid membrane of cells and into the cell interior.

Within each subunit r, p represents the number of cationic amino acid residues present in that subunit. In some embodiments, each p is independently an integer 1 or 2. In certain embodiments, p is 1.

Each cationic amino acid residue comprises a cationic moiety R⁵ at the N-position. It should be recognized that each cationic moiety R⁵ may not only be independently selected within the repeating subunit of the cationic peptide compounds but also throughout the oligopeptide backbone.

A cationic moiety as described herein may be a substituent that has a stable, net positive charge at physiologically relevant pH ranges. For example, physiological pH is at least about 5.5 and typically at least about 6.0. More typically, physiological pH is at least about 6.5. Usually, physiological pH is less than about 8.5 and typically less than about 8.0. More typically, physiological pH is less than about 7.5. A cationic moiety may be characterized, for example, by a threshold pKₐ value for a functional group present in the moiety that is sufficient to produce a positive charge at physiological pH. In certain embodiments, the cationic moiety has a pKₐ value of at least 7.5. In other embodiments, the cationic moiety has a pKₐ between physiological pH and a more acidic pH, for example pH 4.5-5.5. In additional embodiments, substituents with multiple independent pKₐ values are used.

It should be recognized that the tertiary amino lipidated and/or PEGylated cationic peptide compounds described herein may comprise multiple cationic moieties along the oligopeptide backbone in close proximity to one another. When multiple cationic moieties are present along the oligopeptide backbone, the protonated or deprotonated state of certain cationic moieties may influence the pKₐ values of other cationic moieties in close proximity. By this mechanism, the pKₐ of a particular cationic moiety in the cationic peptide compounds described herein may be altered with respect to its pKₐ value as measured in isolation. For example, protonation of one amine in the oligopeptide backbone, having a pKₐ˜8, may lower the pKₐ value of a nearby cationic moiety to pKₐ˜5-6 from a normal value of ˜7.5.

The capability of the tertiary amino lipidated and/or PEGylated cationic peptide compounds of the present disclosure to accommodate multiple cationic moieties at varying proximity to one another enables these cationic peptide compounds, and any complexes thereof, to respond with high sensitivity to variations in physiological pH. The sensitivity of these compounds and their complexes to changes in physiological pH may be important for facilitating delivery and release of nucleic acids into the cytosol following endosomal transport (endosome compartment pH˜4.5 to 5.5).

Cationic, or positively charged, moieties may include, for example, nitrogen-based substituents, such as those containing the following functional groups: amino, guanidino, hydrazido, and amidino. These functional groups can be either aromatic, saturated cyclic, or aliphatic. In some embodiments, each R⁵ is independently aminoalkyl, alkylaminoalkyl, aminoalkylaminoakyl, guanidinoalkyl, N-heterocyclylalkyl, or N-heteroaryl. In other embodiments, each R⁵ is independently

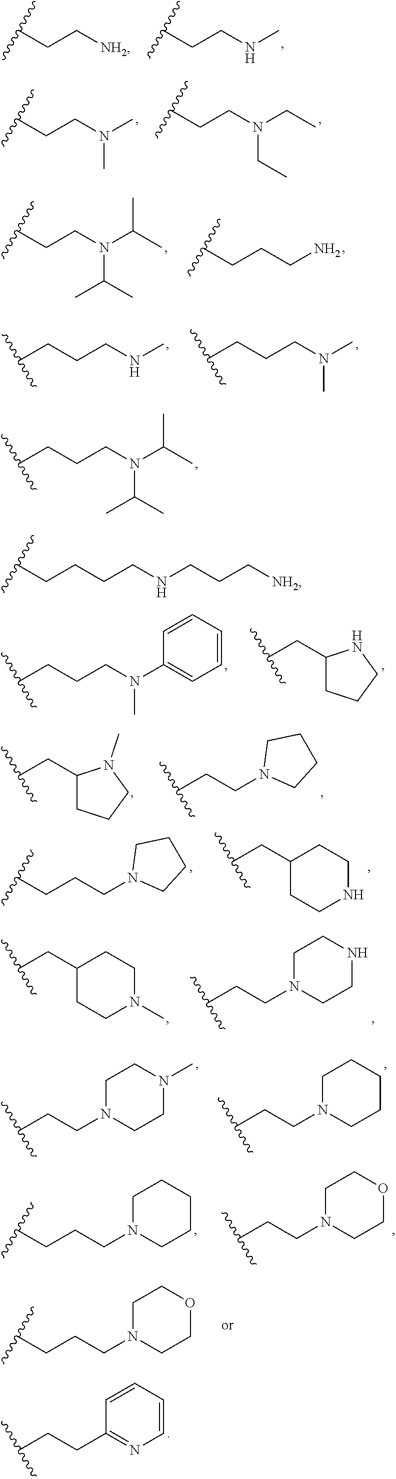

In certain embodiments, each R⁵ is independently selected from the group consisting of:

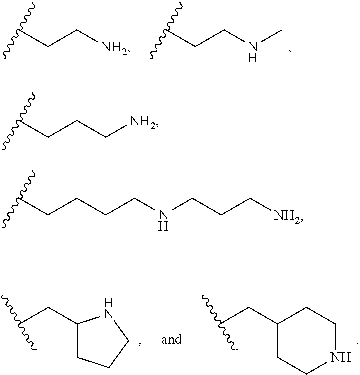

In still other embodiments, each R⁵ is

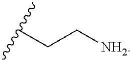 [SMILES: *CCN]

In certain embodiments, each R⁵ is a —(C₁-C₄alkylene)-NR^{5a}R^{5b}, wherein R^{5a} and R^{5b} are independently H, C₁-C₃ alkyl, or C₆-C₁₀ aryl, or R^{5a} and R^{5b} are taken together with the nitrogen atom to which they are attached to form a 4-7 membered N-heterocycloalkyl ring, wherein the N-heterocycloalkyl ring may optionally include one additional heteroatom selected from the group consisting of N and O In certain embodiments, each R⁵ is

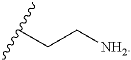 [SMILES: *CCN]

In some embodiments, each R⁵ is independently

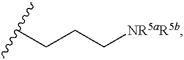

wherein R^{5a} and R^{5b} are independently H, C₁-C₃ alkyl, or C₆-C₁₀ aryl, or R^{5a} and R^{5b} are taken together with the nitrogen atom to which they are attached to form a 4-7 membered N-heterocycloalkyl ring, wherein the N-heterocycloalkyl ring may optionally include one additional heteroatom selected from the group consisting of N and O. In certain embodiments, each R⁵ is independently selected from the group consisting of

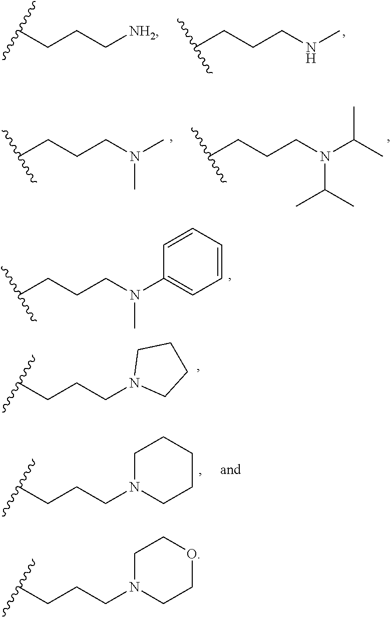

In still certain other embodiments, each R⁵ is

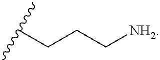 [SMILES: *CCCN]

In yet further embodiments wherein a cationic residue is the terminal residue of the entire peptide compound, additional cationic moieties R⁵ which are which are not compatible with the synthesis or deprotection conditions (such as acid-labile linkers) or for which a suitable protecting group strategy is not available (e.g. polyamines) may be utilized. For example, in some embodiments, the cationic moiety R⁵ of the terminal cationic residue is a polyamine. In some embodiments wherein R⁵ of the terminal residue is a polyamine, the polyamine is

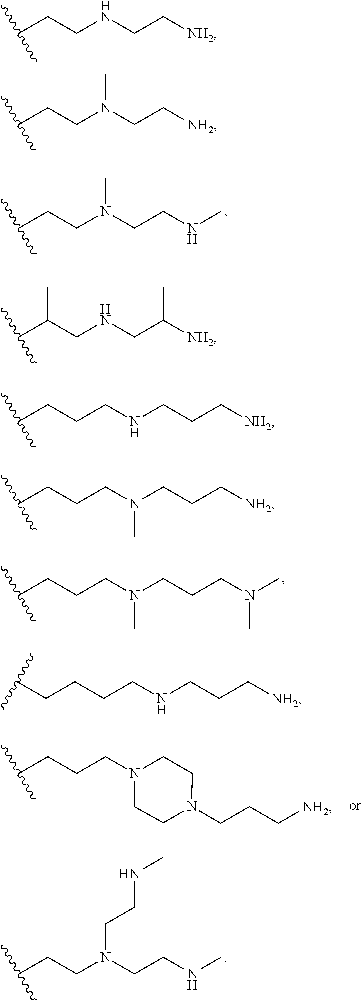

In certain embodiments, the polyamine is selected from the group consisting of

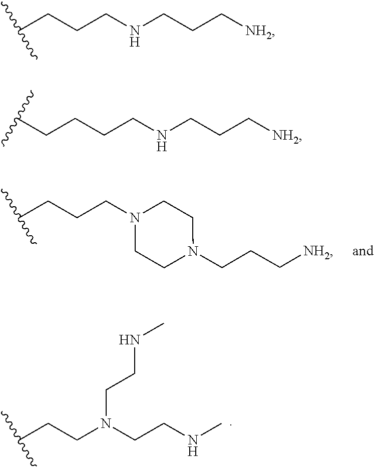 [SMILES: *CCCNCCCN ; *CCCCNCCCN ; *CCCN1CCN(CCCN)CC1 ; "and" ; *CCN(CCNC)CCNC]

In other embodiments, the cationic moiety R⁵ of the terminal cationic residue is a hydroxyalkyl, a hydroxyether, an alkoxyalkyl, or a hydroxylheteroalkyl. In certain embodiments, the cationic moiety R⁵ of the terminal cationic residue is

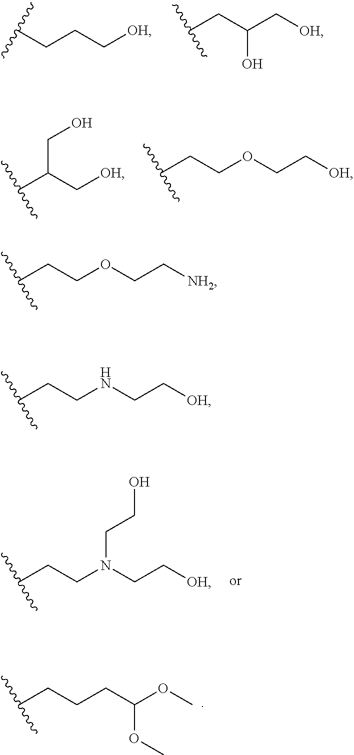

In still further embodiments, the cationic moiety R⁵ of the terminal cationic residue is

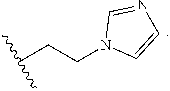 [SMILES: *CCn1ccnc1]

It should be further recognized that an unsubstituted nitrogen atom in the peptide chain, that is, wherein R⁵ is hydrogen, may also serve as an ionizable cationic moiety under physiological conditions. In some embodiments, the cationic moiety R⁵ is a hydrogen atom.

### Neutral Spacer Moieties

Within the oligopeptide backbone of tertiary amino lipidated and/or PEGylated cationic peptide compounds, the cationic amino acid residues may be optionally interleaved with neutral spacer amino acid residues, possessing a neutral spacer moiety at the N-position. The neutral amino acid residues may be useful to modulate the spatial distribution of the positive charge in the tertiary amino lipidated and/or PEGylated cationic peptide compounds for improved electrostatic interactions with the polyanionic compounds, including polynucleotides, to be complexed with the cationic peptide compounds.

In each repeating subunit of the tertiary amino lipidated and/or PEGylated cationic peptide compounds, a neutral amino acid residue may present on either N- or C-terminal end of the cationic amino acid residue as one or more R⁴ groups. In some embodiments wherein a subunit r comprises a neutral spacer moiety R⁴, the corresponding o and/or q for each neutral spacer moiety present represent the respective numbers of neutral spacer residues bonded to the N- and C-terminal ends of the cationic amino acid residue(s) within the subunit r. In some embodiments, each o is independently an integer 0, 1, 2, 3 or 4. In other embodiments, each q is independently an integer 0, 1, 2, 3 or 4.

Each neutral spacer amino acid residue comprises a neutral spacer moiety R⁴ at the N-position. As with the cationic moieties described herein, it should be recognized that each neutral spacer moiety R⁴ is independently selected within the repeating subunit of the cationic peptide compounds as well as amongst the repeating subunits r of the oligopeptide backbone.

It should also be recognized that neutral spacer moieties may include any substituents that are neutral, or have zero net charge, at physiologically relevant pH ranges. In some embodiments, each neutral moiety R⁴ is independently a C₁-C₅ straight chain alkyl, a C₁-C₄-alkyl substituted by cycloalkyl, heterocyclylalkyl, alkylaryl, arylalkyl, alkylheteroaryl, heteroarylalkyl, alkoxy, alkoxyalkyl, or hydroxyalkyl, wherein each cycloalkyl, heterocyclylalkyl, alkylaryl, arylalkyl, alkylheteroaryl, heteroarylalkyl, alkoxy, alkoxyalkyl, or hydroxyalkyl is optionally substituted with one or more substituents —OH, halo, or alkoxy. In still some embodiments, each neutral spacer moiety R⁴ is independently selected from the group consisting of: —CH₃,

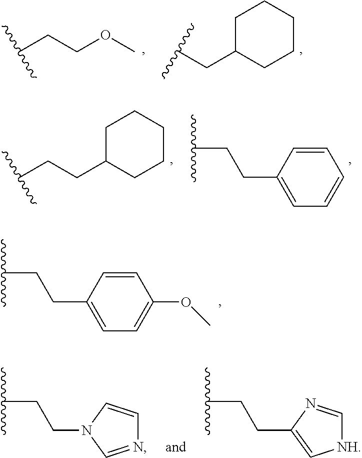

In certain embodiments, each neutral spacer moiety R⁴ is

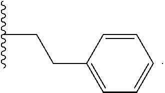 [SMILES: *CCc1ccccc1]

In still other embodiments, the neutral spacer moiety may include substituents that may become anionic (i.e., have a negative charge) at physiologically relevant pH ranges. For example, in some embodiments, the spacer moiety R⁴ is

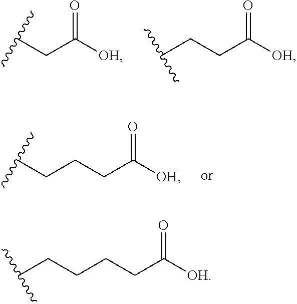 [SMILES: *CC(=O)O ; *CCC(=O)O ; *CCCC(=O)O ; "or" ; *CCCCC(=O)O]

### Lipid Moieties

In addition to the optional interleaving of neutral amino acid residues with cationic amino acid residues within the oligopeptide backbone of tertiary amino lipidated and/or PEGylated cationic peptide compounds, N-lipidated amino acid residues, possessing a lipid moiety at the N-position, may also optionally be interleaved with the cationic (and optional neutral spacer) amino acid residues. In some embodiments wherein the tertiary amino lipidated and/or PEGylated cationic peptide compound comprises N-lipidated amino acid residues, the tertiary amino lipidated and/or PEGylated cationic peptide compound is N-lipidated. Similar to the neutral amino acid residues, the N-lipidated amino acid residues within the oligopeptide backbone may be useful to modulate the spatial distribution of the positive charge in the tertiary amino lipidated and/or PEGylated cationic peptide compounds as well as augment their lipophilicity for improved encapsulation of polyanionic materials and endocellular delivery. The spacing of lipids along the peptoid backbone may also influence the lipid fluidity/crystallinity which is known to influence cellular uptake and endosomal release.

As with the neutral spacer residues, in each repeating subunit of the tertiary amino lipidated and/or PEGylated cationic peptide compounds, the N-lipidated amino acid residue may present on either N- or C-terminal end or both ends of the cationic amino acid residue as one or more R⁴ groups. In some embodiments wherein a subunit r comprises a lipid moiety R⁴, the corresponding o and/or q for each lipid moiety present may also represent the respective numbers of lipidated residues bonded to the N- and C-terminal ends of the cationic amino acid residue(s) within the subunit r. In some embodiments, each o is independently an integer 0, 1, 2, 3 or 4. In other embodiments, each q is independently an integer 0, 1, 2, 3 or 4.

Each N-lipidated amino acid residue comprises a lipid moiety R⁴ at the N-position. As with the cationic and neutral moieties described herein, it should be recognized that each lipid moiety R⁴ is independently selected within the repeating subunit of the cationic peptide compounds as well as amongst the repeating subunits r of the oligopeptide backbone.

Suitable lipid moieties may include, for example, optionally substituted branched or straight chain aliphatic moieties, or optionally substituted moieties derived from natural lipid compounds, including fatty acids, sterols, and isoprenoids.

In some embodiments, the lipid moieties may include branched or straight chain aliphatic moieties having from about 6 to about 50 carbon atoms or from about 10 to about 50 carbon atoms, optionally comprising one or more heteroatoms, and optionally comprising one or more double or triple bonds (i.e., saturated or mono- or poly-unsaturated). In certain embodiments, the lipid moieties may include optionally substituted aliphatic, straight chain or branched moieties, each hydrophobic tail independently having from about 8 to about 30 carbon atoms or from about 6 to about 30 carbon atoms. In certain embodiments, the lipid moieties may include, for example, aliphatic carbon chains derived from fatty acids and fatty alcohols. In some embodiments, each lipid moiety R⁴ is independently C₈-C₂₄-alkyl or C₈-C₂₄-alkenyl, wherein the C₈-C₂₄-alkenyl is optionally mono- or poly-unsaturated. In some embodiments, each lipid moiety R⁴ is a C₆-C₁₈ alkyl or C₆-C₁₈ alkenyl. In certain embodiments, each lipid moiety R⁴ is a C₆-C₁₈ alkyl or C₆-C₁₈ alkenyl, wherein the C₆-C₁₈ alkenyl is mono-unsaturated. In certain other embodiments, each lipid moiety R⁴ is a C₆-C₁₇ alkyl or C₆-C₁₇ alkenyl. In certain embodiments, each lipid moiety R⁴ is C₈-C₁₂ alkyl. In still other embodiments, each lipid moiety R⁴ is a C₁₀-alkyl, such as n-decyl. In some embodiments, each R⁴ is independently selected from the group consisting of 2-ethylhex-1-yl, caprylyl, caproyl, oleyl, stearyl, linoleyl, myristyl, and lauryl. In other embodiments, each lipid moiety R⁴ is independently selected from the group consisting of 2-ethylhex-1-yl, caproyl, oleyl, stearyl, linoleyl, myristyl, and lauryl. In certain embodiments, each lipid moiety R⁴ is independently selected from the group consisting of 2-ethylhex-1-yl, caproyl, oleyl, myristyl, and lauryl.

In yet other embodiments which may be combined with any of the preceding embodiments, each lipid moiety R⁴ is independently,

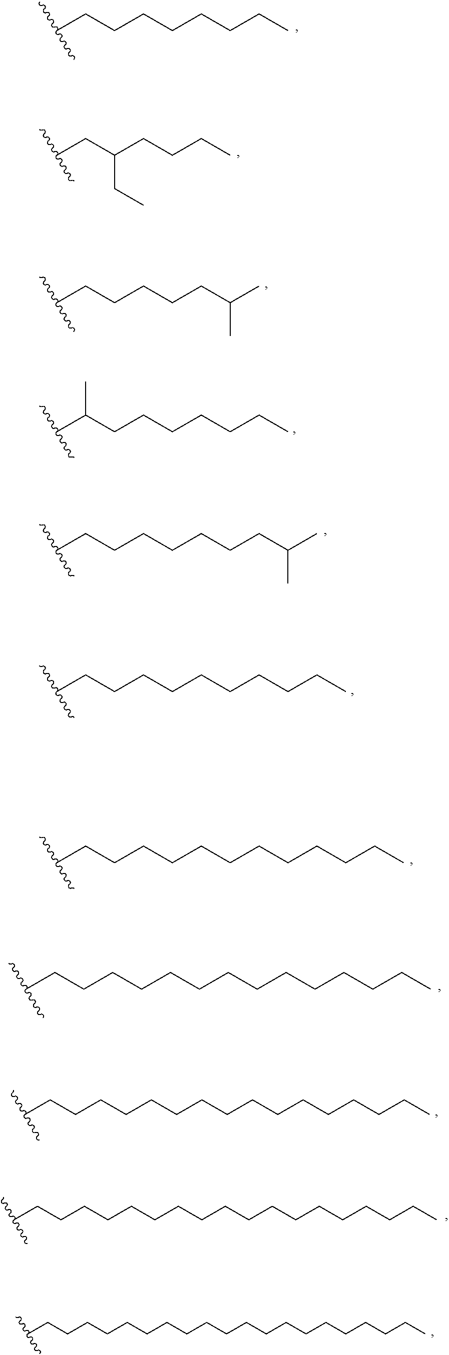

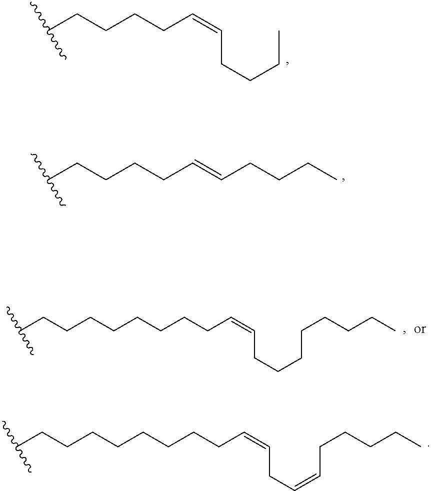 [SMILES: *CCCC/C=C\CCCC ; *CCCC/C=C/CCCC ; *CCCCCCCC/C=C\CCCCCCCC ; *CCCCCCCC/C=C\C/C=C\CCCCC]

In still yet other embodiments which may be combined with any of the preceding embodiments, each lipid moiety R⁴ is independently a lipid of the formula

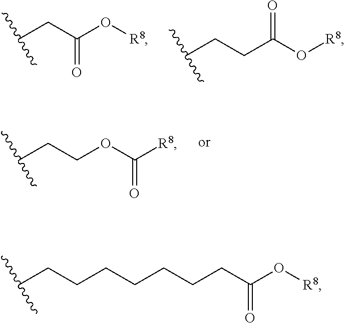

wherein R⁸ is a branched or straight chain aliphatic moieties having from about 6 to about 50 carbon atoms or from about 10 to about 50 carbon atoms, optionally comprising one or more heteroatoms, and optionally comprising one or more double or triple bonds. In certain embodiments, each lipid moiety R⁴ is independently

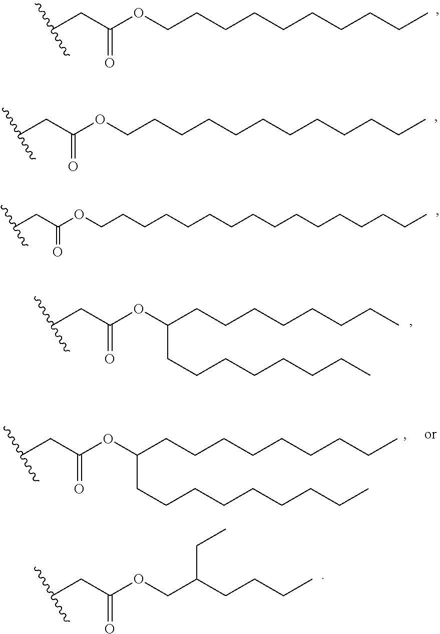

Natural lipid moieties employed in the practice of the present invention can be derived from, for example, phospholipids, glycerides (such as di- or tri-glycerides), glycosylglycerides, sphingolipids, ceramides, and saturated and unsaturated sterols, isoprenoids, and other like natural lipids.

Other suitable lipid moieties may include lipophilic carbocyclic or aromatic groups such as optionally substituted aryl, cycloalkyl, cycloalkylalkyl, or arylalkyl moieties, including for example naphthalenyl or ethylbenzyl, or lipids comprising ester functional groups including, for example, sterol esters and wax esters. In still other embodiments, the lipid moiety R⁴ is

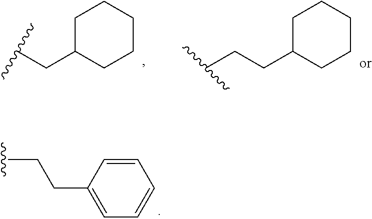

### Structural Motifs

In some embodiments, the tertiary amino lipidated and/or PEGylated cationic peptide compounds of the present disclosure may comprise particular sequences or arrangements of the cationic, neutral spacer, and lipidated amino acid residues with respect to one another, which can be described similar to the various classifications of linear copolymers (random, block, alternating, periodic, stereoblock, etc.).

For example, in some embodiments wherein the tertiary amino lipidated and/or PEGylated cationic compounds comprise generic moieties R^{cation}, R^{neutral}, and R^{lipid}, the amino acid residues may be arranged in random sequences, in alternating sequences or block sequences. It should be understood that the representations of R^{cation} and R^{neutral} are equivalent to amino acid residues comprising a cationic moiety R⁵ and neutral moiety R⁴, respectively. The generic moiety R^{lipid} may be understood to include both amino acid residues having a lipid moiety R⁴ and lipid moiety R³, depending upon the position of the N-lipidated residue in the tertiary amino lipidated and/or PEGylated cationic peptide compounds.

One example of an alternating sequence of the cationic and neutral spacer amino acid residues may be represented by R^{cation}—R^{neutral}—R^{cation}—R^{neutral}, or R^{neutral}—R^{cation}—R^{neutral}—R^{cation}. Another example of an alternating sequence of cationic and lipidated amino acid residues may be represented by R^{cation}—R^{lipid}—R^{cation}—R^{lipid} or R^{neutral}—R^{cation}—R^{neutral}—R^{cationic} Some examples of a block sequence may be represented by R^{cation}—R^{cation}—R^{cation}—R^{neutral}—R^{neutral}—R^{neutral}, R^{neutral}—R^{neutral}—R^{neutral}—R^{cation}—R^{cation}—R^{cation}, R^{cation}—R^{cation}—R^{cation}—R^{lipid}—R^{lipid}—R^{lipid}, or R^{lipid}—R^{lipid}—R^{lipid}—R^{cation}—R^{cation}—R^{cation}. In still some embodiments, the cationic moieties, neutral spacer moieties and lipidated moieties may be arranged in block sequences and alternating sequences for different repeating subunit segments within the overall oligopeptide compound. For example, in some embodiments, there may be repeating motifs of larger oligomer units, such as R^{cation}—R^{neutral}—R^{neutral}—R^{cation}—R^{neutral}—R^{neutral}, within the cationic peptide compound. In other embodiments, the cationic peptide compound may comprise a block of N-lipidated residues combined with a block of alternating cationic and neutral residues.

As described herein, the tertiary amino lipidated and/or PEGylated cationic peptides of the present disclosure comprise at least one cationic amino acid residue having a cationic moiety R⁵. In some embodiments, the backbone of the tertiary amino lipidated and/or PEGylated cationic peptide compound comprises a “cationic domain” or “cationic block”. The cationic domain may be understood broadly as a segment of sequential amino acid residues within the oligopeptide chain having a plurality of cationic moieties R⁵, e.g., at least two cationic residues. In some embodiments, the tertiary amino lipidated and/or PEGylated cationic peptide compound comprises a cationic domain wherein the cationic domain comprises at least two, at least three or at least four cationic amino acid residues.

The cationic domain may include, for example, a block of amino acid residues having a plurality of R⁵ cationic moieties in a contiguous, linear sequence (e.g., R⁵R⁵R⁵ or R^{cation}R^{cation}R^{cation}). In some embodiments, the tertiary amino lipidated and/or PEGylated cationic peptide compound comprises a “cationic domain”, wherein the tertiary amino lipidated and/or PEGylated cationic peptide compound comprises at least two, at least three, or at least four contiguous amino acid residues having R⁵ cationic moieties.

In other embodiments, the “cationic domain” may include a block of adjacent amino acid residues wherein the plurality of cationic residues feature additional non-cationic (i.e., neutral or lipidated) residues spaced in between two cationic residues, such that none of the cationic residues within the block and directly bonded to another cationic residue. For example, in some embodiments, the tertiary amino lipidated and/or PEGylated cationic peptide compound comprises a “cationic domain”, wherein the cationic domain comprises at least two cationic amino acid residues having a cationic moiety R⁵, and wherein each of the at least two cationic amino acid residues within the cationic domain are separated by at least one or at least two amino acid residues having a neutral spacer or lipid moiety R⁴. In some embodiments, the non-cationic residues are interleaved at regularly spaced intervals of uniform length between two cationic residues within the cationic domain. In some embodiments wherein the cationic domain comprises non-cationic residues interleaved between cationic residues, the cationic domain may be described as a domain comprising repeating (dimer, trimer, tetramer, etc.) subunits. These subunits may include but are not limited to —R^{cation}R^{neutral}—, R^{cation}R^{neutral}R^{neutral}—, R^{cation}R^{lipid}—, —R^{cation}R^{lipid}R^{lipid}—, —R^{lipid}R^{cation}R^{lipid}—, —R^{lipid}R^{lipid}R^{cation}—, —R^{cation}R^{neutral}R^{lipid}—, or —R^{cation}R^{lipid}R^{neutral}. In other embodiments, the non-cationic residues are interspersed between two cationic residues within the cationic domain with varying numbers of non-cationic residues between each pair of cationic residues. In certain embodiments wherein the cationic domain comprises non-cationic residues interspersed between cationic residues, each pair of cationic residues are independently separated by at least one non-cationic residue. In some embodiments, at least one of the one or more tertiary amino lipidated and/or PEGylated cationic peptide compounds comprises at least one trimer subunit —R^{cation}—R^{neutral}—R^{neutral}. In other embodiments, at least one of the one or more tertiary amino lipidated and/or PEGylated cationic peptide compounds comprises at least one trimer subunit —R^{cation}—R^{neutral}—R^{lipid}.

In some embodiments wherein the tertiary amino lipidated and/or PEGylated cationic peptide compound comprises a cationic domain, each R⁵ is

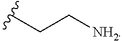

In other embodiments wherein the tertiary amino lipidated and/or PEGylated cationic peptide comprises a cationic domain, each cationic moiety R⁵ is

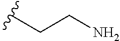

and each neutral spacer moiety is

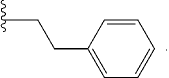

In certain embodiments wherein the tertiary amino lipidated and/or PEGylated cationic peptide comprises a cationic domain comprising one or more dimer or trimer subunits, each cationic moiety R⁵ is

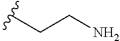

and each neutral spacer moiety is

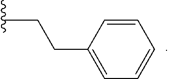

In still other embodiments, the tertiary amino lipidated and/or PEGylated cationic peptide comprises a cationic domain comprising one or more trimer subunits —R^{cation}R^{neutral}R^{neutral}, wherein each cationic moiety R⁵ is

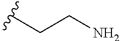

and each neutral spacer moiety is

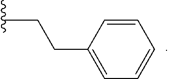

In yet other embodiments, the tertiary amino lipidated and/or PEGylated cationic peptide comprises a at least one trimer subunit —R^{cation}R^{neutral}R^{lipid}—, wherein each cationic moiety R⁵ is

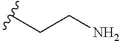

and each neutral spacer moiety is

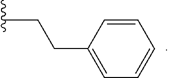

As described herein, the tertiary amino lipidated and/or PEGylated cationic peptide compounds of the present disclosure may comprise one or more different cationic moieties (R^{cation1}, R^{cation2}, R^{cation3}, etc.), one or more different neutral spacer moieties (R^{neutral1}, R^{neutral2}, R^{neutral3}, etc.), and/or one or more different lipid moieties (R^{lipid1}, R^{lipid2}, R^{lipid3}, etc.). It should be recognized that the above examples of sequences or arrangements of the generic cationic and neutral spacer amino acid residues are not intended to be limiting to the peptide compounds of the present disclosure. The cationic, neutral spacer, and lipid moieties, which may be present in random, alternating or block sequences as generally described above, may also be present in particular sequences of individual cationic moieties, neutral moieties, and lipid moieties within a larger block or alternating structural motif. For example, some exemplary arrangements of residues may include but are not limited to the following: R^{cation1}R^{neutral1}R^{neutral1}R^{lipid1}R^{cation1}R^{neutral1}R^{neutral1}R^{lipid1}R^{cation1}R^{neutral1}R^{neutral1}, R^{cation1}R^{neutral1}R^{neutral1}R^{cation1}R^{neutral1}R^{neutral1}R^{cation1}R^{neutral1}R^{neutral1}R^{ipid1}R^{lipid1}, R^{lipid1}R^{cation1}R^{neutral1}R^{lipid1}R^{cation1}R^{neutral1}—R^{lipid2}R^{cation2}R^{neutral2}R^{lipid2}R^{cation2}R^{neutral2}, R^{lipid1}R^{lipid2}R^{lipid3}R^{lipid4}R^{neutral1}R^{neutral2}R^{cation1}R^{neutral1}R^{neutral2}R^{lipid1}R^{lipid2}R^{lipid3}R^{lipid4}, or R^{lipid1}R^{lipid2}R^{lipid1}R^{lipid2}R^{cation1}R^{neutral1}R^{cation1}R^{neutral1}R^{lipid1}R^{lipid2}R^{lipid1}R^{lipid2}.

### Side Chain Functionalization and Peptide Conjugate Compounds

As described above, the oligopeptide backbone principally comprises amino acid residues having a side chain moiety found on naturally- or non-naturally-occurring amino acids (R^{a} and R^{b}) with cationic, neutral spacer, or lipid moieties at the N-positions (R⁴ and R⁵).

In one aspect, the R^{a}, R^{b}, R⁴, and R⁵ groups of the tertiary amino lipidated and/or PEGylated cationic peptide compounds of the present disclosure may further comprise reactive linker groups that can be used to covalently bond to a therapeutic agent and/or targeting element, such as a small molecule or antibody. These additional functionalities may also comprise cationic groups which are not compatible with the synthesis or deprotection conditions (such as acid-labile linkers) or for which a suitable protecting group strategy is not available (ex. polyamines). The reactive group(s) can, for example, be appended to an existing side chain R^{a} and/or R^{b} or to the cationic moiety at R⁵, neutral spacer or lipid moiety at R⁴ at the N-position through chemical methods known in the art. Alternatively, if the R^{a}, R^{b}, R⁴, or R⁵ group with the desired reactive moiety is commercially obtainable as the corresponding free amine, the corresponding free amine can be incorporated during the general synthesis of peptide chain (e.g., during submonomer synthesis).

In some embodiments, the tertiary amino lipidated and/or PEGylated cationic peptide comprises one or more amino acid residues comprising a reactive group or linker group. Suitable reactive groups may include but are not limited to esters, amides, isocyanates, thiols or “click” chemistry compatible moieties (e.g., azido, alkynyl).

In another aspect, the present disclosure further provides as a cationic compound a tertiary amino lipidated and/or PEGylated cationic peptide compound, wherein the peptide compound is covalently bound, or conjugated, to a therapeutic agent and/or targeting element. In certain embodiments, the therapeutic agent and/or targeting element is a small molecule, a peptide sequence, an antibody or an antibody fragment, an aptamer, a mono- or oligo-saccharide (e.g., galactose), or a glycan.

Linker groups or linkages that are sensitive to or labile under certain physiological conditions environments may be useful to enable targeted delivery to certain organs or cells followed by selective release of the therapeutic agent and/or polyanionic material in(to) the cell. Moreover, labile linkages may also promote elimination and clearance of the tertiary amino lipidated and/or PEGylated cationic compounds from the cell (or organ, or whole body) following delivery of polyanionic material. In certain embodiments, the linker group is selected such that the covalent bond between the tertiary amino lipidated and/or PEGylated cationic peptide compound and the therapeutic agent or targeting element is hydrolytically labile, chemically labile, pH labile, photolabile, thermally labile, or enzymatically cleavable.

### Terminal (Capping) Residues and Protecting End Groups

As described herein, the tertiary amino lipidated and/or PEGylated cationic peptide compounds of the present disclosure comprise an oligopeptide backbone that is capped at its N- and/or C-terminus by amino acid residues that are N-substituted with lipid moieties (“N-lipidated”) and/or N-substituted with oligoethylene glycol and/or polyethylene glycol (“N-PEGylated”). The N-lipidation of the cationic peptide compounds confers favorable lipophilicity to the compounds as well as any complexes formed between the compounds and polyanionic species. N-PEGylation provides greater control over particle formation and aggregation in vivo. The terminal amino and carboxylic acid moieties of the N- and C-terminus may be further capped with protecting end groups.

As described herein, the tertiary amino lipidated and/or PEGylated cationic peptide compounds may comprise amino acid residues that are N-lipidated and/or N-PEGylated. The cationic peptide compounds provided herein comprise at least one amino acid residue that is N-lipidated or N-PEGylated. In some embodiments, the tertiary amino lipidated and/or PEGylated cationic peptide compound is a tertiary amino lipidated cationic peptide compound. In other embodiments, the tertiary amino lipidated and/or PEGylated cationic peptide compound is a tertiary amino PEGylated cationic peptide compound. In still yet other embodiments, the tertiary amino lipidated and/or PEGylated cationic peptide compounds of the present disclosure comprise both N-lipidated and N-PEGylated amino acid residues. In some embodiments, the tertiary amino lipidated and/or PEGylated cationic peptide compound is a tertiary amino lipidated and PEGylated cationic peptide compound.

### Lipid Moieties

As described above, the tertiary amino lipidated and/or PEGylated cationic peptide compounds may comprise amino acid residues at the N-terminus and/or the C-terminus, wherein the amino acid residues are N-substituted with a lipid moiety, or “N-lipidated”. The incorporation of N-lipidated amino acid residues at the N- and/or C-terminus of the cationic peptide compounds described herein increase the lipophilicity of the compounds. The increased lipophilicity of the cationic peptide compounds enhances their affinity for hydrophobic environments, such as the lipid bilayer of the cell membrane, thus increasing the propensity of the tertiary amino lipidated and/or PEGylated cationic peptide compounds, and any complexes thereof with polyanionic compounds, to be transported into the cell.

In some embodiments, the tertiary amino lipidated and/or PEGylated cationic peptide compound is N-lipidated. In some embodiments, the tertiary amine lipidated and/or PEGylated cationic peptide compounds of the present disclosure comprise N-lipidated amino acid residues at the N-terminus. In other embodiments, the tertiary amine lipidated and/or PEGylated cationic peptide compounds of the present disclosure comprise N-lipidated amino acid residues at the C-terminus. In certain embodiments, the tertiary amine lipidated and/or PEGylated cationic peptide compounds of the present disclosure comprise N-lipidated amino acid residues at the N- and C-termini.

In some embodiments, the number of N-lipidated amino acid residues at the N-terminus of the cationic peptide compounds described herein is represented by n. In other embodiments, the number of N-lipidated amino acid residues at the N-terminus of the cationic peptide compounds described herein is represented by s.

In some embodiments, n is an integer from 0 to 8. In certain embodiments, n is an integer from 0 to 5. In other embodiments, n is an integer 0, 1, 2, 3, 4, 5, 6, or 7. In some embodiments, n is an integer less than or equal to 4. In yet other embodiments, n is an integer 1, 2, 3, or 4. In certain embodiments, n is 4. In some embodiments, s is an integer from 0 to 8. In certain embodiments, s is an integer from 0 to 5. In other embodiments, s is an integer 0, 1, 2, 3, 4, 5, 6, or 7. In some embodiments, s is an integer less than or equal to 4. In still other embodiments, s is an integer 1, 2, 3, or 4. In certain embodiments, s is 4. In some embodiments wherein the tertiary amino lipidated and/or PEGylated cationic peptide compound is N-lipidated, at least one of n or s is nonzero. In some embodiments, n is nonzero. In certain embodiments, n is nonzero and s is 0. In other embodiments, s is nonzero. In certain embodiments, s is nonzero and n is 0. In certain embodiments, both n and s are nonzero.

In some embodiments, the sum of n and s is an integer from 1 to 8, from 2 to 7, or from 4 to 6. In other embodiments, the sum of n and s is at least 2, at least 3, or at least 4.

In some embodiments, the tertiary amino lipidated and/or PEGylated cationic peptide compound comprises a block of N-lipidated residues, or “N-lipid block” wherein the tertiary amino lipidated and/or PEGylated cationic peptide comprises at least two, at least three, or at least four N-lipidated residues adjacent to one another (e.g., R^{1ipid}R^{lipid}R^{lipid}) In some embodiments, the tertiary amino lipidated and/or PEGylated cationic peptide compound comprises a block of N-lipidated residues, wherein n is at least 2, at least 3, or at least 4. In other embodiments, the tertiary amino lipidated and/or PEGylated cationic peptide compound comprises a block of N-lipidated residues wherein s is at least 2, at least 3, or at least 4.

The N-lipidated amino acid residues in the tertiary amino lipidated and/or PEGylated cationic peptides of the present disclosure are N-substituted with lipid moieties R³. Lipid moieties of the present disclosure may include hydrophobic or lipophilic moieties that are neutral (i.e., having no charge or a net charge of zero). The lipid moieties of the tertiary amino lipidated and/or PEGylated cationic compounds described herein may be either naturally or synthetically derived. Each R³ is independently a lipid moiety, which may be the same or different. In some embodiments, each R³ is the same. In other embodiments, each R³ is different. In still yet other embodiments wherein the sum of n and s is at least 2 (that is, n+s≥2), at least two of the lipid moieties R³ are different from one another.

It should be recognized that particular sequences or arrangements of N-lipidated amino acid residues may be especially useful for improving complexation with and delivery of nucleic acids. For example, in some embodiments wherein the tertiary amino lipidated and/or PEGylated cationic compounds comprise a set of mixed N-lipidated amino acid residues having one of two different lipid moieties R^{3a} and R^{3b}, the N-lipidated amino acid residues may be arranged on either N- or C-terminus in an alternating or block sequences. One example of an alternating sequence of N-lipidated amino acid residues may be represented by R^{3a}—R^{3b}—R^{3a}—R^{3b} or R^{3b}—R^{3a}—R^{3b}—R^{3a}. An example of a block sequence may be represented by R^{3a}—R^{3a}—R^{3b}—R^{3b} or R^{3b}—R^{3b}—R^{3a}—R^{3a}. In other embodiments, the sequence of N-lipidated amino acid residues may be ordered at random. It should be recognized that the above examples of sequences or arrangements of two N-lipidated amino acid residues are not intended to be limiting. Moreover, the tertiary amino lipidated and/or PEGylated cationic peptide compounds of the present disclosure may comprise two or more different lipid moieties of R³, which may be present in random, alternating or block sequences as generally described above.

Suitable lipid moieties may include, for example, optionally substituted branched or straight chain aliphatic moieties, or optionally substituted moieties derived from natural lipid compounds, including fatty acids, sterols, and isoprenoids.

In some embodiments, the lipid moieties may include branched or straight chain aliphatic moieties having from about 6 to about 50 carbon atoms or from about 10 to about 50 carbon atoms, optionally comprising one or more heteroatoms, and optionally comprising one or more double or triple bonds (i.e., saturated or mono- or poly-unsaturated). In certain embodiments, the lipid moieties may include optionally substituted aliphatic, straight chain or branched moieties, each hydrophobic tail independently having from about 8 to about 30 carbon atoms or from about 6 to about 30 carbon atoms. In certain embodiments, the lipid moieties may include, for example, aliphatic carbon chains derived from fatty acids and fatty alcohols. In some embodiments, each R³ is independently C8-C24-alkyl or C8-C24-alkenyl, wherein the C₈-C₂₄-alkenyl is optionally mono- or poly-unsaturated. In some embodiments, each R³ is a C₆-C₁₈ alkyl or C₆-C₁₈ alkenyl. In certain embodiments, each lipid moiety R³ is a C₆-C₁₈ alkyl or C₆-C₁₈ alkenyl, wherein the C₆-C₁₈ alkenyl is mono-unsaturated. In certain other embodiments, each lipid moiety R³ is a C₆-C₁₇ alkyl or C₆-C₁₇ alkenyl. In certain embodiments, each R³ is C₈-C₁₂ alkyl. In still other embodiments, each R³ is a C₁₀-alkyl, such as n-decyl. In some embodiments, each R³ is independently selected from the group consisting of 2-ethylhex-1-yl, caprylyl, caproyl, oleyl, stearyl, linoleyl, myristyl, and lauryl. In other embodiments, each lipid moiety R³ is independently selected from the group consisting of 2-ethylhex-1-yl, caproyl, oleyl, stearyl, linoleyl, myristyl, and lauryl. In certain embodiments, each lipid moiety R³ is independently selected from the group consisting of 2-ethylhex-1-yl, caproyl, oleyl, myristyl, and lauryl. In other embodiments, each R³ is independently selected from the group consisting of oleyl, stearyl, linoleyl, myristyl, and lauryl.

In yet other embodiments which may be combined with any of the preceding embodiments, each R³ is independently

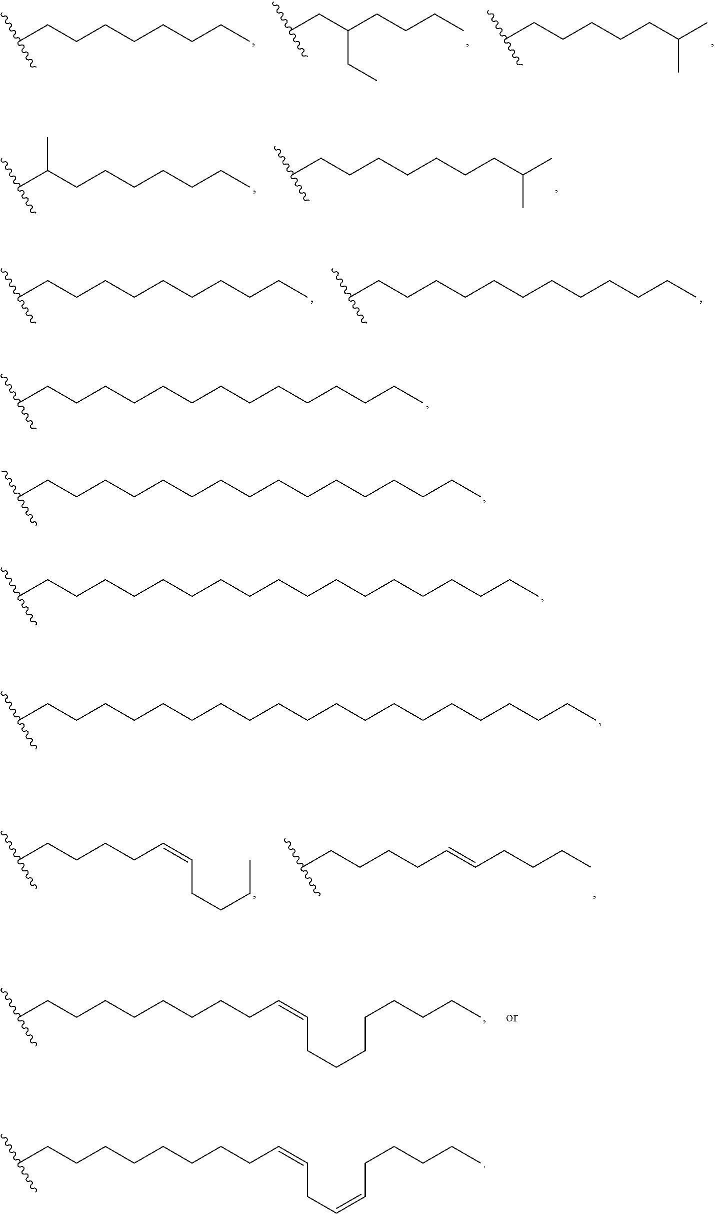

In some embodiments, each R³ is independently a branched or straight chain aliphatic moiety having from about 6 to 50 carbon atoms. In certain embodiments, each R³ is independently a straight chain aliphatic moiety. In certain other embodiments, each R³ is independently a branched aliphatic moiety. In still yet other embodiments wherein the sum of n and s is at least 2 (that is, n+s≥2), each R³ is independently a branched or straight chain aliphatic moiety and at least two of the lipid moieties R³ are different from one another (R^{3a} and R^{3b}). In some embodiments, the lipid moieties R³ are one of two different lipid moieties R^{3a} and R^{3b}. In other embodiments wherein the sum of n and s is at least 2 (that is, n+s≥2), at least one R³ is or comprises a branched aliphatic moiety and at least one R³ is or comprises a straight chain aliphatic moiety. In some embodiments, at least one lipid moiety R³ (or R^{3a} or R^{3b}) is a branched aliphatic moiety. In certain embodiments which may be combined with any of the preceding embodiments, at least one lipid moiety R³ (or R^{3a} or R^{3b}) is 2-ethylhex-1-yl.

In still yet other embodiments which may be combined with any of the preceding embodiments, each R³ is independently a lipid of the formula

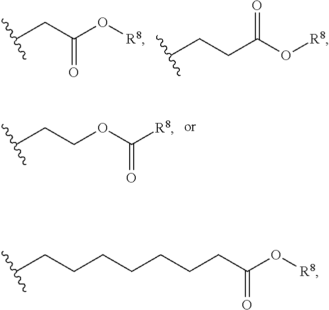

wherein R⁸ is a branched or straight chain aliphatic moieties having from about 6 to about 50 carbon atoms or from about 10 to about 50 carbon atoms, optionally comprising one or more heteroatoms, and optionally comprising one or more double or triple bonds. In certain embodiments, each R³ is independently

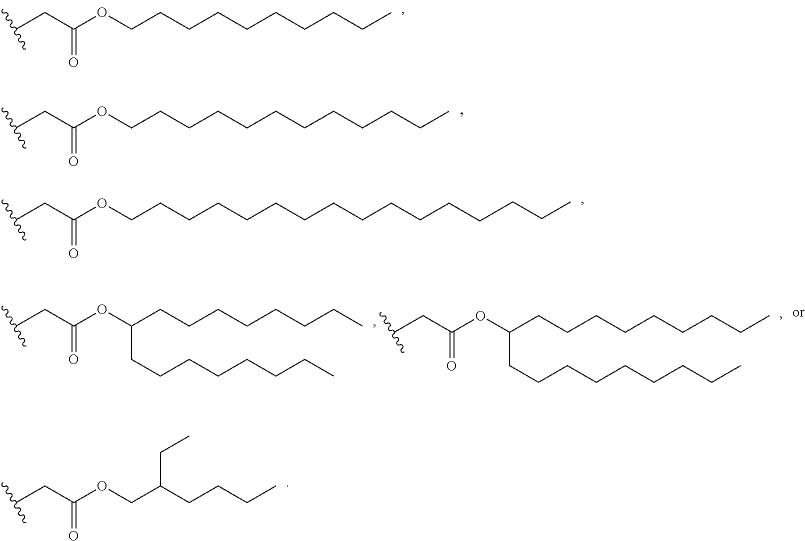

Natural lipid moieties employed in the practice of the present invention can be derived from, for example, phospholipids, glycerides (such as di- or tri-glycerides), glycosylglycerides, sphingolipids, ceramides, and saturated and unsaturated sterols, isoprenoids, and other like natural lipids.

Other suitable lipid moieties may include lipophilic carbocyclic or aromatic groups such as optionally substituted aryl, cycloalkyl, cycloalkylalkyl, or arylalkyl moieties, including for example naphthalenyl or ethylbenzyl, or lipids comprising ester functional groups including, for example, sterol esters and wax esters. In still other embodiments, the lipid moiety R⁴ is

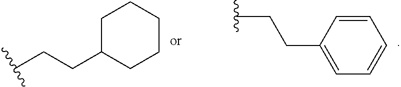

### Ethylene Glycol Moieties

The tertiary amino lipidated and/or PEGylated cationic peptides of the present disclosure may comprise capping amino acid residues at the N- and/or C-terminus which are N-substituted by oligomers or polymers of ethylene glycol, that is, N-substituted with oligoethylene glycol and/or polyethylene glycol. The incorporation of oligo- and/or polyethylene glycol moieties into the tertiary amino lipidated and/or PEGylated cationic peptide compounds described herein may facilitate particle stability of complexes formed with nucleic acids and prevent particle aggregation in vivo.

It should be recognized that the term “PEGylated” is used herein to describe cationic peptide compounds comprising terminal amino acid residues which may be N-substituted with oligoethylene glycol, or polyethylene glycol, or a combination thereof. In some embodiments, the tertiary amino lipidated and/or PEGylated cationic peptide compounds provided herein are N-PEGylated.

In some embodiments, the tertiary amino lipidated and/or PEGylated cationic peptide compound comprises N-PEGylated amino acid residues at the N-terminus, as shown in the fragment of Formula (I) below:

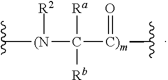

In certain embodiments wherein the tertiary amino lipidated and/or PEGylated cationic peptide compound comprises N-PEGylated amino acid residues at the N-terminus, m represents the number of N-PEGylated amino acid residues at the N-terminus, and each R² is independently an ethylene glycol moiety of the formula —CH₂CH₂O(CH₂CH₂O)ᵤR^{2a}, wherein R^{2a} is —H or C₁-C₄-alkyl. In some embodiments, R^{2a} is —H, —CH₃, or CH₂CH₃. In certain embodiments, R^{2a} is —H. In other embodiments, R^{2a} is —CH₃. In still yet other embodiments, R^{2a} is —CH₂CH₃.

In some embodiments, m is an integer from 0 to 15, an integer from 0 to 10, an integer from 0 to 3, an integer from 4 to 10, or an integer from 4 to 15. In some embodiments, each u is independently an integer from 1 to 200, an integer from 2 to 200, an integer 2 to 100, an integer from 2 to 50, an integer from 50 to 200, an integer from 50 to 100, an integer from 100 to 200, or an integer from 150 to 200.

In certain embodiments, m is an integer from 0 to 3, and each u is an integer from 20 to 200, or optionally from 30 to 50. In certain embodiments, m is an integer from 0 to 3, and u is an integer from 40 to 45. In still yet other embodiments, m is 1, and u is an integer from 40 to 45. In other embodiments, m is an integer from 4 to 10, and each u is an integer from 2 to 10. In certain embodiments, m is an integer from 4 to 10, and u is an integer from 2 to 5. In still yet other embodiments, m is an integer from 7 to 10, and u is 3. In other embodiments, m is an integer from 4 to 15, and each u is an integer from 2 to 10. In certain embodiments, m is an integer from 4 to 15, and u is an integer from 2 to 5. In some embodiments, m is an integer from 10 to 15, and u is an integer from 1 to 10. In certain embodiment, m is an integer from 10 to 15, and u is an integer from 1 to 5. In other embodiments, m is an integer from 10 to 15, and u is an integer from 2 to 10. In certain embodiments, m is an integer from 10 to 15, and u is an integer from 2 to 5.

In some embodiments, the tertiary amino lipidated and/or PEGylated cationic peptide compound comprises N-PEGylated amino acid residues at the C-terminus, as shown in the fragment of Formula (I) below:

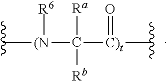

In certain embodiments wherein the tertiary amino lipidated and/or PEGylated cationic peptide compound comprises N-PEGylated amino acid residues at the C-terminus, t represents the number of N-PEGylated amino acid residues at the N-terminus, and each R⁶ is independently an ethylene glycol moiety of the formula —CH₂CH₂O(CH₂CH₂O)^{v}R^{6a}, wherein R^{6a} is —H or C₁-C₄-alkyl. In some embodiments, R^{6a} is —H, —CH₃, or CH₂CH₃. In certain embodiments, R^{6a} is —H. In other embodiments, R^{6a} is —CH₃. In still yet other embodiments, R^{6a} is —CH₂CH₃.

In some embodiments, t is an integer from 0 to 15, an integer from 0 to 10, an integer from 0 to 3, an integer from 4 to 10, or an integer from 4 to 15. In some embodiments, each v is independently an integer from 1 to 200, an integer from 2 to 200, an integer 2 to 100, an integer from 2 to 50, an integer from 50 to 200, an integer from 50 to 100, an integer from 100 to 200, or an integer from 150 to 200.

In some embodiments, t is an integer from 0 to 3, and each v is an integer from 30 to 50. In certain embodiments, t is an integer from 0 to 3, and v is an integer from 40 to 45. In still yet other embodiments, t is 1, and v is an integer from 40 to 45. In other embodiments, t is an integer from 4 to 10, and each v is an integer from 2 to 10. In certain embodiments, t is an integer from 4 to 10, and v is an integer from 2 to 5. In still yet other embodiments, t is an integer from 7 to 10, and v is 3. In other embodiments, t is an integer from 4 to 15, and each v is an integer from 2 to 10. In certain embodiments, t is an integer from 4 to 15, and v is an integer from 2 to 5. In some embodiments, t is an integer from 10 to 15, and v is an integer from 1 to 10. In certain embodiment, t is an integer from 10 to 15, and v is an integer from 1 to 5. In other embodiments, t is an integer from 10 to 15, and v is an integer from 2 to 10. In certain embodiments, t is an integer from 10 to 15, and v is an integer from 2 to 5.

In some embodiments wherein the tertiary amino lipidated and/or PEGylated cationic peptide compound is N-PEGylated, at least one of m or t is nonzero. In some embodiments, m is nonzero. In other embodiments, t is nonzero. In certain embodiments, both m and t are nonzero.

N-Lipidated and/or N-PEGylated

As described herein, the tertiary amino lipidated and/or PEGylated cationic peptide compounds of the present disclosure may comprise amino acid residues that are N-lipidated and/or N-PEGylated. The cationic peptide compounds provided herein comprise at least one amino acid residue that is N-lipidated or N-PEGylated. In some embodiments of the tertiary amino lipidated and/or PEGylated cationic peptide compound, at least one of m, n, s, or t is nonzero.

In some embodiments, the tertiary amino lipidated and/or PEGylated cationic peptide compound is a tertiary amino lipidated cationic peptide compound wherein at least one of n and s is nonzero. In certain embodiments, both n and s are nonzero. In still certain embodiments, the tertiary amino lipidated and/or PEGylated cationic peptide compound of formula (I) is a tertiary amino lipidated cationic peptide compound of formula (Ia):

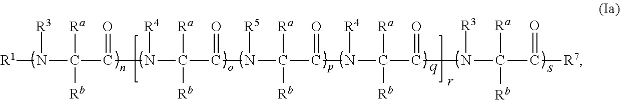

wherein at least one of n and s is nonzero, and wherein R¹, R³, R⁴, R⁵, R⁷, R^{a}, R^{b}, o, p, q, and r are as defined for formula (I). In certain embodiments of the compound of formula (Ia), both n and s are nonzero.

In other embodiments, the tertiary amino lipidated and/or PEGylated cationic peptide compound is a tertiary amino PEGylated cationic peptide compound wherein at least one of m and t is nonzero. In certain embodiments, both m and t are nonzero. In certain embodiments, both m and t are nonzero. In other embodiments, the tertiary amino lipidated and/or PEGylated cationic peptide compound of formula (I) is a tertiary amino PEGylated cationic peptide compound of formula (Ib):

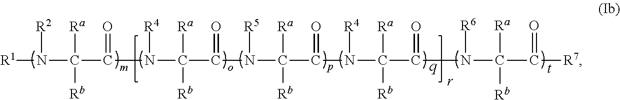

wherein at least one of m and t is nonzero, and wherein R¹, R², R⁴, R⁵, R⁶, R⁷, R^{a}, R^{b}, o, p, q, and r are as defined for formula (I). In certain embodiments of the compound of formula (Ib), both m and t are nonzero.

In still yet other embodiments, the tertiary amino lipidated and/or PEGylated cationic peptide compounds of the present disclosure comprise both N-lipidated and N-PEGylated amino acid residues. In some embodiments, the tertiary amino lipidated and/or PEGylated cationic peptide compound is a tertiary amino lipidated and PEGylated cationic peptide compound. In certain embodiments wherein the tertiary amino lipidated and/or PEGylated cationic peptide compound is a tertiary amino lipidated and PEGylated cationic peptide compound, at least one of m and t is nonzero and at least one of n and s is nonzero.

In still further embodiments, the tertiary amino lipidated and/or PEGylated cationic peptide compound of formula (I) is a tertiary amino lipidated and PEGylated cationic peptide compound of formula (Ic):

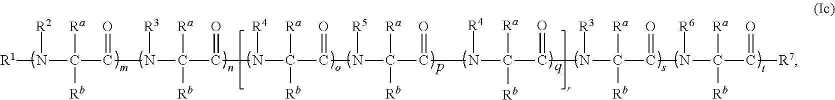

wherein at least one of m and t is nonzero, and at least one of n and s is nonzero, and wherein R¹, R², R³, R⁴, R⁵, R⁶, R⁷, R^{a}, R^{b}, o, p, q, and r are as defined for formula (I).

In other embodiments, the tertiary amino lipidated and/or PEGylated cationic peptide compounds of the present disclosure may also comprise N-substituted amino acid residues having other neutral shielding polymers or moieties at the N- or C-terminus that provide a similar shielding effect as N-PEGylated residues. These may include such examples as hydroxylalkyls, hyaluronic acid, polysaccharides, polyphosphates and polyphosphoesters, poly(vinyl pyrrolidone), polyols, hydrophilic polypeptides, or other synthetic hydrophilic polymers.

### N- and C-Terminal End Groups

The tertiary amino lipidated and/or PEGylated cationic peptide compounds of the present disclosure may be provided with the N-terminus and C-terminus in their free amine and free acid forms, respectively, or in protected forms. In some embodiments, the tertiary amino lipidated and/or PEGylated cationic peptide compound comprises further protecting or end capping groups at the N- and/or C-terminal residues.

In some embodiments, the tertiary amino lipidated and/or PEGylated cationic peptide compound comprises a protecting or end capping group R¹ at the N-terminal residue. In some embodiments, R¹ is —H, alkyl, alkylaryl, —COR^{1a}, —CH₂—COR^{1a}, a cationic moiety, a lipid moiety, or an oligoethylene glycol or polyethylene glycol moiety, wherein R^{1a} is —H, —OH, alkyl, aryl, alkylaryl, —O-alkyl, —O-alkylaryl, or a lipid moiety. In certain embodiments, R¹ is —H, alkyl, alkylaryl, —COR^{1a}, or a lipid moiety, wherein R^{1a} is —H, —OH, alkyl, aryl, alkylaryl, —O-alkyl, or —O-alkylaryl. In certain embodiments wherein R¹ is a lipid moiety, the lipid moiety is not a phospholipid. In still other embodiments wherein R¹ is alkyl or —COR^{1a} wherein Ria is alkyl, the alkyl is optionally substituted by —OH or halo.

In other embodiments, the tertiary amino lipidated and/or PEGylated cationic peptide compound comprises a protecting or end capping group R⁷ at the C-terminal residue. In some embodiments, R⁷ is —H, alkyl, acyl, —OH, —OR^{7a}, —NH₂, —NHR^{7a}, a cationic moiety, a lipid moiety, or an oligoethylene glycol or polyethylene glycol moiety, wherein R^{7a} is alkyl, acyl, or a lipid moiety. In some embodiments, R⁷ is —H, alkyl, acyl, —OH, —OR^{7a}, —NH₂, —NHR^{7a}, or a lipid moiety, wherein R^{7a} is alkyl, acyl, or a lipid moiety. In certain embodiments wherein R⁷ is a lipid moiety, the lipid moiety is not a phospholipid.

In still further embodiments, the N- and C-terminus of the tertiary amino lipidated and/or PEGylated cationic peptide compound may be selected or further modified such that R¹ and R⁷ possess additional functional groups such as reactive linker groups.

In some embodiments, R1 and/or R⁷ of the tertiary amino lipidated and/or PEGylated cationic peptide independently comprises one or more reactive linker groups. Suitable reactive groups may include but are not limited to esters, amides, isocyanates, thiols or “click” chemistry compatible moieties (e.g., azido, alkynyl).

Reactive linker groups may in turn be used to covalently bind other useful compounds, including targeting elements or therapeutic agents, to the peptide compound. In some embodiments, the tertiary amino lipidated and/or PEGylated cationic peptide compound is covalently bound, or conjugated, to a therapeutic agent and/or targeting element. In certain embodiments, the therapeutic agent and/or targeting element is a small molecule, an antibody or an antibody fragment.

In addition, additional functional groups for R¹ and R⁷ may also comprise cationic groups which are not compatible with the synthesis or deprotection conditions (such as acid-labile linkers) or for which a suitable protecting group strategy is not available (e.g. polyamines). In yet further embodiments, R¹ is a polyamine. In some embodiments wherein R¹ is a polyamine, the polyamine is

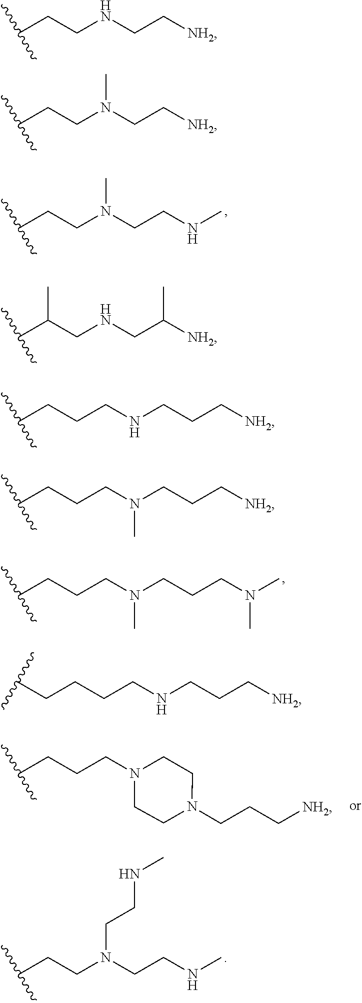

In certain embodiments, the polyamine is selected from the group consisting of

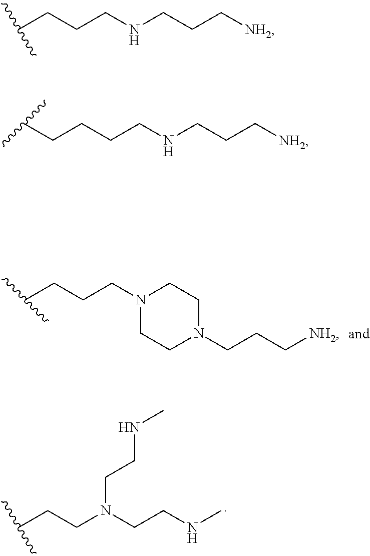 [SMILES: *CCCNCCCN ; *CCCCNCCCN ; *CCCN1CCN(CCCN)CC1 ; *CCN(CCNC)CCNC]

As described above, linker groups or linkages that are sensitive to or labile under certain physiological conditions environments may facilitate targeted delivery polyanionic material to specific cells and improve certain pharmacokinetic properties such as elimination. In certain embodiments, the linker group is selected such that the covalent bond between the tertiary amino lipidated and/or PEGylated cationic peptide compound and the therapeutic agent or targeting element is hydrolytically labile, chemically labile, pH labile, photolabile, thermally labile, or enzymatically cleavable.

Combinations of Tertiary Amino Lipidated and/or PEGylated Cationic Peptide Compounds

The complexes and compositions of the present disclosure may comprise one or more cationic compounds. In some embodiments, the complexes described herein may comprise one or more tertiary amino lipidated and/or PEGylated cationic peptide compounds of formula (I). The use of mixtures and combinations of multiple cationic peptide compounds of the present disclosure in a single complex may enable the preparation of formulations tailored for specific pharmacokinetic and pharmacodynamics properties. Pharmacokinetic and pharmacodynamics properties of relevance may include but are not limited to biodistribution, immunogenicity, formulation stability, encapsulation percentage, transfection efficiency, plasma half-life, etc. Different combinations of these properties may be required for different applications.

For example, in certain embodiments, the complex or composition may comprise a combination of one or more tertiary amino lipidated and/or PEGylated cationic peptide compounds which are cation-rich with one or more tertiary amino lipidated and/or PEGylated cationic peptide compounds which are highly lipidated and/or contain at least one PEG moiety. In theory, such a combination could confer improved delivery of polyanionic compounds by providing greater charge stabilization (via the cation-rich peptide compounds) along with greater lipophilic shielding (by virtue of the lipidated peptide compounds). It should further be recognized that the individual amounts of each of the individual tertiary amino lipidated and/or PEGylated cationic peptide compounds may be adjusted to achieve the desired properties.

Synthesis of Tertiary Amino Lipidated and/or PEGylated Cationic Peptide Compounds

In another aspect, provided herein are methods of preparing the tertiary amino lipidated and/or PEGylated cationic peptide compounds described herein.

The tertiary amino lipidated and/or PEGylated cationic peptide compounds of the present disclosure may be synthesized without the need for additional linker species to conjugate a lipid moiety to the oligopeptide core. For the tertiary amino lipidated and/or PEGylated cationic peptide compounds described herein, the lipid and ethylene glycol moieties are covalently and directly bound to nitrogen atoms within the peptide itself, that is to say, at the amide nitrogen or N-position in the amino acid residues. Thus, the tertiary amino lipidated and/or PEGylated cationic peptides may be synthesized entirely by methods known in the art for producing N-substituted residues in a peptide chain.

The tertiary amino lipidated and/or PEGylated cationic peptide compounds may be prepared by a series of acylation (amidation) and nucleophilic displacement (amination) reactions for each amino acid residue to be added, regardless of whether the residue has a cationic, neutral, lipid, or oligo-/polyethylene glycol moiety at the N-position. The cationic peptide compounds described herein are synthesized by sequential addition of individual residues to the peptide chain. The sequential addition of residues may be carried out in repetition until the desired sequence and length of amino acids is achieved.

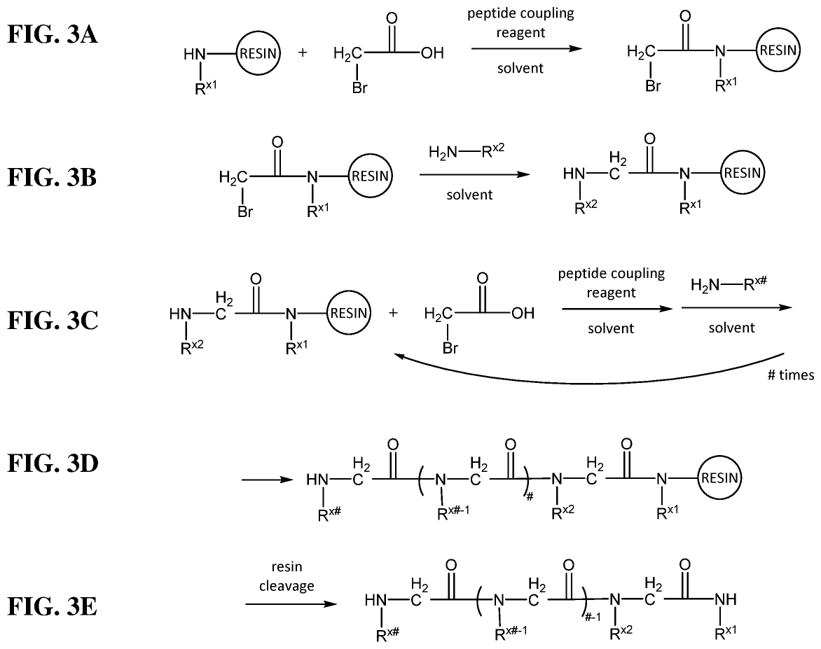

The compounds of the present invention can be synthesized by both solid-phase and solution-phase methods. An exemplary method for preparing N-substituted peptides, including the tertiary amino lipidated and/or PEGylated cationic peptide compounds described herein, by solid-phase synthesis is discussed below and shown in FIGS. 3A-3E.

As shown in FIG. 3A, a solid resin support having a terminal secondary amine is provided at the beginning of the synthesis. An acylating agent is added to the terminal secondary amine with a suitable peptide coupling reagent and solvent to form an amide bond between the terminal secondary amine and the acylating agent. The acylating agent is preferably an acetylating agent. The acylating agent comprises at least two suitable leaving groups to facilitate amidation and subsequent amination at the α-carbon. In certain embodiments, the acylating agent is a haloacetic acid. In other embodiments, the acylating agent is bromoacetic acid.

In FIG. 3B, the acylation product produced in FIG. 3A is reacted with the desired substituted primary or secondary amine to provide the corresponding N-substituted terminal amino acid residue. The selected primary or secondary amine displaces a leaving group, such as bromine in bromoacetic acid, on the α-carbon to produce the corresponding amination product. In some embodiments, the primary or secondary amine is an amine selected from the group consisting of NHR^{p}R², NHR^{p}R³, NHR^{p}R⁴, NHR^{p}R⁵, and NHR^{p}R⁶, wherein R^{p} is —H or a protecting group, and wherein R², R³, R⁴, R⁵, and R⁶ are as defined for the tertiary amino lipidated and/or PEGylated cationic peptide compound as described herein.

The amidation and amination reactions are repeated in series (FIG. 3C) until the desired peptide sequence is achieved (FIG. 3D). It should be recognized that the method of preparing the tertiary amino lipidated and/or PEGylated cationic peptide compound as described herein may further comprise protection and deprotection steps to prevent any undesired reactions with reactive moieties in the peptide chain over the course of the successive amidation/amination reactions. For example, protecting groups R^{p} may be added to the side chains at the α-carbon(s) and/or N-substituent(s) anywhere along the peptide compounds described herein during synthesis. Suitable protecting groups R^{p} may include any protecting groups known in the art, particularly those that are suitable for peptide synthesis in orthogonal protection schemes, such as Boc/Bzl or Fmoc/tBu.

The incorporation of end groups at the N- and C-termini (R¹ and R⁷) as well as functionalization of end groups and/or side chains of amino acid residues (R^{a}, R^{b}, R⁴, R⁵) along the oligopeptide backbone can be carried out by methods known in the art. A person having ordinary skill in the art would recognize that the selection of suitable methods and the timing of the additional functionalization step(s) relative to the overall solid-phase synthesis will depend upon the end groups and/or linker groups to be added, as well as compatibility of said methods with other moieties and/or protecting groups present on the peptide compounds.

The desired peptide compound is cleaved from the solid resin support (FIG. 3E) under suitable reaction conditions, such as acidic conditions including hydrochloric acid, hydrobromic acid, or trifluoroacetic acid, depending upon any protection scheme utilized in the synthesis as described above. Cleavage of the tertiary amino lipidated and/or PEGylated cationic peptide compound from the solid resin support produces the corresponding free cationic peptide compound in solution.

Further steps may be undertaken to isolate and purify the cationic peptide compound from solution, including for example filtration of the peptide-containing solution from the solid resin support and lyophilization of the isolated filtrate to provide a solid product.

Due to the acidic conditions employed to effect resin cleavage, it should be recognized that the tertiary amino lipidated and/or PEGylated cationic peptide compound may exist in the corresponding acid salt form. For example, in some embodiments, the tertiary amino lipidated and/or PEGylated cationic peptide compound is a salt form thereof. In certain embodiments, the salt form is an acid addition salt. In some embodiments, the salt of the tertiary amino lipidated and/or PEGylated cationic peptide compound is a hydrochloride salt (hydrochloric acid addition salt), a hydrobromide salt (hydrobromic acid addition salt, or a trifluoroacetate salt (trifluoroacetic acid addition salt). In certain embodiments, the salt of the tertiary amino lipidated and/or PEGylated cationic peptide compound is a trifluoroacetic acid addition salt, or trifluoroacetate salt, of the tertiary amino lipidated and/or PEGylated cationic peptide compound.

The acid addition salt form of the tertiary amino lipidated and/or PEGylated cationic peptide compound may be further modified via methods in the art (e.g., ion exchange) in order to obtain one or more pharmaceutically acceptable salt forms. The phrase “pharmaceutically acceptable” indicates that the substance or composition must be compatible chemically and/or toxicologically, with the other ingredients comprising a formulation, and/or the subject to which/whom the formulation is being administered. The term “pharmaceutically acceptable acid addition salt” may include but is not limited to those pharmaceutically acceptable salts formed with inorganic acids such as hydrochloric acid, hydrobromic acid, hydroiodic acid, sulfuric acid, nitric acid, carbonic acid, phosphoric acid, and organic acids selected from aliphatic, cycloaliphatic, aromatic, aryl-aliphatic, heterocyclic, carboxylic, and sulfonic classes of organic acids such as formic acid, acetic acid, propionic acid, gluconic acid, lactic acid, pyruvic acid, oxalic acid, maleic acid, malonic acid, succinic acid, fumaric acid, tartaric acid, citric acid, aspartic acid, ascorbic acid, glutamic acid, benzoic acid, phenylacetic acid, methanesulfonic acid “mesylate”, ethanesulfonic acid, p-toluenesulfonic acid, and salicyclic acid.

In some embodiments, the salt of the tertiary amino lipidated and/or PEGylated cationic peptide compound is a pharmaceutically acceptable salt. In certain embodiments, the salt is a hydrochloride salt, hydrobromide salt, hydroiodide salt, nitrate salt, sulfate salt, bisulfate salt, phosphate salt, acid phosphate salt, formate salt, acetate salt, propionate salt, gluconate salt, lactate salt, pyruvate salt, oxalate salt, maleate salt, malonate salt, succinate salt, fumarate salt, tartrate salt, bitartrate salt, citrate salt, aspartate salt, ascorbate salt, glutamate salt, benzoate salt, methanesulfonate salt, ethanesulfonate, p-toluenesulfonate salt, or salicylate salt.

### Lipitoids

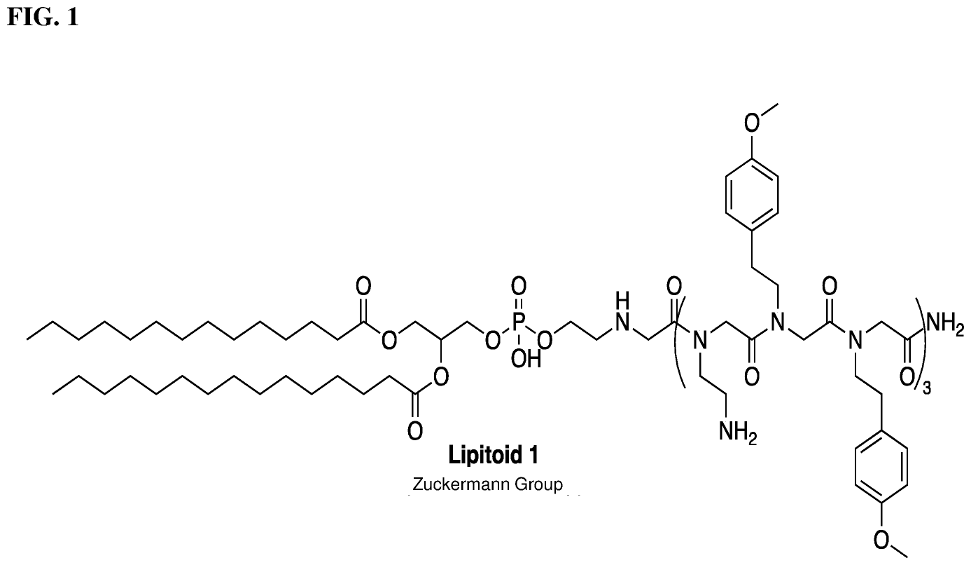

In some embodiments, the one or more cationic compounds of the complexes and compositions described herein comprises one or more cationic peptoid-phospholipid conjugate constructs, also known as lipitoids. Lipitoids are N-substituted polyglycine compounds (also known as “peptoids”) having a combination of cationic and/or neutral side chains at the N-positions of glycine residues along peptoid backbone, which are further conjugated to a single terminal phospholipid group of the peptoid chain. Lipitoids and their synthesis are generally described in Simon R. J. et al., Proc. Natl. Acad. Sci. U.S.A. 89, 9367-9371 (1992); Lobo, B. A.; Vetro, J. A.; Suich, D. M.; Zuckermann, R. N.; Middaugh, C. R. Structure/Function Analysis of Peptoid/Lipitoid:DNA Complexes. Journal of Pharmaceutical Sciences 2003, 92 (9), 1905-1918; and Utku, Y.; Dehan, E.; Ouerfelli, O.; Piano, F.; Zuckermann, R. N.; Pagano, M.; Kirshenbaum, K. A Peptidomimetic SiRNA Transfection Reagent for Highly Effective Gene Silencing. Molecular BioSystems 2006, 2 (6-7), 312. An exemplary lipitoid is the nonamer “Lipitoid 1” as shown in FIG. 1. The general structure of a lipitoid compound is shown in formula (II) below, wherein x is an integer from 1 to 100, each R⁹ is independently a lipid moiety and each R¹⁰ is independently a cationic or neutral moiety.

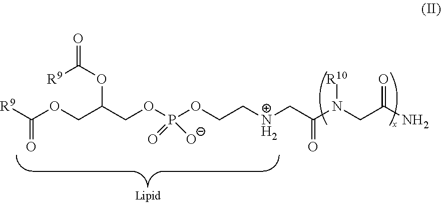

In formula (II) above, x is an integer from 1 to 100. Particular numbers of residues may be especially suitable for use in the complexes and compositions described herein. For example, in some embodiments, x is an integer from 1 to 50, from 2 to 25, or from 3 to 15.

As shown in Formula (II) above, R⁹ represent lipid moieties, including but not limited to include branched or straight chain aliphatic moieties having from about 6 to about 50 carbon atoms or from about 10 to about 50 carbon atoms, optionally comprising one or more heteroatoms, and optionally comprising one or more double or triple bonds (i.e., saturated or mono- or poly-unsaturated). In certain embodiments, the lipid moieties R⁹ may include optionally substituted aliphatic, straight chain or branched moieties, each hydrophobic tail independently having from about 8 to about 30 carbon atoms or from about 6 to about 30 carbon atoms. In certain embodiments, the lipid moieties may include, for example, aliphatic carbon chains derived from fatty acids and fatty alcohols. In some embodiments, each lipid moiety R⁹ is independently C₈-C₂₄-alkyl or C₈-C₂₄-alkenyl, wherein the C₈-C₂₄-alkenyl is optionally mono- or poly-unsaturated. In other embodiments, each lipid moiety R⁹ is independently selected from the group consisting of oleyl, stearyl, linoleyl, myristyl, and lauryl. In yet other embodiments which may be combined with any of the preceding embodiments, each lipid moiety R⁹ is independently

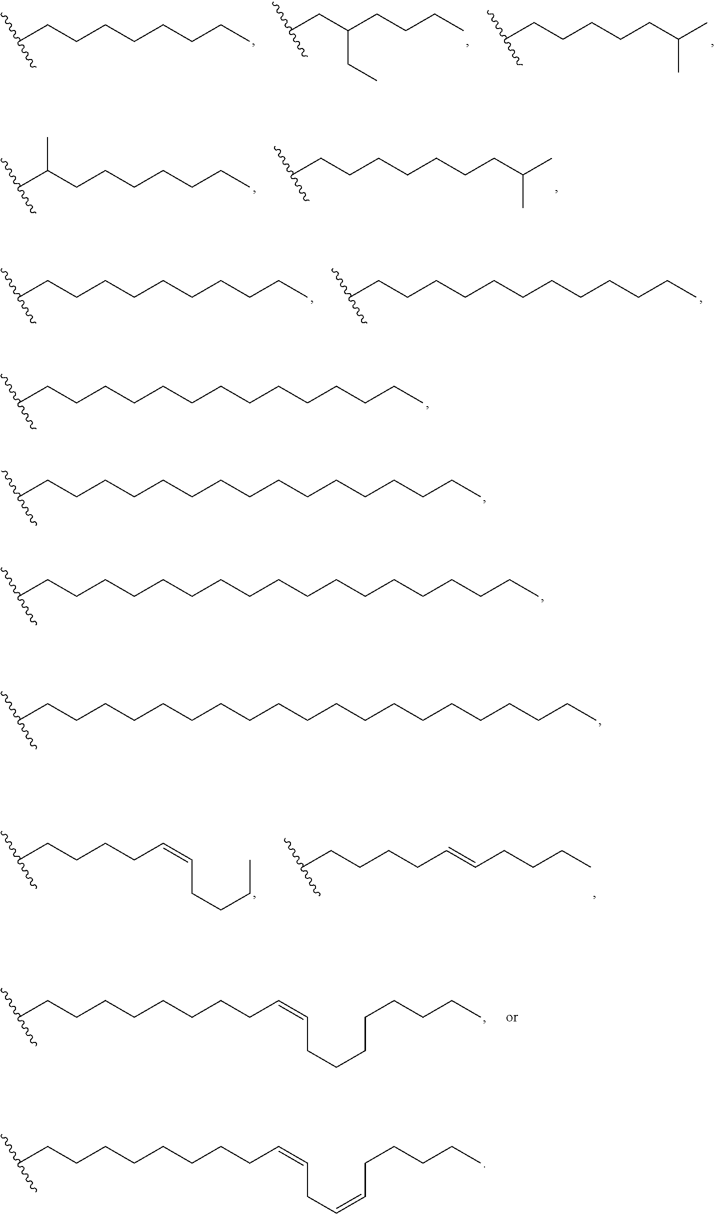

The group(s) R¹⁰ at the N-position of the amino glycine residue includes cationic and neutral moieties similar to those described above for the tertiary amino lipidated and/or PEGylated cationic peptide compounds.

Cationic, or positively charged, moieties may include, for example, nitrogen-based substituents, such as those containing the following functional groups: amino, guanidino, hydrazido, and amidino. These functional groups can be either aromatic, saturated cyclic, or aliphatic. In some embodiments of the lipitoid, each cationic moiety is independently aminoalkyl, alkylaminoalkyl, aminoalkylaminoakyl, guanidinoalkyl, or N-heterocyclylalkyl. In other embodiments, each cationic moiety is independently

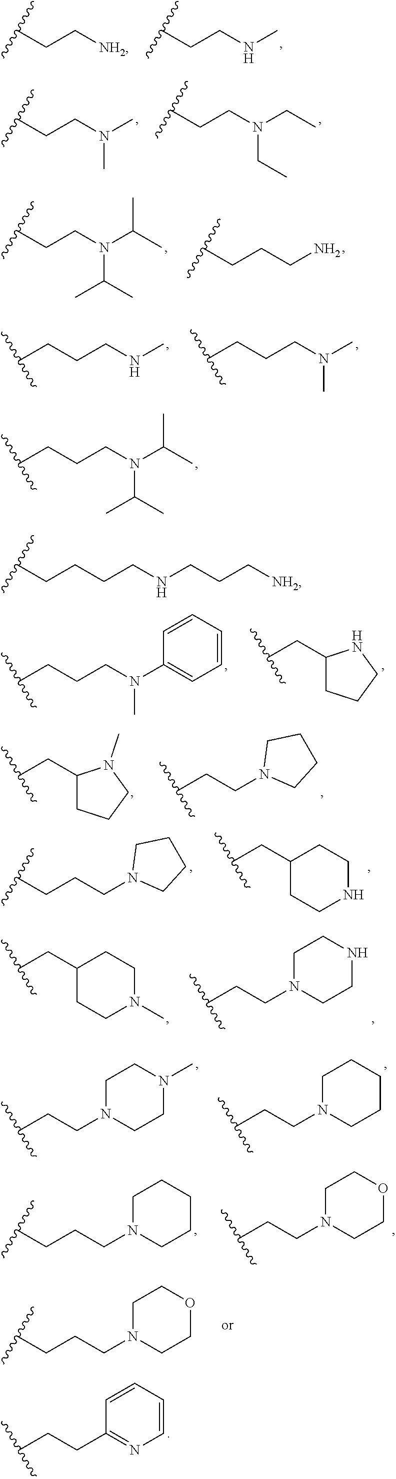

In certain embodiments, each cationic moiety is independently selected from the group consisting of:

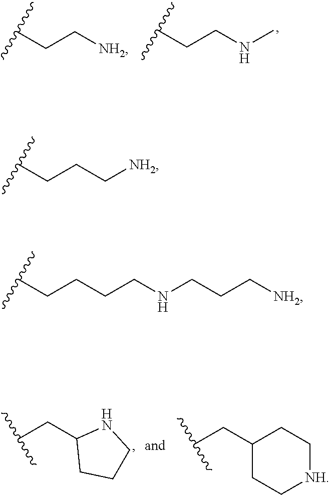

In still other embodiments, each cationic moiety is

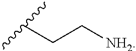 [SMILES: *CCN]

Neutral moieties may include but are not limited to a C₁-C₄-alkyl substituted by cycloalkyl, heterocyclylalkyl, alkylaryl, arylalkyl, alkylheteroaryl, heteroarylalkyl, alkoxy, alkoxyalkyl, or hydroxyalkyl, wherein each cycloalkyl, heterocyclylalkyl, alkylaryl, arylalkyl, alkylheteroaryl, heteroarylalkyl, alkoxy, alkoxyalkyl, or hydroxyalkyl is optionally substituted with one or more substituents —OH, halo, or alkoxy. In some embodiments of the lipitoid, each neutral moiety is independently selected from the group consisting of:

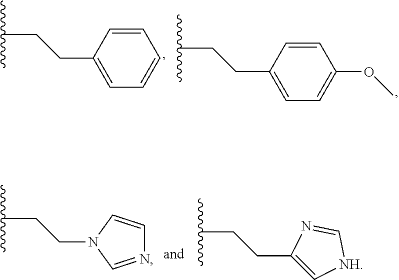

In certain embodiments, each neutral spacer moiety is

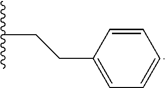 [SMILES: *CCc1ccccc1]

In still other embodiments, each neutral spacer moiety is

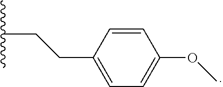 [SMILES: *CCc1ccc(OC)cc1]

In still other embodiments, each cationic moiety is

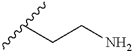 [SMILES: *CCN]

and each neutral spacer moiety is

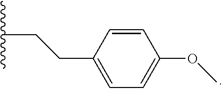 [SMILES: *CCc1ccc(OC)cc1]

In yet further embodiments, the one or more lipitoids comprise Lipitoid 1.

Similar to the tertiary amino lipidated and/or PEGylated cationic peptide compounds above, the lipitoids described herein may inclhe amino acid residues may be arranged in random sequences, or repeating motifs in alternating sequences or block sequences. In some embodiments, the lipitoid of formula (II) may comprise repeating (dimer, trimer, tetramer, etc.) subunits within the chain of x residues. These subunits may include but are not limited to —R^{cation}R^{neutral}— or —R^{cation}R^{neutral}R^{neutral}— wherein R^{cation} is an amino acid residue comprising a cationic moiety and each R^{neutral} is an amino acid residue comprising a neutral spacer moiety. In other embodiments, the lipitoid of formula (II) comprises at least one trimer subunit —R^{cation}—R^{neutral}—R^{neutral}, and wherein R^{cation} is an amino acid residue comprising a cationic moiety and each R^{neutral} is an amino acid residue comprising a neutral spacer moiety.

In some embodiments wherein the lipitoid of formula (II) comprises a cationic domain, each cationic moiety R¹⁰ is

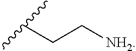 [SMILES: *CCN]

In other embodiments of the foregoing embodiments, each cationic moiety R¹⁰ is

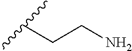 [SMILES: *CCN]

and each neutral spacer moiety R¹⁰ is

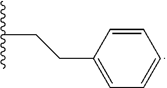 [SMILES: *CCc1ccccc1]

In certain embodiments, the lipitoid of formula (II) comprises one or more dimer or trimer subunits, wherein each cationic moiety is

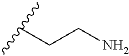 [SMILES: *CCN]

and each neutral spacer moiety is

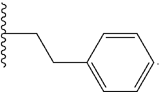 [SMILES: *CCc1ccccc1]

In still other embodiments, the lipidoid of formula (II) comprises a cationic domain comprising one or more trimer subunits —R^{cation}R^{neutral}R^{neutral}—, wherein R^{cation} is an amino acid residue comprising a cationic moiety and each R^{neutral} is an amino acid residue comprising a neutral spacer moiety, wherein each cationic moiety is

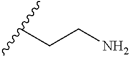 [SMILES: *CCN]

and wherein each neutral spacer moiety is

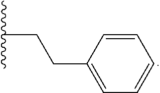 [SMILES: *CCc1ccccc1]

As with the tertiary amino lipidated and/or PEGylated cationic peptide compounds, the complexes and compositions described herein may comprise one or more lipitoids. In certain embodiments, the one or more cationic compounds comprises one or more lipitoids. In still other embodiments, the one or more cationic compounds comprises Lipitoid 1.

### Lipid-Like Cationic Compounds

In still other embodiments, the one or more cationic compounds of the complexes and compositions described herein may comprise one or more lipid-like cationic compounds.

Lipid-like cationic compounds are amphiphilic compounds comprising one or more ionizable (cationic) nitrogen-containing moieties and one or more lipophilic tails. Lipid-like cationic compounds are alternately referred to as “cationic lipids” or “ionizable lipids”. The ionizable (cationic) moieties in the lipid-like cationic compounds may include neutral tertiary amines that are ionized to positively charged moieties at suitable physiological pH levels. Alternatively, the ionizable (cationic) atoms may be quaternary ammonium groups provided as positively charged group with a suitable counterion. Lipophilic tails may include, for example, include branched or straight chain aliphatic moieties having from about 6 to about 50 carbon atoms or from about 10 to about 50 carbon atoms, optionally comprising one or more heteroatoms, and optionally comprising one or more double or triple bonds (i.e., saturated or mono- or poly-unsaturated).

Examples of lipid-like cationic compounds and are described in US. Patent Publications US2018/0147166 A1 and shown as Formula (III) below.

In some embodiments, the one or more cationic compounds comprise one or more lipid-like cationic compounds of formula (III):

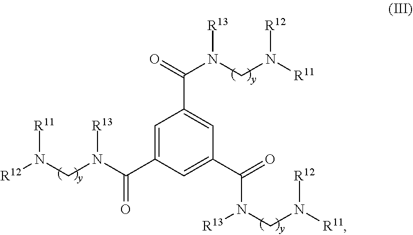

wherein each R¹¹ is independently substituted or unsubstituted alkyl; each R¹² is independently substituted or unsubstituted alkyl; each R¹³ is independently hydrogen or substituted or unsubstituted alkyl; and each y is independently an integer from 1 to 8.

### PEGylated Compounds

As described herein, the complexes and compositions of the present disclosure comprise one or more PEGylated compounds, such as PEGylated lipids or compounds possessing PEG (and optionally also lipid) functionalities. The PEGylated compounds described herein may be alternatively referred to as “PEG compounds”. The incorporation of PEGylated compounds into the complex comprising one or more cationic compounds and one or more polyanionic cargoes facilitates the endocellular uptake of cationic compound-cargo by deterring particle aggregation, shielding particle surface charge and providing protection against endogenous degradation pathways, including for example attack by nucleases.

### PEGylated Lipids

In some embodiments, the complexes and compositions described herein comprise one or more PEGylated compounds, wherein the one or more PEGylated compounds comprise one or more PEGylated lipids. It should be recognized that the term “PEGylated lipid” may also be interchangeably referred to as a “PEG lipid” or “PEG-modified lipid”. As described herein, PEGylated lipid may be understood to include any lipid or lipid-like compounds covalently bound to a polyethylene glycol moiety.

Suitable lipid moieties for the PEGylated lipid may include, for example, optionally substituted branched or straight chain aliphatic moieties, or optionally substituted moieties derived from natural lipid compounds, including fatty acids, sterols, and isoprenoids.

In some embodiments, the lipid moieties may include branched or straight chain aliphatic moieties having from about 6 to about 50 carbon atoms or from about 10 to about 50 carbon atoms, optionally comprising one or more heteroatoms, and optionally comprising one or more double or triple bonds (i.e., saturated or mono- or poly-unsaturated). In certain embodiments, the lipid moieties may include optionally substituted aliphatic, straight chain or branched moieties, each hydrophobic tail independently having from about 8 to about 30 carbon atoms or from about 6 to about 30 carbon atoms. In certain embodiments, the lipid moieties may include, for example, aliphatic carbon chains derived from fatty acids and fatty alcohols. In some embodiments, each lipid moiety is independently C₈-C₂₄-alkyl or C₈-C₂₄-alkenyl, wherein the C₈-C₂₄-alkenyl is optionally mono- or poly-unsaturated.

Natural lipid moieties employed in the practice of the present invention can be derived from, for example, phospholipids, glycerides (such as di- or tri-glycerides), glycosylglycerides, sphingolipids, ceramides, and saturated and unsaturated sterols, isoprenoids, and other like natural lipids.

Other suitable lipid moieties may include lipophilic aromatic groups such as optionally substituted aryl or arylalkyl moieties, including for example naphthalenyl or ethylbenzyl, or lipids comprising ester functional groups including, for example, sterol esters and wax esters.

In other embodiments, the one or more PEG lipids are selected from the group consisting of a PEG-modified phosphatidylethanolamine, a PEG-modified phosphatidic acid, a PEG-modified ceramide, a PEG-modified dialkylamine, a PEG-modified diacylglycerol, a PEG-modified dialkylglycerol, and any combinations thereof.

In some embodiments, the PEG lipid comprises a PEG-modified sterol. In certain embodiments, the PEG lipid comprises PEG-modified cholesterol.

In some embodiments, the PEG lipid is a PEG-modified ceramide. In certain embodiments, the PEG-modified ceramine is selected from the group consisting of N-octanoyl-sphingosine-1-{succinyl[methoxy(polyethylene glycol)]} and N-palmitoyl-sphingosine-1-{succinyl[methoxy(polyethylene glycol)]}, and any combination thereof.

In some embodiments, the phospholipid of the PEG-modified phospholipid is selected from the group consisting of 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-distearoyl-sn-glycero-3-phosphocholine (DSPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C 16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-dioleoyl-sn-glycero-3-phosphoethanolamine (DOPE), 1,2-dipalmitoyl-sn-glycero-3-phosphoethanolamine (DPPE), 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1,2-dioleoyl-sn-glycero-3-phospho-rac-(1-glycerol) sodium salt (DOPG), sphingomyelin, and mixtures thereof. In certain embodiments, the phospholipid is DOPE.

In some embodiments, the one or more PEG lipids comprise a PEG-modified phosphatidylethanol. In some embodiments, the PEG lipid is a PEG-modified phosphatidylethanol selected from the group consisting of PEG-modified DMPE (DMPE-PEG), PEG-modified DSPE (DSPE-PEG), PEG-modified DPPE (DPPE-PEG), and PEG-modified DOPE (DOPE-PEG).

In certain embodiments, the PEG lipid is selected from the group consisting of dimyristoylglycerol-polyethylene glycol (DMG-PEG), distearoylglycerol-polyethylene glycol (DSG-PEG), dipalmitoylglycerol-polyethylene glycol (DPG-PEG), and dioleoylglycerol-polyethylene glycol (DOG-PEG). In certain embodiments, the PEG lipid is DMG-PEG.

It should be further recognized that particular molecular weights of the PEG chain in the foregoing PEG lipids may be especially advantageous for incorporation into the complexes of the present disclosure. For example, in some embodiments, the PEG chain has a molecular weight between 350 and 6,000 g/mol, between 1,000 and 5,000 g/mol, or between 2,000 and 5,000 g/mol. In certain embodiments, the PEG chain of the PEG lipid has a molecular weight of about 350 g/mol, 500 g/mol, 600 g/mol, 750 g/mol, 1,000 g/mol, 2,000 g/mol, 3,000 g/mol, 5,000 g/mol, or 10,000 g/mol. In certain other embodiments, the PEG chain of the PEG lipid has a molecular weight of about 500 g/mol, 750 g/mol, 1,000 g/mol, 2,000 g/mol or 5,000 g/mol. For example, in certain embodiments, the PEGylated lipid is dimyristoylglycerol-polyethylene glycol 2000 (DMG-PEG 2000).

### Tertiary Amino Lipidated and PEGylated Cationic Peptides

In still further embodiments, the one or more PEGylated compounds comprise a tertiary amino PEGylated cationic peptide compounds of formula (I) comprising at least one oligo- or polyethylene glycol moiety. By virtue of their flexibility in accommodating both lipid and PEG moieties along the backbone of the peptide chain and depending upon the nature of their specific substituents, the tertiary amino lipidated and/or PEGylated cationic peptide compounds of formula (I) of the present disclosure may serve not only as cationic delivery vehicles but also as suitable PEGylated compounds to stabilize the cationic compound-polyanionic compound complex. It should be recognized that tertiary amino PEGylated cationic peptide compounds of formula (I) may be combined with other classes of cationic compounds such as lipitoids or lipid-like compounds as well as other tertiary amino lipidated and/or PEGylated cationic peptide compounds of formula (I).

In some embodiments, the PEGylated compound is a tertiary amino lipidated and/or PEGylated cationic peptide compound of formula (I) as described herein, wherein at least one of m and t is nonzero (i.e., N-PEGylated). In still further embodiments, the PEGylated compound is a tertiary amino lipidated and/or PEGylated cationic peptide compound of formula (I) wherein m and t are independently an integer from 0 to 15, and wherein at least one of m and t is nonzero. In some embodiments, the PEGylated compound is a tertiary amino PEGylated cationic peptide compound of formula (Ib). In yet other embodiments, the PEGylated compound is a tertiary amino lipidated and PEGylated cationic compound of formula (Ic).

As with the PEGylated lipids described above, particular molecular weights of the PEG chain in the foregoing tertiary amino lipidated and PEGylated cationic peptide compounds may be especially advantageous for incorporation into the complexes of the present disclosure. For example, in some embodiments, the PEG chain has a molecular weight between 350 and 6,000 g/mol, between 1,000 and 5,000 g/mol, or between 2,000 and 5,000 g/mol. In certain embodiments, the PEG chain of the tertiary amino lipidated and PEGylated cationic peptide compound has a molecular weight of about 350 g/mol, 500 g/mol, 600 g/mol, 750 g/mol, 1,000 g/mol, 2,000 g/mol, 3,000 g/mol, or 5,000 g/mol. In certain other embodiments, the PEG chain of the PEG lipid has a molecular weight of about 500 g/mol, 750 g/mol, 1,000 g/mol, 2,000 g/mol or 5,000 g/mol.

However, it should be recognized that the tertiary amino lipidated and/or PEGylated cationic peptide compounds of formula (I) may comprise several short oligoethylene glycol moieties in lieu of fewer longer polyethylene glycol moieties and provide similar particle stabilization to the complex. In some embodiments wherein the PEGylated compound is an N-PEGylated cationic peptide compound of formula (I), tertiary amino lipidated and/or PEGylated cationic peptide compound comprises at least one ethylene glycol moiety R² of the formula —CH₂CH₂O(CH₂CH₂O)ᵤR^{2a}, wherein each R^{2a} is independently —H or C1-C₄-alkyl. In other embodiments, at least one ethylene glycol moiety R⁶ of the formula —CH₂CH₂O(CH₂CH₂O)ᵥR^{6a}, wherein each R^{6a} is independently —H or C₁-C₄-alkyl. In some embodiments which may be combined with any of the preceding embodiments wherein m is nonzero, m is an integer from 0 to 15, wherein each u is independently an integer from 1 to 200. In still further embodiments which may be combined with any of the preceding embodiments wherein t is nonzero, t is an integer from 0 to 15, wherein each v is independently an integer from 1 to 200.

In some embodiments, the PEGylated compound is a tertiary amino lipidated and/or PEGylated cationic peptide compound of formula (I) as described herein, wherein at least one of m and t is nonzero and at least one of n and s is nonzero (i.e., N-lipidated). In certain embodiments wherein the N-PEGylated cationic peptide compound of formula (I) is also N-lipidated, at least one of n and s is nonzero. In some embodiments, the sum of n and s is at least 1, 2, 3, or 4. In certain embodiments, s is 4. In yet further embodiments, n is 4.

### Polyanionic Compounds and Other Delivery Cargoes

As described herein the complexes and compositions of the present disclosure may comprise one or more polyanionic compounds, such as nucleic acids, complexed with the cationic compounds. The complexes and compositions of the present disclosure comprising one or more cationic compounds, such as tertiary amino lipidated and/or PEGylated cationic peptide compounds, and the one or more PEGylated lipids with the polyanionic compounds may be used to deliver the polyanionic compounds, such as nucleic acids, to the cell interior. In addition to polyanionic cargoes, the complexes and compositions provided herein may also be utilized for the endocellular delivery of a wide array of non-anionic cargoes. Depending upon the polyanionic and non-anionic compounds or cargoes to be delivered into cells, the complexes and compositions provided herein may be useful in a number of different applications, including for example vaccination and gene therapies.

In some embodiments, the one or more polyanionic compounds comprises a nucleic acid. Nucleic acids, as used herein, include naturally occurring nucleic acids such as DNA, RNA, and/or hybrids thereof as well as unnaturally occurring variations with unnatural backbone and modified backbone linkages such as phosphorothioate, unnatural and modified bases, and unnatural and modified termini. Exemplary nucleic acids include genomic DNA, cDNA, mRNA, miRNA, and siRNA.

The nucleic acids may be recombinantly produced or chemically synthesized molecules. A nucleic acid may be single-stranded, double-stranded, triple stranded, and quadruple stranded as well as in more complicated three-dimensional forms including single and double stranded regions.

Depending upon the type of nucleic acid, the length of the nucleic acid (defined in nucleotide units or base pairs (bp) as appropriate) may vary. In some embodiments wherein the nucleic acid is mRNA, the mRNA may have from 100 to 10,000 nucleotide units, or from 1,000 to 3,000 nucleotide units. In other embodiments wherein the nucleic acid is DNA, the DNA may have from 5,000 bp to 20,000 bp, or about 10,000 bp.

In some embodiments wherein the nucleic acid is an mRNA, the mRNA is an mRNA encoding a protein or a peptide. In some embodiments wherein the nucleic acid is an mRNA, the mRNA is an mRNA encoding a peptide, including an oligopeptide or a polypeptide. In certain embodiments, the mRNA is an mRNA encoding a polypeptide. In yet further embodiments, the mRNA is an mRNA encoding a protein. In other embodiments, the mRNA is an mRNA encoding a peptide. As described above, the mRNA may be naturally occurring (e.g., isolated tumor RNA) or may be synthetic (e.g., produced by in vitro transcription). For synthetic or unnaturally occurring variations of mRNA, the mRNA may comprise an unnatural backbone with modified backbone linkages such as phosphorothioate, unnatural and modified bases, and/or unnatural and modified termini. In certain embodiments wherein the nucleic acid is an mRNA, the mRNA may comprise special sequences such as self-amplifying sequences or internal ribosome entry sites.

In some embodiments, the combined delivery of two or more particular nucleic acids together may be especially useful for therapeutic applications. For example, in some embodiments, the one or more polyanionic compounds includes a combination of sgRNA (single guide RNA) as a CRISPR sequence and mRNA encoding Cas9. In still further embodiments, the nucleic acids may also be complexed with proteins such as with the CRISPR/Cas9 ribonucleoprotein complex.

In some embodiments, the one or more polyanionic compounds may include anionic or polyanionic compounds that are not nucleic acids. Suitable anionic compounds may include but are not limited to proteins, polyphosphates, or heparins. In some embodiments, the one or more polyanionic compounds comprises one or more proteins. In one embodiments, the one or more polyanionic compounds comprises Cas9 protein. In other embodiments, the one or more polyanionic compounds comprises polyphosphates. In yet other embodiments, the one or more polyanionic compounds comprises heparins or other glycosaminoglycan derivatives.

In addition to the suitability of the complexes and compositions provided herein to deliver polyanionic cargoes into cells, it should be acknowledged that the complexes and compositions described herein may also be utilized to deliver other non-anionic agents or cargoes (including hydrophobic compounds) into cells. These other cargoes may include but are not limited to, for example, small molecule active agents (as a standalone therapeutic or in combination with another agent, such as a nucleic acid) and/or immunological adjuvants. It should be further recognized that these other cargo molecules may or may not be combined with any of the polyanionic compounds described herein for complexation. In some embodiments, the complexes described herein comprise endosomal escape modulators, TLR agonists, and chemotherapeutics. In other embodiments, the complexes of the present disclosure comprise adjuvants or immune co-stimulators. For example, in some embodiments, the complex or composition comprises immunological adjuvants selected from the group consisting of CpG oligodeoxynucleotides (ODNs), lipopolysaccharides (LPS), and any combinations thereof.

### Additional Components

The complexes and compositions of the present disclosure may comprise one or more additional components including polyethylene glycol and/or targeting agent.

In some embodiments, the complexes and composition comprises one or more additional components, wherein the one or more additional components comprises polyethylene glycol (PEG). It should be further recognized that particular molecular weights of the polyethylene glycol may be particularly suitable for incorporation into the complexes and compositions of the present disclosure. For example, in some embodiments, the polyethylene glycol has a molecular weight between 350 and 6,000 g/mol, between 1,000 and 5,000 g/mol, or between 2,000 and 5,000 g/mol. In certain embodiments, the polyethylene glycol has a molecular weight of about 350 g/mol, 500 g/mol, 600 g/mol, 750 g/mol, 1,000 g/mol, 2,000 g/mol, 3,000 g/mol, 5,000 g/mol or 10,000 g/mol. In certain other embodiments, the polyethylene glycol has a molecular weight of about 500 g/mol, 750 g/mol, 1,000 g/mol, 2,000 g/mol or 5,000 g/rmol. In some embodiments wherein the composition comprises polyethylene glycol as an additional component, it should be recognized that the one or more polyethylene glycol polymers of different molecular weights may be included in the complexes and composition.

In other embodiments, the complex or composition comprises one or more additional components, wherein the one or more additional components comprises a targeting agent. In certain embodiments, the targeting agent is a small molecule, an antibody or an antibody fragment.

It should be recognized that the compositions as described herein may be prepared as a formulation for administration. In some embodiments, the formulation is a solution, a suspension, a colloidal suspension, a spray, or an aerosol. In other embodiments, the formulation is a lipid complex formulation. In some embodiments, the formulation is an enteral formulation, a parenteral formulation or a topical formulation. In some embodiments, the formulation is an injectable formulation, such as an intravenous formulation, a subcutaneous formulation, an intramuscular formulation, an intradermal formulation, an intraocular formulation, an intrathecal formulation, or an intratumoral formulation. In other embodiments, the formulation is an oral formulation. In still other embodiments, the formulation is a mucosal formulation, including for example a nasal formulation, an intra-anal formulation, a buccal formulation, or an intravaginal formulation, etc.

It should be further recognized that the components used in preparing the complexes and formulations of the present disclosure, as well as the process parameters for making said complexes or formulations, may be adapted depending upon whether the complexes and/or formulations are intended for immediate use (“just-in-time”) or will be placed in storage for future use. In particular, considerations for storage may include the temperature at which the complexes and/or formulations are kept (e.g., at room temperature, at 4° C., at −20° C., at −78° C.) and the duration of storage.

Accordingly, suitable excipients may include but are not limited to those which facilitate administration or improve storage stability (e.g., cryoprotectant). For example, in addition to components described above, the formulations described herein may comprise pharmaceutically acceptable excipients, such as carriers, solvents, dispersants, diluents, fillers, stabilizers, preservatives, etc. In still other embodiments, the composition may comprise pharmaceutically acceptable excipients or carriers, including for example, solvents, diluents, buffers, and viscosity modulators.

However, in contrast to standard lipid nanoparticle formulations, the complexes and compositions of the present disclosure do not comprise any standalone structural lipids (such as cholesterol or related sterols) or standalone phospholipids (e.g., DOPE) unless conjugated to a polyethylene glycol moiety (PEG-modified) as described above.

### Properties of the Complexes and Composition

As described herein, the complexes and compositions of the present disclosure are primarily composed of one or more cationic compounds, one or more PEG compounds and one or more polyanionic compounds, such as nucleic acids, for endocellular delivery. In contrast to many other existing lipid-based formulations, such as standard lipid nanoparticle formulations, the complexes and compositions of the present disclosure do not include structural lipids (such as cholesterol) or phospholipids (such as DOPE). These additional co-components, typically used for providing structural stability and improved cellular membrane permeability, constitute a significant proportion of the overall formulation. Traditional lipid nanoparticle formulations comprise a large quantity of inactive ingredients and, thus, require larger volumes of formulation to be administered in order to deliver a certain quantity of nucleic acid, for example, to elicit the desired therapeutic or prophylactic effect. In contrast, while formulations comprising only cationic compounds complexed with polyanionic cargoes to neutralize charge contain a greater fraction of the polyanionic material by weight, these cationic compound-polyanionic compound complexes suffer from particle instability and aggregation without the benefit any additional co-components.

The complexes and compositions of the present disclosure overcome the disadvantages of the prior lipid formulations by combining the cationic compounds and polyanionic compounds with a small proportion of PEGylated lipid, thereby stabilizing the cationic compound-polyanionic compound complex, protecting against in vivo degradation, and promoting endocytosis without deleteriously reducing the weight proportion of the active polyanionic material in the complex and, thus, the overall composition.

In one aspect, the complexes and compositions of the present disclosure may be characterized by the mass percentage (or weight percentage) of each of the individual components present in the complex. Alternatively, the complexes and compositions may be characterized by the combined mass percentage of two or more components with respect to the total mass of the complex. For example, in some embodiments, the one or more polyanionic compounds, the one or more PEG compounds, and the one or more cationic compounds have a combined mass percentage of at least 90% w/w, at least 95% w/w, or at least 99% w/w of the complex. In certain embodiments, the one or more polyanionic compounds, the one or more PEG compounds, and the one or more cationic compounds have a combined mass percentage of at least 90% w/w of the complex. In some embodiments wherein the complex comprises one or more tertiary amino lipidated and/or PEGylated cationic peptide compounds or salt thereof, the one or more one or more polyanionic compounds, the one or more PEG compounds, and the one or more tertiary amino lipidated and/or PEGylated cationic peptide compounds or salt thereof have a combined mass percentage of at least 90% w/w, at least 95% w/w, or at least 99% w/w of the complex. In certain embodiments, the one or more polyanionic compounds, the one or more PEG compounds, and the one or more tertiary amino lipidated and/or PEGylated cationic peptide compounds or salt thereof have a combined mass percentage of at least 90% w/w of the complex. In other embodiments wherein the complex comprises a complex comprising one or more lipitoids, the one or more polyanionic compounds, the one or more PEG compounds, and the one or more lipitoids have a combined mass percentage of at least 90% w/w, at least 95% w/w, or at least 99% w/w of the complex. In certain embodiments, the one or more polyanionic compounds, the one or more PEG compounds, and the one or more lipitoids have a combined mass percentage of at least 90% w/w of the complex.

In some embodiments wherein the complex or composition comprises one or more additional components, such as polyethylene glycol polymer or a targeting agent, the one or more additional components are present at a mass percentage of less than or equal to 10% of the complex.

In another aspect, the complexes and compositions of the present disclosure can be characterized by mass ratios of individual components to one another. For example, as described herein, the complexes of the present disclosure comprise a low mass percentage of PEGylated compounds relative to the mass percentage of cationic delivery vehicle. In some embodiments wherein the complex or composition comprises one or more cationic compounds and one or more PEG compounds, the one or more cationic peptide compounds and the one or more PEG compounds are present at a mass ratio between 90:10 and 99:1, between 92:8 and 98:2, between 95:5 and 99:1, or between 95:5 and 98:2. In certain embodiments wherein the complex or composition comprises one or more tertiary amino lipidated and/or PEGylated cationic peptide compounds, the one or more tertiary amino lipidated and/or PEGylated cationic peptide compounds and the one or more PEG compounds are present at a mass ratio between 90:10 and 99:1, between 92:8 and 98:2, between 95:5 and 99:1, or between 95:5 and 98:2. In still other embodiments wherein the complex or composition comprises one or more lipitoids, the one or more lipitoids and the one or more PEG compounds are present at a mass ratio between 90:10 and 99:1, between 92:8 and 98:2, between 95:5 and 99:1, or between 95:5 and 98:2.

In some embodiments wherein the complex or composition comprises one or more cationic compounds and one or more PEG lipids, the one or more cationic peptide compounds and the one or more PEG lipids are present at a mass ratio between 90:10 and 99:1, between 92:8 and 98:2, between 95:5 and 99:1, or between 95:5 and 98:2. In certain embodiments wherein the complex or composition comprises one or more tertiary amino lipidated and/or PEGylated cationic peptide compounds, the one or more tertiary amino lipidated and/or PEGylated cationic peptide compounds and the one or more PEG lipids are present at a mass ratio between 90:10 and 99:1, between 92:8 and 98:2, between 95:5 and 99:1, or between 95:5 and 98:2. In still other embodiments wherein the complex or composition comprises one or more lipitoids, the one or more lipitoids and the one or more PEG lipids are present at a mass ratio between 90:10 and 99:1, between 92:8 and 98:2, between 95:5 and 99:1, or between 95:5 and 98:2.

The complexes and compositions comprising the complexes as described herein may be characterized by the ratio of the total cationic charge on the one or more cationic compounds, for example, the number cationic groups on the tertiary amino lipidated and/or PEGylated cationic peptide, to the number of anionic phosphate groups on the nucleic acid. In some embodiments, the complexes comprise the one or more cationic compounds and the one or more polyanionic compounds at a cation: anion charge ratio of between 0.5:1 and 50:1, between 0.5:1 and 20:1, between 0.5:1 and 10:1, between 0.5:1 and 5:1, between 1:1 and 20:1, between 1:1 and 10:1, between 1:1 and 5:1, between 2:1 and 20:1, between 2:1 and 10:1, or between 2:1 and 5:1. In certain embodiments, the complexes comprise one or more tertiary amino lipidated and/or PEGylated cationic peptide and one or more polyanionic compounds, such as a nucleic acid, at a cation: anion charge ratio of between 0.5:1 and 50:1, between 0.5:1 and 20:1, between 0.5:1 and 10:1, between 0.5:1 and 5:1, between 1:1 and 20:1, between 1:1 and 10:1, between 1:1 and 5:1, between 2:1 and 20:1, between 2:1 and 10:1, or between 2:1 and 5:1. In certain other embodiments, the complexes comprise one or more lipitoids and one or more polyanionic compounds at a cation: anion charge ratio of between 0.5:1 and 50:1, between 0.5:1 and 20:1, between 0.5:1 and 10:1, between 0.5:1 and 5:1, between 1:1 and 20:1, between 1:1 and 10:1, between 1:1 and 5:1, between 2:1 and 20:1, between 2:1 and 10:1, or between 2:1 and 5:1.

In other embodiments, the complex comprises one or more cationic compounds and one or more nucleic acids at a cation: anion charge ratio of between 2:1 and 5:1. In some embodiments, the complex comprises one or more tertiary amino lipidated and/or PEGylated cationic peptide compounds and one or more nucleic acids at a cation: anion charge ratio of between 2:1 and 5:1. In yet other embodiments, the complex comprises one or more lipitoids and one or more nucleic acids at a cation: anion charge ratio of between 2:1 and 5:1. In some embodiments, the complex comprises one or more cationic compounds and one or more nucleic acids at a cation:anion charge ration of 3:1. In other embodiments, the complex comprises one or more tertiary amino lipidated and/or PEGylated cationic peptide compounds and one or more nucleic acids at a cation: anion charge ratio of 3:1. In still other embodiments, the complex comprises one or more lipitoids and one or more nucleic acids at a cation:anion charge ratio of 3:1.

Alternatively, the complexes and compositions may be characterized by the relative mass ratio of the cationic compound(s) to the polyanionic compound(s) and/or other cargoes in the complex. Mass ratios of the components in the complex can be readily calculated based upon the known concentrations and volumes of stock solutions of each component used in preparing the complex. Moreover, if non-anionic cargoes are present in the complex, mass ratios may provide a more accurate representation of the relative amounts of cationic compounds to the overall cargo than cation:anion charge ratios, which would not account for non-anionic material.

In some embodiments, the complex comprises one or more cationic compounds and one or more polyanionic compounds and/or non-anionic compounds at a mass ratio of between 0.5:1 and 50:1, between 0.5:1 and 20:1, between 0.5:1 and 10:1, between 0.5:1 and 5:1, between 1:1 and 20:1, between 1:1 and 10:1, between 1:1 and 5:1, between 2:1 and 20:1, between 2:1 and 10:1, or between 2:1 and 5:1. In certain embodiments, the complex comprises one or more tertiary amino lipidated and/or PEGylated cationic peptide compounds and one or more polyanionic compounds and/or non-anionic compounds at a mass ratio of between 0.5:1 and 50:1, between 0.5:1 and 20:1, between 0.5:1 and 10:1, between 0.5:1 and 5:1, between 1:1 and 20:1, between 1:1 and 10:1, between 1:1 and 5:1, between 2:1 and 20:1, between 2:1 and 10:1, or between 2:1 and 5:1. In other embodiments, the complex comprises one or more lipitoids and one or more polyanionic compounds and/or non-anionic compounds at a mass ratio of between 0.5:1 and 50:1, between 0.5:1 and 20:1, between 0.5:1 and 10:1, between 0.5:1 and 5:1, between 1:1 and 20:1, between 1:1 and 10:1, between 1:1 and 5:1, between 2:1 and 20:1, between 2:1 and 10:1, or between 2:1 and 5:1.

In other embodiments, the complex comprises one or more cationic compounds and the one or more polyanionic compounds and/or non-anionic compounds at a mass ratio of between 2:1 and 5:1. In certain embodiments, the complex comprises one or more tertiary amino lipidated and/or PEGylated cationic peptide compounds and the one or more polyanionic compounds and/or non-anionic compounds at a mass ratio of between 2:1 and 5:1. In some embodiments, the complex comprises one or more lipitoids and the one or more polyanionic compounds and/or non-anionic compounds at a mass ratio of between 2:1 and 5:1. In still yet other embodiments, the complex comprises one or more cationic compounds and the one or more polyanionic compounds and/or non-anionic compounds at a mass ratio of 3:1. In certain embodiments, the complex comprises one or more tertiary amino lipidated and/or PEGylated cationic peptide compounds and the one or more polyanionic compounds and/or non-anionic compounds at a mass ratio of 3:1. In yet further embodiments, the complex comprises one or more lipitoids and the one or more polyanionic compounds and/or non-anionic compounds at a mass ratio of 3:1.

In certain embodiments wherein the complex comprises one or more nucleic acids as part of the one or more polyanionic compounds, the complex may comprise one or more cationic compounds and the one or more nucleic acids at a mass ratio of between 0.5:1 and 50:1, between 0.5:1 and 20:1, between 0.5:1 and 10:1, between 0.5:1 and 5:1, between 1:1 and 20:1, between 1:1 and 10:1, between 1:1 and 5:1, between 2:1 and 20:1, between 2:1 and 10:1, or between 2:1 and 5:1. In some embodiments, the complex comprises one or more tertiary amino lipidated and/or PEGylated cationic peptide compounds and one or more nucleic acids at a mass ratio of between 0.5:1 and 50:1, between 0.5:1 and 20:1, between 0.5:1 and 10:1, between 0.5:1 and 5:1, between 1:1 and 20:1, between 1:1 and 10:1, between 1:1 and 5:1, between 2:1 and 20:1, between 2:1 and 10:1, or between 2:1 and 5:1. In other embodiments, the complex comprises one or more lipitoids and one or more nucleic acids at a mass ratio of between 0.5:1 and 50:1, between 0.5:1 and 20:1, between 0.5:1 and 10:1, between 0.5:1 and 5:1, between 1:1 and 20:1, between 1:1 and 10:1, between 1:1 and 5:1, between 2:1 and 20:1, between 2:1 and 10:1, or between 2:1 and 5:1. In some embodiments, the complex comprises one or cationic compounds and one or more nucleic acids at a mass ratio of between 2:1 and 5:1. In certain embodiments, the complex comprises one or more tertiary amino lipidated and/or PEGylated cationic peptide compounds and one or more nucleic acids at a mass ratio of between 2:1 and 5:1. In certain other embodiments, the complex comprises one or lipitoids and one or more nucleic acids at a mass ratio of between 2:1 and 5:1. In still yet other embodiments, the complex comprises one or cationic compounds and one or more nucleic acids at a mass ratio of 3:1. In certain embodiments, the complex comprises one or more tertiary amino lipidated and/or PEGylated cationic peptide compounds and one or more nucleic acids at a mass ratio of 3:1. In certain other embodiments, the complex comprises one or more lipitoids and one or more nucleic acids at a mass ratio of 3:1.

### Methods of Preparing the Complexes and Compositions

Complexes of a cationic compound and a polyanionic compound and compositions thereof can be prepared through a variety of physical and/or chemical methods to modulate their physical, chemical, and biological properties. These typically involve rapid combination of the cationic compound (e.g., an tertiary amino lipidated and/or PEGylated cationic peptide) in water or a water-miscible organic solvent with the desired polyanionic compound (e.g., oligonucleotide) in water or an aqueous buffer solution. These methods can include simple mixing of the components by pipetting, or microfluidic mixing processes such as those involving T-mixers, vortex mixers, or other chaotic mixing structures. The compositions of the present disclosure may be prepared in a similar fashion.

In one aspect, the present disclosure provides a method of preparing the complexes and compositions as described herein, comprising contacting one or more cationic compounds with one or more polyanionic compounds and one or more PEG compounds, such as PEG lipids. In some embodiments, provided herein is a method of preparing a composition, comprising contacting one or more tertiary amino lipidated and/or PEGylated cationic peptide compounds with one or more polyanionic compounds and one or more PEG compounds. In other embodiments, provided herein is a method of preparing a composition, comprising contacting one or more lipitoids with one or more polyanionic compounds and one or more PEG compounds.

In some embodiments, the method of preparing a complex comprising one or more cationic compounds, one or more PEG compounds and one or more polyanionic compounds, or a composition thereof, comprises contacting a solution comprising the one or more cationic compounds with a solution comprising the one or more polyanionic compounds and a solution comprising the one or more PEG compounds. In certain embodiments, the polyanionic compound comprises a nucleic acid. In some embodiments, the one or more cationic compounds comprise one or more tertiary amino lipidated and/or PEGylated cationic peptide compounds. In other embodiments, the one or more cationic compounds comprise one or more lipitoids.

Additional components in the complexes and composition, such as the additional components of polyethylene glycol and/or a targeting agent or excipients, may be admixed and combined with the composition before, during or after the principal components of the cationic compounds, the polyanionic compounds and the PEG compounds have been combined.

The particular process conditions for preparing the complexes and compositions described herein may be adjusted or selected accordingly to provide the desired physical properties of the compositions. For example, parameters for mixing the components of the compositions which may influence the final compositions may include but are not limited to order of mixing, temperature of mixing, mixing speed/rate, flow rate, concentrations of stock solutions, mass ratio of components (e.g., peptide:cargo), and solvents.

### Methods of Using Complexes and Compositions Thereof

As described above, the compositions described herein facilitate the delivery of polyanionic compounds to cells, particularly endocellular environments. As such, the compositions may find use in a number of clinical applications as well as research applications. The delivery of a polyanionic compound to a cell may be used for clinical applications such as those related to prophylactic, diagnostic, and/or therapeutic methods. For example, in some embodiments, suitable clinical applications may include vaccination, cancer immunotherapy, protein replacement therapy, and/or in vivo gene editing, ex vivo cell therapy transfection, ex vivo stem cell induction. Methods of delivering a polyanionic compound to a cell may also be useful for research or non-clinical applications, including for biological assays and reagents.

In another aspect, provided herein are methods of delivering a polyanionic compound to a cell. In some embodiments, the method of delivering a polyanionic compound to a cell comprises contacting the cell with a complex comprising one or more cationic compounds, one or more PEGylated compounds, and the polyanionic compound. In other embodiments, the method of delivering a polyanionic compound to a cell comprises contacting the cell with a complex, comprising one or more tertiary amino lipidated and/or PEGylated cationic peptide compounds, one or more PEGylated compounds and the polyanionic compound. In some embodiments of the foregoing methods, the contacting is by endocytosis. In still other embodiments, provided herein is a method of delivering a polyanionic compound to a cell comprising contacting the cell with a composition comprising a complex comprising one or more cationic compounds, one or more PEGylated compounds and the polyanionic compound. In other embodiments, provided herein is a method comprising contacting a cell with a composition comprising a complex, comprising one or more tertiary amino lipidated and/or PEGylated cationic peptide compounds, one or more PEGylated compounds and the polyanionic compound.

In some embodiments, the methods of delivering a polyanionic compound to a cell comprise contacting the cell with a complex comprising one or more cationic compounds, one or more PEGylated compounds and the polyanionic compound, wherein the cell is contacted in vitro, ex vivo, or in vivo. In certain embodiments, the methods of delivering a polyanionic compound to a cell comprise contacting the cell with a complex comprising one or more tertiary amino lipidated and/or PEGylated cationic peptide compounds, one or more PEGylated compounds and the polyanionic compound, wherein the cell is contacted in vitro, ex vivo, or in vivo. In other embodiments, the methods of delivering a polyanionic compound to a cell comprise contacting the cell with a composition comprising a complex comprising one or more cationic compounds, one or more PEGylated compounds and the polyanionic compound, wherein the cell is contacted in vitro, ex vivo, or in vivo. In certain embodiments, the methods of delivering a polyanionic compound to a cell comprise contacting the cell with a composition comprising a complex comprising one or more tertiary amino lipidated and/or PEGylated cationic peptide compounds, one or more PEGylated compounds and the polyanionic compound, wherein the cell is contacted in vitro, ex vivo, or in vivo.

In some embodiments wherein the cell is contacted in vitro, the cell is a HeLa, HepG2, or JAWSII cell. In other embodiments wherein the cell is contacted in vivo, the complex, or composition thereof, or formulation thereof of the present disclosure are administered to a mammalian subject. A mammalian subject may include but is not limited to a human or a mouse subject. In yet other embodiments wherein the cell is contacted ex vivo, the cell is obtained from a human or mouse subject.

In some embodiments of the foregoing methods wherein the cell is contacted in vivo, the complexes and compositions as described herein may be administered by injection. In certain embodiments, the complexes and compositions thereof as described herein may be administered by injection (intravenous (IV), subcutaneous (SC), intramuscular (IM), intrathecal, or intratumoral.) In some embodiments, the complexes and compositions are administered by intravenous (IV), subcutaneous (SC), intramuscular (IM), intrathecal, or intratumoral injection. In other embodiments, the complexes and compositions as described herein are administered by bolus injection or intravenous infusion. In other embodiments wherein the cell is contacted in vivo, the complexes and compositions of the present disclosure are administered by nasal or oral inhalation. In some embodiments wherein the cell is contacted in vivo, the complexes and compositions described herein are administered orally. In still other embodiments wherein the cell is contacted in vivo, the complexes and compositions are administered via absorption into the mucous membrane (including topical, intra-anal, buccal, intravaginal, etc.).

In another aspect, provided herein are methods of delivering a polyanionic compound to one or more target organs (e.g., lung, spleen, liver, kidney, inguinal and/or mesenteric lymph nodes) in a subject. It should be recognized that the organ specificity (for example, targeting the lungs) may be influenced by the selection of the one or more cationic compounds (e.g., tertiary amino lipidated and/or PEGylated compounds of formula (I) having at least 2+ charge, having a pKₐ within a certain range, etc.) or the mass ratios of the cationic compounds to their cargoes (e.g., a higher peptoid:cargo ratio).

It should be understood that clinical applications, such as the diagnostic, prophylactic and therapeutic examples disclosed above, may involve dosing regimens (e.g., dosage levels and time courses for administration) which may be varied as appropriate to the specific complexes and compositions being used, the route of administration, the subject to which the complexes and compositions are being administered, and/or the desired physiological effect. For example, in some embodiments, the methods of the present disclosure comprise administering the complex or composition at a dose of 0.001 mg/kg to about 1 mg/kg of bodyweight.

### ENUMERATED EMBODIMENTS

The following enumerated embodiments are representative of some aspects of the invention.
1. A composition, comprising complexes of:
   (i) one or more polyanionic compounds;
   (ii) one or more PEG compounds; and
   (iii) one or more cationic compounds, wherein the one or more cationic compounds are selected from the group consisting of:
      (a) one or more tertiary amino lipidated and/or PEGylated cationic peptide compounds of formula (I) or salts thereof:

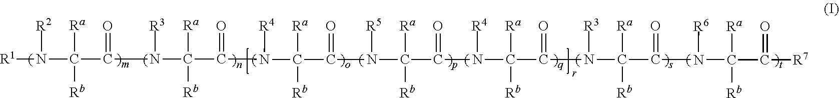

wherein:
m is 0;
n is an integer from 0 to 5;
s is an integer from 0 to 5;
t is 0;
   wherein at least one of n and s is nonzero;
r is an integer from 1 to 20;
each o is independently an integer 0, 1, or 2;
each q is independently an integer 0, 1, or 2;
each p is independently an integer 1 or 2;
R¹ is —H, alkyl, alkylaryl, —COR^{1a} or a lipid moiety,
   wherein R^{1a} is —H, —OH, alkyl, aryl, alkylaryl, —O-alkyl, or —O-alkylaryl;
each R² is independently an ethylene glycol moiety of the formula —CH₂CH₂O(CH₂CH₂O)ᵤCH₃, and wherein each u is independently an integer from 2 to 200;
each R³ is independently a lipid moiety;
each R⁴ is independently a neutral spacer moiety or a lipid moiety;
each R⁵ is independently a cationic moiety;
each R⁶ is independently an ethylene glycol moiety of the formula —CH₂CH₂O(CH₂CH₂O)ᵥCH₃, and wherein each v is independently an integer from 2 to 200;
R⁷ is —H, alkyl, acyl, —OH, —OR^{7a}, —NH₂, —NHR^{7a}, or a lipid moiety, wherein R^{7a} is alkyl, acyl, or a lipid moiety; and
each R^{a} and R^{b} are independently —H, C₁-C₄-alkyl, or a side chain moiety found on a naturally- or non-naturally-occurring amino acid;
   (b) one or more lipitoids of formula (II):

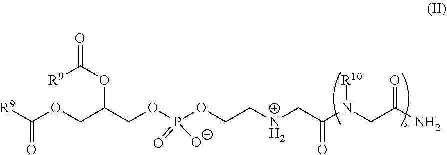

wherein:
x is a integer from 1 to 100;
each R⁹ is independently a lipid moiety; and
each R¹⁰ is independently a cationic or neutral spacer moiety;
   (c) one or more lipid-like compounds of formula (III):

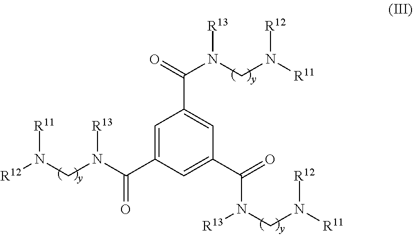

wherein:
each R¹¹ is independently substituted or unsubstituted alkyl;
each R¹² is independently substituted or unsubstituted alkyl;
each R¹³ is independently hydrogen or substituted or unsubstituted alkyl; and
each y is independently an integer from 1 to 8;
and
   (d) any combination thereof;
and wherein the one or more cationic compounds, the one or more PEG compounds and the one or more polyanionic compounds have a combined mass percentage of at least 90% w/w of the complexes.
2. The composition of embodiment 1, wherein the one or more cationic compounds are one or more tertiary amino lipidated and/or PEGylated cationic peptide compounds of formula (I).
3. The composition of embodiment 1 or embodiment 2, wherein the one or more tertiary amino lipidated and/or PEGylated cationic peptide compounds comprises a block of N-lipidated amino acid residues, wherein either n or s is at least 2.
4. The composition of any one of embodiments 1 to 3, wherein each R³ is independently C₄-C₂₂-alkyl or C₄-C₂₂-alkenyl, and wherein the C₄-C₂₂-alkenyl is optionally mono- or poly-unsaturated.
5. The composition of any one of embodiments 1 to 4, wherein each R³ is independently C₈-C₁₂ alkyl.
6. The composition of any one of embodiments 1 to 4, wherein each R³ is independently selected from the group consisting of 2-ethylhex-1-yl, caproyl, oleyl, stearyl, linoleyl, myristyl, and lauryl.
7. The composition of any one of embodiments 1 to 6, wherein at least one of the one or more tertiary amino lipidated and/or PEGylated cationic peptide compounds comprises a cationic domain comprising at least two cationic amino acid residues having cationic moieties R⁵.
8. The composition of any one of embodiments 1 to 7, wherein each R⁵ is independently aminoalkyl, alkylaminoalkyl, aminoalkylaminoakyl, guanidinoalkyl, N-heterocyclylalkyl or N-heteroaryl.
9. The composition of any one of embodiments 1 to 8, wherein each R⁵ is independently selected from the group consisting of:

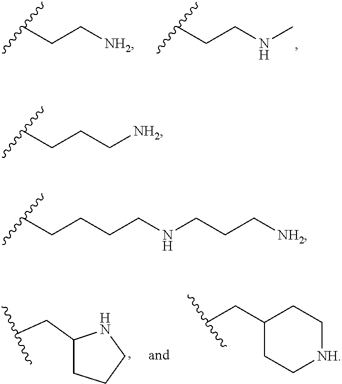

10. The composition of any one of embodiments 1 to 9, wherein each R⁵ is

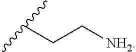 [SMILES: *CCN]

11. The composition of any one of embodiments 1 to 10, wherein each neutral spacer moiety R⁴ is independently a C₁-C₄-alkyl substituted by cycloalkyl, heterocyclylalkyl, alkylaryl, arylalkyl, alkylheteroaryl, heteroarylalkyl, alkoxy, alkoxyalkyl, or hydroxyalkyl, wherein each cycloalkyl, heterocyclylalkyl, alkylaryl, arylalkyl, alkylheteroaryl, heteroarylalkyl, alkoxy, alkoxyalkyl, or hydroxyalkyl is optionally substituted with one or more substituents —OH, halo, or alkoxy.
12. The composition of any one of embodiments 1 to 11, wherein each neutral spacer moiety R⁴ is independently selected from the group consisting of:

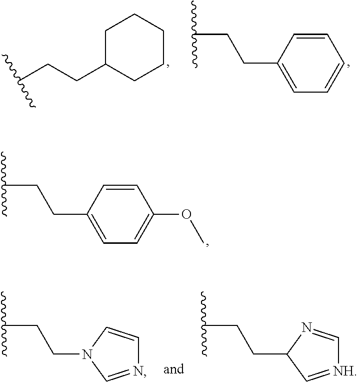

13. The composition of any one of embodiments 1 to 12, wherein each neutral spacer moiety R⁴ is

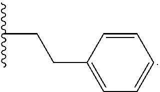 [SMILES: *CCc1ccccc1]

14. The composition of any one of embodiments 1 to 13, wherein at least one of the one or more tertiary amino lipidated and/or PEGylated cationic peptide compounds comprises a cationic domain comprising at least two amino acid residues having R⁵ cationic moieties and wherein each of the at least two cationic amino acid residues within the cationic domain are separated by at least one amino acid residue having a neutral spacer or lipid moiety R⁴.
15. The composition of any one of embodiments 1 to 14, wherein at least one of the one or more tertiary amino lipidated and/or PEGylated cationic peptide compounds comprises at least one trimer subunit —R^{cation}—R^{neutral}—R^{neutral}, and wherein R^{cation} is an amino acid residue comprising a cationic moiety R⁵ and each R^{neutral} is an amino acid residue comprising a neutral spacer moiety R⁴.
16. The composition of any one of embodiments 1 to 15, wherein each cationic moiety R⁵ is

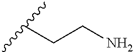 [SMILES: *CCN]

and each neutral spacer moiety R⁴ is

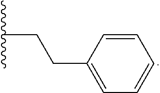 [SMILES: *CCc1ccccc1]

17. The composition of any one of embodiments 1 to 16, wherein R^{a} and R^{b} are independently selected from the group consisting of —H and —CH₃.
18. The composition of any one of embodiments 1 to 17, wherein R^{a} and R^{b} are —H.
19. The composition of any one of embodiments 1 to 18, wherein the one or more cationic peptide compounds and one or more PEG compounds are present at a mass ratio of peptide compound(s)-to-PEG compound(s) between 90:10 and 99:1.
20. The composition of any one of embodiments 1 to 19, wherein the one or more cationic peptide compounds and the one or more PEG compounds are present at a mass ratio of peptide compounds-to-PEG compound(s) between 95:5 and 98:2.
21. The composition of any one of embodiments 1 to 20, wherein the one or more PEG compounds comprises DMG-PEG2000.
22. The composition of any one of embodiments 1 to 21, wherein the one or more PEG compounds comprises one or more tertiary amino lipidated and/or PEGylated cationic peptide compounds of formula (I) or salts thereof:

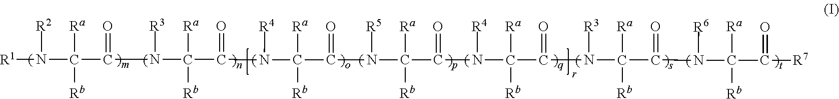

wherein:
m is an integer from 0 to 10;
n is an integer from 0 to 5;
s is an integer from 0 to 5;
t is an integer from 0 to 10;
   wherein at least one of m and t is nonzero;
r is an integer from 1 to 20;
each o is independently an integer 0, 1, or 2;
each q is independently an integer 0, 1, or 2;
each p is independently an integer 1 or 2;
R¹ is —H, alkyl, alkylaryl, —COR^{1a} or a lipid moiety,
wherein R^{1a} is —H, —OH, alkyl, aryl, alkylaryl, —O-alkyl, or —O-alkylaryl;
each R² is independently an ethylene glycol moiety of the formula —CH₂CH₂O(CH₂CH₂O)ᵤCH₃, and wherein each u is independently an integer from 2 to 200;
each R³ is independently a lipid moiety;
each R⁴ is independently a neutral spacer moiety or a lipid moiety;
each R⁵ is independently a cationic moiety;
each R⁶ is independently an ethylene glycol moiety of the formula —CH₂CH₂O(CH₂CH₂O)ᵥCH₃, and wherein each v is independently an integer from 2 to 200;
R⁷ is-H, alkyl, acyl, —OH, —OR^{7a}, —NH₂, —NHR^{7a}, or a lipid moiety,
wherein R^{7a} is alkyl, acyl, or a lipid moiety; and
each R^{a} and R^{b} are independently —H, C₁-C₄-alkyl, or a side chain moiety found on a naturally- or non-naturally-occurring amino acid.
23. The composition of embodiment 22, wherein
   when m is an integer from 0 to 3, each u is independently an integer from 20 to 200, optionally from 30 to 50; and
   when m is an integer from 4 to 10, each u is independently an integer from 2 to 10.
24. The composition of embodiment 22 or embodiment 23, wherein
   when t is an integer from 0 to 3, each v is independently an integer from 30 to 50;
   when t is an integer from 4 to 10, each v is independently an integer from 2 to 10.
25. The composition of any one of embodiments 22 to 24, wherein
   m is 1, and u is an integer from 40 to 45; or
   t is 1, and v is an integer from 40 to 45.
26. The composition of any one of embodiments 1 to 25, wherein the one or more polyanionic compounds comprises a nucleic acid.
27. The composition of any one of embodiments 1 to 26, the one or more polyanionic compounds comprises a nucleic acid and wherein the mass ratio of the one or more cationic peptide compounds to the nucleic acid is between 0.5:1 and 20:1.
28. The composition of any one of embodiments 1 to 27, wherein the one or more polyanionic compounds comprises a nucleic acid and wherein the nucleic acid is an mRNA encoding a polypeptide.
29. The composition of any one of embodiments 1 to 28, wherein the one or more polyanionic compounds comprises a nucleic acid and wherein the nucleic acid is an mRNA encoding a protein.
30. The composition of any one of embodiments 1 to 29, wherein the complexes further comprise one or more second agents.
31. The composition of any one of embodiments 1 to 30, wherein the one or more second agents are selected from the group consisting of polyethylene glycol, a targeting element, and a combination thereof.
32. The composition of any one of embodiments 1 to 31, wherein the complexes further comprise one or more small molecule active agents or drug substances.
33. The composition of any one of embodiments 1 to 31, wherein the complexes are suspended in an aqueous solution.
34. A method of delivering a polyanionic compound to a cell comprising contacting the cell with the composition of any one of embodiments 1 to 33.
35. The method of embodiment 34, wherein the contacting is by endocytosis.
36. The method of embodiment 34 or embodiment 35, wherein the cell is contacted in vivo or in vitro.
37. The method of any one of embodiments 35 to 36, wherein the polyanionic compound comprises the nucleic acid which is the mRNA encoding a polypeptide and the cell expresses the polypeptide after being contacted with the composition.
38. The method of any one of embodiments 34 to 37, wherein the polyanionic compound comprises the nucleic acid which is the mRNA encoding a protein and the cell expresses the protein after being contacted with the composition.
39. A method of preparing a composition of any one of embodiments 1 to 33, comprising contacting the one or more cationic peptide compounds with the one or more PEG compounds and the one or more polyanionic compounds.
40. The method of embodiment 39, comprising contacting a solution comprising the one or more cationic peptide compounds and one or more PEG compounds with a solution comprising the one or more polyanionic compounds.

### EXAMPLES

The present disclosure is described in further detail in the following examples which are not in any way intended to limit the scope of the disclosure as claimed. The attached figures are meant to be considered as integral parts of the specification and description of the disclosure. The following examples are offered to illustrate, but not to limit the claimed disclosure.

### Example 1—Synthesis of Exemplary Tertiary Amino Lipidated Cationic Peptides for Nucleic Acid Delivery

The following example describes the general protocol for synthesis of the tertiary amino lipidated and/or PEGylated cationic peptide compounds of formula (I) as described herein.

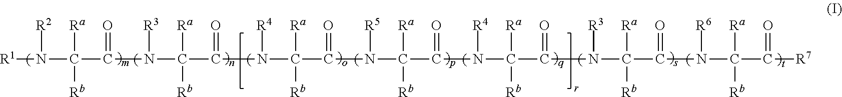

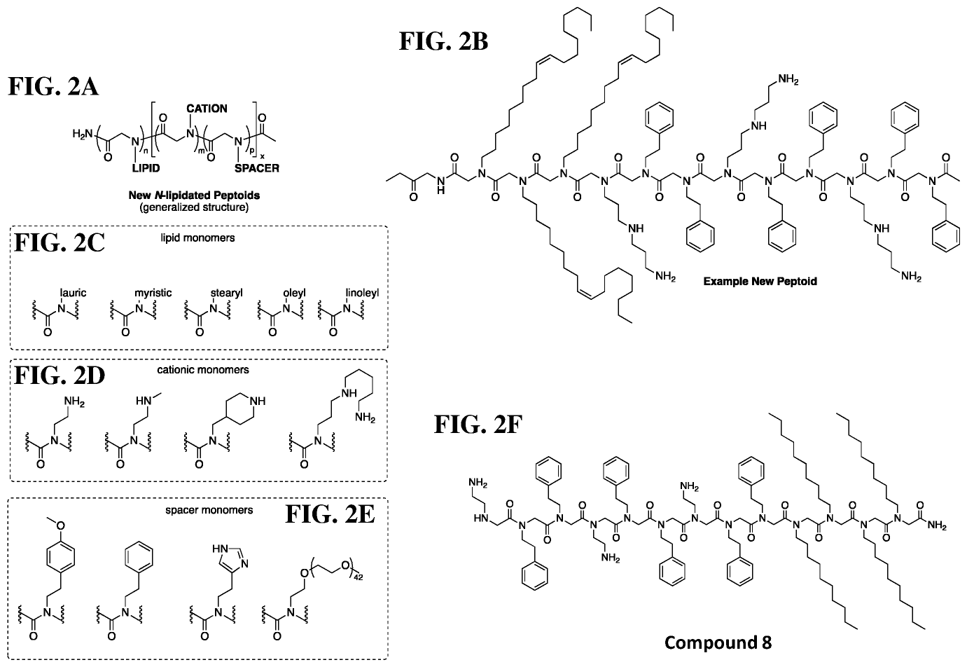

In the description provided below, all R^{a} and R^{b} are —H. All polymers are synthesized using bromoacetic acid and primary amines. FIGS. 2B-2E provides some of the exemplary substituents of the primary amines at R², R³, R⁴, R⁵, and R⁶ to prepare the tertiary amino lipidated and/or PEGylated cationic peptide compounds of the present disclosure.

An Fmoc-Rink amide resin is used as the solid support. The Fmoc group on the resin is deprotected with 20% (v/v) piperidine-dimethylformamide (DMF). The amino resin is then amidated with bromoacetic acid. This is followed by amination of the α-carbon by nucleophilic displacement of the bromide with a primary amine. The two steps are successively repeated to produce the desired cationic peptide sequence.

All reactions and washings are performed at room temperature unless otherwise noted. Washing of the resin refers to the addition of a wash solvent (usually DMF or dimethylsulfoxide (DMSO)) to the resin, agitating the resin so that a uniform slurry is obtained, followed by thorough draining of the solvent from the resin. Solvents are removed by vacuum filtration through the fritted bottom of the reaction vessel until the resin appeared dry. In all the syntheses, resin slurries are agitated via bubbling argon up through the bottom of the fritted vessel.

Initial Resin Deprotection. A fritted reaction vessel is charged with Fmoc-Rink amide resin. DMF is added to the resin and this solution is agitated to swell the resin. The DMF is then drained. The Fmoc group is removed by adding 20% piperidine in DMF to the resin, agitating the resin, and draining the resin. 20% piperidine in DMF is added to the resin and agitated for 15 minutes and then drained. The resin is then washed with DMF, six times.

Acylation/Amidation. The deblocked amine is then acylated by adding bromoacetic acid in DMF to the resin followed by N,N-diisoprooplycarbodiimide (DIC) in DMF (FIG. 3A). This solution is agitated for 30 minutes at room temperature and then drained. This step is repeated a second time. The resin is then washed with DMF twice and DMSO once. This is one completed reaction cycle.

Nucleophilic Displacement/Amination. The acylated resin is treated with the desired primary or secondary amine to undergo nucleophilic displacement at the bromine leaving group on the α-carbon (FIG. 3B). This acylation/displacement cycle is repeated (FIG. 3C) until the desired peptide sequence is obtained (FIG. 3D).

Peptide Cleavage from Resin. The dried resin is placed in a glass scintillation vial containing a teflon-coated micro stir bar, and 95% trifluoroacetic acid (TFA) in water is added. The solution is stirred for 20 minutes and then filtered through solid-phase extraction (SPE) column fitted with a polyethylene frit into a polypropylene conical centrifuge tube.

The resin is washed with 1 mL 95% TFA. The combined filtrates are then lyophilized three times from 1:1 acetonitrile:water. The lyophilized peptide (FIG. 3E) is redissolved in absolute ethanol at a concentration of 5 mg/mL or in DMSO at a concentration of 10 mg/mL.

Purification and Characterization. The redissolved crude peptide is purified by preparative HPLC. The purified peptide is characterized by LC-MS analysis.

### Example 2—Synthesis and Characterization of Representative Amino Lipidated Peptoids

Amino lipidated peptoids were synthesized by the submonomer method described above in Example 1 with bromoacetic acid and N,N′-diisopropylcarbodiimide (DIC). Polystyrene-supported MBHA Fmoc-protected Rink amide (200 mg representative scale, 0.64 mmol/g loading, Protein Technologies) resin was used as a solid support. For bromoacetylation, resin was combined with a 1:1 mixture of 2 M bromoacetic acid and 2M N,N′-diisopropylcarbodiimide (DIC) for 5 minutes. Amine displacement was carried out using a 1M solution of amine in DMF for 1 hour. Following synthesis, crude peptoids were cleaved from resin using 5 mL of a mixture of 95:2.5:2.5 trifluoroacetic acid (TFA):water:triisopropylsilane for 40 minutes at room temperature. Resin was removed by filtration and the filtrate concentrated using a Biotage V10 evaporator. The crude peptoids were further concentrated by lyophilization from a 25% solution of MeCN in water. Purity and identity were assayed with a Waters Acquity UPLC system with Acquity Diode Array UV detector and Waters SQD2 mass spectrometer on a Waters Acquity UPLC Peptide BEH C18 Column over a 5-95% gradient. Select crude peptoids were purified by preparative Waters Prep150LC system with Waters 2489 UV/Visible Detector on a Waters XBridge BEH300 Prep C4 column using a 5-40% acetonitrile in water with 0.1% TFA gradient over 30 minutes.

Table 1A shows representative amino lipidated peptoids prepared by the method described in Example 2. Table 1B provides characterization data for the amino lipidated peptoid compounds 1-72 prepared in Table 1A, including the predicted molecular weight, retention time (in minutes, determined by UPLC-UV measurement at λ=218 nm), and primary observed mass-to-charge ratio (m/z, MH⁺, by electrospray ionization-mass spectrometry). For each of amino lipidated and PEGylated peptoid compounds 48, 56, 64, and 72, the mass spectra contained several distributions of peaks due to the polydispersity of the PEG moiety (average molecular weight 2000 g/mol) attached to the peptoid. The mass-to-charge ratio peak reported for these amino lipidated and PEGylated peptoid compounds is the central value of the MH₂²⁺ distribution of peaks, with an effective monomer separation δ−22 m/z (—OCH₂CH₂—, ethylene glycol molecular weight 44 g/mol).

TABLE 1A

| # | Compound Structure |
|---|---|
| 1 | 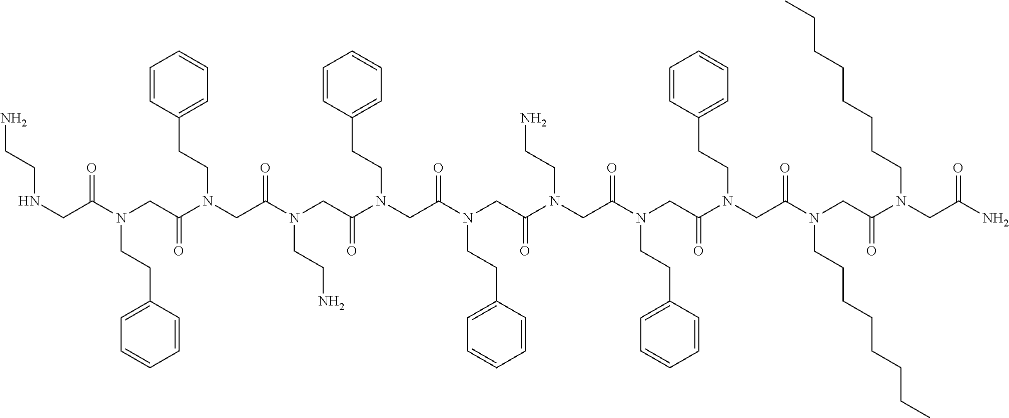 [SMILES: CCCCCCCCN(CC(N)=O)C(=O)CN(CCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CNCCN] |
| 2 | 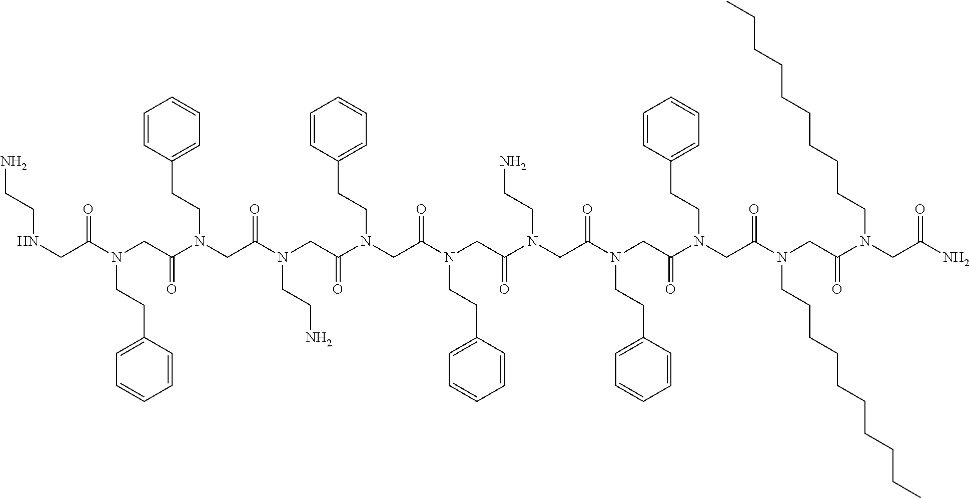 [SMILES: CCCCCCCCCCN(CC(N)=O)C(=O)CN(CCCCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CNCCN] |
| 3 | 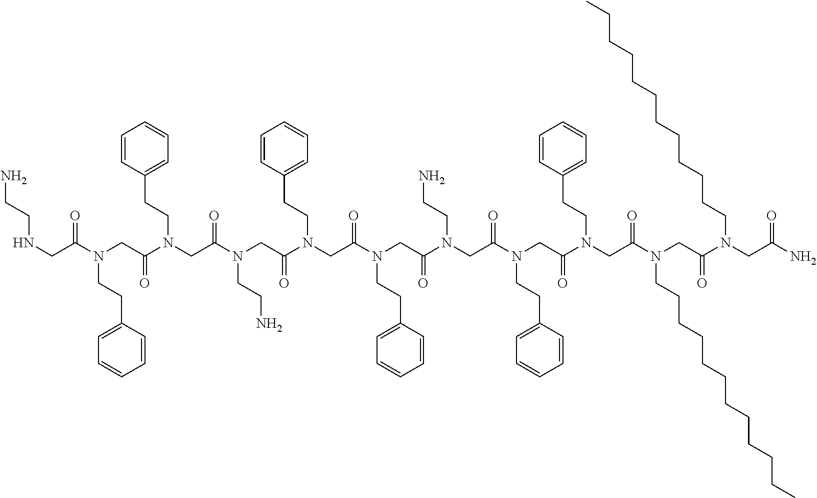 [SMILES: CCCCCCCCCCCCN(CC(N)=O)C(=O)CN(CCCCCCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CNCCN] |
| 4 | 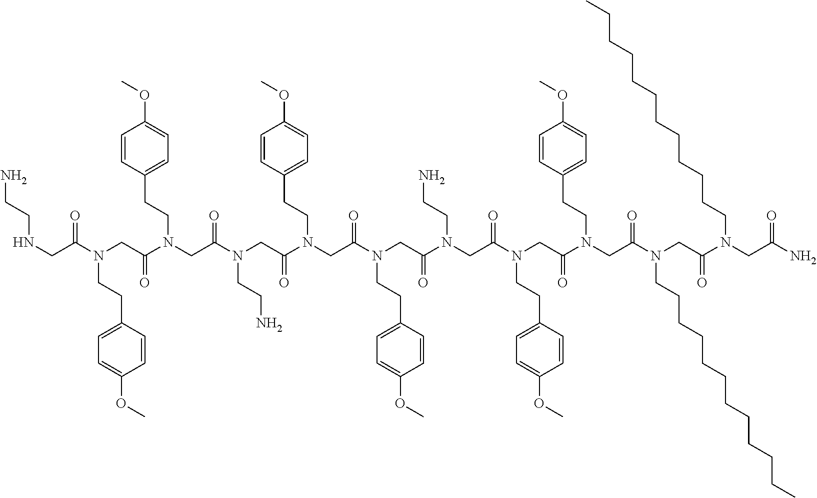 [SMILES: CCCCCCCCCCCCN(CC(N)=O)C(=O)CN(CCCCCCCCCCCC)C(=O)CN(CCc1ccc(OC)cc1)C(=O)CN(CCc1ccc(OC)cc1)C(=O)CN(CCN)C(=O)CN(CCc1ccc(OC)cc1)C(=O)CN(CCc1ccc(OC)cc1)C(=O)CN(CCN)C(=O)CN(CCc1ccc(OC)cc1)C(=O)CN(CCc1ccc(OC)cc1)C(=O)CNCCN] |
| 5 | 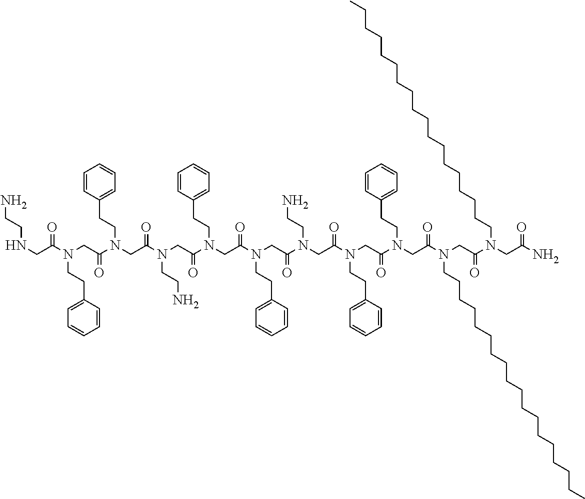 [SMILES: CCCCCCCCCCCCCCCCCCN(CC(N)=O)C(=O)CN(CCCCCCCCCCCCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CNCCN] |
| 6 | 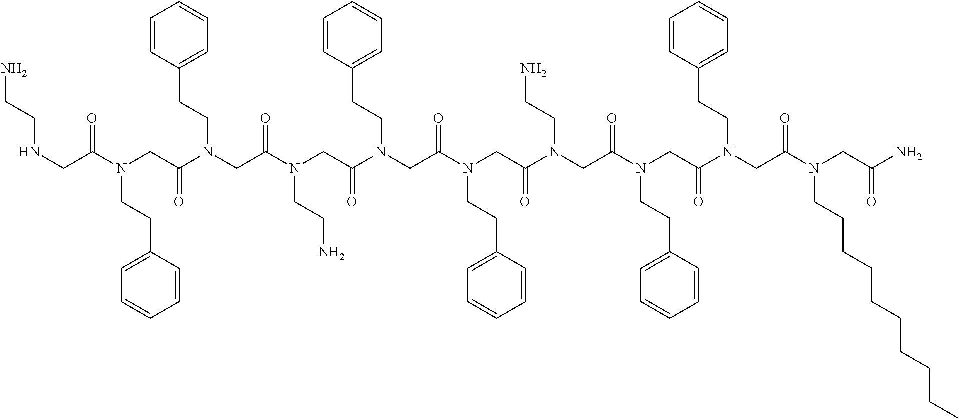 [SMILES: CCCCCCCCCCN(CC(N)=O)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CNCCN] |
| 7 | 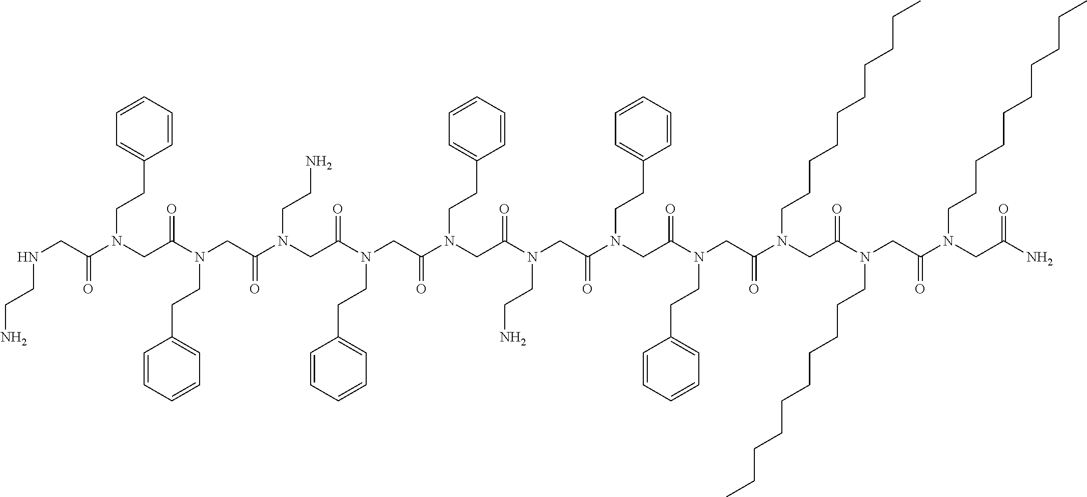 [SMILES: CCCCCCCCCCN(CC(N)=O)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CNCCN] |
| 8 | 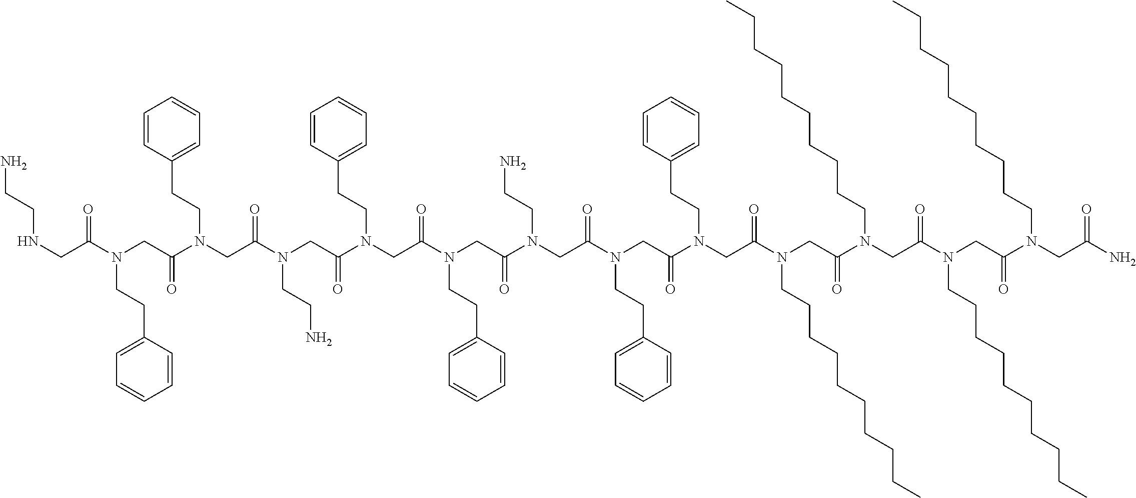 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CNCCN] |
| 9 | 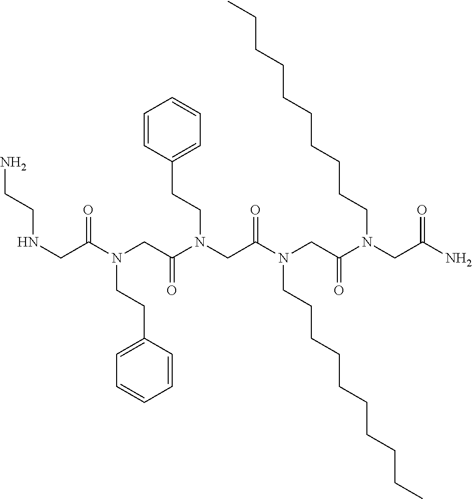 [SMILES: CCCCCCCCCCN(CC(N)=O)C(=O)CN(CCCCCCCCCC)C(=O)CN(CCc1ccccc1)C(=O)CN(CCc1ccccc1)C(=O)CNCCN] |
| 10 | 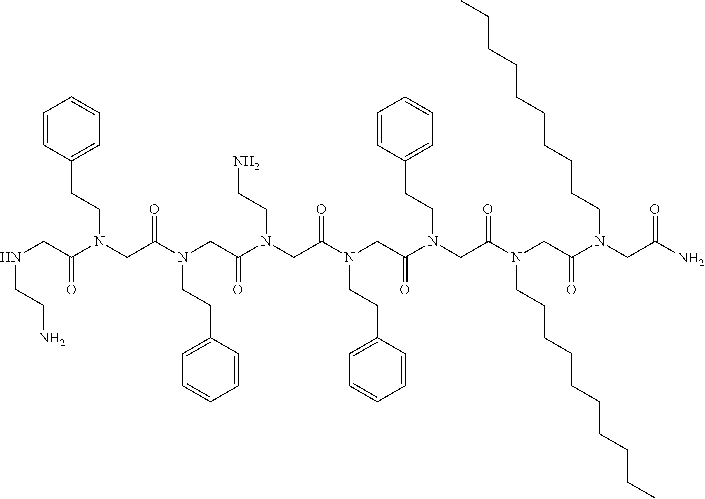 [SMILES: CCCCCCCCCCN(CC(N)=O)C(=O)CN(CCCCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CNCCN] |
| 11 | 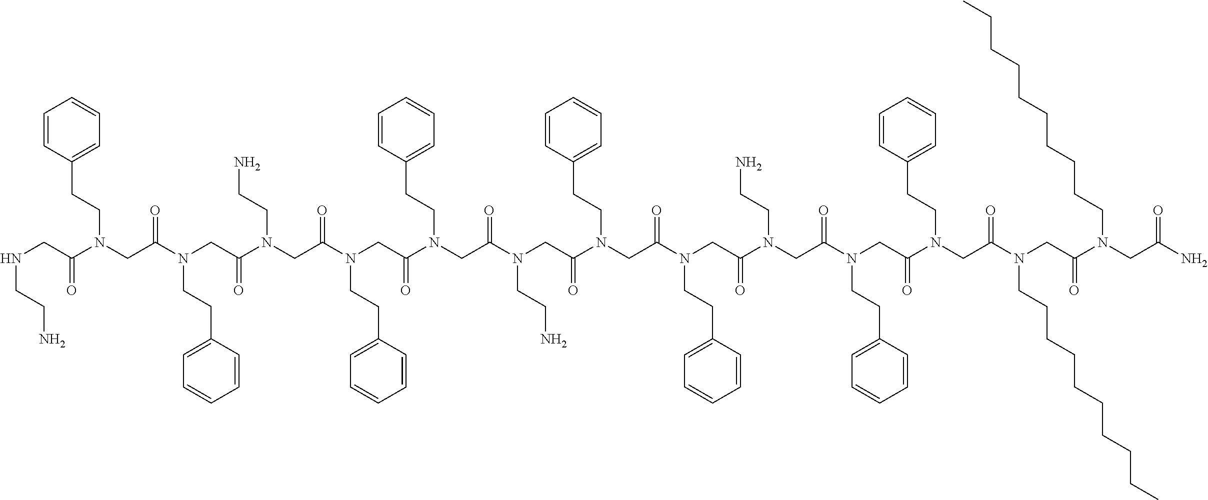 [SMILES: CCCCCCCCCCN(CC(N)=O)C(=O)CN(CCCCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CNCCN] |
| 12 | 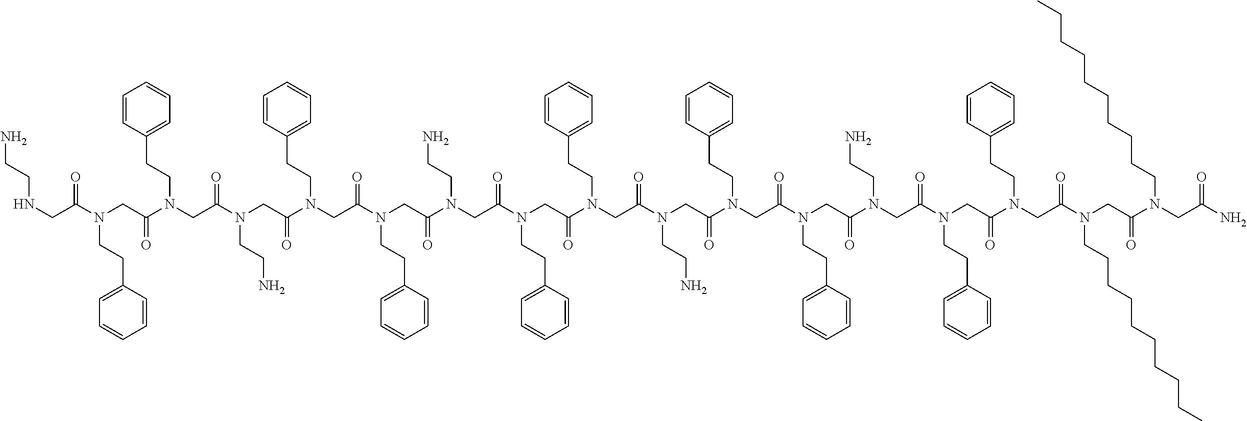 [SMILES: CCCCCCCCCCN(CC(N)=O)C(=O)CN(CCCCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CNCCN] |
| 13 | 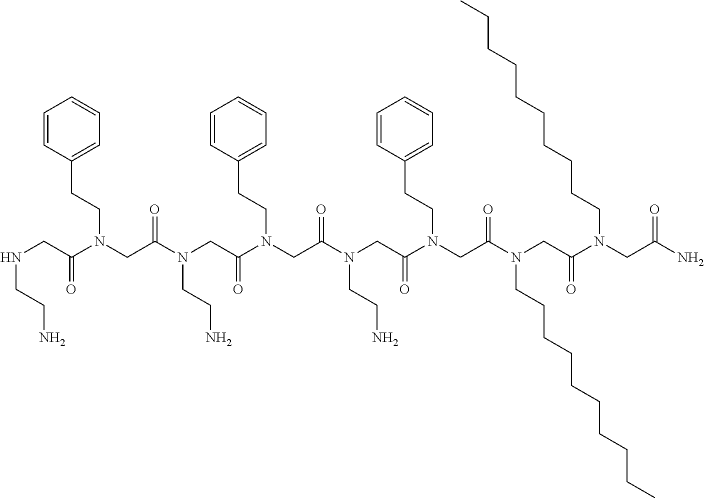 [SMILES: CCCCCCCCCCN(CC(N)=O)C(=O)CN(CCCCCCCCCC)C(=O)CN(CCc1ccccc1)C(=O)CN(CCN)C(=O)CN(CCc1ccccc1)C(=O)CN(CCN)C(=O)CN(CCc1ccccc1)C(=O)CNCCN] |
| 14 | 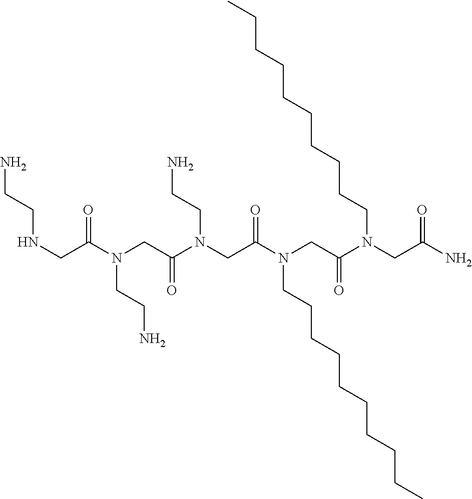 [SMILES: CCCCCCCCCCN(CC(N)=O)C(=O)CN(CCCCCCCCCC)C(=O)CN(CCN)C(=O)CN(CCN)C(=O)CNCCN] |
| 15 | 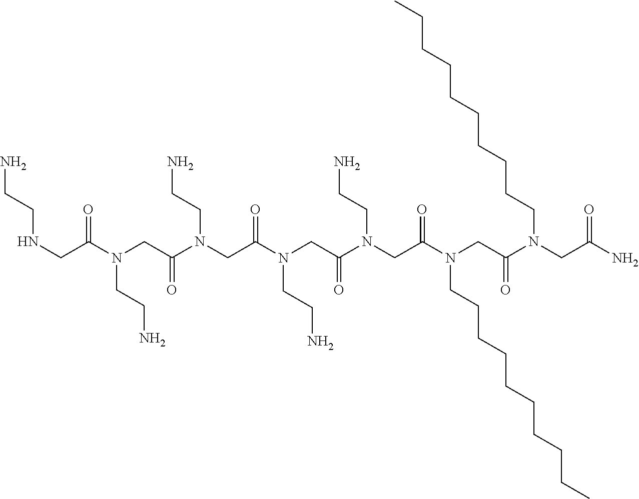 [SMILES: CCCCCCCCCCN(CC(N)=O)C(=O)CN(CCCCCCCCCC)C(=O)CN(CCN)C(=O)CN(CCN)C(=O)CN(CCN)C(=O)CN(CCN)C(=O)CNCCN] |
| 16 | 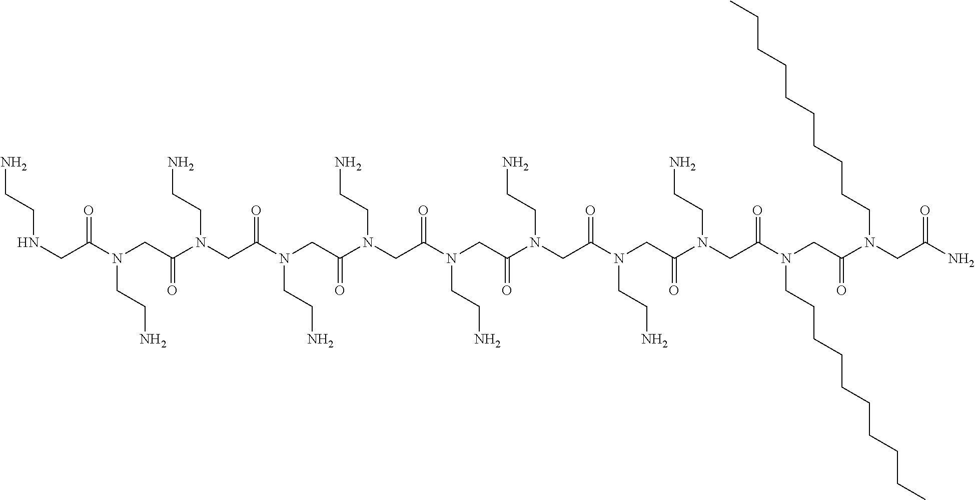 [SMILES: CCCCCCCCCCN(CC(N)=O)C(=O)CN(CCCCCCCCCC)C(=O)CN(CCN)C(=O)CN(CCN)C(=O)CN(CCN)C(=O)CN(CCN)C(=O)CN(CCN)C(=O)CN(CCN)C(=O)CN(CCN)C(=O)CN(CCN)C(=O)CNCCN] |
| 17 | 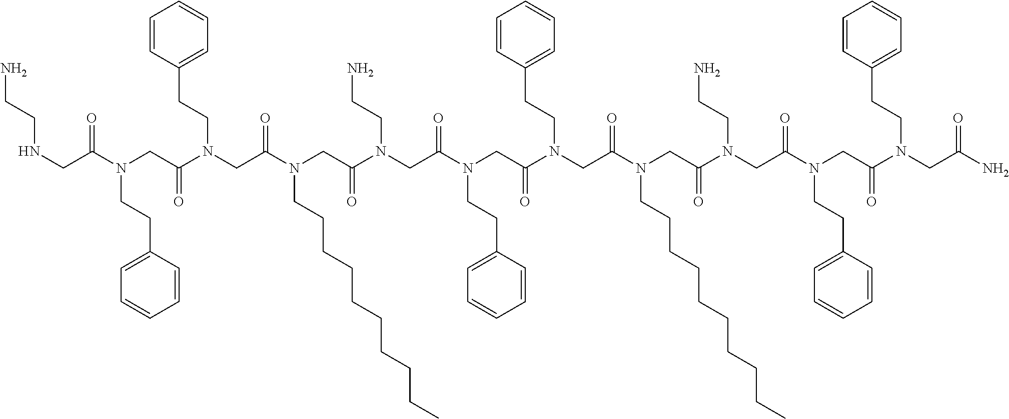 [SMILES: CCCCCCCCCCN(CC(=O)N(CCN)CC(=O)N(CCc1ccccc1)CC(=O)N(CCc1ccccc1)CC(=O)N(CCCCCCCCCC)CC(=O)N(CCN)CC(=O)N(CCc1ccccc1)CC(=O)N(CCc1ccccc1)CC(N)=O)C(=O)CN(CCc1ccccc1)C(=O)CN(CCc1ccccc1)C(=O)CNCCN] |
| 18 | 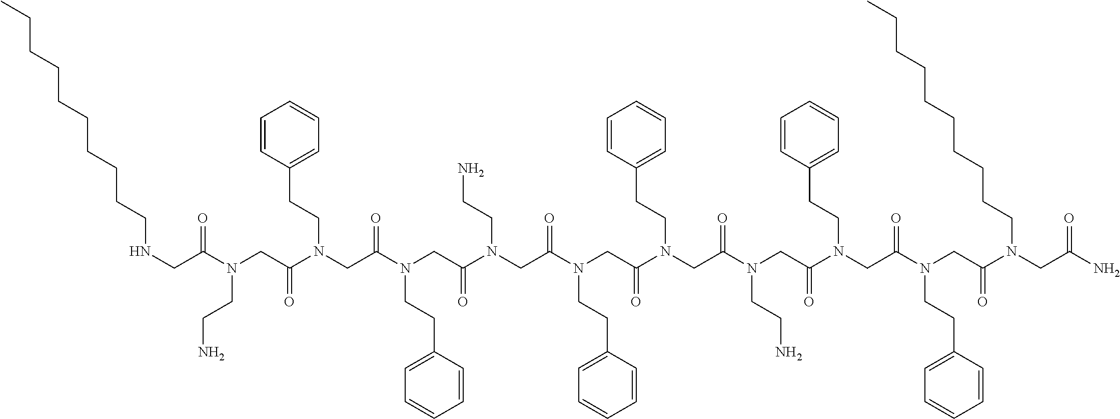 [SMILES: CCCCCCCCCCNCC(=O)N(CCN)CC(=O)N(CCc1ccccc1)CC(=O)N(CCc1ccccc1)CC(=O)N(CCN)CC(=O)N(CCc1ccccc1)CC(=O)N(CCc1ccccc1)CC(=O)N(CCN)CC(=O)N(CCc1ccccc1)CC(=O)N(CCc1ccccc1)CC(=O)N(CCCCCCCCCC)CC(N)=O] |
| 19 | 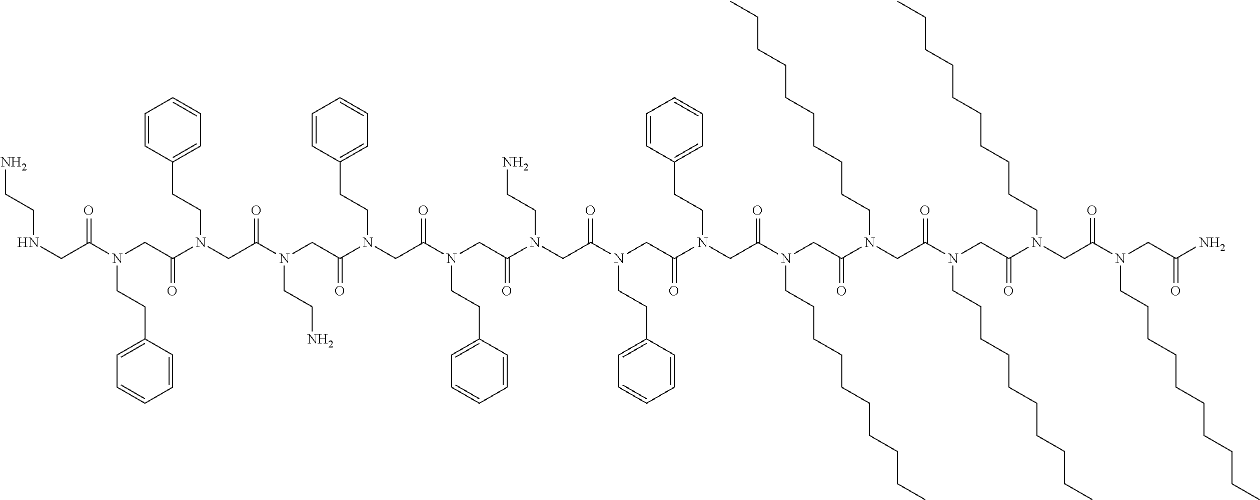 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CNCCN] |
| 20 | 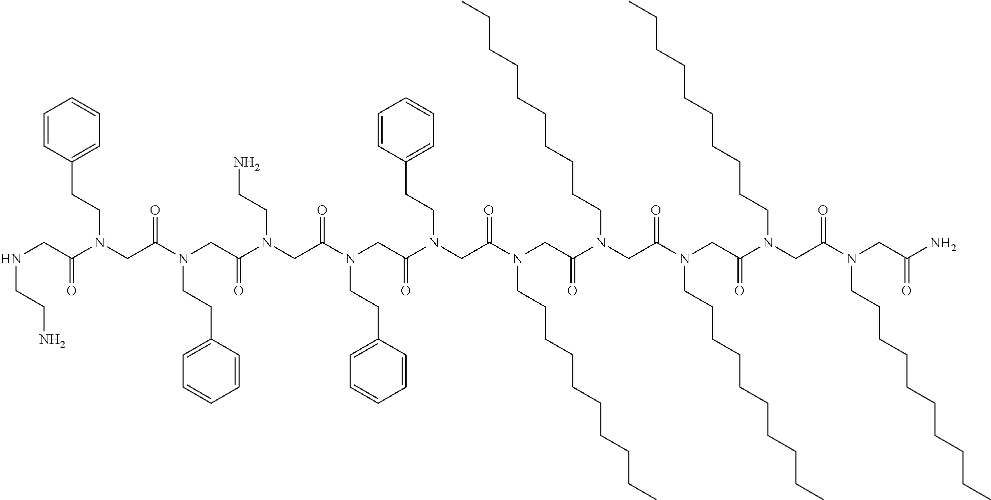 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CNCCN] |
| 21 | 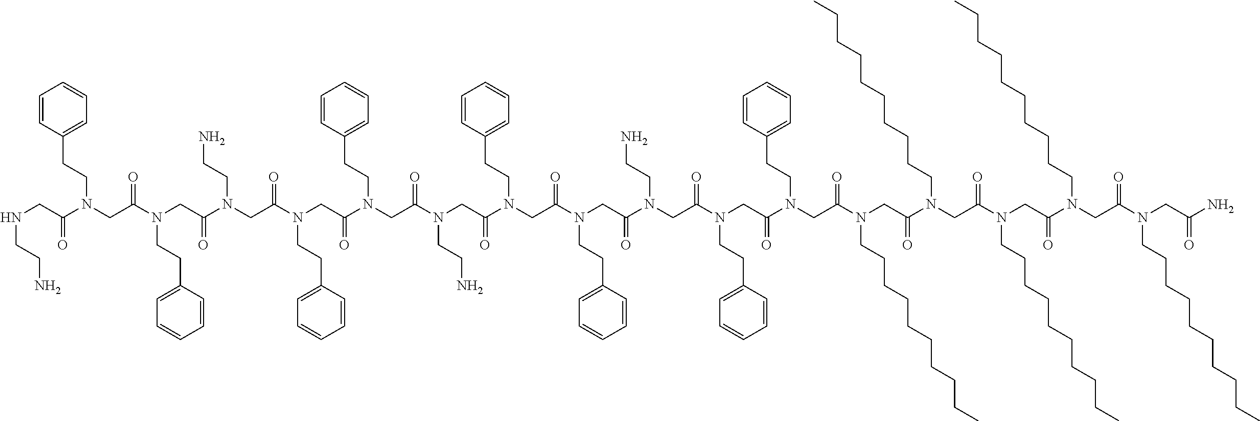 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CNCCN] |
| 22 | 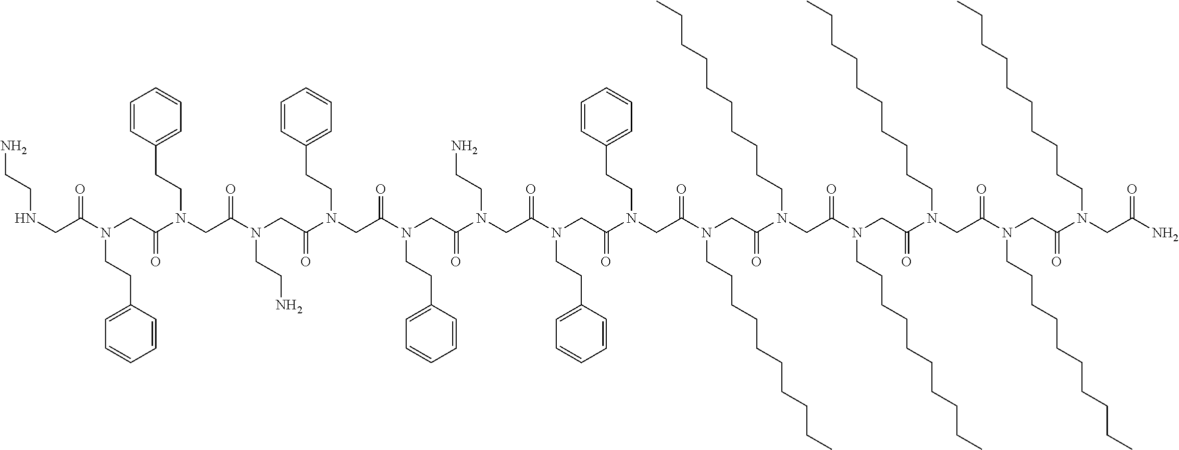 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CNCCN] |
| 23 | 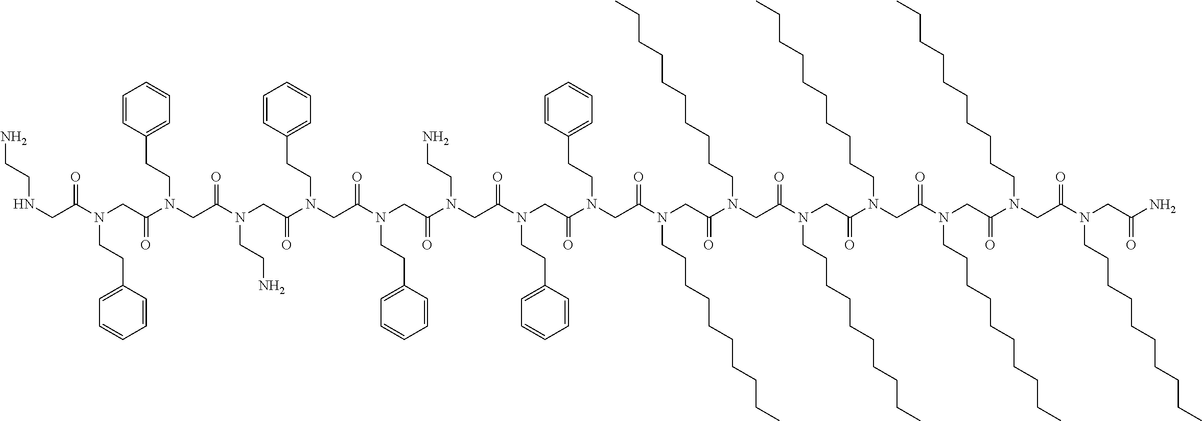 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CNCCN] |
| 24 | 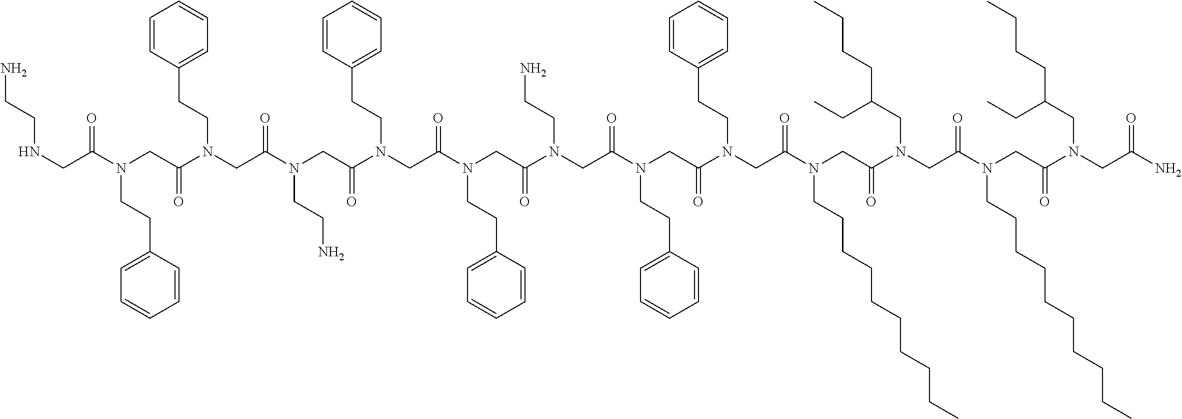 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CNCCN] |
| 25 | 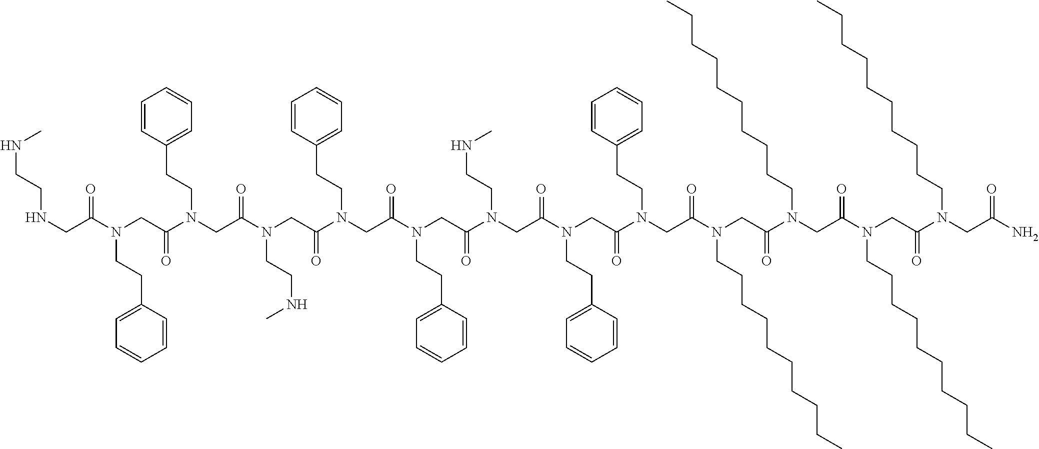 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCNC)C(=O)CN(CCc1ccccc1)C(=O)CN(CCc1ccccc1)C(=O)CN(CCNC)C(=O)CN(CCc1ccccc1)C(=O)CN(CCc1ccccc1)C(=O)CNCCNC] |
| 26 | 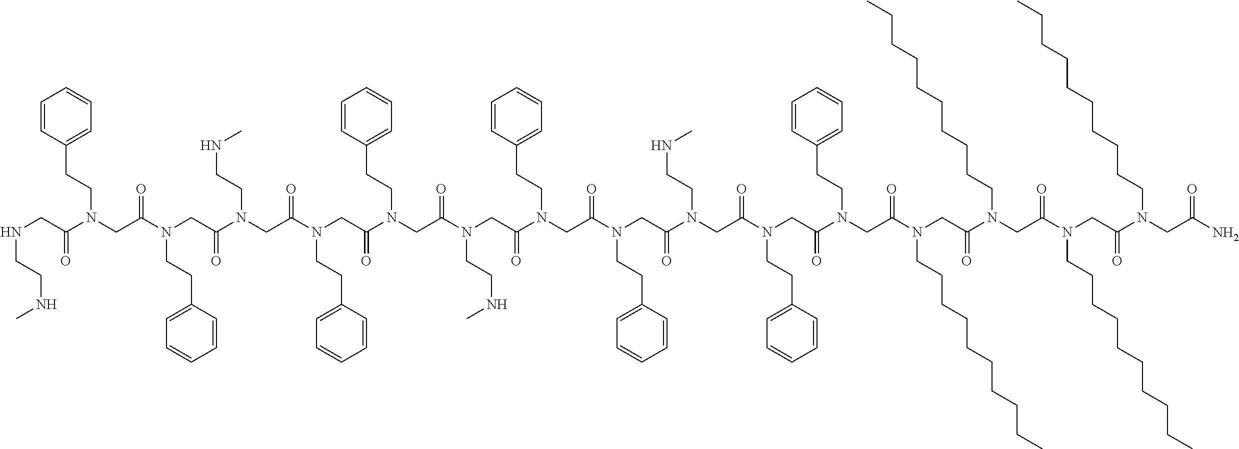 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCNC)C(=O)CN(CCc1ccccc1)C(=O)CN(CCc1ccccc1)C(=O)CN(CCNC)C(=O)CN(CCc1ccccc1)C(=O)CN(CCc1ccccc1)C(=O)CN(CCNC)C(=O)CN(CCc1ccccc1)C(=O)CN(CCc1ccccc1)C(=O)CNCCNC] |
| 27 | 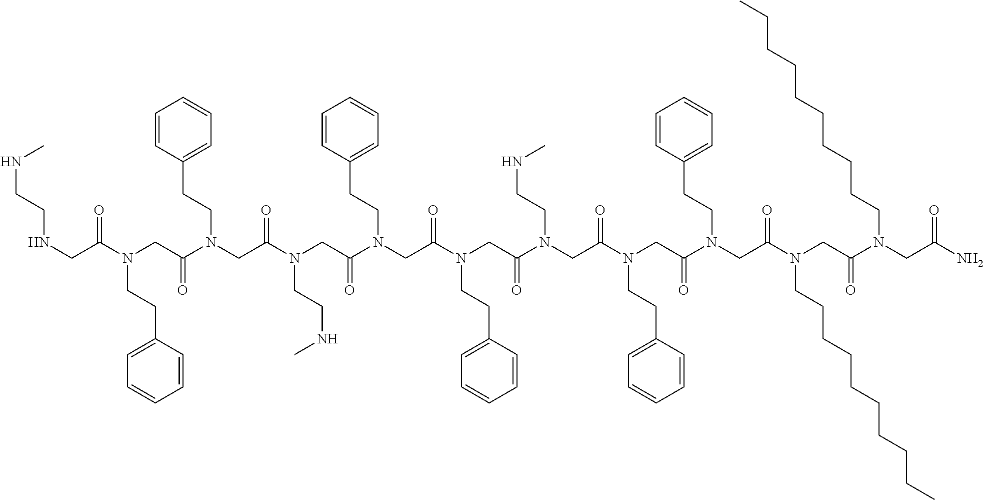 [SMILES: CCCCCCCCCCN(CC(N)=O)C(=O)CN(CCCCCCCCCC)C(=O)CN(CCc1ccccc1)C(=O)CN(CCc1ccccc1)C(=O)CN(CCNC)C(=O)CN(CCc1ccccc1)C(=O)CN(CCc1ccccc1)C(=O)CN(CCNC)C(=O)CN(CCc1ccccc1)C(=O)CN(CCc1ccccc1)C(=O)CNCCNC] |
| 28 | 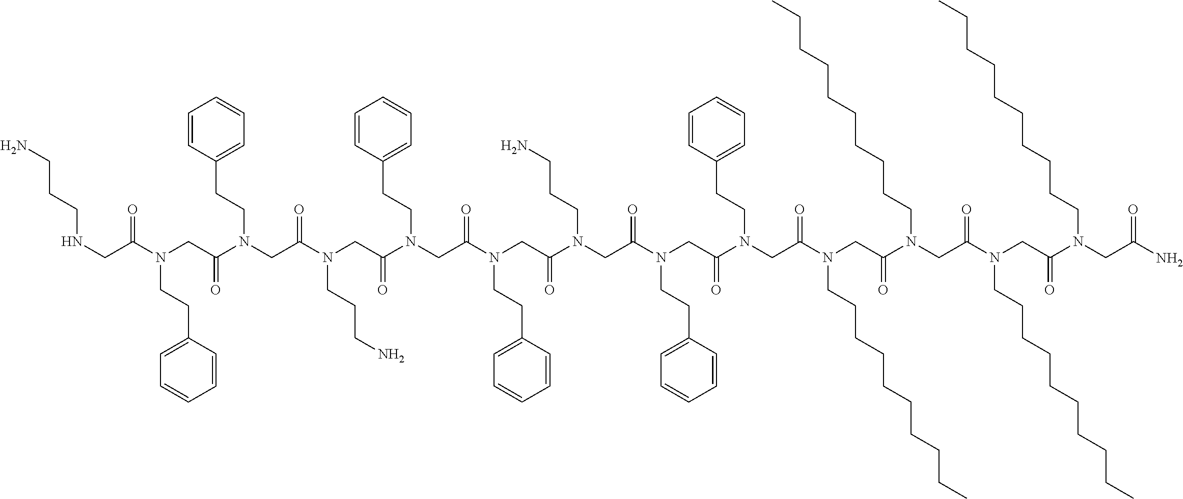 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCCN)C(=O)CN(CCc1ccccc1)C(=O)CN(CCc1ccccc1)C(=O)CN(CCCN)C(=O)CN(CCc1ccccc1)C(=O)CN(CCc1ccccc1)C(=O)CNCCCN] |
| 29 | 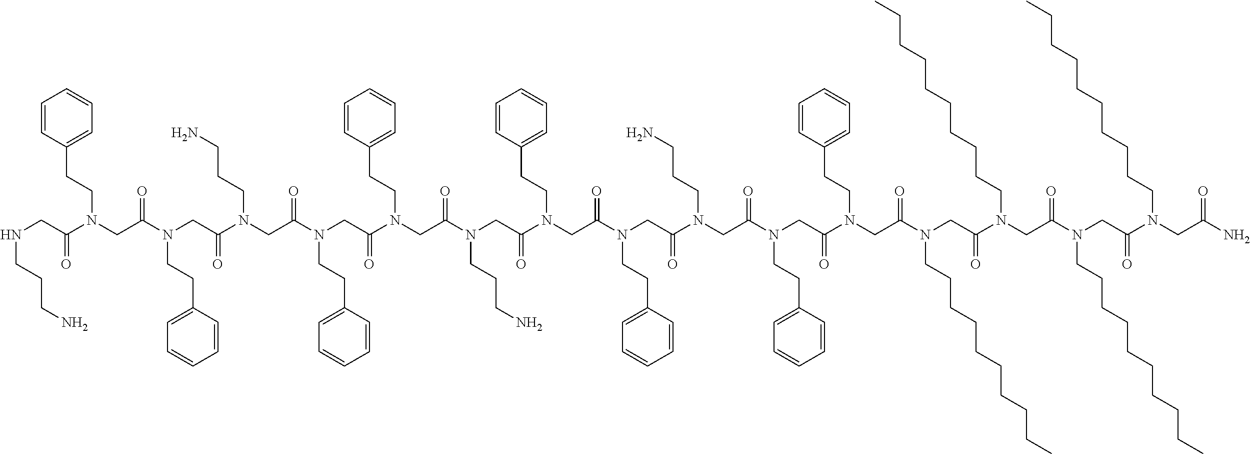 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCCN)C(=O)CN(CCc1ccccc1)C(=O)CN(CCc1ccccc1)C(=O)CN(CCCN)C(=O)CN(CCc1ccccc1)C(=O)CN(CCc1ccccc1)C(=O)CN(CCCN)C(=O)CN(CCc1ccccc1)C(=O)CN(CCc1ccccc1)C(=O)CNCCCN] |
| 30 | 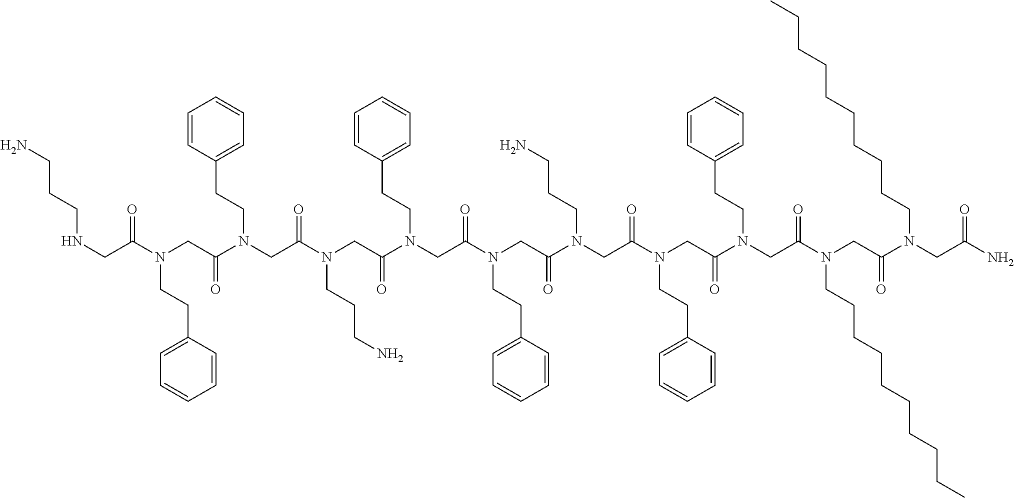 [SMILES: CCCCCCCCCCN(CC(N)=O)C(=O)CN(CCCCCCCCCC)C(=O)CN(CCc1ccccc1)C(=O)CN(CCc1ccccc1)C(=O)CN(CCCN)C(=O)CN(CCc1ccccc1)C(=O)CN(CCc1ccccc1)C(=O)CN(CCCN)C(=O)CN(CCc1ccccc1)C(=O)CN(CCc1ccccc1)C(=O)CNCCCN] |
| 31 | 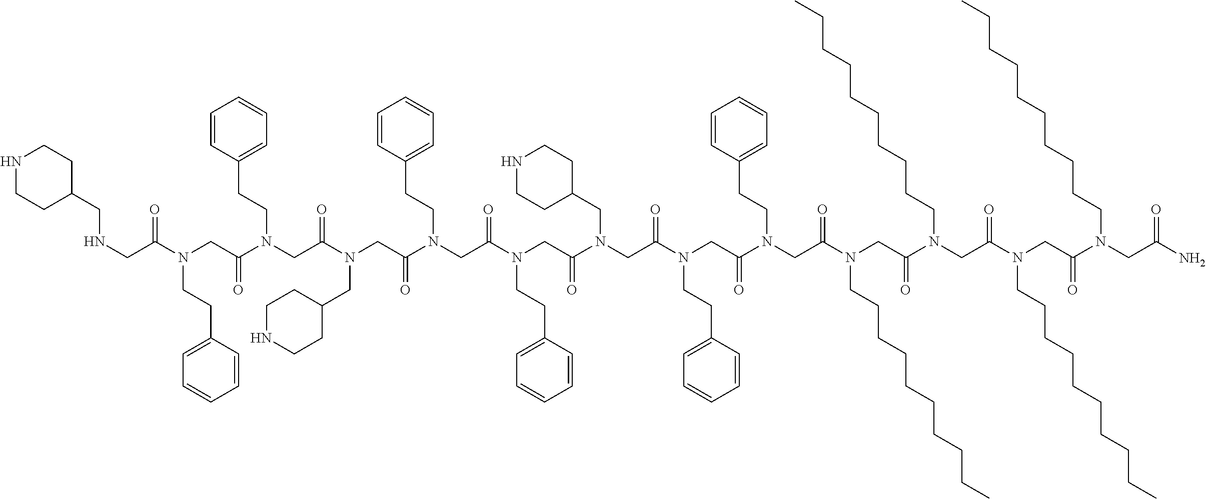 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CC1CCNCC1)C(=O)CN(CCc1ccccc1)C(=O)CN(CCc1ccccc1)C(=O)CN(CC1CCNCC1)C(=O)CN(CCc1ccccc1)C(=O)CN(CCc1ccccc1)C(=O)CNCC1CCNCC1] |
| 32 | 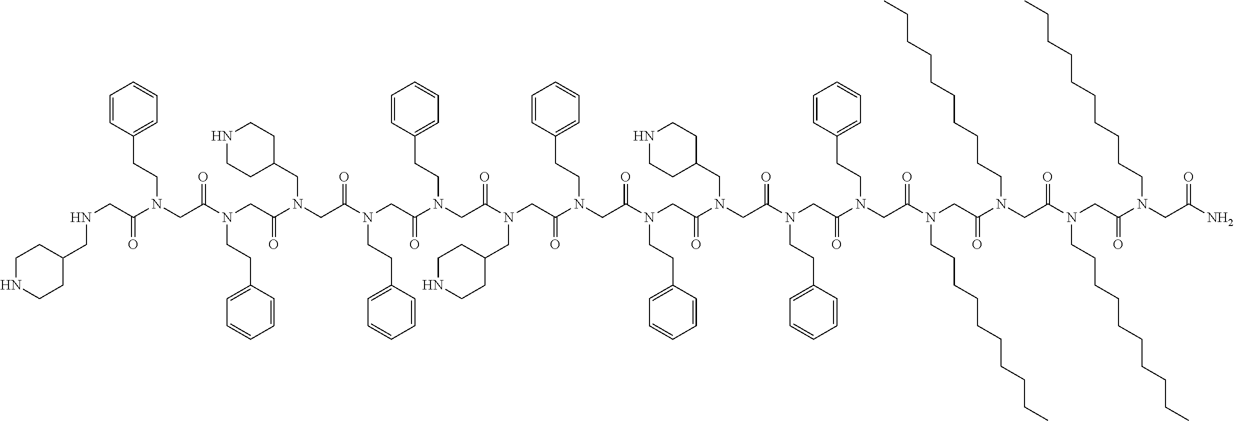 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CC1CCNCC1)C(=O)CN(CCc1ccccc1)C(=O)CN(CCc1ccccc1)C(=O)CN(CC1CCNCC1)C(=O)CN(CCc1ccccc1)C(=O)CN(CCc1ccccc1)C(=O)CN(CC1CCNCC1)C(=O)CN(CCc1ccccc1)C(=O)CN(CCc1ccccc1)C(=O)CNCC1CCNCC1] |
| 33 | 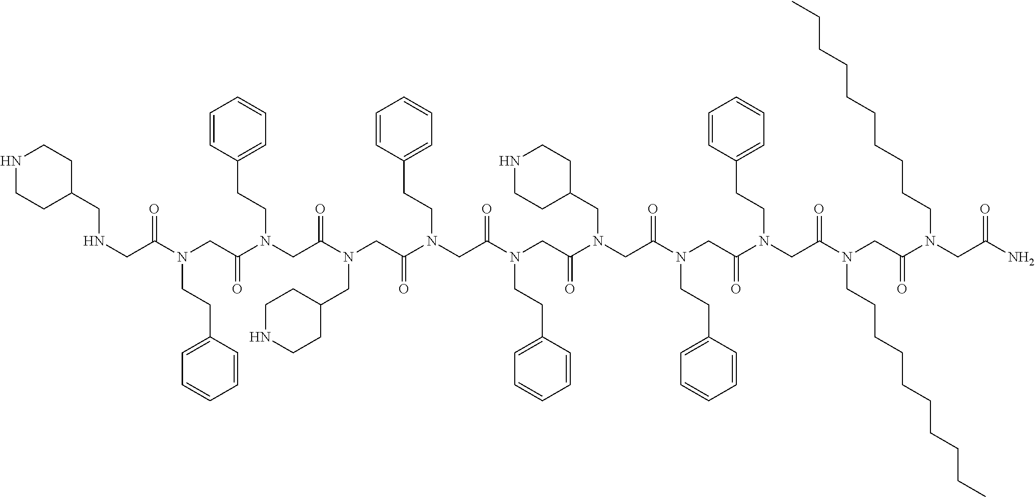 [SMILES: CCCCCCCCCCN(CC(N)=O)C(=O)CN(CCCCCCCCCC)C(=O)CN(CCc1ccccc1)C(=O)CN(CCc1ccccc1)C(=O)CN(CC1CCNCC1)C(=O)CN(CCc1ccccc1)C(=O)CN(CCc1ccccc1)C(=O)CN(CC1CCNCC1)C(=O)CN(CCc1ccccc1)C(=O)CN(CCc1ccccc1)C(=O)CNCC1CCNCC1] |
| 34 | 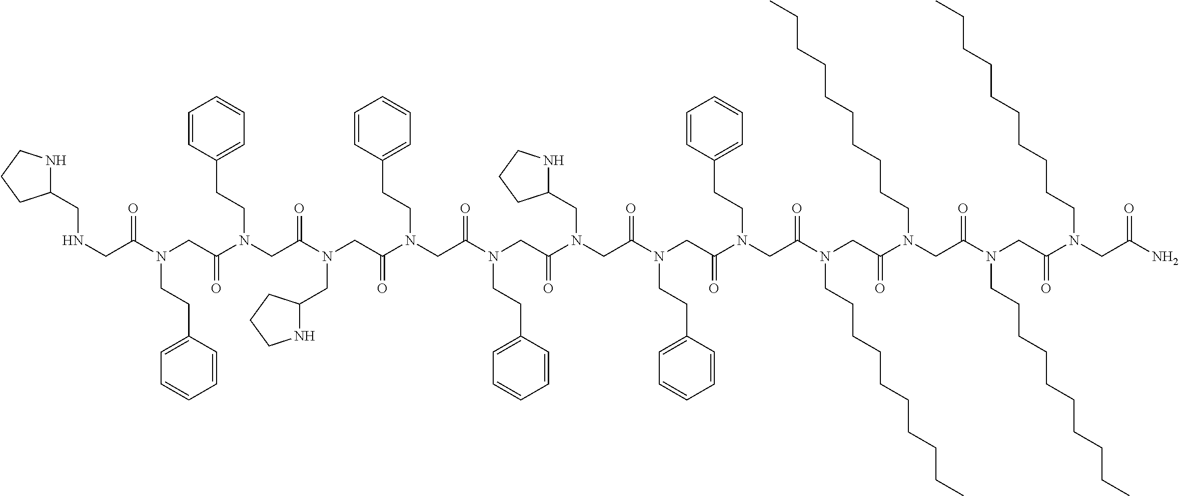 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CC1CCCN1)C(=O)CN(CCc1ccccc1)C(=O)CN(CCc1ccccc1)C(=O)CN(CC1CCCN1)C(=O)CN(CCc1ccccc1)C(=O)CN(CCc1ccccc1)C(=O)CNCC1CCCN1] |
| 35 | 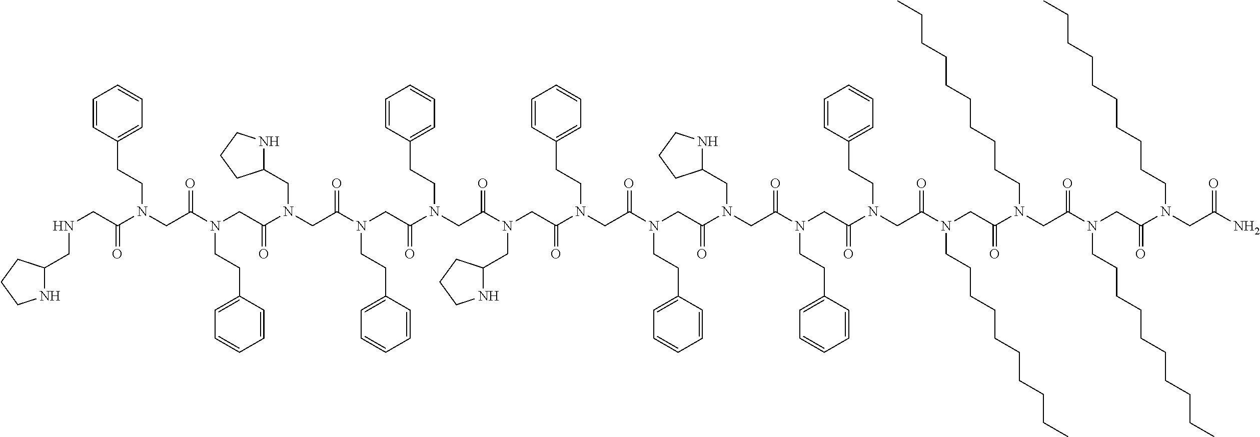 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CC1CCCN1)C(=O)CN(CCc1ccccc1)C(=O)CN(CCc1ccccc1)C(=O)CN(CC1CCCN1)C(=O)CN(CCc1ccccc1)C(=O)CN(CCc1ccccc1)C(=O)CN(CC1CCCN1)C(=O)CN(CCc1ccccc1)C(=O)CN(CCc1ccccc1)C(=O)CNCC1CCCN1] |
| 36 | 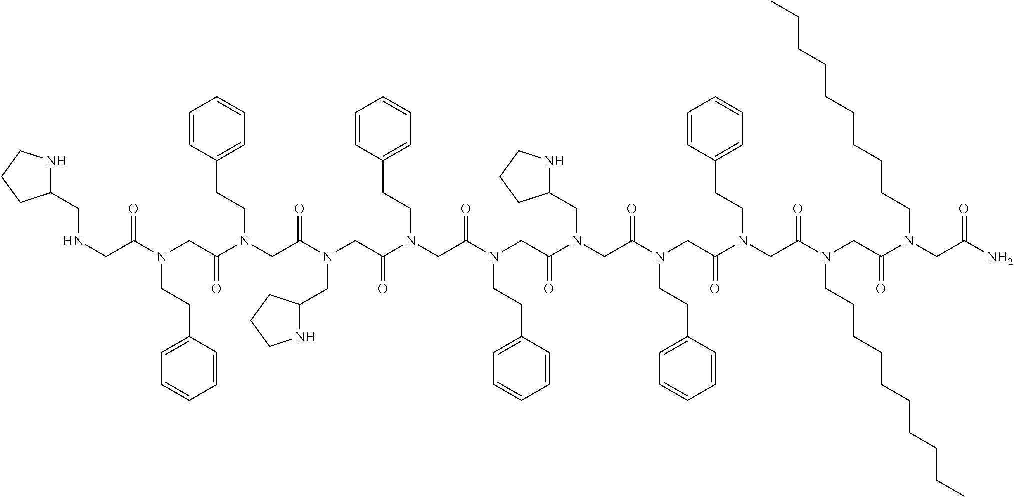 [SMILES: CCCCCCCCCCN(CC(N)=O)C(=O)CN(CCCCCCCCCC)C(=O)CN(CCc1ccccc1)C(=O)CN(CCc1ccccc1)C(=O)CN(CC1CCCN1)C(=O)CN(CCc1ccccc1)C(=O)CN(CCc1ccccc1)C(=O)CN(CC1CCCN1)C(=O)CN(CCc1ccccc1)C(=O)CN(CCc1ccccc1)C(=O)CNCC1CCCN1] |
| 37 | 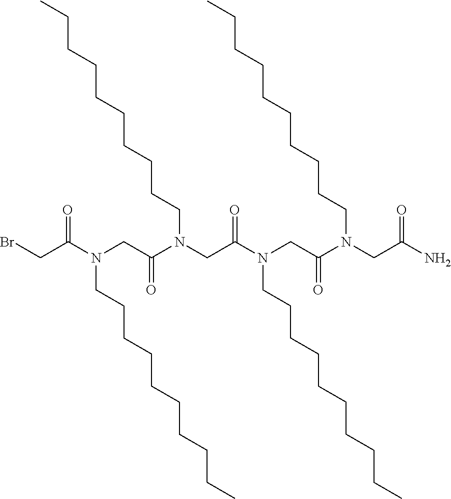 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CBr] |
| 38 | 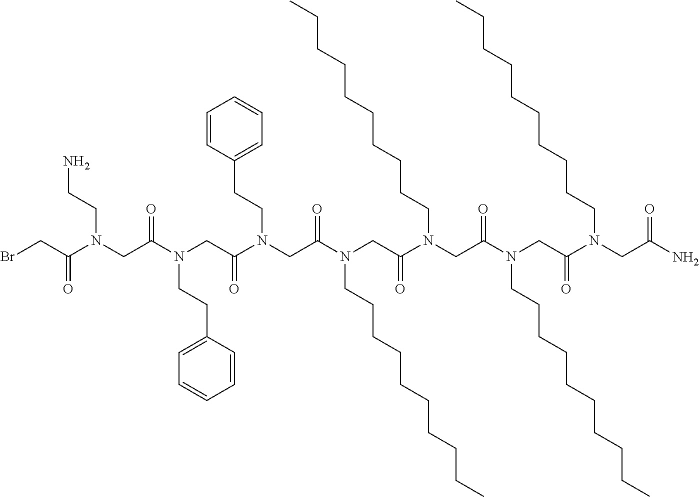 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CBr] |
| 39 | 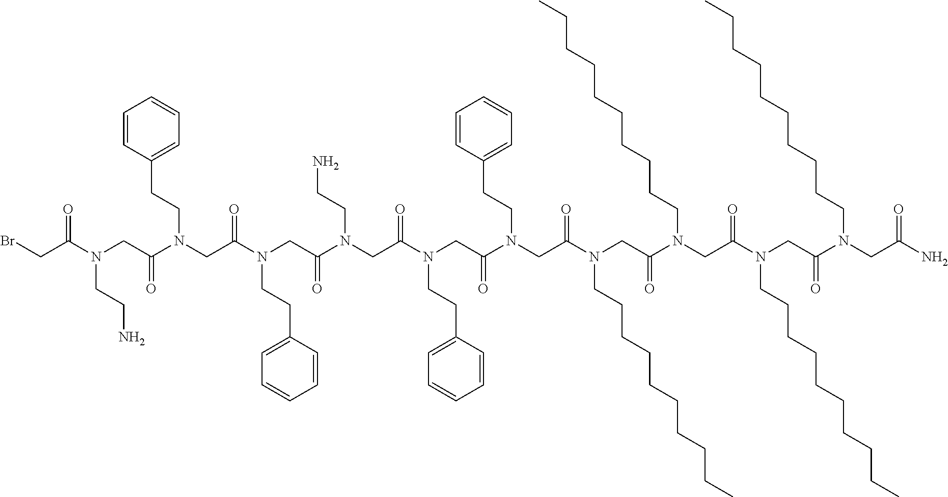 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CBr] |
| 40 | 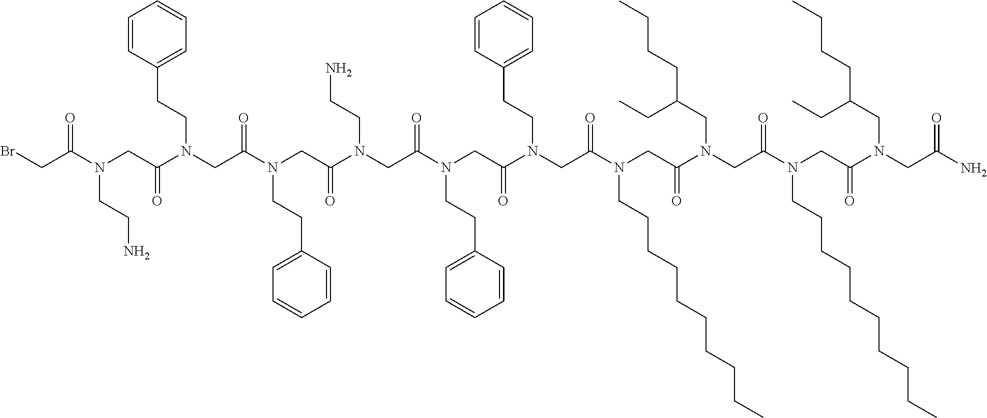 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CBr] |
| 41 | 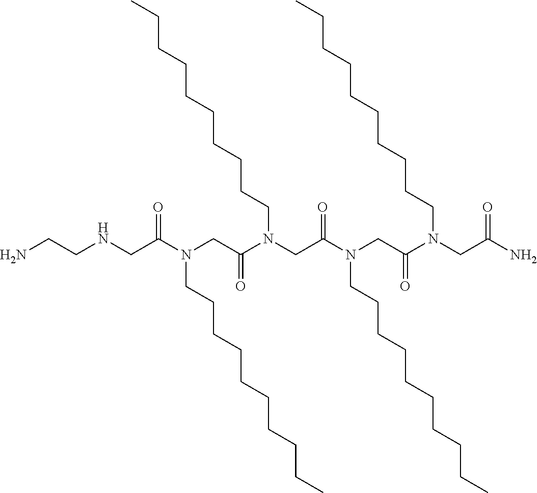 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CNCCN] |
| 42 | 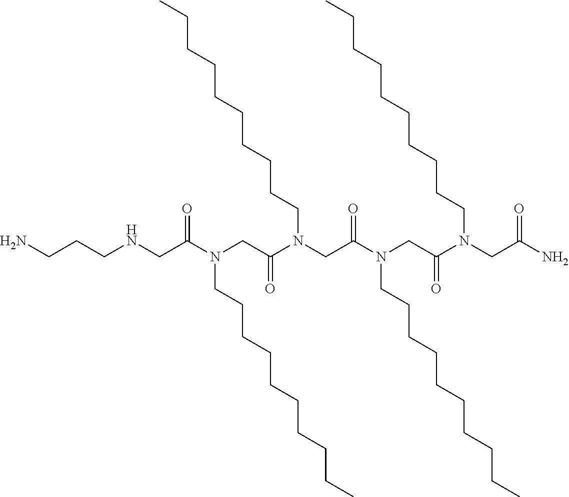 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CNCCCN] |
| 43 | 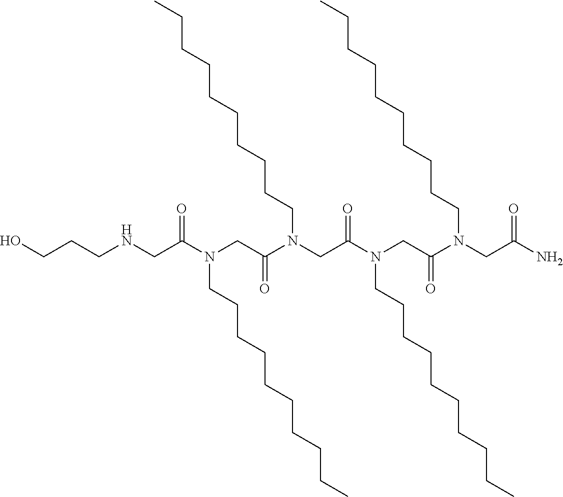 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CNCCCO] |
| 44 | 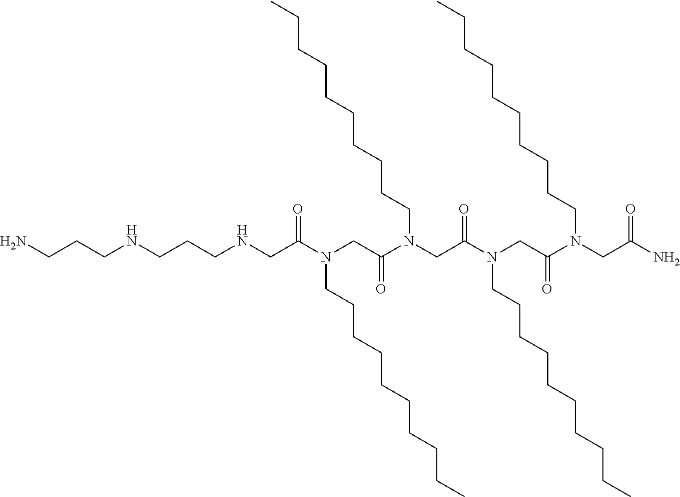 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CNCCCNCCCN] |
| 45 | 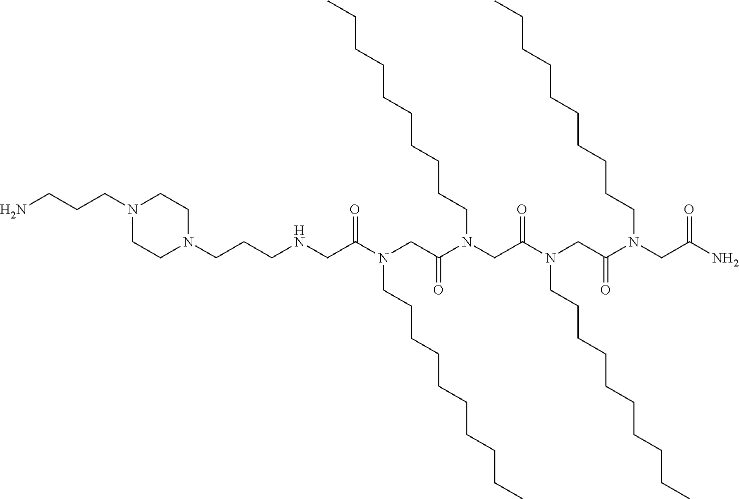 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CNCCCN1CCN(CCCN)CC1] |
| 46 | 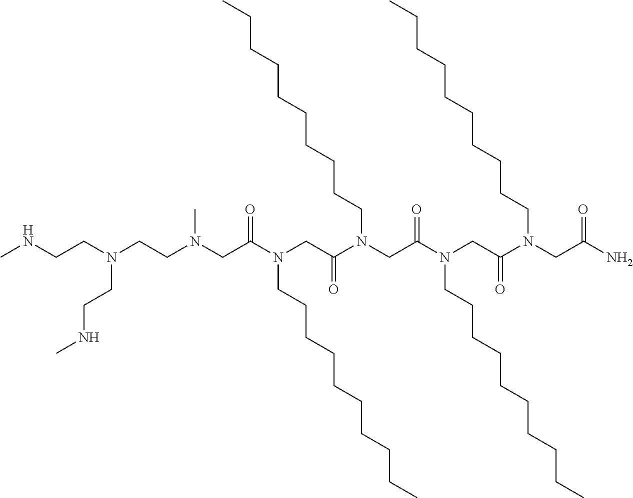 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(C)CCN(CCNC)CCNC] |
| 47 | 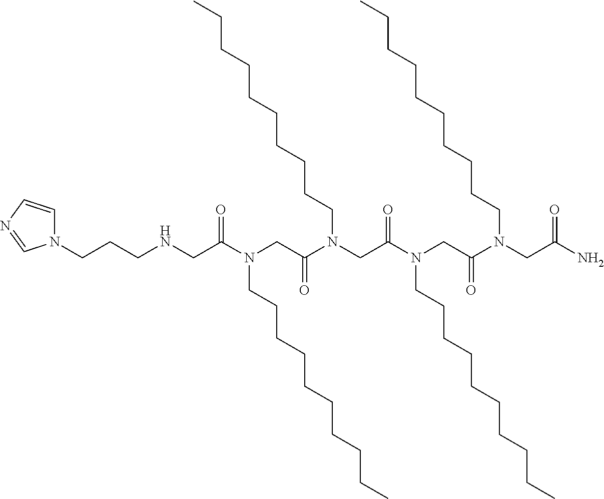 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CNCCCn1ccnc1] |
| 48 | 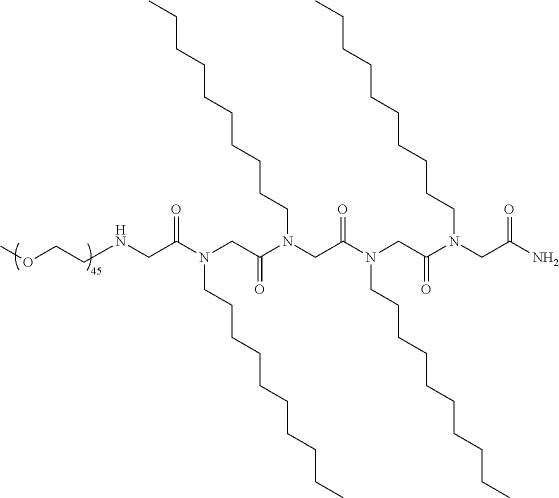 |
| 49 | 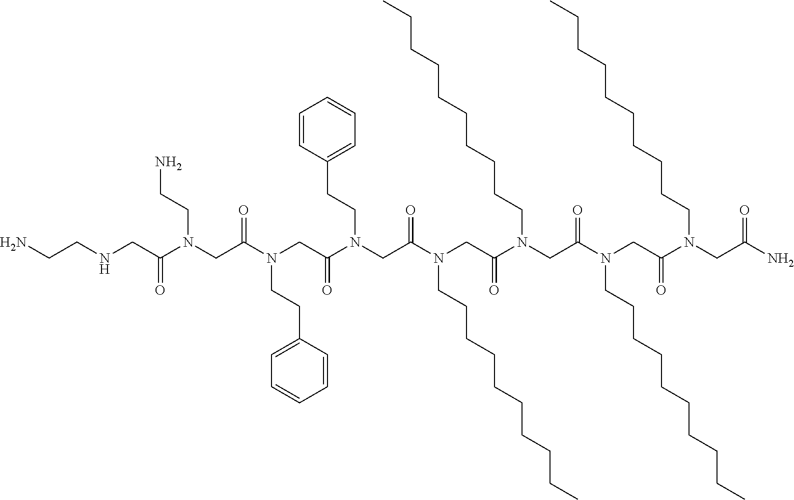 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CNCCN] |
| 50 | 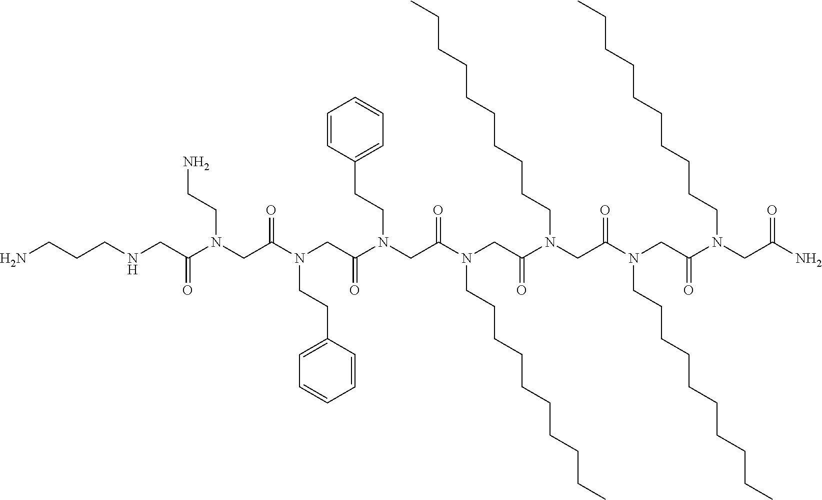 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CNCCCN] |
| 51 | 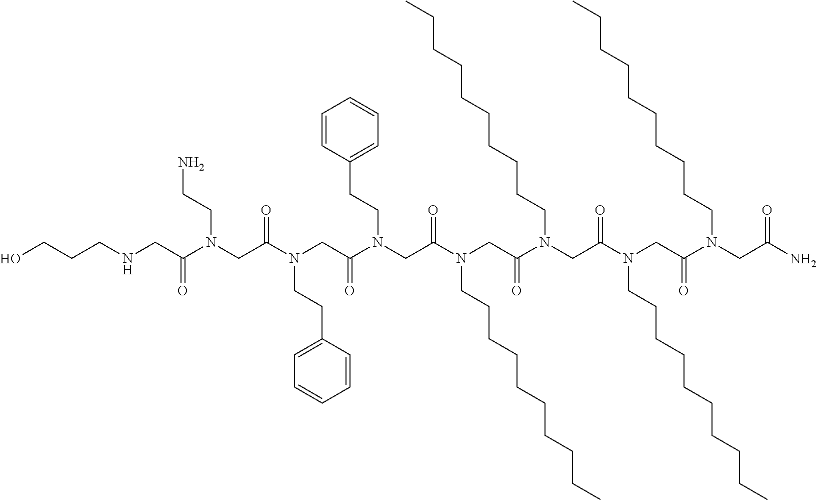 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CNCCCO] |
| 52 | 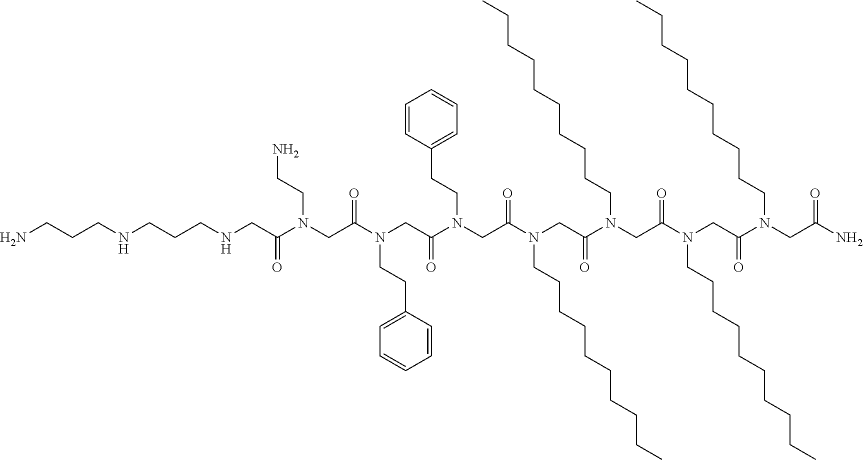 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CNCCCNCCCN] |
| 53 | 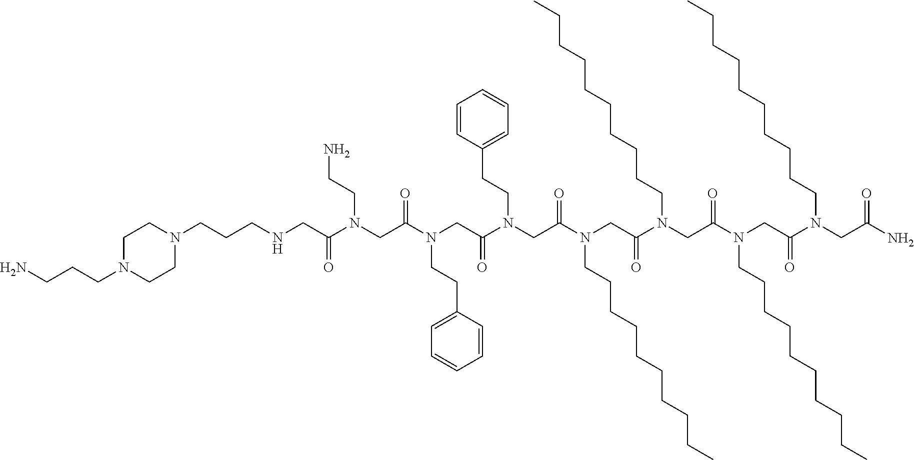 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CNCCCN1CCN(CCCN)CC1] |
| 54 | 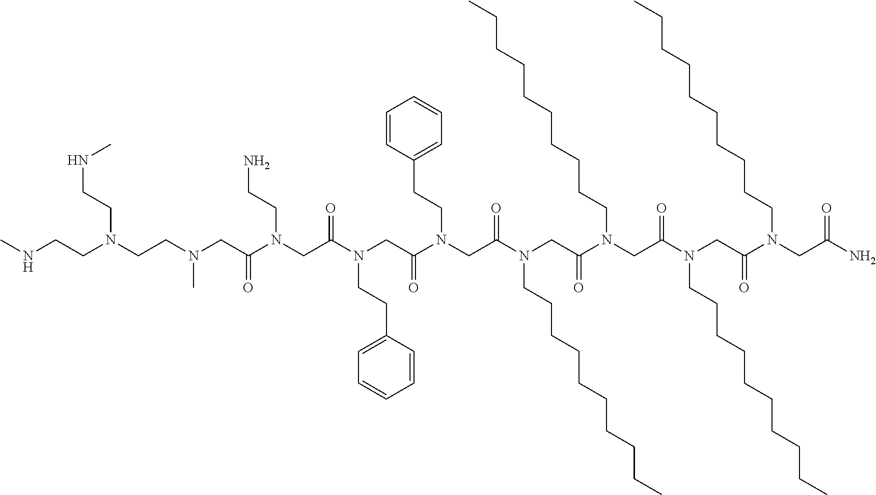 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CN(C)CCN(CCNC)CCNC] |
| 55 | 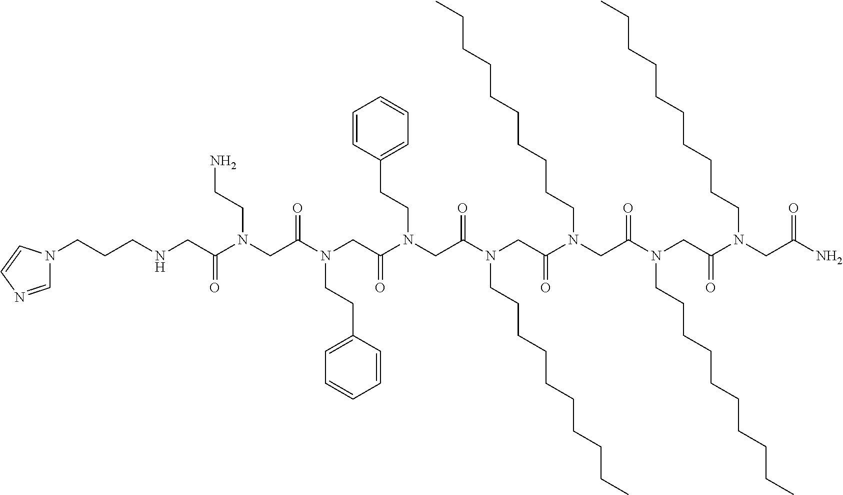 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CNCCCn1ccnc1] |
| 56 | 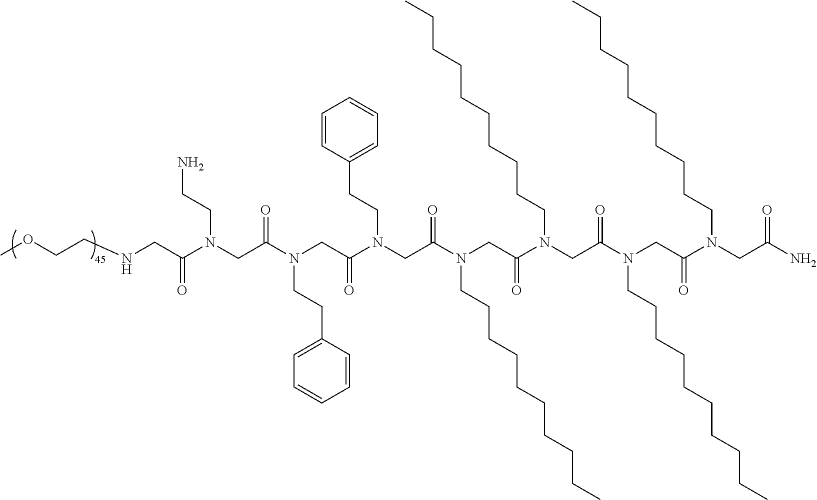 |
| 57 | 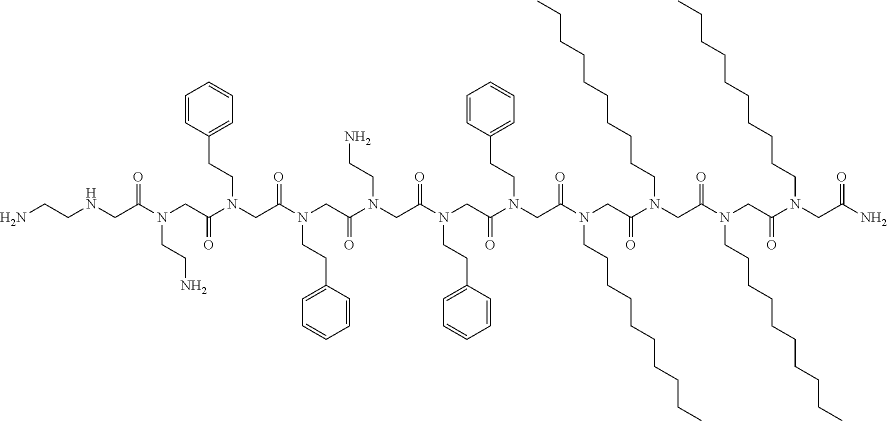 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CNCCN] |
| 58 | 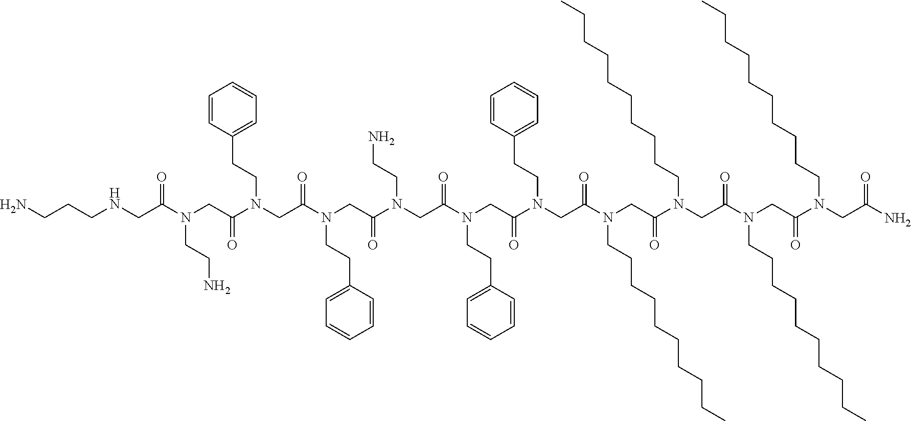 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CNCCCN] |
| 59 | 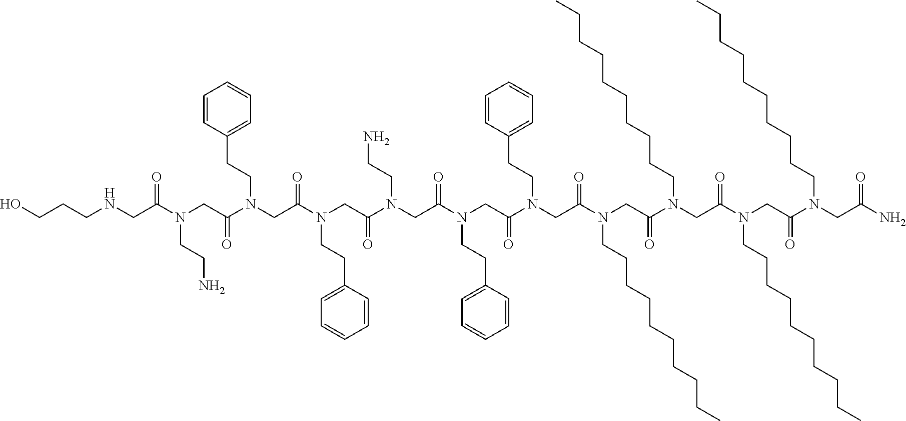 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CNCCCO] |
| 60 | 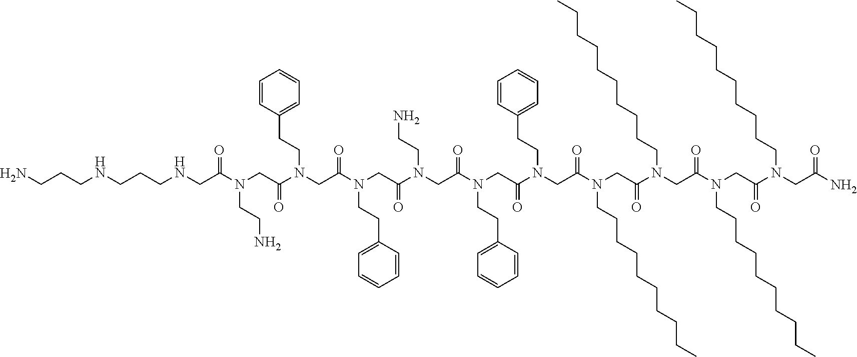 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CNCCCNCCCN] |
| 61 | 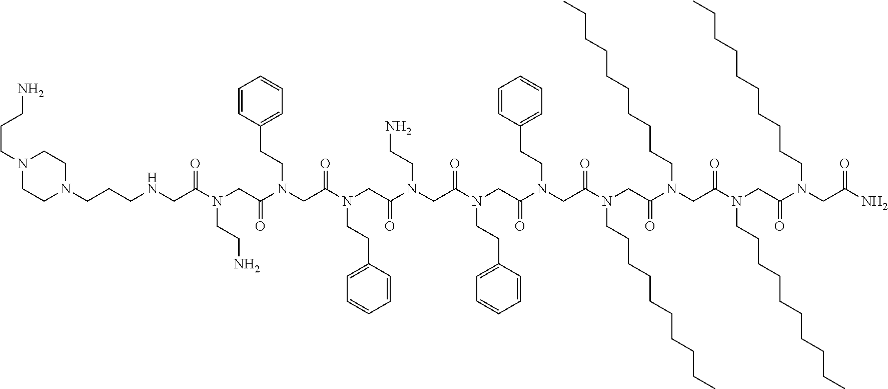 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CNCCCN1CCN(CCCN)CC1] |
| 62 | 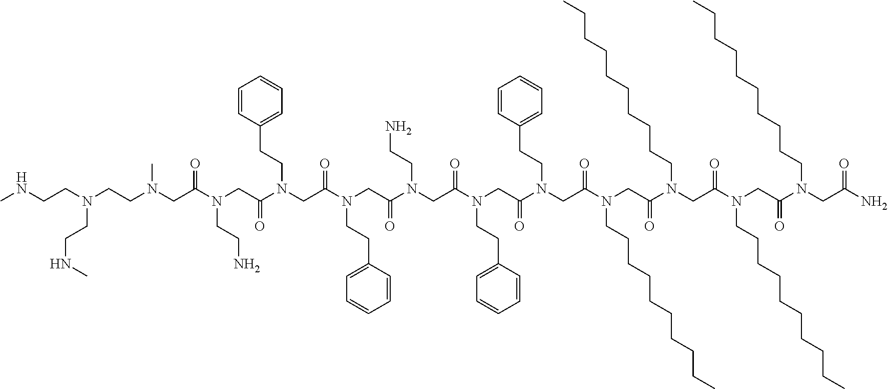 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CN(C)CCN(CCNC)CCNC] |
| 63 | 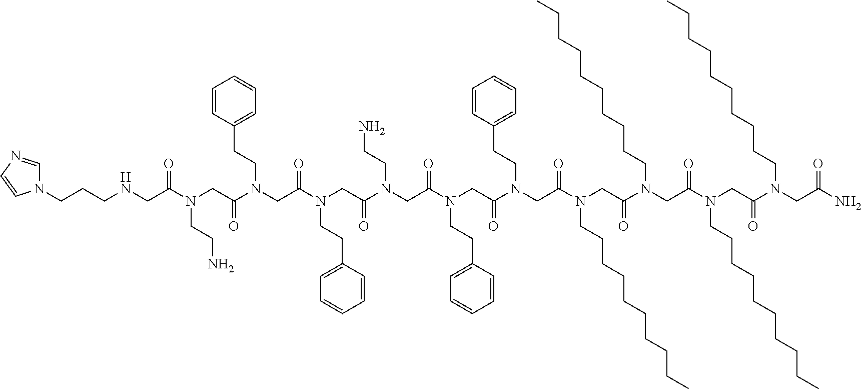 [SMILES: CCCCCCCCCCN(CC(N)=O)C(=O)CN(CCCCCCCCCC)C(=O)CN(CCCCCCCCCC)C(=O)CN(CCCCCCCCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CNCCCn1ccnc1] |
| 64 | 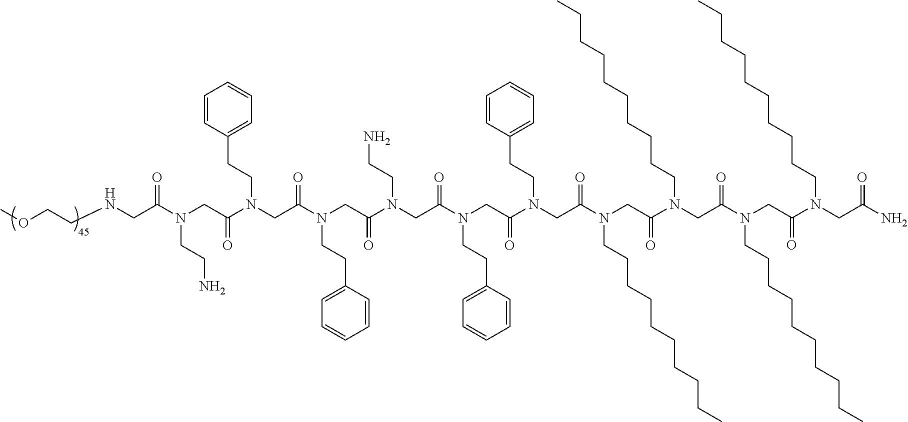 |
| 65 | 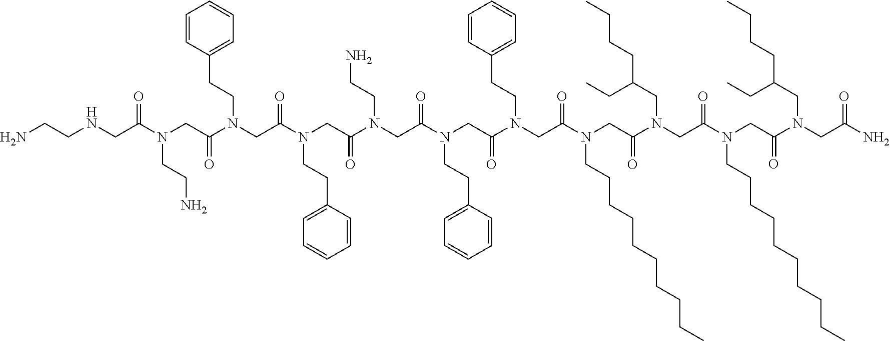 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CNCCN] |
| 66 | 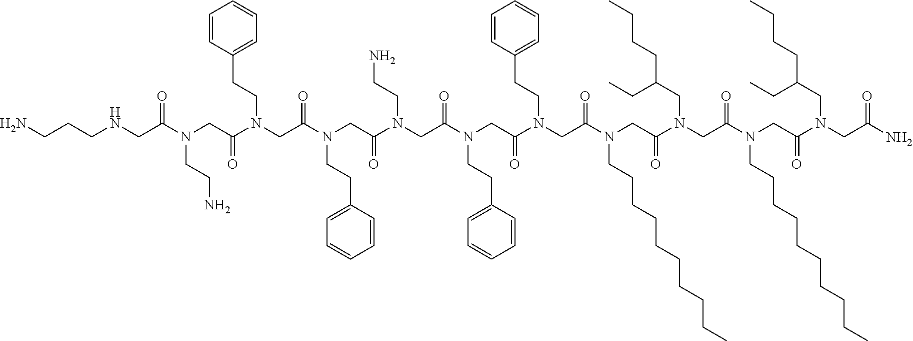 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CNCCCN] |
| 67 | 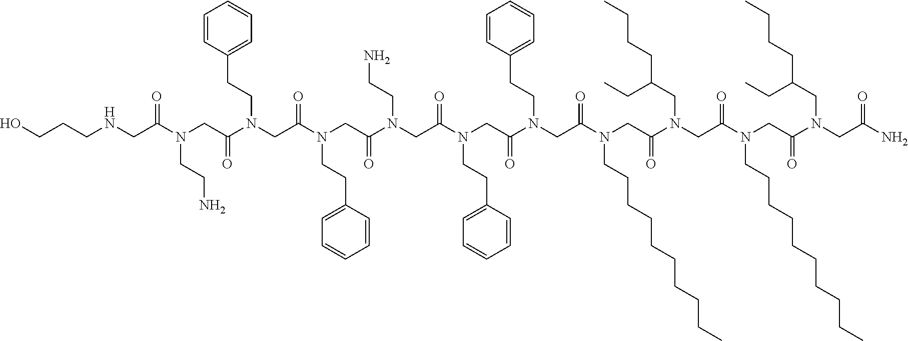 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CNCCCO] |
| 68 | 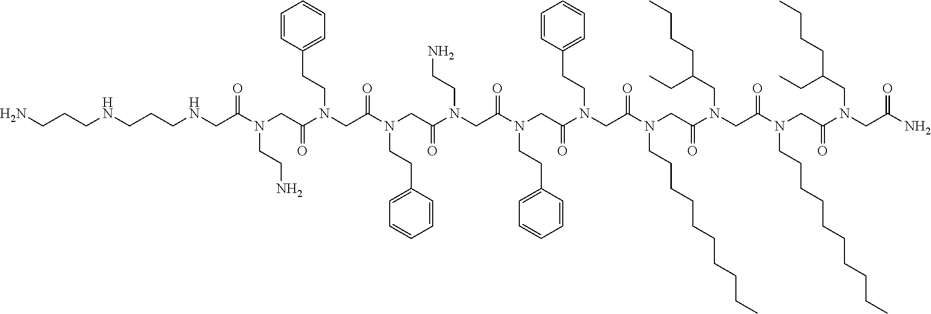 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CNCCCNCCCN] |
| 69 | 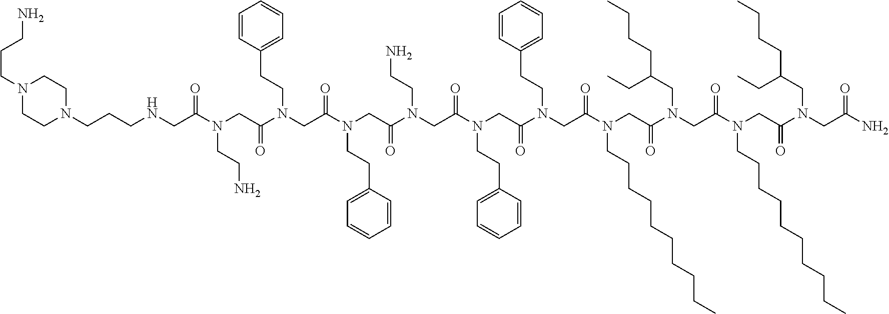 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CNCCCN1CCN(CCCN)CC1] |
| 70 | 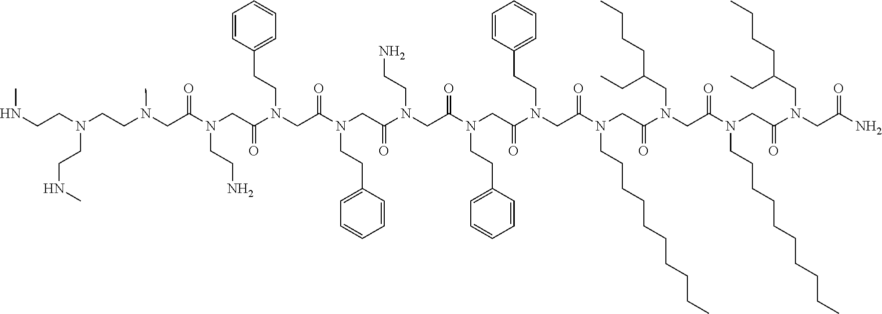 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CN(C)CCN(CCNC)CCNC] |
| 71 | 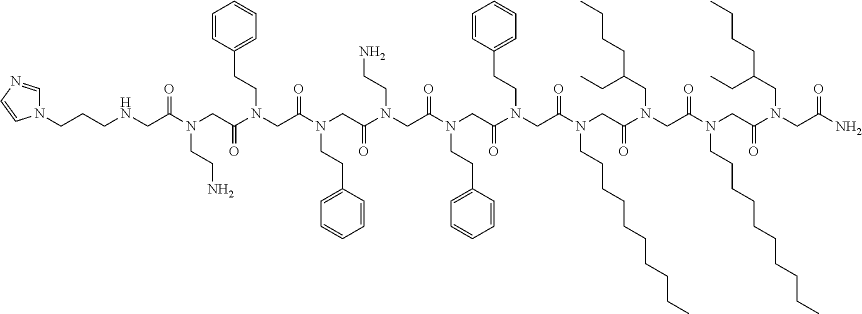 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CN(CCc1ccccc1)C(=O)CN(CCc1ccccc1)C(=O)CN(CCN)C(=O)CN(CCc1ccccc1)C(=O)CN(CCc1ccccc1)C(=O)CN(CCN)C(=O)CNCCCn1ccnc1] |
| 72 | 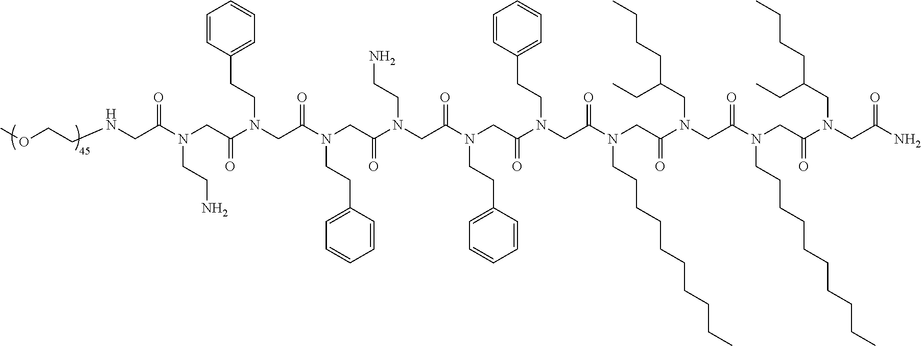 |
| 73 | 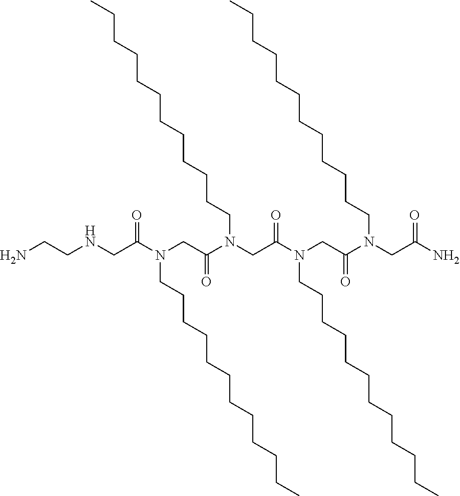 [SMILES: CCCCCCCCCCCCN(CC(N)=O)C(=O)CN(CCCCCCCCCCCC)C(=O)CN(CCCCCCCCCCCC)C(=O)CN(CCCCCCCCCCCC)C(=O)CNCCN] |
| 74 | 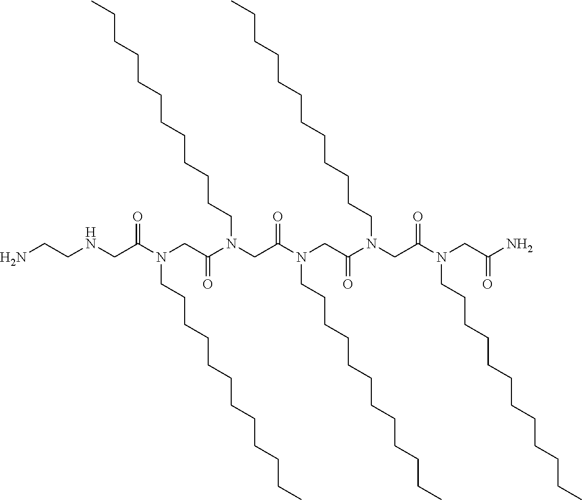 [SMILES: CCCCCCCCCCCCN(CC(N)=O)C(=O)CN(CCCCCCCCCCCC)C(=O)CN(CCCCCCCCCCCC)C(=O)CN(CCCCCCCCCCCC)C(=O)CN(CCCCCCCCCCCC)C(=O)CNCCN] |
| 75 | 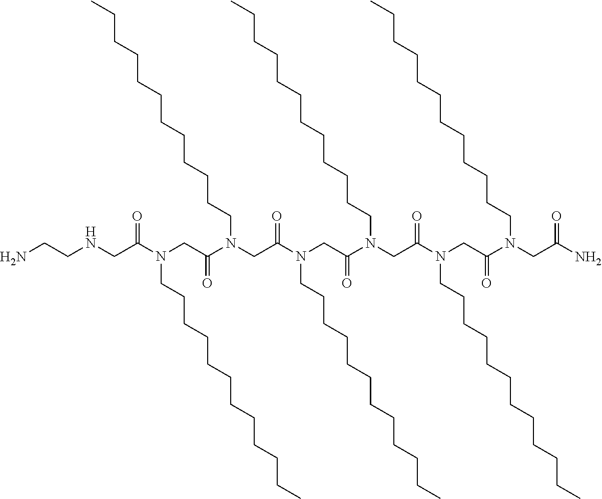 [SMILES: CCCCCCCCCCCCN(CC(N)=O)C(=O)CN(CCCCCCCCCCCC)C(=O)CN(CCCCCCCCCCCC)C(=O)CN(CCCCCCCCCCCC)C(=O)CN(CCCCCCCCCCCC)C(=O)CN(CCCCCCCCCCCC)C(=O)CNCCN] |
| 76 | 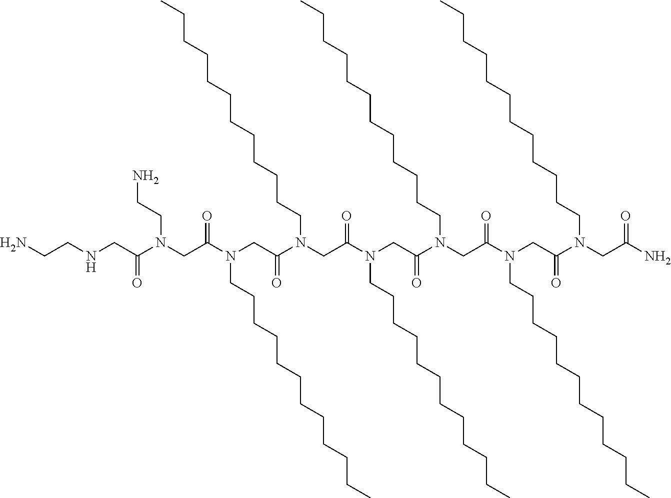 [SMILES: CCCCCCCCCCCCN(CC(N)=O)C(=O)CN(CCCCCCCCCCCC)C(=O)CN(CCCCCCCCCCCC)C(=O)CN(CCCCCCCCCCCC)C(=O)CN(CCCCCCCCCCCC)C(=O)CN(CCCCCCCCCCCC)C(=O)CN(CCN)C(=O)CNCCN] |
| 77 | 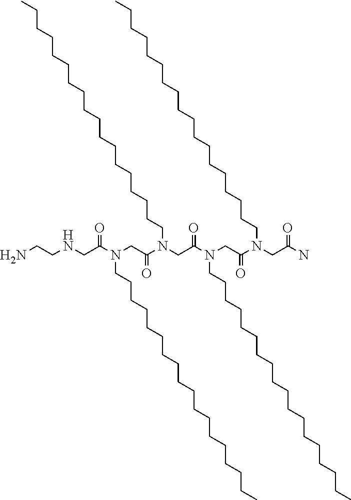 [SMILES: CCCCCCCCCCCCCCCCCCN(CC(N)=O)C(=O)CN(CCCCCCCCCCCCCCCCCC)C(=O)CN(CCCCCCCCCCCCCCCCCC)C(=O)CN(CCCCCCCCCCCCCCCCCC)C(=O)CNCCN] |
| 78 | 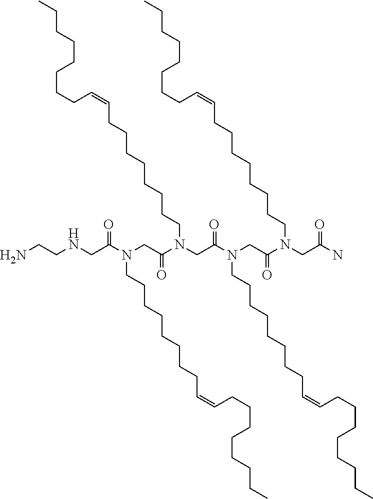 [SMILES: CCCCCCCC/C=C\CCCCCCCCN(CC(N)=O)C(=O)CN(CCCCCCCC/C=C\CCCCCCCC)C(=O)CN(CCCCCCCC/C=C\CCCCCCCC)C(=O)CN(CCCCCCCC/C=C\CCCCCCCC)C(=O)CNCCN] |
| 79 | 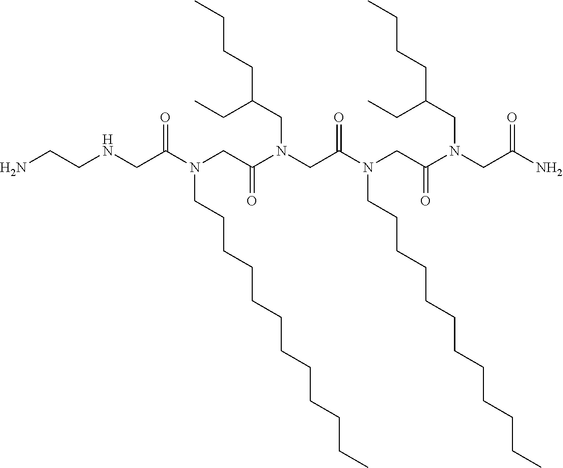 [SMILES: CCCCCCCCCCCCN(CC(=O)N(CC(=O)N(CCCCCCCCCCCC)CC(=O)N(CC(N)=O)CC(CC)CCCC)CC(CC)CCCC)C(=O)CNCCN] |
| 80 | 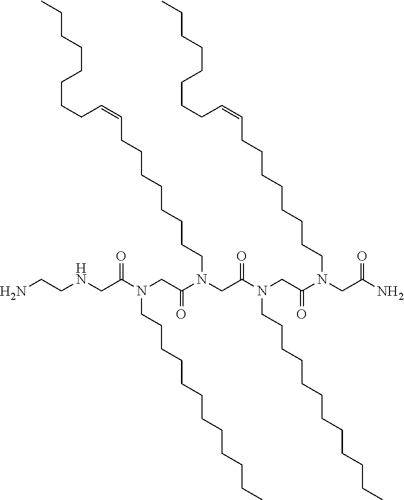 [SMILES: CCCCCCCC/C=C\CCCCCCCCN(CC(N)=O)C(=O)CN(CCCCCCCCCCCC)C(=O)CN(CCCCCCCC/C=C\CCCCCCCC)C(=O)CN(CCCCCCCCCCCC)C(=O)CNCCN] |
| 81 | 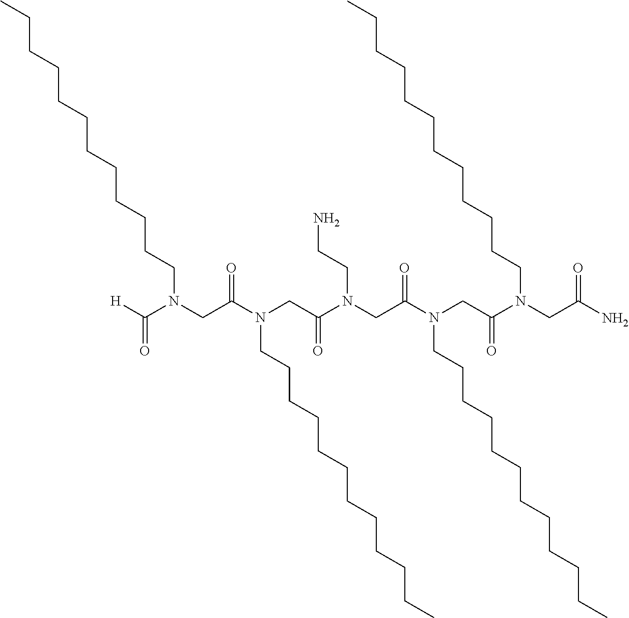 [SMILES: CCCCCCCCCCCCN(C=O)CC(=O)N(CCCCCCCCCCCC)CC(=O)N(CCN)CC(=O)N(CCCCCCCCCCCC)CC(=O)N(CCCCCCCCCCCC)CC(N)=O] |
| 82 | 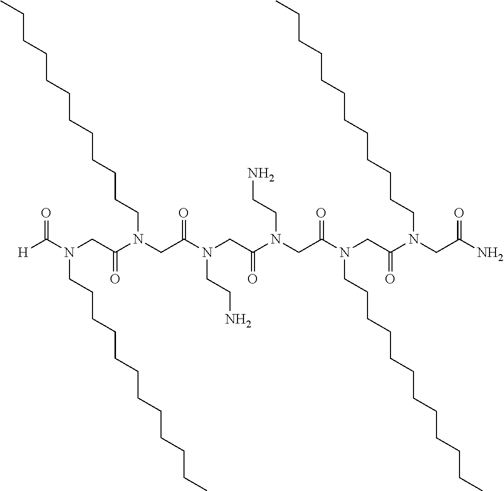 [SMILES: CCCCCCCCCCCCN(C=O)CC(=O)N(CCCCCCCCCCCC)CC(=O)N(CCN)CC(=O)N(CCN)CC(=O)N(CCCCCCCCCCCC)CC(=O)N(CCCCCCCCCCCC)CC(N)=O] |
| 83 | 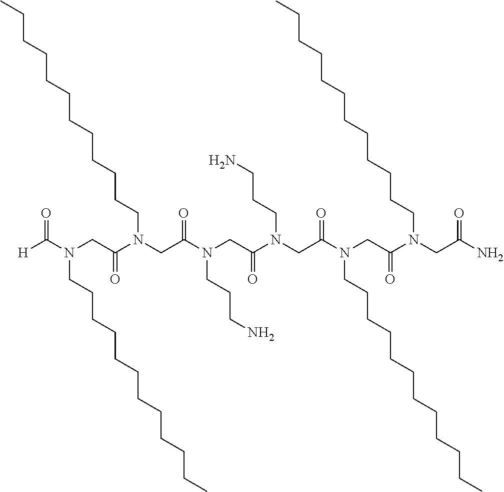 [SMILES: CCCCCCCCCCCCN(C=O)CC(=O)N(CCCCCCCCCCCC)CC(=O)N(CCCN)CC(=O)N(CCCN)CC(=O)N(CCCCCCCCCCCC)CC(=O)N(CCCCCCCCCCCC)CC(N)=O] |
| 84 | 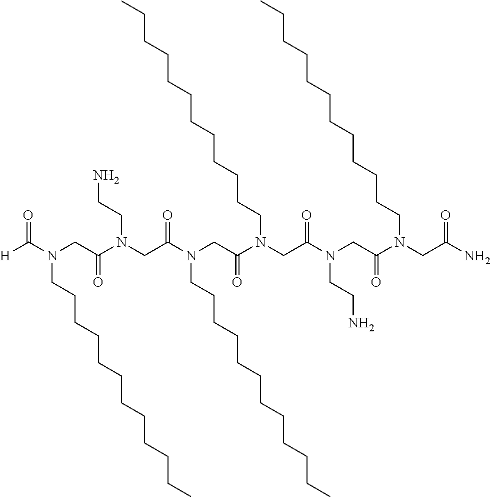 [SMILES: CCCCCCCCCCCCN(C=O)CC(=O)N(CCN)CC(=O)N(CCCCCCCCCCCC)CC(=O)N(CCCCCCCCCCCC)CC(=O)N(CCN)CC(=O)N(CCCCCCCCCCCC)CC(N)=O] |
| 85 | 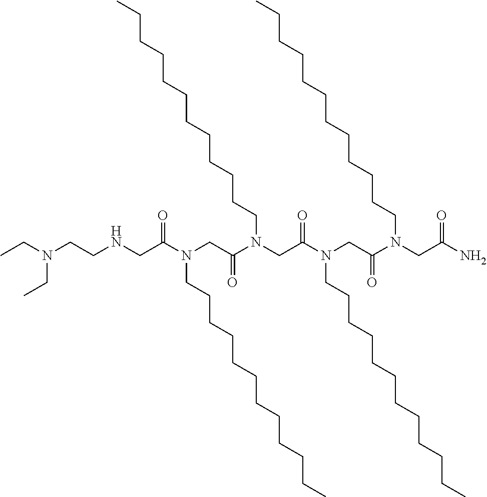 [SMILES: CCCCCCCCCCCCN(CC(N)=O)C(=O)CN(CCCCCCCCCCCC)C(=O)CN(CCCCCCCCCCCC)C(=O)CN(CCCCCCCCCCCC)C(=O)CNCCN(CC)CC] |
| 86 | 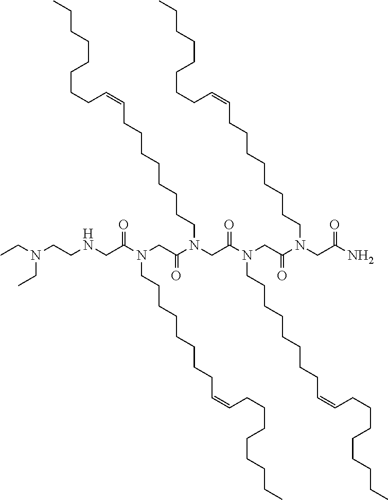 [SMILES: CCCCCCCC/C=C\CCCCCCCCN(CC(N)=O)C(=O)CN(CCCCCCCC/C=C\CCCCCCCC)C(=O)CN(CCCCCCCC/C=C\CCCCCCCC)C(=O)CN(CCCCCCCC/C=C\CCCCCCCC)C(=O)CNCCN(CC)CC] |
| 87 | 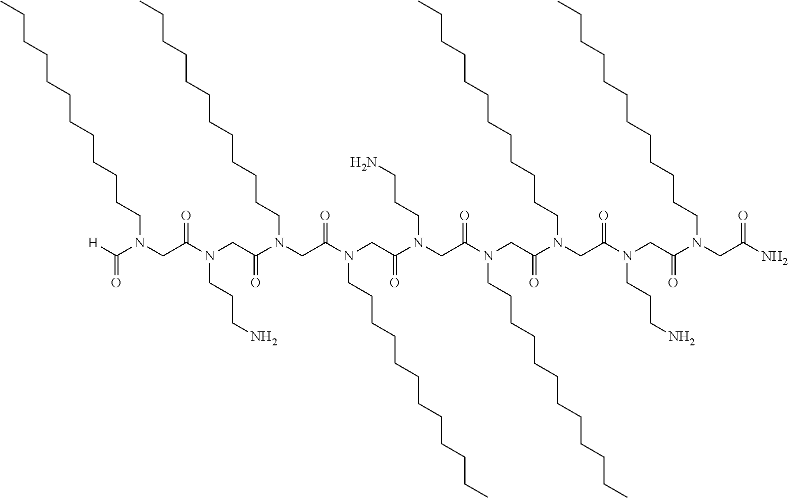 [SMILES: CCCCCCCCCCCCN(C=O)CC(=O)N(CCCN)CC(=O)N(CCCCCCCCCCCC)CC(=O)N(CCCCCCCCCCCC)CC(=O)N(CCCN)CC(=O)N(CCCCCCCCCCCC)CC(=O)N(CCCCCCCCCCCC)CC(=O)N(CCCN)CC(=O)N(CCCCCCCCCCCC)CC(N)=O] |
| 88 | 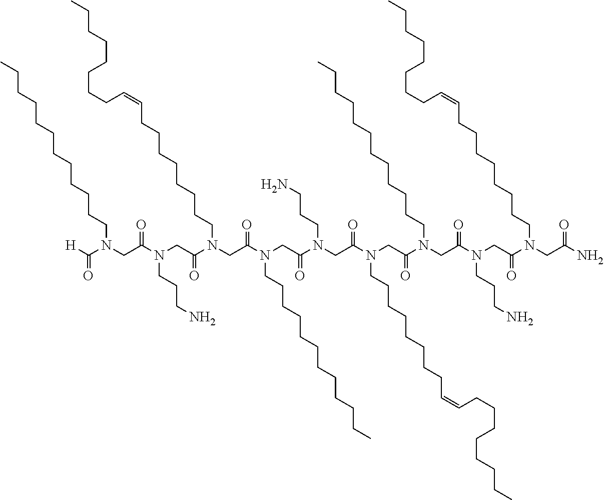 [SMILES: CCCCCCCC/C=C\CCCCCCCCN(CC(N)=O)C(=O)CN(CCCN)C(=O)CN(CCCCCCCCCCCC)C(=O)CN(CCCCCCCC/C=C\CCCCCCCC)C(=O)CN(CCCN)C(=O)CN(CCCCCCCCCCCC)C(=O)CN(CCCCCCCC/C=C\CCCCCCCC)C(=O)CN(CCCN)C(=O)CN(C=O)CCCCCCCCCCCC] |
| 89 | 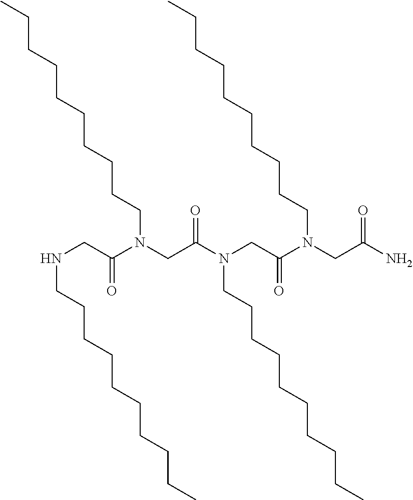 [SMILES: CCCCCCCCCCNCC(=O)N(CCCCCCCCCC)CC(=O)N(CCCCCCCCCC)CC(=O)N(CCCCCCCCCC)CC(N)=O] |
| 90 | 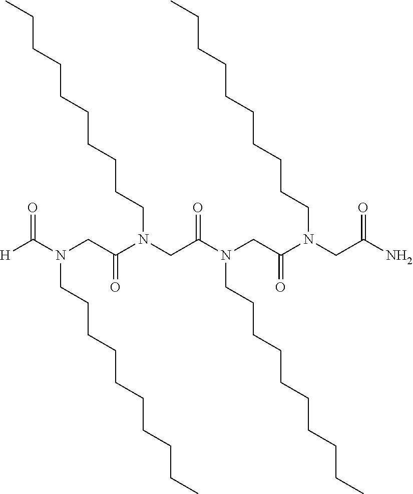 [SMILES: CCCCCCCCCCN(C=O)CC(=O)N(CCCCCCCCCC)CC(=O)N(CCCCCCCCCC)CC(=O)N(CCCCCCCCCC)CC(N)=O] |
| 91 | 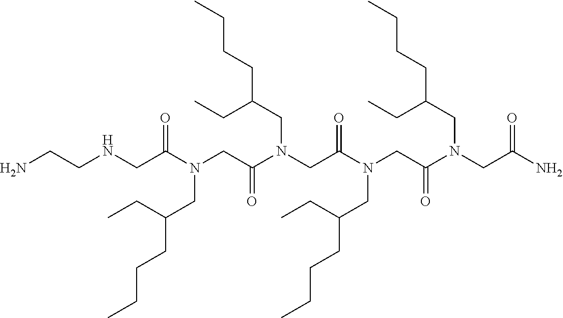 [SMILES: CCCCC(CC)CN(CC(N)=O)C(=O)CN(CC(CC)CCCC)C(=O)CN(CC(CC)CCCC)C(=O)CN(CC(CC)CCCC)C(=O)CNCCN] |
| 92 | 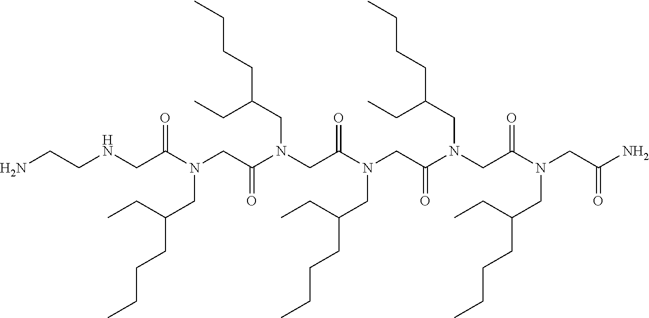 [SMILES: CCCCC(CC)CN(CC(N)=O)C(=O)CN(CC(CC)CCCC)C(=O)CN(CC(CC)CCCC)C(=O)CN(CC(CC)CCCC)C(=O)CN(CC(CC)CCCC)C(=O)CNCCN] |
| 93 | 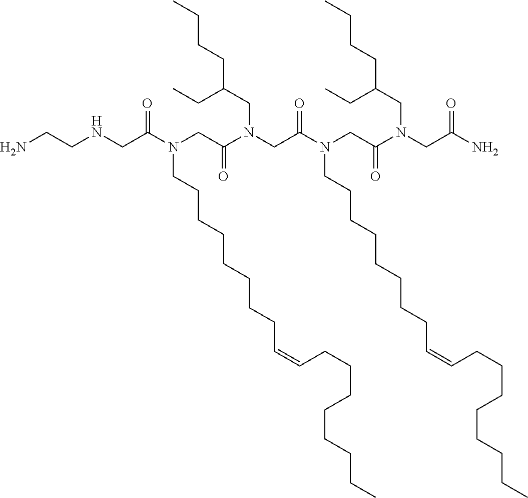 [SMILES: CCCCCCCC/C=C\CCCCCCCCN(CC(=O)N(CC(=O)N(CCCCCCCC/C=C\CCCCCCCC)CC(=O)N(CC(N)=O)CC(CC)CCCC)CC(CC)CCCC)C(=O)CNCCN] |
| 94 | 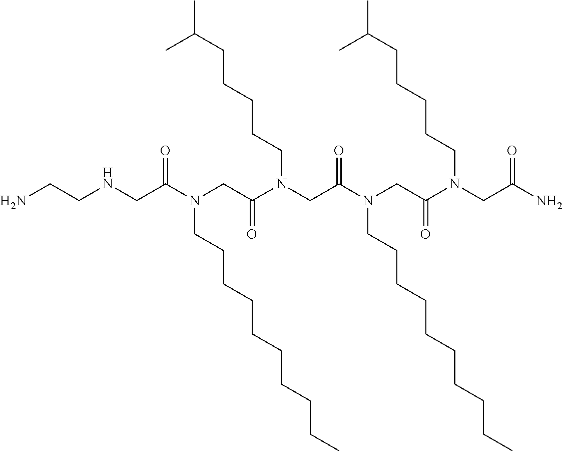 [SMILES: CCCCCCCCCCN(CC(=O)N(CCCCCC(C)C)CC(=O)N(CCCCCCCCCC)CC(=O)N(CCCCCC(C)C)CC(N)=O)C(=O)CNCCN] |
| 95 | 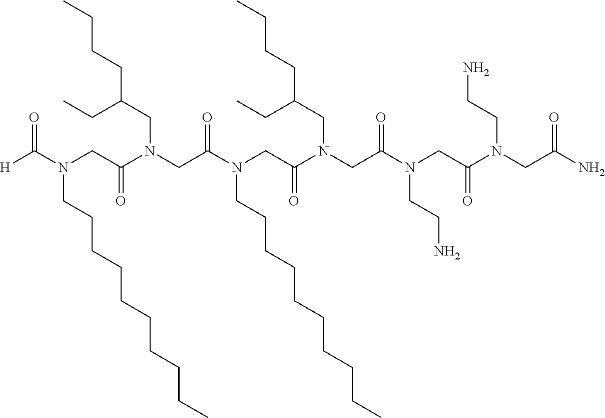 [SMILES: CCCCCCCCCCN(C=O)CC(=O)N(CC(=O)N(CCCCCCCCCC)CC(=O)N(CC(=O)N(CCN)CC(=O)N(CCN)CC(N)=O)CC(CC)CCCC)CC(CC)CCCC] |
| 96 | 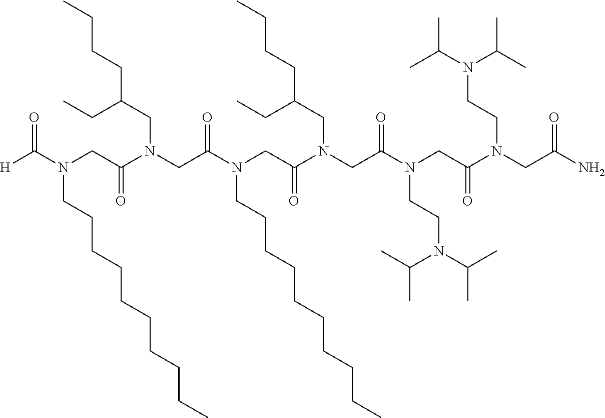 [SMILES: CCCCCCCCCCN(C=O)CC(=O)N(CC(=O)N(CCCCCCCCCC)CC(=O)N(CC(=O)N(CCN(C(C)C)C(C)C)CC(=O)N(CCN(C(C)C)C(C)C)CC(N)=O)CC(CC)CCCC)CC(CC)CCCC] |
| 97 | 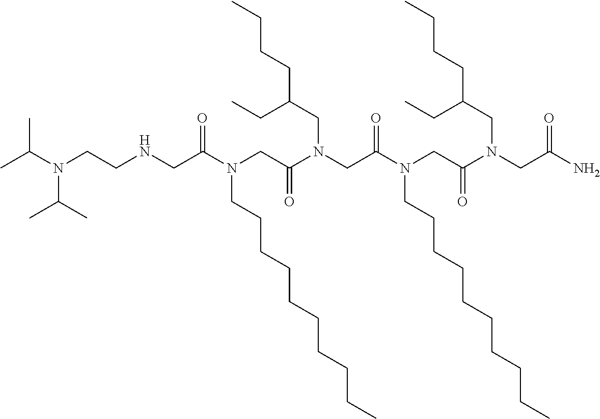 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CNCCN(C(C)C)C(C)C] |
| 98 | 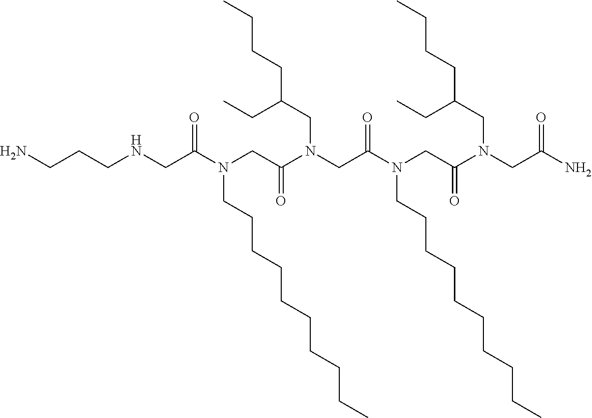 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CNCCCN] |
| 99 | 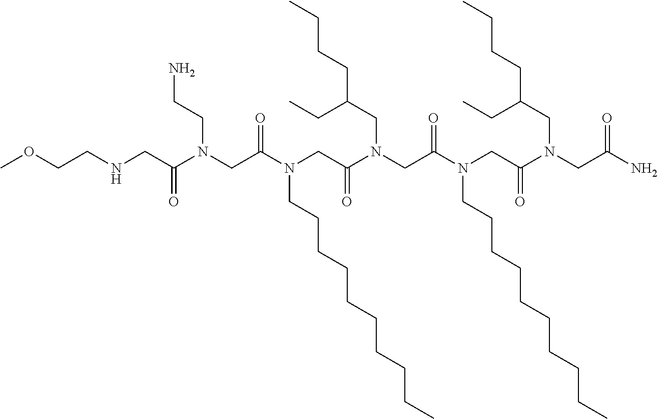 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CN(CCN)C(=O)CNCCOC] |
| 100 | 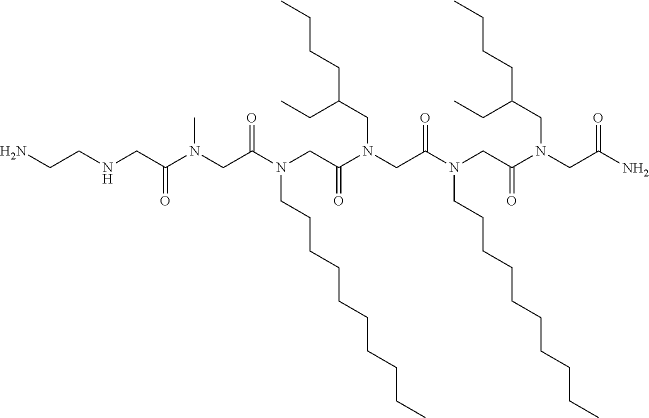 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CN(C)C(=O)CNCCN] |
| 101 | 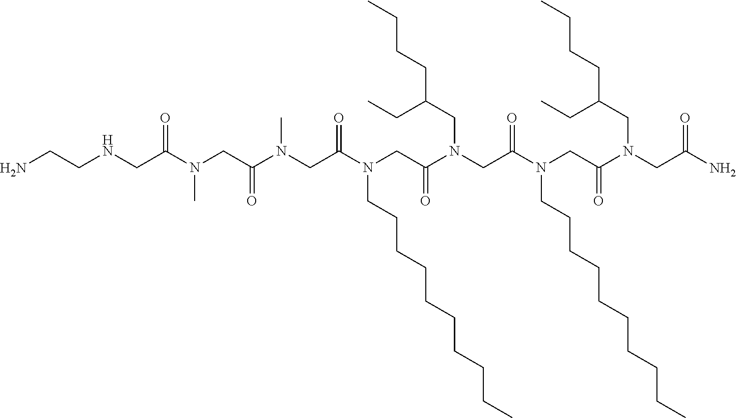 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CN(C)C(=O)CN(C)C(=O)CNCCN] |
| 102 | 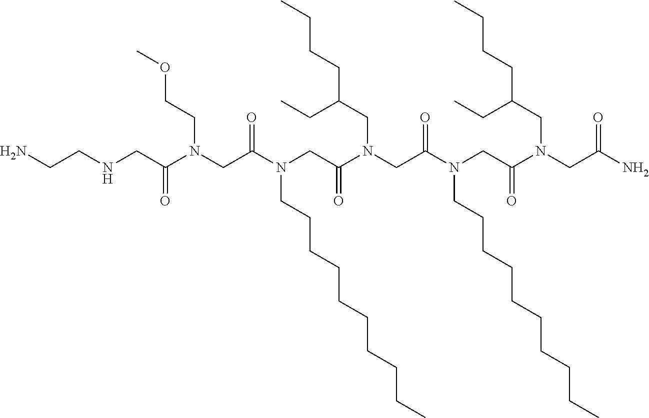 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CN(CCOC)C(=O)CNCCN] |
| 103 | 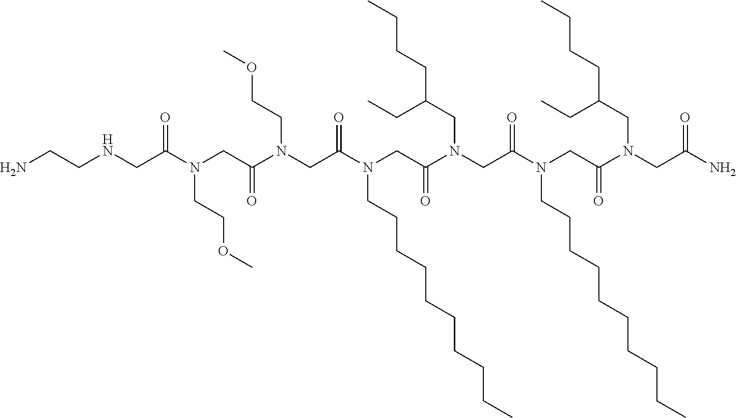 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CN(CCOC)C(=O)CN(CCOC)C(=O)CNCCN] |
| 104 | 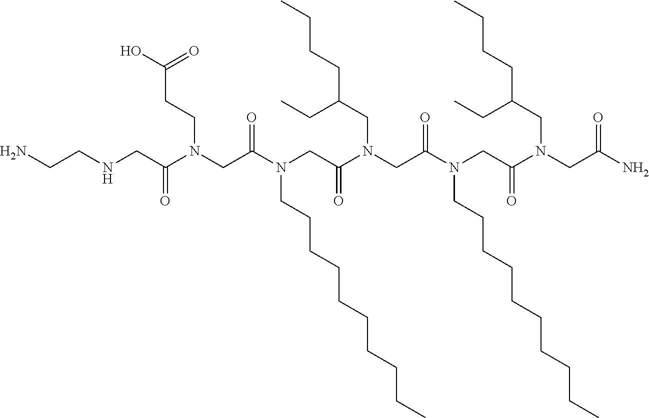 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CN(CCC(=O)O)C(=O)CNCCN] |
| 105 | 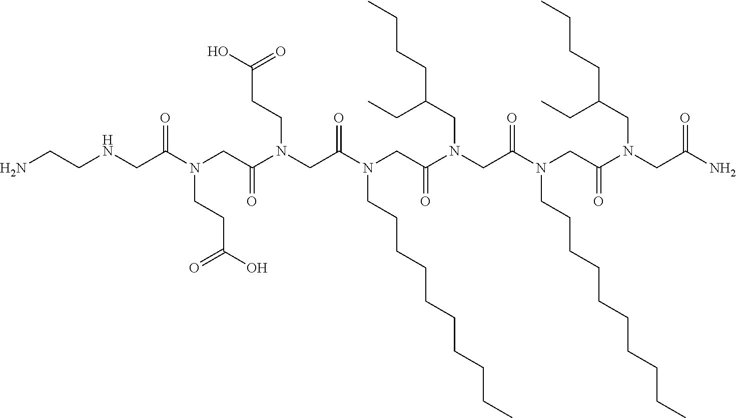 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CN(CCC(=O)O)C(=O)CN(CCC(=O)O)C(=O)CNCCN] |
| 106 | 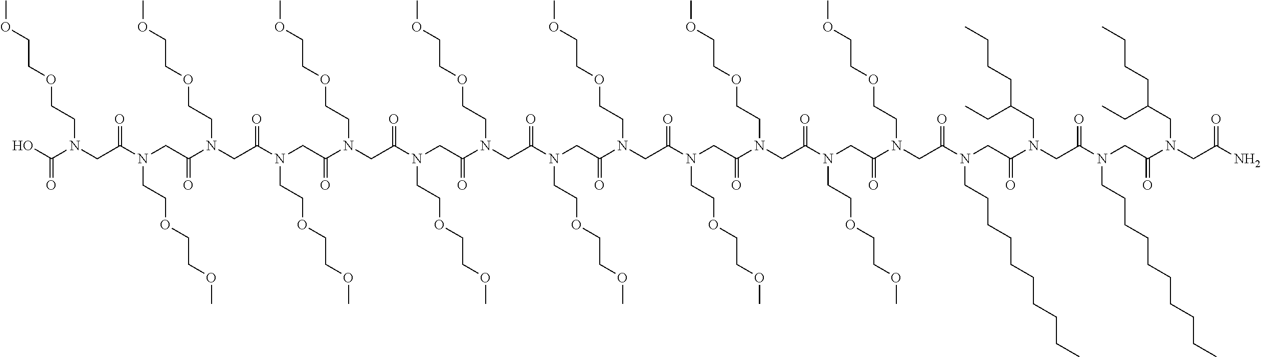 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CN(CCOCCOC)C(=O)CN(CCOCCOC)C(=O)CN(CCOCCOC)C(=O)CN(CCOCCOC)C(=O)CN(CCOCCOC)C(=O)CN(CCOCCOC)C(=O)CN(CCOCCOC)C(=O)CN(CCOCCOC)C(=O)CN(CCOCCOC)C(=O)CN(CCOCCOC)C(=O)CN(CCOCCOC)C(=O)CN(CCOCCOC)C(=O)CN(CCOCCOC)C(=O)O] |
| 107 | 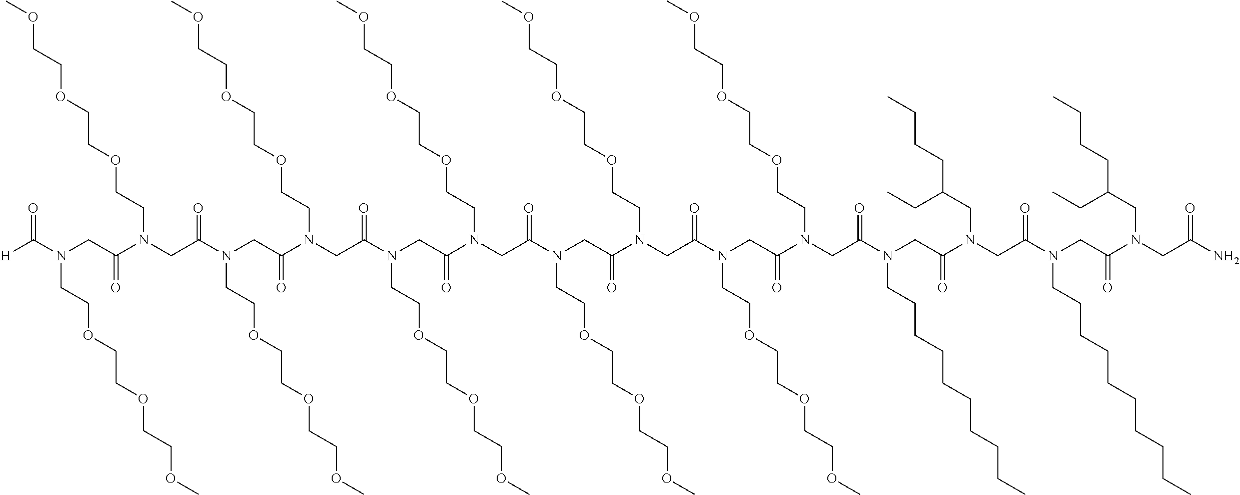 [SMILES: CCCCCCCCCCN(CC(=O)N(CC(=O)N(CCCCCCCCCC)CC(=O)N(CC(N)=O)CC(CC)CCCC)CC(CC)CCCC)C(=O)CN(CCOCCOCCOC)C(=O)CN(CCOCCOCCOC)C(=O)CN(CCOCCOCCOC)C(=O)CN(CCOCCOCCOC)C(=O)CN(CCOCCOCCOC)C(=O)CN(CCOCCOCCOC)C(=O)CN(CCOCCOCCOC)C(=O)CN(CCOCCOCCOC)C(=O)CN(CCOCCOCCOC)C(=O)CN(C=O)CCOCCOCCOC] |
| 108 | 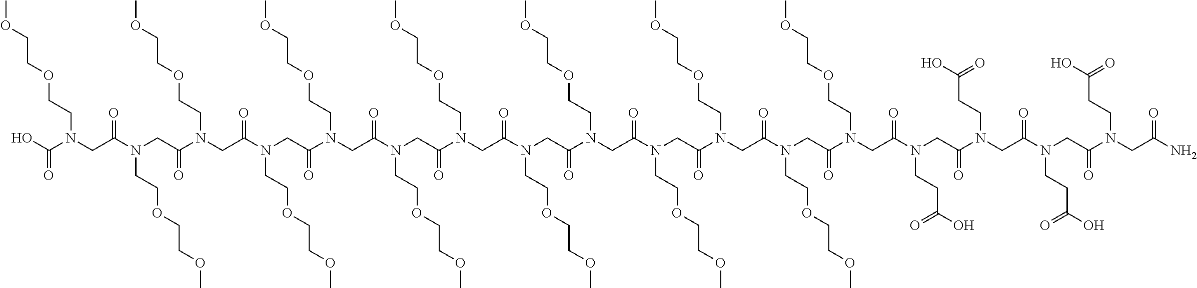 [SMILES: COCCOCCN(CC(=O)N(CCOCCOC)CC(=O)N(CCOCCOC)CC(=O)N(CCOCCOC)CC(=O)N(CCOCCOC)CC(=O)N(CCOCCOC)CC(=O)N(CCOCCOC)CC(=O)N(CCOCCOC)CC(=O)N(CCOCCOC)CC(=O)N(CCOCCOC)CC(=O)N(CCOCCOC)CC(=O)N(CCOCCOC)CC(=O)N(CCOCCOC)CC(=O)N(CCC(=O)O)CC(=O)N(CCC(=O)O)CC(=O)N(CCC(=O)O)CC(=O)N(CCC(=O)O)CC(N)=O)C(=O)O] |
| 109 | 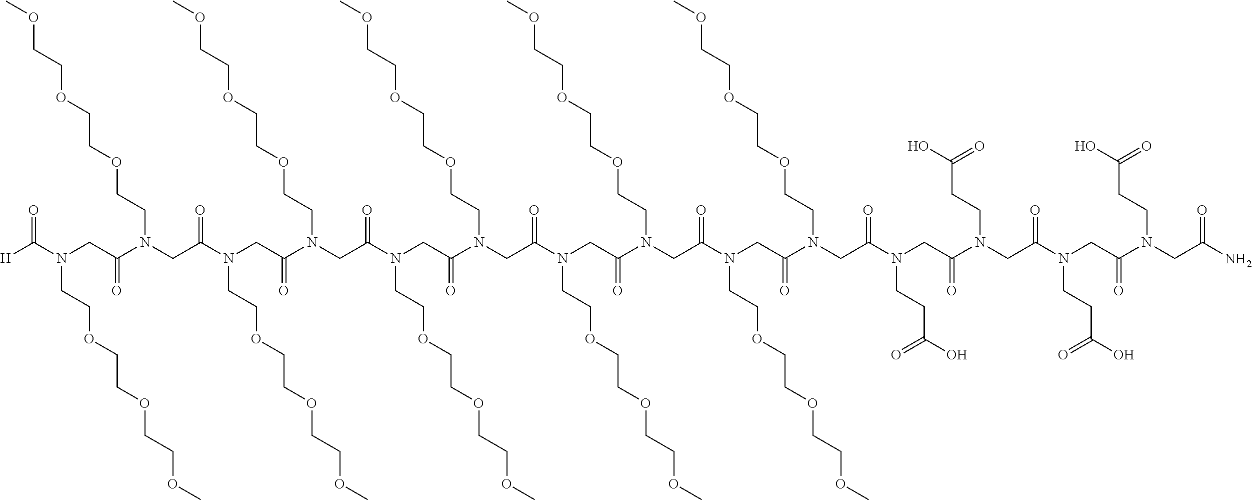 [SMILES: COCCOCCOCCN(C=O)CC(=O)N(CCOCCOCCOC)CC(=O)N(CCOCCOCCOC)CC(=O)N(CCOCCOCCOC)CC(=O)N(CCOCCOCCOC)CC(=O)N(CCOCCOCCOC)CC(=O)N(CCOCCOCCOC)CC(=O)N(CCOCCOCCOC)CC(=O)N(CCOCCOCCOC)CC(=O)N(CCOCCOCCOC)CC(=O)N(CCC(=O)O)CC(=O)N(CCC(=O)O)CC(=O)N(CCC(=O)O)CC(=O)N(CCC(=O)O)CC(N)=O] |
| 110 | 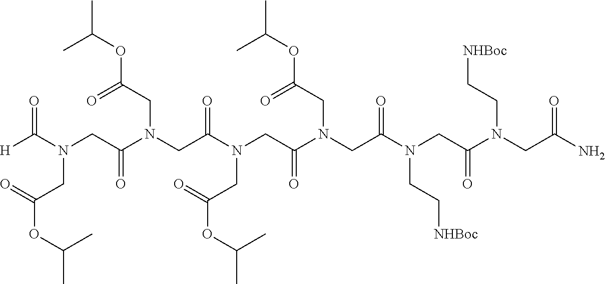 [SMILES: CC(C)OC(=O)CN(C=O)CC(=O)N(CC(=O)OC(C)C)CC(=O)N(CC(=O)OC(C)C)CC(=O)N(CC(=O)OC(C)C)CC(=O)N(CCNC(=O)OC(C)(C)C)CC(=O)N(CCNC(=O)OC(C)(C)C)CC(N)=O] |
| 111 | 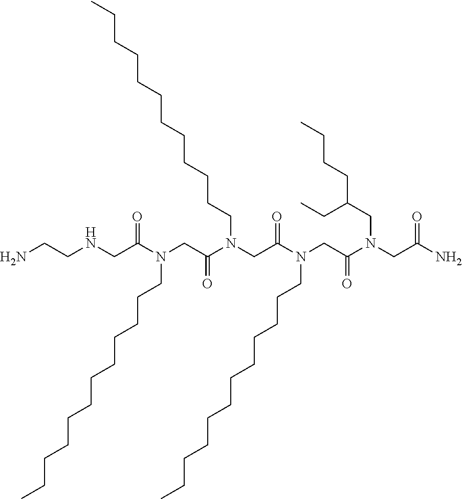 [SMILES: CCCCCCCCCCCCN(CC(=O)N(CCCCCCCCCCCC)CC(=O)N(CCCCCCCCCCCC)CC(=O)N(CC(N)=O)CC(CC)CCCC)C(=O)CNCCN] |
| 112 | 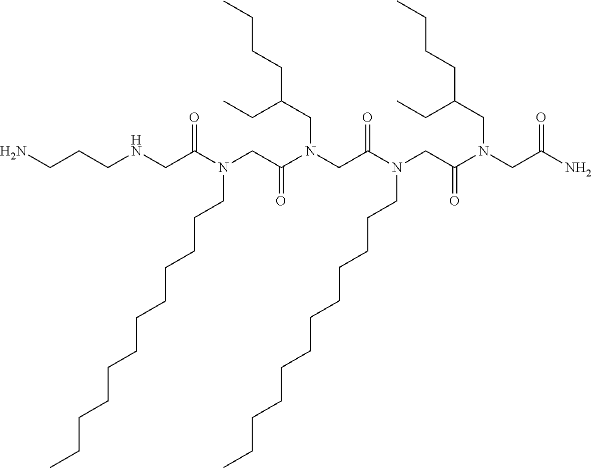 [SMILES: CCCCCCCCCCCCN(CC(=O)N(CC(=O)N(CCCCCCCCCCCC)CC(=O)N(CC(N)=O)CC(CC)CCCC)CC(CC)CCCC)C(=O)CNCCCN] |
| 113 | 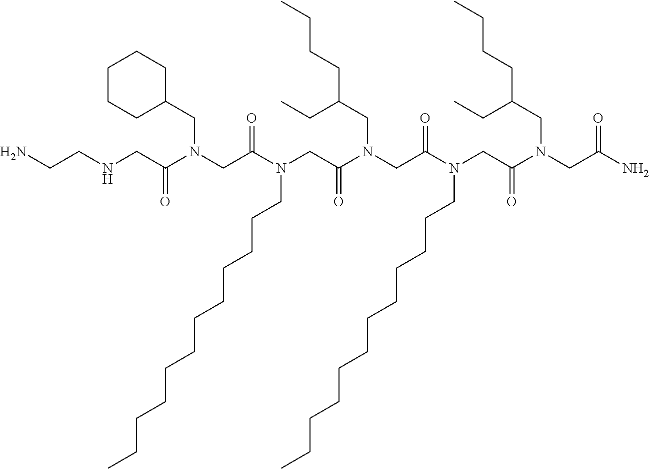 [SMILES: CCCCCCCCCCCCN(CC(=O)N(CC(N)=O)CC(CC)CCCC)C(=O)CN(CC(CC)CCCC)C(=O)CN(CCCCCCCCCCCC)C(=O)CN(CC1CCCCC1)C(=O)CNCCN] |
| 114 | 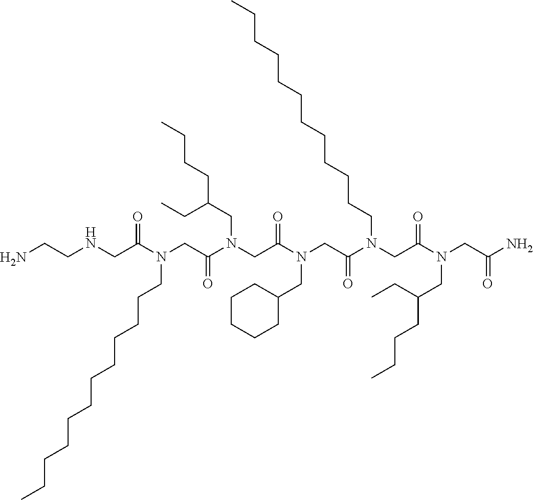 [SMILES: CCCCCCCCCCCCN(CC(=O)N(CC(=O)N(CC(=O)N(CCCCCCCCCCCC)CC(=O)N(CC(N)=O)CC(CC)CCCC)CC1CCCCC1)CC(CC)CCCC)C(=O)CNCCN] |
| 115 | 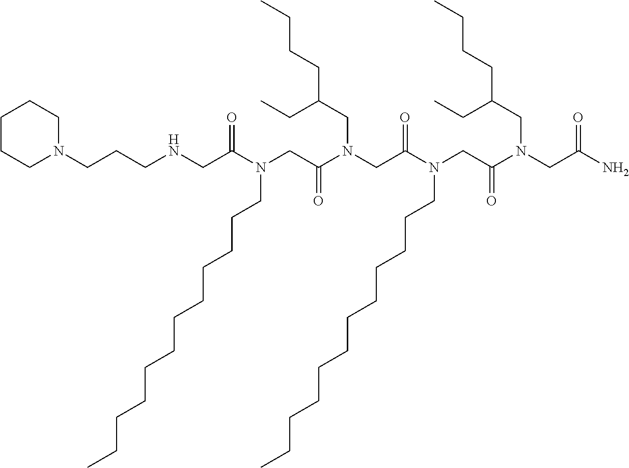 [SMILES: CCCCCCCCCCCCN(CC(=O)N(CC(=O)N(CCCCCCCCCCCC)CC(=O)N(CC(N)=O)CC(CC)CCCC)CC(CC)CCCC)C(=O)CNCCCN1CCCCC1] |
| 116 | 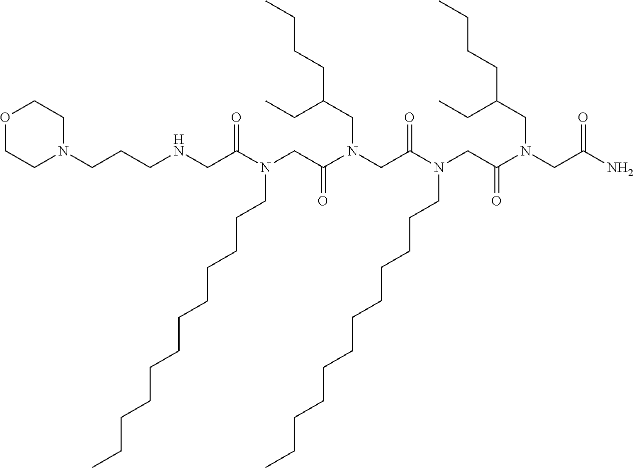 [SMILES: CCCCCCCCCCCCN(CC(=O)N(CC(=O)N(CCCCCCCCCCCC)CC(=O)N(CC(N)=O)CC(CC)CCCC)CC(CC)CCCC)C(=O)CNCCCN1CCOCC1] |
| 117 | 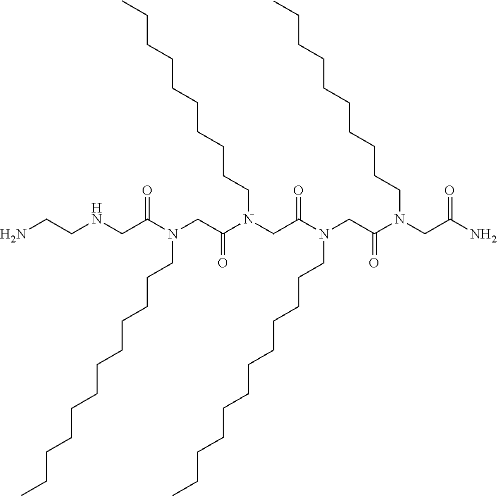 [SMILES: CCCCCCCCCCCCN(CC(=O)N(CCCCCCCCCC)CC(=O)N(CCCCCCCCCCCC)CC(=O)N(CCCCCCCCCC)CC(N)=O)C(=O)CNCCN] |
| 118 | 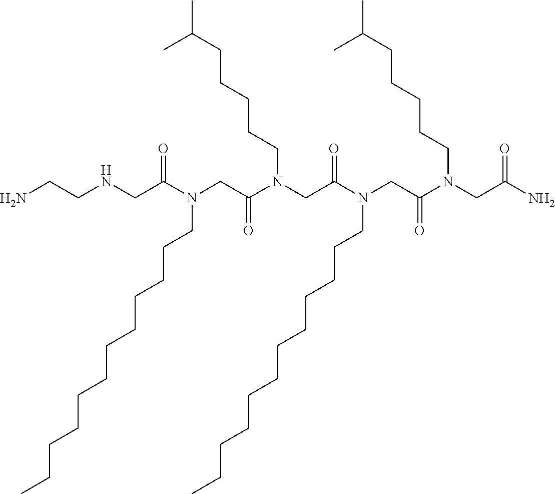 [SMILES: CCCCCCCCCCCCN(CC(=O)N(CCCCCC(C)C)CC(=O)N(CCCCCCCCCCCC)CC(=O)N(CCCCCC(C)C)CC(N)=O)C(=O)CNCCN] |

TABLE 1B

| Compound | Predicted Molecular | Retention | |
|---|---|---|---|
| # | Weight (g/mol) | Time (min) | m/z (MH⁺) |
| 1 | 1623.15 | 4.43 | 1623.9 |
| 2 | 1679.26 | 4.76 | 1680.11 |
| 3 | 1735.37 | 5.03 | 1736.2 |
| 4 | 1915.53 | 4.97 | 1916.2 |
| 5 | 1903.69 | 5.88 | 1904.3 |
| 6 | 1481.94 | 4.17 | 1482.9 |
| 7 | 1876.58 | 5.32 | 1877.26 |
| 8 | 2073.91 | 5.62 | 2074.3 |
| 9 | 834.2 | 4.54 | 834.8 |
| 10 | 1256.73 | 4.67 | 1257.7 |
| 11 | 2101.79 | 4.78 | 2103.3 |
| 12 | 2524.32 | 4.81 | 2524.5 |
| 13 | 1195.65 | 4.13 | 1196 |
| 14 | 712.04 | 3.43 | 712.7 |
| 15 | 912.28 | 3.23 | 912.87 |
| 16 | 1312.76 | 3.17 | 1313.2 |
| 17 | 1679.26 | 4.6 | 1680.1 |
| 18 | 1679.26 | 4.62 | 1680.1 |
| 19 | 2271.23 | 5.96 | 2271.57 |
| 20 | 1848.7 | 6.02 | 1849.21 |
| 21 | 2693.76 | 5.88 | 2694.1 |
| 22 | 2468.55 | 6.2 | 2469.62 |
| 23 | 2665.87 | 6.42 | 2666.7 |
| 24 | 2017.8 | 5.41 | 2018.02 |
| 25 | 2115.99 | 5.63 | 2116.41 |
| 26 | 2552.54 | 5.58 | 2553.77 |
| 27 | 1721.34 | 4.76 | 1721.81 |
| 28 | 2115.99 | 5.58 | 2116.4 |
| 29 | 2552.54 | 5.52 | 2552.81 |
| 30 | 1721.34 | 4.7 | 1722.11 |
| 31 | 2236.18 | 5.54 | 2236.66 |
| 32 | 2712.8 | 5.46 | 2713.72 |
| 33 | 1841.54 | 4.64 | 1842.2 |
| 34 | 2194.1 | 5.65 | 2194.43 |
| 35 | 2656.7 | 5.6 | 2656.74 |
| 36 | 1799.46 | 4.79 | 1800.27 |
| 37 | 927.25 | 6.06 | 928.8 |
| 38 | 1349.78 | 5.98 | 1351.8 |
| 39 | 1772.31 | 5.87 | 1774.1 |
| 40 | 1716.2 | 5.66 | 1718.1 |
| 41 | 906.44 | 5.39 | 908 |
| 42 | 920.47 | 5.36 | 921.6 |
| 43 | 921.45 | 5.62 | 922.9 |
| 44 | 977.56 | 5.23 | 978 |
| 45 | 1046.67 | 5.16 | 1047.7 |
| 46 | 1034.66 | 5.23 | 1035.1 |
| 47 | 971.52 | 5.39 | 972.6 |
| 48 | 2859.78 | 5.61 | 1408.8 |
| | (average) | | (MH₂⁺², |
| | | | distribution, |
| | | | δ = 22.06) |
| 49 | 1328.97 | 5.51 | 1330 |
| 50 | 1343 | 5.43 | 1343.9 |
| 51 | 1343.98 | 5.62 | 1345 |
| 52 | 1400.09 | 5.35 | 1401.1 |
| 53 | 1469.2 | 5.33 | 1470.2 |
| 54 | 1457.19 | 5.38 | 1457.3 |
| 55 | 1394.04 | 5.42 | 1395.1 |
| 56 | 3282.31 | 5.63 | 1664.2 |
| | (average) | | (MH₂⁺², |
| | | | distribution, |
| | | | δ = 22.08) |
| 57 | 1751.5 | 5.51 | 1752.3 |
| 58 | 1765.53 | 5.48 | 1766.4 |
| 59 | 1766.51 | 5.61 | 1767.3 |
| 60 | 1822.62 | 5.4 | 1824.4 |
| 61 | 1891.73 | 5.38 | 1892.5 |
| 62 | 1879.72 | 5.42 | 1880.5 |
| 63 | 1816.57 | 5.47 | 1817.3 |
| 64 | 3704.84 | 5.59 | 1408.8 |
| | (average) | | (MH₂⁺², |
| | | | distribution, |
| | | | δ = 22.04) |
| 65 | 1695.39 | 5.21 | 1697.2 |
| 66 | 1709.42 | 5.23 | 1710.3 |
| 67 | 1710.4 | 5.36 | 1711.3 |
| 68 | 1766.51 | 5.14 | 1767.3 |
| 69 | 1835.62 | 5.12 | 1836.4 |
| 70 | 1823.61 | 5.17 | 1823.4 |
| 71 | 1760.47 | 5.22 | 1761.2 |
| 72 | 3648.73 | 5.37 | 1803.0 |
| | (average) | | (MH₂⁺², |
| | | | distribution, |
| | | | δ = 22.06) |

### Example 3—Formulation of Representative Amino Lipidated Peptoids with Oligonucleotides to Form Nanoparticle Compositions

The following example describes the general protocol for the formulation of the tertiary amino lipidated and/or PEGylated cationic peptide compounds of formula (I) as described herein with oligonucleotides.

In standard formulations, the amino lipidated peptoid is dissolved in anhydrous ethanol at a concentration of 0.5 mg/mL (for in vitro experiments) or 5 mg/mL (for in vivo experiments). The resulting solutions are stable at room temperature, but should be stored at −20° C. The nucleic acid cargo is dissolved in DNAse or RNAse-free water at a final concentration of 0.2 mg/mL (for in vitro experiments) or 1 mg/mL (for in vivo experiments). These solutions should be stored at −20° C., or at −78° C. for longer time periods.

To prepare nanoparticle formulations, the amino lipidated peptoid is mixed by pipetting with nucleic acid at a mass ratio between approximately 1:1 (peptoid:cargo) and 20:1. Prior to formulation, amino lipidated peptoid and nucleic acid cargo were diluted in ethanol and acidic buffer (PBS adjusted to pH 5.5 with 0.1 M HCl) respectively to a volume ratio of 1:3 and to target a final cargo concentration of approximately 0.05 mg/mL to 0.2 mg/mL.

### Example 4—Preparation and Characterization of Physical Properties of Representative mRNA/Peptoid Formulations

The exemplary amino lipidated cationic peptoids described in Example 2 were combined with firefly luciferase (Fluc) mRNA to form nanoparticle compositions to be evaluated for therapeutic and/or prophylactic purposes in vitro or in vivo. The formulations were prepared according to the protocol of Example 3 and mixed by simple pipetting.

The mRNA/peptoid formulation at a 5:1 mass ratio of peptoid:cargo were evaluated by dynamic light scattering (DLS) in order to determine the volume average particle size/diameter (nm) of the mRNA/peptoid complex and the size polydispersity index (PDI) within the formulation. The percentage of mRNA encapsulation for each formulation of the exemplary compounds were determined by fluorescence of the Qubit RNA HS (Invitrogen) dye before and after lysis of particles by Triton X-100. The results are shown in Table 2 below.

TABLE 2

| Compound | Size (nm) | Size PDI | % Encapsulation |
|---|---|---|---|
| 1 | 356.9 | 0.047 | 90.1 |
| 2 | 150 | 0.128 | 100.3 |
| 3 | 255.3 | 0.247 | 95.9 |
| 4 | 326.7 | 0.191 | 99.0 |
| 5 | 364.7 | 0.308 | 88.3 |
| 6 | 448.6 | 0.348 | 92.4 |
| 7 | 296.7 | 0.177 | 88.4 |
| 8 | 234 | 0.077 | 82.5 |
| 9 | 367.9 | 0.122 | 73.9 |
| 10 | 157 | 0.009 | 92.6 |
| 11 | 354.9 | 0.038 | 87.8 |
| 12 | 224.4 | 0.211 | 86.5 |
| 13 | 822.6 | 0.447 | 90.4 |
| 14 | 554 | 0.233 | 77.3 |
| 15 | 1064 | 1 | 76.9 |
| 16 | 1486 | 1 | 85.4 |
| 17 | 472.8 | 0.37 | 90.3 |
| 18 | 593 | 0.762 | 88.5 |
| 19 | 276 | 0.158 | 70.9 |
| 20 | 326.6 | 0.37 | 72.2 |
| 21 | 386.5 | 0.174 | 65.4 |
| 22 | 748.2 | 0.538 | 65.4 |
| 23 | NA | NA | 3.9 |
| 24 | 161 | 0.135 | 87.0 |
| 25 | 773.3 | 0.021 | 8.4 |
| 26 | NA | NA | 35.7 |
| 27 | 438.7 | 0.606 | 50.9 |
| 28 | 308 | 0.085 | 52.0 |
| 29 | 343.4 | 0.237 | 24.8 |
| 30 | 282.8 | 0.455 | 8.4 |
| 31 | 348.5 | 0.404 | 35.7 |
| 32 | 247.5 | 0.274 | 50.9 |
| 33 | 435.7 | 0.048 | 82.0 |
| 34 | 348.4 | 0.295 | 58.0 |
| 35 | 359.8 | 0.486 | 38.0 |
| 36 | 374.4 | 0.127 | 71.5 |

### Example 5—Effect of PEG on Formulation Stability and Transfection Efficiency

In order to evaluate the effect of variable amounts of PEG incorporation on the formulation stability (i.e., particle size) and transfection efficiency of peptoid formulations, three formulations were prepared using compound 8 alone or combined with DMG-PEG 2000 at 2% w/w or 5% w/w. Particles were formulated using 5 μg mRNA at 0.2 μg/μL concentration. Immediately following formulation, the particles were added to PBS (pH 7.4).

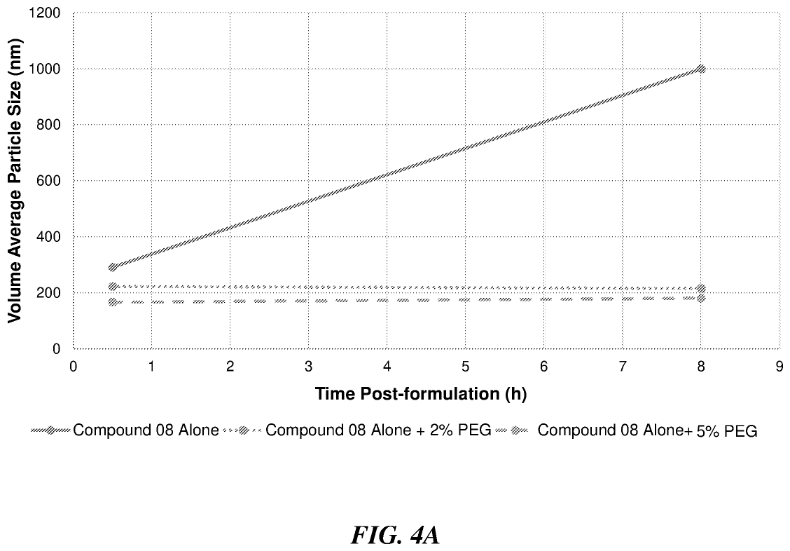

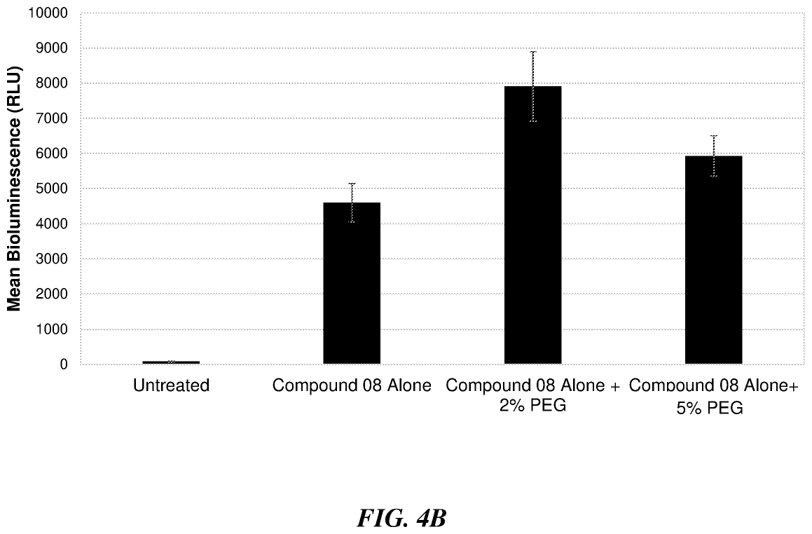

The volume average particle size (nm) of the three formulations was evaluated over immediately following formulation and addition to PBS buffer (0.5 h) and 8 hours after formulation (8 h) of by the same method as in Example 4 above. The measured number average particle size is depicted in FIG. 4A.

The transfection efficacy was also evaluated for the three formulations of compound 8 alone, with 2% DMG-PEG 2000 or with 5% DMG-PEG 2000. FIG. 4B shows the observed mean bioluminescence (RLU) of HeLa cells 8 hours after treatment with 100 ng mRNA/well approximately 30 minutes post-formulation. Untreated HeLa cells were used as a control for the bioluminescence measurements.

### Example 6—Storage Stability

In order to evaluate the storage stability of the formulations containing amino lipidated peptoid compounds with 2% w/w PEG incorporation, a formulation of amino lipidated peptoid compound 28 with Fluc mRNA and 2% w/w DMG-PEG2000 was prepared either 7 days prior, 3 days prior, or 1 hour prior to injection. In the interim, the formulations were stored at 4° C. These three formulations were injected via tail vein injection into three mice each.

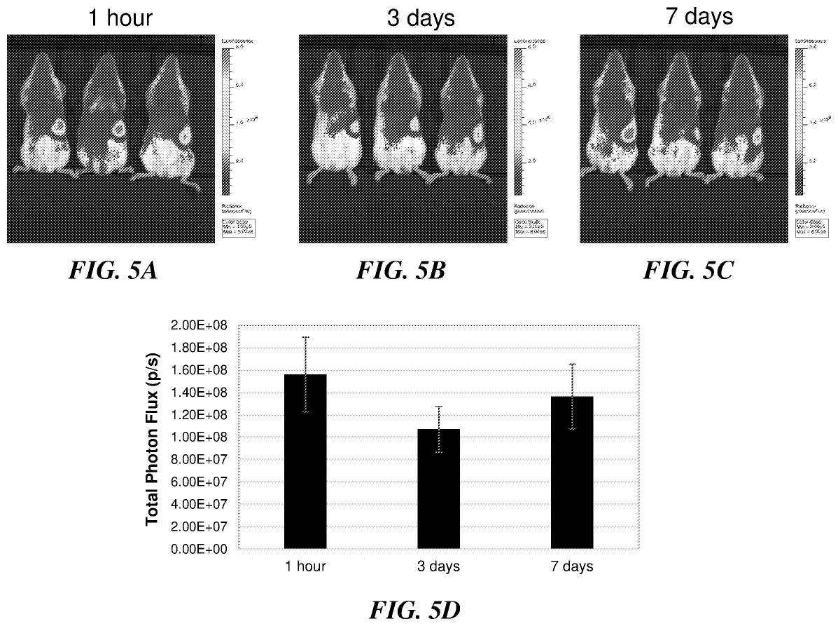

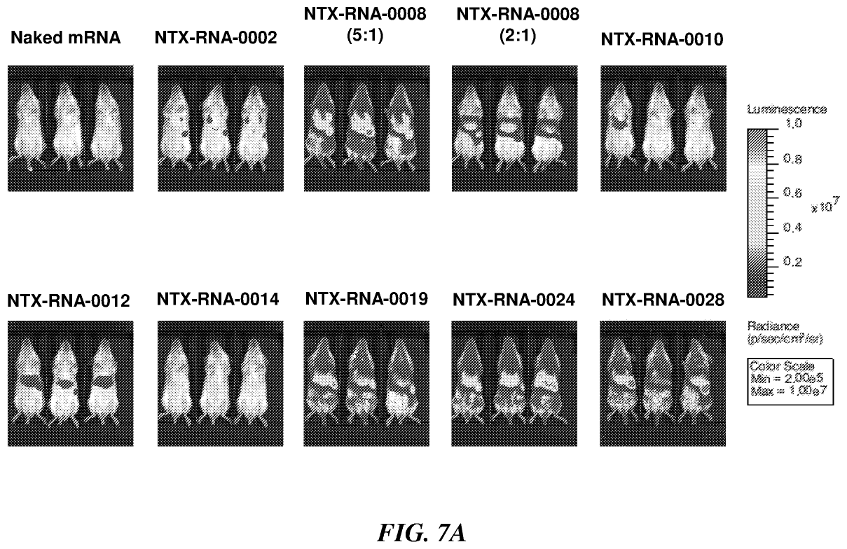

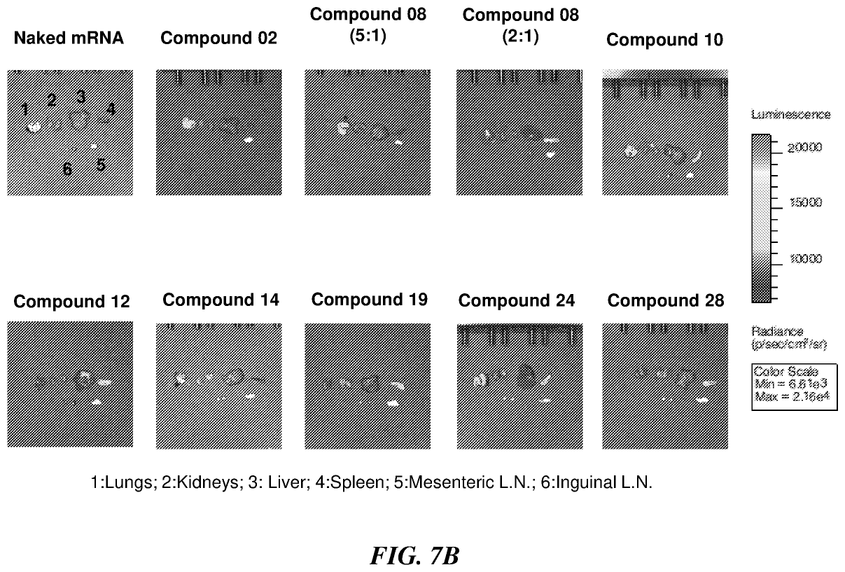

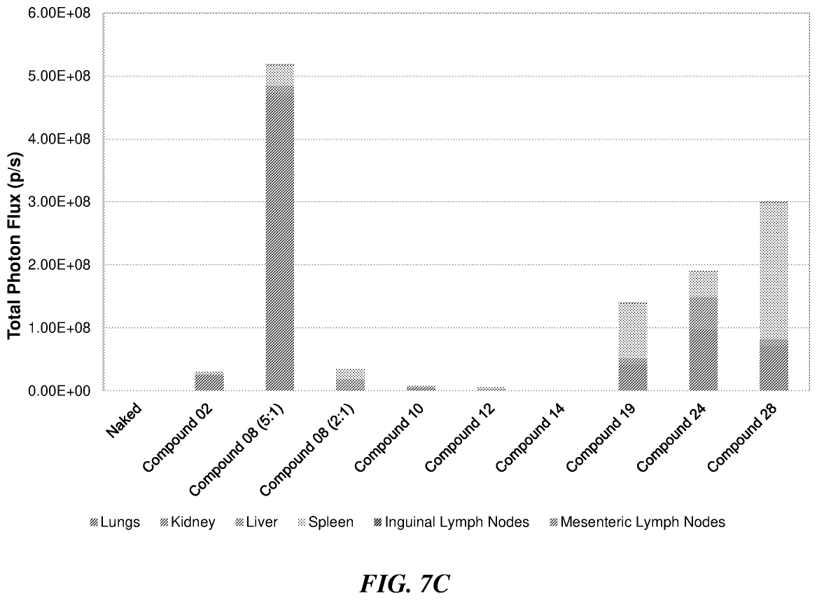

The storage stability of the formulations was evaluated by recording whole body distribution luminescence (or photon flux) 8 hours post-injection. FIGS. 5A-5C show the whole body luminescence after 1 hour (FIG. 5A), 3 days (FIG. 5B) and 7 days (FIG. 5C) of storage. FIG. 5D shows a plot of the total photon flux observed for each of the time points.

### Example 7—In Vitro Expression of Firefly Luciferase (Fluc) Following Treatment with Representative Fluc mRNA/Amino Lipidated Peptoid Formulations Alone (No PEG) or Containing 2% (w/w) DMG-PEG2000 (2% PEG)

The efficacy of mRNA/amino lipidated peptoid formulations was evaluated in vitro based on their ability to deliver the firefly luciferase (Fluc) reporter gene to cultured cells. Amino lipidated peptoids were combined with Fluc mRNA at a 5:1 w/w ratio, and the resulting particles were added to cultured HeLa cells at a dose of 100 ng/well (in 100 μL total volume). The resulting luciferase expression (RLU) was measured by a luminescence plate reader after 6 hours and 24 hours of treatment. Table 3 below shows the observed luciferase expression for the mRNA/amino lipidated peptoid formulations at the two time points.

TABLE 3
In vitro expression of firefly luciferase
(Fluc) following treatment with representative
Fluc mRNA/amino lipidated peptoid formulations

| | Luciferase Expression (RLU) | |
|---|---|---|
| Compound | 6 h | 24 h |
| 1 | 1.22E+02 | 4.00E+01 |
| 2 | 2.35E+02 | 1.35E+02 |
| 3 | 2.27E+02 | 2.99E+02 |
| 4 | 1.96E+02 | 1.13E+02 |
| 5 | 9.60E+01 | 5.36E+02 |
| 6 | 4.30E+01 | 3.60E+01 |
| 7 | 6.45E+03 | 9.71E+03 |
| 8 | 1.10E+04 | 1.33E+04 |
| 9 | 1.52E+02 | 8.57E+01 |
| 10 | 1.88E+03 | 7.01E+02 |
| 11 | 1.15E+03 | 1.22E+03 |
| 12 | 4.04E+02 | 5.71E+02 |
| 13 | 3.67E+00 | 9.67E+00 |
| 14 | 3.20E+01 | 1.43E+01 |
| 15 | 3.93E+01 | 1.90E+01 |
| 16 | 7.33E+00 | 1.67E+01 |
| 17 | 6.84E+02 | 5.63E+02 |
| 18 | 5.74E+02 | 8.49E+02 |
| 19 | 8.20E+03 | 1.09E+04 |
| 20 | 9.91E+03 | 1.55E+04 |
| 21 | 3.45E+03 | 1.24E+04 |
| 22 | 6.63E+01 | 6.77E+01 |
| 23 | 5.60E+01 | 9.47E+01 |
| 24 | 1.79E+04 | 3.43E+04 |
| 25 | 3.93E+02 | 2.07E+03 |
| 26 | 5.00E+00 | 1.07E+01 |
| 27 | 9.88E+02 | 7.40E+02 |
| 28 | 9.59E+02 | 4.97E+03 |
| 29 | 1.64E+03 | 6.72E+03 |
| 30 | 2.79E+03 | 1.29E+04 |
| 31 | 5.84E+02 | 2.93E+03 |
| 32 | 4.25E+02 | 2.00E+03 |
| 33 | 4.61E+03 | 1.14E+04 |
| 34 | 1.95E+03 | 2.71E+03 |
| 35 | 1.22E+03 | 1.60E+03 |
| 36 | 9.34E+03 | 7.78E+03 |

In addition to evaluating nanoparticle complexes formed between mRNA cargos and amino lipidated peptoids alone, multicomponent formulations containing a small amount of PEG-lipid DMG-PEG2000 were also tested for in vitro Fluc mRNA delivery. In these formulations, DMG-PEG2000 was pre-mixed with candidate peptoid at an overall 2% incorporation (w/w), while maintaining the same 5:1 mass ratio of peptoid:mRNA used previously. Table 4 below shows the observed luciferase expression for the mRNA/amino lipidated peptoid formulations with 2% DMG-PEG2000 incorporation at the two time points (6 hours and 24 hours after treatment).

TABLE 4
In vitro expression of firefly luciferase
(Fluc) following treatment with representative
Fluc mRNA/amino lipidated peptoid
formulations with 2% DMG-PEG2000

| | Luciferase Expression (RLU) | |
|---|---|---|
| Compound | 6 h | 24 h |
| 1 | 2.09E+02 | 8.67E+00 |
| 2 | 5.40E+01 | 1.17E+02 |
| 3 | 3.14E+02 | 4.68E+02 |
| 4 | 3.16E+02 | 3.09E+02 |
| 5 | 3.46E+02 | 1.22E+02 |
| 6 | 1.38E+02 | 2.13E+01 |
| 7 | 2.77E+03 | 1.81E+03 |
| 8 | 5.32E+03 | 3.81E+03 |
| 9 | 1.38E+02 | 3.33E+01 |
| 10 | 2.21E+02 | 2.43E+02 |
| 11 | 3.90E+02 | 5.23E+02 |
| 12 | 3.13E+02 | 3.36E+02 |
| 13 | 3.27E+01 | 2.67E+00 |
| 14 | 4.67E+00 | 9.00E+00 |
| 15 | 5.33E+00 | 5.33E+00 |
| 16 | 2.63E+01 | 5.00E+00 |
| 17 | 8.67E+01 | 1.80E+02 |
| 18 | 8.50E+01 | 1.22E+02 |
| 19 | 1.64E+04 | 1.20E+04 |
| 20 | 1.75E+04 | 1.36E+04 |
| 21 | 1.42E+04 | 8.84E+03 |
| 22 | 9.23E+01 | 2.13E+01 |
| 23 | 2.53E+01 | 2.13E+01 |
| 24 | 9.70E+03 | 7.93E+03 |
| 25 | 1.81E+03 | 7.30E+02 |
| 26 | 5.85E+02 | 6.45E+02 |
| 27 | 3.13E+02 | 6.36E+02 |
| 28 | 8.33E+02 | 6.94E+02 |
| 29 | 1.84E+03 | 1.18E+03 |
| 30 | 1.79E+03 | 1.52E+03 |
| 31 | 4.30E+02 | 2.63E+02 |
| 32 | 4.82E+02 | 3.40E+02 |
| 33 | 4.64E+02 | 5.17E+02 |
| 34 | 5.70E+02 | 7.26E+02 |
| 35 | 4.45E+02 | 7.59E+02 |
| 36 | 3.36E+02 | 7.45E+02 |

### Example 8—In Vitro Expression of Firefly Luciferase (Fluc) Following Treatment with Representative Fluc mRNA/Amino Lipidated Peptoid Formulations without DMG-PEG2000 or Containing 2% (w/w) DMG-PEG2000 at Variable Peptoid:Cargo Mass Ratios

The charge ratio dependence of amino lipidated peptoid delivery vehicles was evaluated by formulating particles between Fluc mRNA and delivery vehicles at varying mass ratios. Formulations were varied between 2:1 and 7.5:1 peptoid:mRNA (w/w) ratios (2:1, 3.5:1, 5:1 and 7.5:1). In addition, the role of DMG-PEG2000 was also explored with the mass ratios, either by excluding DMG-PEG2000 or incorporating DMG-PEG2000 at 2% (w/w) relative to amino lipidated peptoid. Fluc expression from these formulations was measured in HeLa cells following treatment for 24 hours.

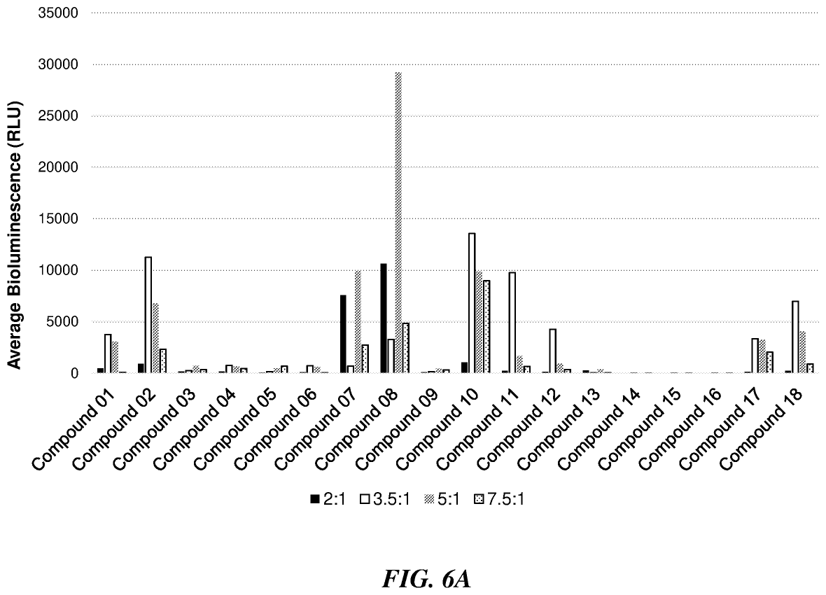

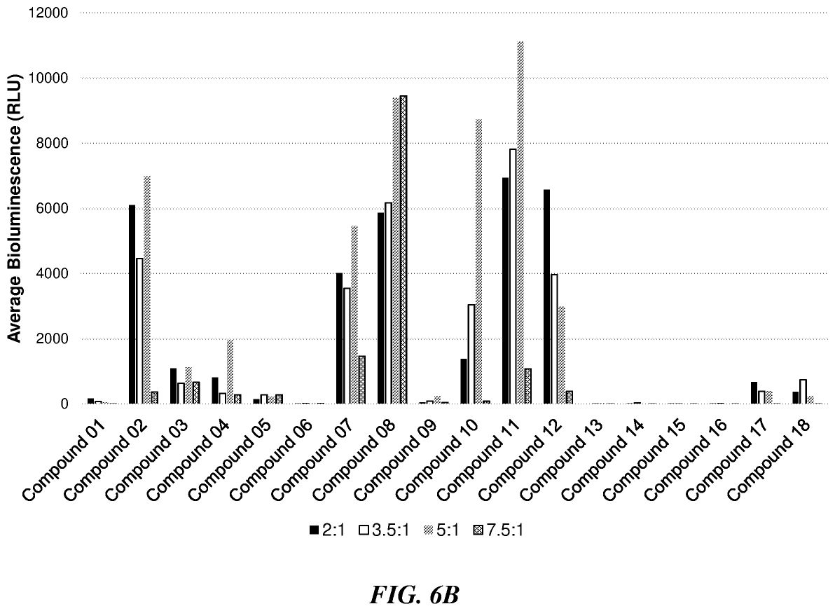

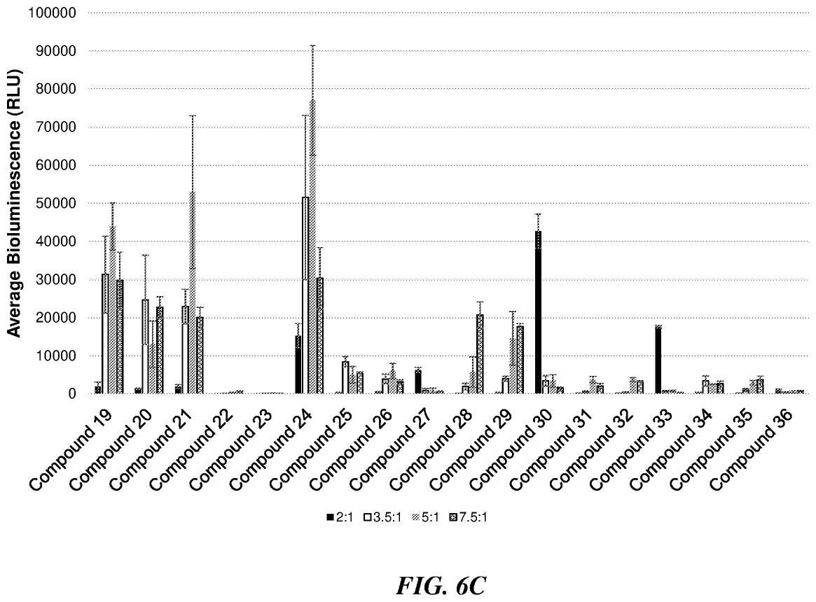

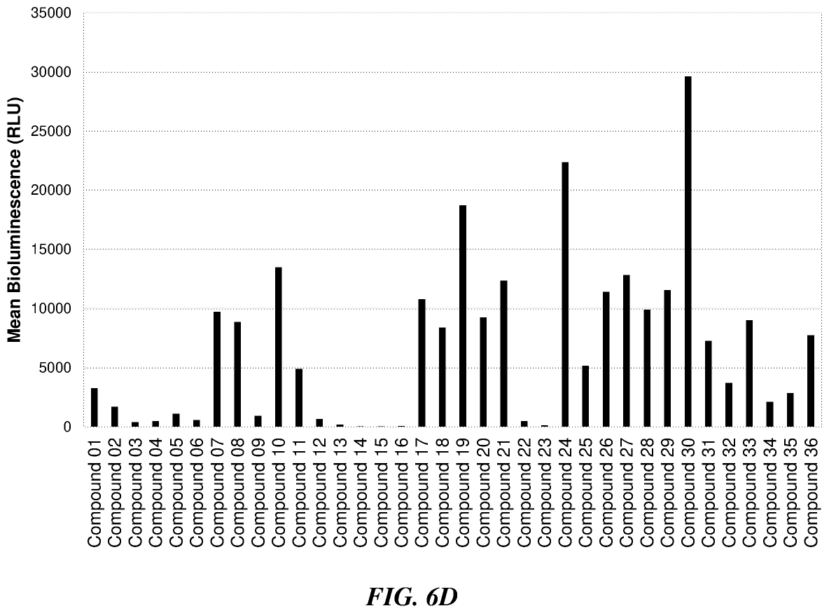

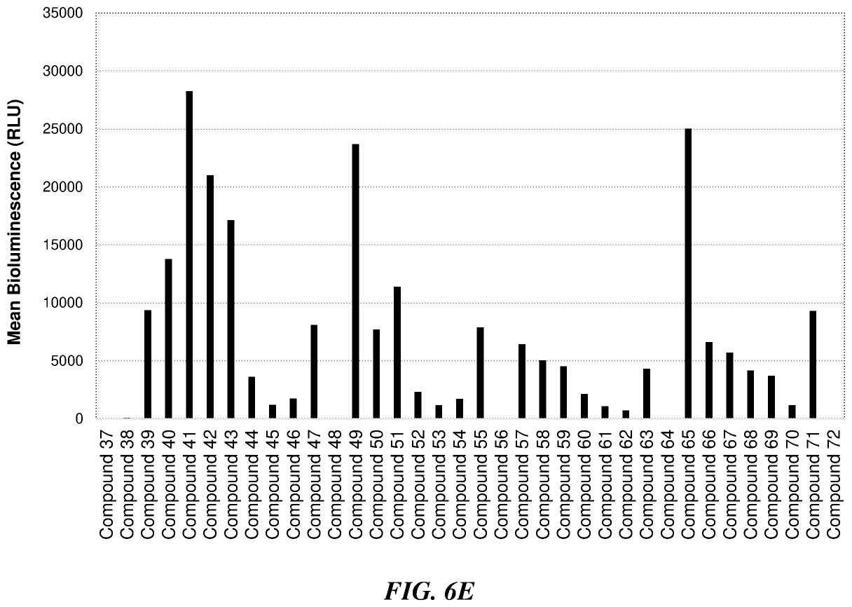

FIG. 6A shows the results for in vitro luciferase expression measurements carried out for exemplary amino lipidated cationic peptoid compounds 1-18 from Example 2 without PEG incorporation. FIGS. 6B and 6C show plots of the average bioluminescence (RLU) for HeLa cells treated with the formulations of exemplary amino lipidated cationic peptoid compounds 1-36, all formulated with 2% w/w DMG-PEG2000. Table 5 shows the observed Fluc expression for HeLa cells treated with formulations containing 2% w/w DMG-PEG2000.

TABLE 5
In vitro expression of firefly luciferase (Fluc) following
treatment with representative Fluc mRNA/amino
lipidated peptoid formulations containing 2% (w/w)
DMG-PEG2000 with variable peptoid:mRNA mass ratio

| | Observed Luciferase Expression (RLU) | | | |
|---|---|---|---|---|
| | for Given Formulation Mass Ratio (+/−) | | | |
| Compound | 2:1 | 3.5:1 | 5:1 | 7.5:1 |
| 1 | 6.82E+02 | 3.12E+02 | 2.31E+02 | 3.35E+01 |
| 2 | 2.45E+04 | 1.80E+04 | 2.81E+04 | 1.49E+03 |
| 3 | 4.42E+03 | 2.56E+03 | 4.53E+03 | 2.69E+03 |
| 4 | 3.27E+03 | 1.32E+03 | 7.84E+03 | 1.14E+03 |
| 5 | 5.81E+02 | 1.15E+03 | 9.21E+02 | 1.14E+03 |
| 6 | 8.18E+01 | 8.18E+01 | 6.97E+01 | 4.56E+01 |
| 7 | 1.61E+04 | 1.43E+04 | 2.20E+04 | 5.92E+03 |
| 8 | 2.36E+04 | 2.48E+04 | 3.78E+04 | 3.80E+04 |
| 9 | 2.28E+02 | 3.67E+02 | 9.91E+02 | 1.82E+02 |
| 10 | 5.57E+03 | 1.23E+04 | 3.51E+04 | 3.57E+02 |
| 11 | 2.79E+04 | 3.14E+04 | 4.48E+04 | 4.36E+03 |
| 12 | 2.64E+04 | 1.60E+04 | 1.20E+04 | 1.58E+03 |
| 13 | 2.68E+01 | 1.74E+01 | 1.39E+02 | 5.36E+00 |
| 14 | 8.31E+01 | 1.42E+02 | 4.29E+01 | 2.82E+01 |
| 15 | 8.85E+01 | 3.62E+01 | 2.01E+01 | 6.70E+00 |
| 16 | 1.01E+02 | 4.16E+01 | 2.55E+01 | 1.47E+01 |
| 17 | 2.72E+03 | 1.56E+03 | 1.60E+03 | 3.75E+01 |
| 18 | 1.53E+03 | 3.00E+03 | 9.99E+02 | 2.41E+01 |
| 19 | 1.90E+03 | 3.13E+04 | 4.39E+04 | 2.98E+04 |
| 20 | 1.31E+03 | 2.47E+04 | 1.31E+04 | 2.27E+04 |
| 21 | 1.92E+03 | 2.29E+04 | 5.29E+04 | 2.01E+04 |
| 22 | 3.53E+01 | 8.83E+01 | 2.50E+02 | 5.80E+02 |
| 23 | 5.47E+01 | 1.42E+02 | 2.07E+02 | 1.55E+02 |
| 24 | 1.53E+04 | 5.16E+04 | 7.71E+04 | 3.03E+04 |
| 25 | 3.86E+02 | 8.41E+03 | 5.03E+03 | 5.66E+03 |
| 26 | 4.91E+02 | 3.93E+03 | 6.06E+03 | 3.19E+03 |
| 27 | 6.27E+03 | 8.40E+02 | 8.69E+02 | 6.41E+02 |
| 28 | 1.26E+02 | 1.93E+03 | 5.75E+03 | 2.07E+04 |
| 29 | 3.67E+02 | 3.99E+03 | 1.46E+04 | 1.77E+04 |
| 30 | 4.27E+04 | 3.43E+03 | 3.46E+03 | 1.47E+03 |
| 31 | 2.07E+02 | 5.70E+02 | 3.77E+03 | 2.05E+03 |
| 32 | 1.82E+02 | 3.09E+02 | 3.77E+03 | 3.37E+03 |
| 33 | 1.76E+04 | 6.62E+02 | 7.46E+02 | 3.12E+02 |
| 34 | 3.67E+02 | 3.40E+03 | 2.54E+03 | 2.66E+03 |
| 35 | 1.72E+02 | 9.94E+02 | 2.92E+03 | 3.69E+03 |
| 36 | 9.24E+02 | 4.18E+02 | 5.17E+02 | 6.71E+02 |

Formulations of amino lipidated peptoid compounds 37-72 with 2% DMG-PEG2000 were also evaluated for their ability to deliver Fluc mRNA into HeLa cells. FIGS. 6D and 6E shows a plot of mean bioluminescence (RLU) observed for HeLa cells treated with various formulations of amino lipidated peptoid compounds 1-72 in combination with Fluc mRNA (50 ng/well, peptoid:mRNA ratio of 5:1) and 2% DMG-PEG2000.

### Example 9—In Vitro Cellular Viability Following Treatment with Representative Fluc mRNA/Amino Lipidated Peptoid Formulations Alone (No PEG) or Containing 2% (w/w) DMG-PEG2000 (2% PEG)

Cellular viability was measured following treatment with Fluc mRNA/amino lipidated peptoid formulations by a traditional MTT viability assay. All peptoid/mRNA formulations were prepared at a 5:1 mass ratio with 2% (w/w) DMG-PEG2000. In this assay, cells were first treated with peptoid formulations at a final mRNA concentration of 100 ng/well for 8 hours. Cells were then left to grow for 48 hours, after which time they were exposed to 1.5 mg/mL 3-(4,5-dimethylthiazol-2-yl)-2,5-diphenyltetrazolium bromide (MTT) for 2 hours, then lysed using a solubilizing solution of 10% Triton-X 100 in acidic isopropanol (0.1N HCl). The signal corresponding to reduced formazan was normalized to untreated cells to provide a relative viability of each treated condition.

Only minor changes in viability were observed for HeLa cells treated with mRNA/amino lipidated peptoid formulations. In general, nearly all conditions maintained at least 80% viability with the only exceptions being formulations of compound 5, 34, and 35. On average, formulations containing 2% DMG-PEG2000 exhibited slightly higher (average of 97% viability versus 89% for PEG-absent formulations). Table 6 shows the relative viability of cells treated with the PEG-absent and 2%-PEG formulations.

TABLE 6
In vitro cellular viability following
treatment with representative Fluc mRNA/amino
lipidated peptoid formulations alone (no PEG) or
containing 2% (w/w) DMG-PEG2000 (2% PEG)

| | Relative Viability (%) | |
|---|---|---|
| Compound | No PEG | 2% PEG |
| 1 | 97.4 | 88.1 |
| 2 | 90.6 | 92.6 |
| 3 | 91.2 | 95.3 |
| 4 | 90.4 | 89.7 |
| 5 | 56.8 | 91.2 |
| 6 | 88.0 | 88.9 |
| 7 | 78.0 | 96.0 |
| 8 | 92.0 | 97.3 |
| 9 | 99.3 | 107.2 |
| 10 | 100.9 | 108.0 |
| 11 | 103.9 | 103.6 |
| 12 | 98.5 | 104.0 |
| 13 | 90.2 | 85.9 |
| 14 | 93.5 | 91.0 |
| 15 | 91.2 | 91.9 |
| 16 | 89.4 | 90.8 |
| 17 | 88.0 | 91.7 |
| 18 | 86.1 | 89.3 |
| 19 | 87.1 | 94.8 |
| 20 | 87.1 | 93.0 |
| 21 | 91.3 | 108.9 |
| 22 | 103.5 | 118.0 |
| 23 | 108.1 | 118.1 |
| 24 | 92.0 | 112.3 |
| 25 | 77.9 | 85.9 |
| 26 | 98.8 | 91.9 |
| 27 | 93.9 | 91.1 |
| 28 | 85.4 | 92.7 |
| 29 | 85.9 | 94.0 |
| 30 | 86.5 | 88.0 |
| 31 | 83.8 | 98.4 |
| 32 | 90.7 | 102.2 |
| 33 | 99.4 | 98.0 |
| 34 | 67.6 | 106.1 |
| 35 | 74.8 | 105.2 |
| 36 | 93.6 | 108.1 |

### Example 10—In Vivo Whole-Body Expression of Firefly Luciferase (Fluc) Following IV Administration of Representative Fluc mRNA/Amino Lipidated Peptoid Formulations

Amino lipidated peptoid delivery vehicles are effective for in vivo administration of Fluc mRNA to Balb/c mice (n=3) through multiple routes of administration. Briefly, representative amino lipidated peptoid/Fluc formulations (prepared at a 5:1 mass ratio of amino lipidated peptoid:mRNA unless noted, with 2% w/w DMG-PEG2000) were administered at a dose of 0.5 mg/kg via a tail-vein injection, and the resulting bioluminescence was quantified after 8 hours. FIG. 7A shows the whole body distribution of the formulations in the Balb/c mice. Table 7 shows the in vivo whole body expression of Fluc in the test mice quantified as total flux.

TABLE 7
In vivo whole-body expression of firefly luciferase
(Fluc) following IV administration of representative
Fluc mRNA/amino lipidated peptoid formulations
(all prepared at a 5:1 mass ratio of peptoid:mRNA)

| | Total Flux |
|---|---|
| Compound | (photons/sec) |
| none | 2.82E+05 |
| 2 | 1.37E+07 |
| 8 | 3.24E+08 |
| 10 | 1.04E+07 |
| 12 | 1.65E+07 |
| 14 | 4.58E+05 |
| 19 | 2.02E+08 |
| 24 | 2.85E+08 |
| 28 | 2.36E+08 |

Organ-specific bioluminescence was quantified by sacrificing the treated animal, dissecting out the organs of interest, and separately quantifying the resulting bioluminescence. The relative expression of each of the highest-performing amino lipidated peptoids varied significantly. For instance, while Compound 8 showed the highest overall expression, nearly all of that expression was localized in the lung. However, other compounds showed preferential localization in other organs such as the spleen in the case of Compound 28, and a mix of liver, spleen, and lung for Compound 24.

FIG. 7B shows the corresponding organ-specific biodistribution (in lungs, kidney, liver, spleen, inguinal lymph nodes and mesenteric lymph nodes) of the formulations. FIG. 7C depicts a bar chart quantifying the organ-specific biodistribution of the formulations as a function of total photon flux. Table 8 shows the total flux measured for liver, lung and spleen samples obtained from the treated mice.

TABLE 8
In vivo biodistribution of firefly luciferase (Fluc) to internal
organs following IV administration of representative
Fluc mRNA/amino lipidated peptoid formulations

| | Total Flux (p/s) | | |
|---|---|---|---|
| Compound | Liver | Lung | Spleen |
| none | 2.61E+04 | 2.76E+04 | 2.01E+04 |
| 2 | 1.40E+06 | 2.46E+07 | 3.39E+06 |
| 8 | 1.04E+07 | 4.68E+08 | 3.28E+07 |
| 10 | 5.85E+05 | 5.14E+06 | 2.17E+06 |
| 12 | 2.74E+06 | 6.23E+05 | 2.36E+06 |
| 14 | 4.47E+04 | 4.17E+04 | 4.89E+04 |
| 19 | 8.37E+06 | 4.17E+07 | 8.75E+07 |
| 24 | 5.16E+07 | 9.63E+07 | 4.03E+07 |
| 28 | 1.10E+07 | 7.08E+07 | 2.16E+08 |

In addition to IV administration, amino lipidated peptoids are also effective for local administration of mRNA through subcutaneous (S.C.) or intramuscular (I.M.) routes of administration. Table 9 shows the total flux observed for a selection of amino lipidated peptoid formulations administered to Balb/c mice subcutaneously or intramuscularly.

TABLE 9
In vivo expression of firefly luciferase (Fluc)
following subcutaneous (S.C.) or intramuscular
(I.M.) administration of representative Fluc
mRNA/amino lipidated peptoid formulations

| | Total Flux (photons/sec) | |
|---|---|---|
| Compound | S.C. (0.1 mpk) | I.M. (0.05 mpk) |
| none | 1.67E+05 | 1.83E+05 |
| 2 | 8.31E+04 | 1.27E+05 |
| 8 | 5.86E+05 | 2.31E+06 |
| 10 | 1.40E+05 | 9.01E+05 |
| 19 | 1.62E+06 | 7.20E+06 |
| 24 | 4.36E+06 | 4.18E+06 |
| 28 | 1.65E+06 | 7.23E+06 |

For these examples, mRNA was administered at a dose of 0.1 milligrams/kilogram (mpk) for subcutaneous or 0.05 mpk for intramuscular. In these cases, the overall expression was higher following intramuscular injections. While the highest expression was observed with Compound 8 for IV administration, Compounds 24 and 28 significantly outperformed that example for IM administration, suggesting that tailoring these structures for different routes of administration is necessary.

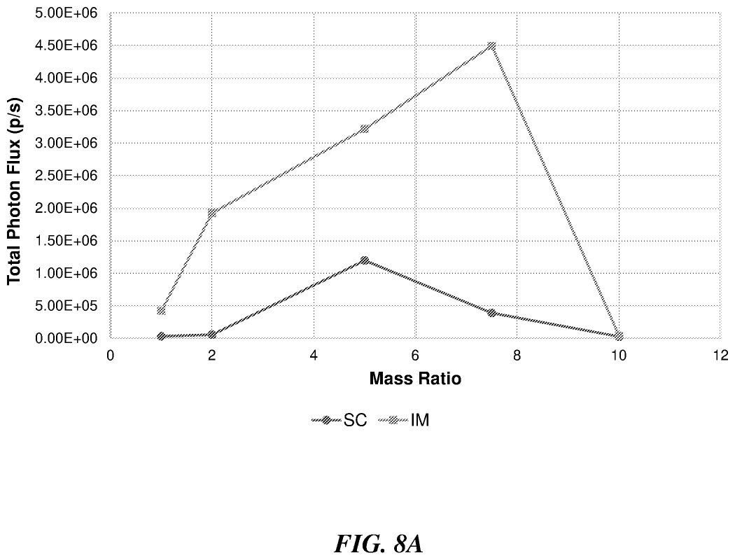

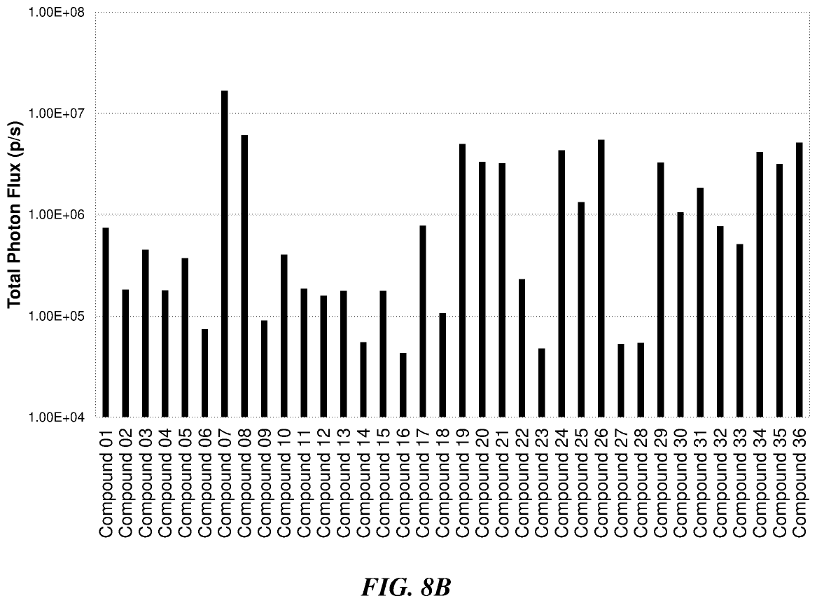

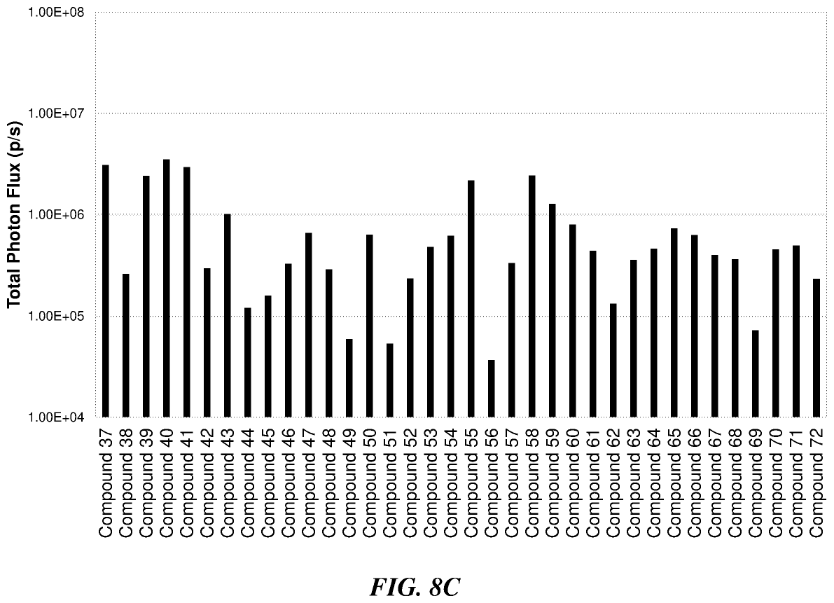

Additional experiments were carried out to assess any variability in biodistribution as a function of the loading mass ratio of amino lipidated peptoid to the Fluc mRNA. FIG. 8A depicts a plot of total photon flux for formulations of amino lipidated peptoid compound 28 with 2% DMG-PEG 2000 and Fluc mRNA at different peptoid:mRNA ratios (1:1, 2:1, 5:1, 7.5:1 and 10:1). As shown in FIG. 8A, maximal total photon fluxes for formulations of compound 28 were observed at 5:1 for subcutaneous injection and 7.5:1 for intramuscular injection.

Evaluation of biodistribution profiles for subcutaneous administration were extended amino lipidated peptoid compounds 1-72. Formulations of amino lipidated peptoid compounds 1-72 with 2% w/w DMG-PEG2000 and Fluc mRNA at a peptoid:mRNA ratio of 5:1 were administered subcutaneously to Balb/c mice. The observed measurement of total photon fluxes for each of the formulations are shown in FIGS. 8B and 8C.

### Example 11—In Vitro Cell Type Transfection Efficiency of Firefly Luciferase (Fluc) in Amino Lipidated Peptoid Formulations with PEG

Cell Culture: HeLa cells were seeded at 10,000 wells in 100 μL DMEM containing 10% FBS and 1% penicillin/streptomycin, 18 hours prior to treatment and left to adhere. Immediately prior to transfection, media was replaced with 100 μL fresh serum-containing DMEM. HepG2 cells were cultured in DMEM containing 10% FBS and 1% penicillin/streptomycin. They were seeded at 10,000 cells/well 18 hours prior to treatment and left to adhere. JAWSII cells were cultured in RPMI media containing 10% FBS, 1% penicillin/streptomycin, 1 mM Sodium Pyruvate, and 5 ng/mL murine GM-CSF.

mRNA preparation: Fluc mRNA for transfection experiments was prepared in-house using standard in vitro transcription (IVT) methods.

mRNA Formulation: Representative amino lipidated peptoid/Fluc formulations (prepared at a 5:1 mass ratio of amino lipidated peptoid:mRNA unless noted) for compounds 24 and 79. The amino lipidated peptoid:mRNA mixtures were combined with commercially available PEG lipids (DMG-PEG2000; 18:0 PEG1000PE; 14:0 PEG2000 PE; 14:0 PEG5000 PE), linear amino PEGylated peptoids (Compounds 48, 56, 64, and 72), or branched amino PEGylated peptoids (Compounds 106, 107, 108, and 109) at weight percentages of 0.01%, 0.02%, 0.05% and 0.1% for the PEGylated compound.

Fluc expression from these formulations was measured in the cells following treatment for 24 hours based on the observed bioluminescence (total photon flux). For ease o comparison, the observed total photon fluxes were normalized to the formulation containing DMG-PEG2000 as a standard condition.

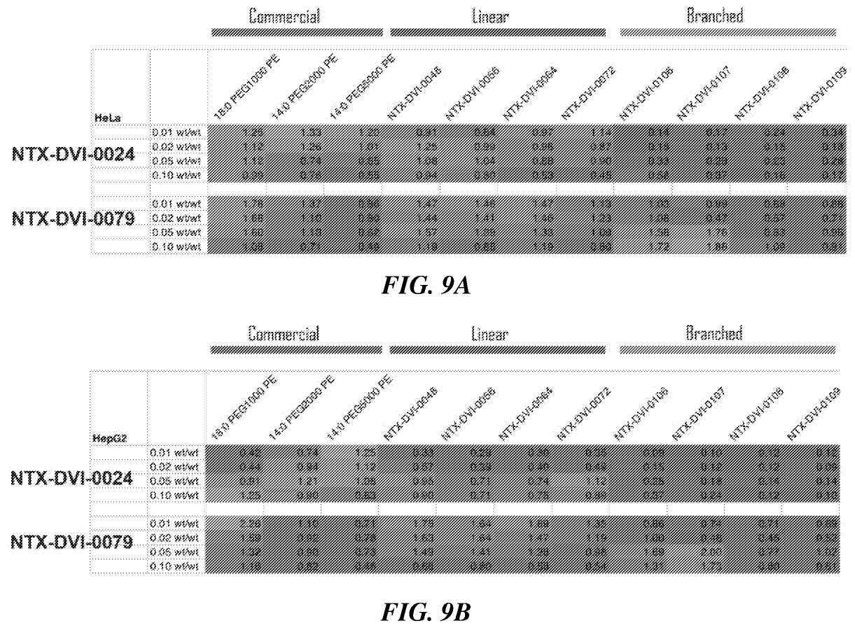

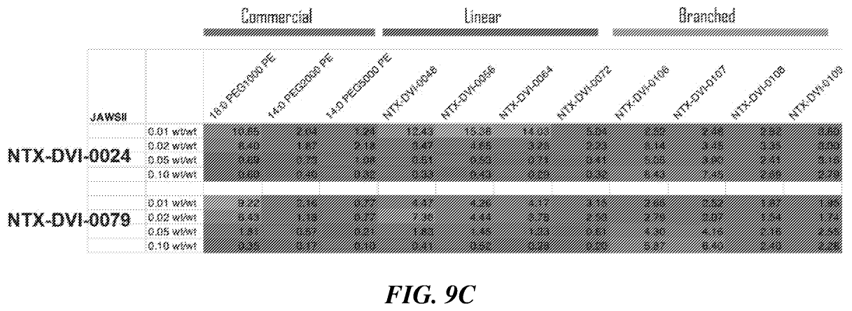

FIGS. 9A-9C depict normalized total photon fluxes (cps) in an in vitro evaluation of transfection efficiency of Fluc mRNA for various cell types (HeLa in FIG. 9A; HepG2 in FIG. 9B; and JAWSII in FIG. 9C).

Linear PEGylated peptoids were observed to show high transfection efficiency at lower weight percentages of incorporation. Branched PEGylated peptoids demonstrated better transfection when incorporated at higher weight percentages than at lower weight percentages.

1. A composition, comprising complexes of:
(i) one or more polyanionic compounds;
(ii) one or more PEG compounds; and
(iii) one or more cationic compounds, wherein the one or more cationic compounds are selected from the group consisting of:
(a) one or more tertiary amino lipidated and/or PEGylated cationic peptide compounds of formula (I) or salts thereof: 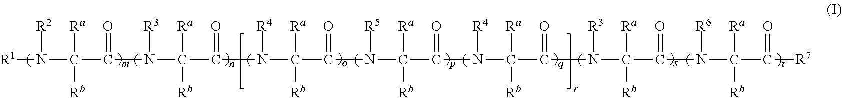
wherein:
m is 0;
n is an integer from 0 to 5;
s is an integer from 0 to 5;
t is 0;
wherein at least one of n and s is nonzero;
r is an integer from 1 to 20;
each o is independently an integer 0, 1, 2, 3 or 4;
each q is independently an integer 0, 1, 2, 3 or 4;
each p is independently an integer 1 or 2;
R¹ is —H, alkyl, alkylaryl, —COR^{1a} or a lipid moiety,
wherein R^{1a} is —H, —OH, alkyl, aryl, alkylaryl, —O-alkyl, or —O-alkylaryl;
each R² is independently an ethylene glycol moiety of the formula —CH₂CH₂O(CH₂CH₂O)ᵤCH₃, and wherein each u is independently an integer from 1 to 200;
each R³ is independently a lipid moiety;
each R⁴ is independently a neutral spacer moiety or a lipid moiety;
each R⁵ is independently a cationic moiety;
each R⁶ is independently an ethylene glycol moiety of the formula —CH₂CH₂O(CH₂CH₂O)ᵥCH₃, and wherein each v is independently an integer from 1 to 200;
R⁷ is-H, alkyl, acyl, —OH, —OR^{7a}, —NH₂, —NHR^{7a}, or a lipid moiety, wherein R^{7a} is alkyl, acyl, or a lipid moiety; and
each R^{a} and R^{b} is independently —H, C₁-C₄-alkyl, or a side chain moiety found on a naturally- or non-naturally-occurring amino acid;
(b) one or more lipitoids of formula (II): 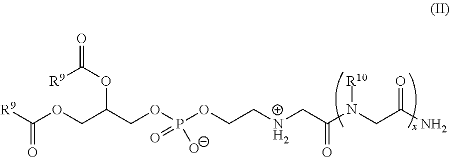
wherein:
x is a integer from 1 to 100;
each R⁹ is independently a lipid moiety; and
each R¹⁰ is independently a cationic or neutral spacer moiety;
(c) one or more lipid-like compounds of formula (III): 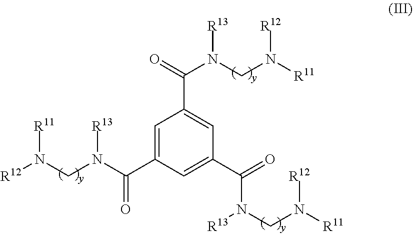
wherein:
each R¹¹ is independently substituted or unsubstituted alkyl;
each R¹² is independently substituted or unsubstituted alkyl;
each R¹³ is independently hydrogen or substituted or unsubstituted alkyl; and
each y is independently an integer from 1 to 8;
and
(d) any combination thereof;
and wherein the one or more cationic compounds, the one or more PEG compounds and the one or more polyanionic compounds have a combined mass percentage of at least 90% w/w of the complexes.

2. The composition of claim 1, wherein the one or more cationic compounds are one or more tertiary amino lipidated and/or PEGylated cationic peptide compounds of formula (I).

3. The composition of claim 1, wherein the one or more tertiary amino lipidated and/or PEGylated cationic peptide compounds comprises a block of N-lipidated amino acid residues, wherein either n or s is at least 2.

4. (canceled)

5. The composition of claim 1, wherein each R³ is independently C₄-C₂₂-alkyl or C₄-C₂₂-alkenyl, and wherein the C₄-C₂₂-alkenyl is optionally mono- or poly-unsaturated.

6.-10. (canceled)

11. The composition of claim 1, wherein at least one of the one or more tertiary amino lipidated and/or PEGylated cationic peptide compounds comprises a cationic domain comprising at least two cationic amino acid residues having cationic moieties R⁵.

12. The composition of claim 1, wherein each R⁵ is independently aminoalkyl, alkylaminoalkyl, aminoalkylaminoakyl, guanidinoalkyl, N-heterocyclylalkyl or N-heteroaryl.

13. The composition of claim 1, wherein each R⁵ is independently selected from the group consisting of:

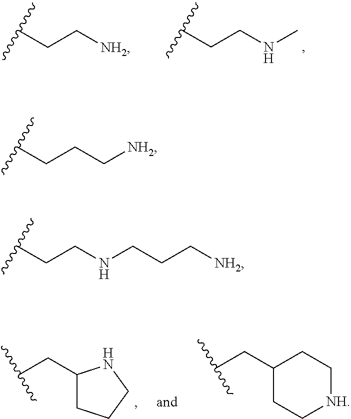

14.-17. (canceled)

18. The composition of claim 1, wherein each neutral spacer moiety R⁴ is independently a C₁-C₅ straight chain alkyl, a C₁-C₄-alkyl substituted by cycloalkyl, heterocyclylalkyl, alkylaryl, arylalkyl, alkylheteroaryl, heteroarylalkyl, alkoxy, alkoxyalkyl, or hydroxyalkyl, wherein each cycloalkyl, heterocyclylalkyl, alkylaryl, arylalkyl, alkylheteroaryl, heteroarylalkyl, alkoxy, alkoxyalkyl, or hydroxyalkyl is optionally substituted with one or more substituents —OH, halo, or alkoxy.

19.-20. (canceled)

21. The composition of claim 1, wherein at least one of the one or more tertiary amino lipidated and/or PEGylated cationic peptide compounds comprises a cationic domain comprising at least two amino acid residues having R⁵ cationic moieties and wherein each of the at least two cationic amino acid residues within the cationic domain are separated by at least one amino acid residue having a neutral spacer or lipid moiety R⁴.

22.-25. (canceled)

26. The composition of claim 1, wherein R^{a} and R^{b} are —H.

27. The composition of claim 1, wherein the one or more cationic peptide compounds and one or more PEG compounds are present at a mass ratio of peptide compound(s)-to-PEG compound(s) between 90:10 and 99:1.

28. (canceled)

29. The composition of claim 1, wherein the one or more PEG compounds comprises DMG-PEG2000.

30. The composition of claim 1, wherein the one or more PEG compounds comprises one or more tertiary amino lipidated and/or PEGylated cationic peptide compounds of formula (I) or salts thereof: 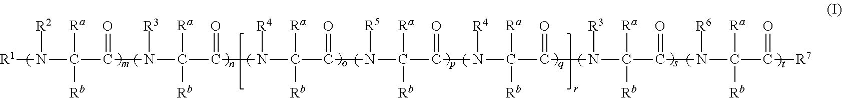
wherein:
m is an integer from 0 to 15;
n is an integer from 0 to 5;
s is an integer from 0 to 5;
t is an integer from 0 to 15;
wherein at least one of m and t is nonzero;
r is an integer from 1 to 20;
each o is independently an integer 0, 1, 2, 3 or 4;
each q is independently an integer 0, 1, 2, 3 or 4;
each p is independently an integer 1 or 2;
R¹ is —H, alkyl, alkylaryl, —COR^{1a} or a lipid moiety, wherein R^{1a} is —H, —OH, alkyl, aryl, alkylaryl, —O-alkyl, or —O-alkylaryl;
each R² is independently an ethylene glycol moiety of the formula —CH₂CH₂O(CH₂CH₂O)ᵤCH₃, and wherein each u is independently an integer from 1 to 200;
each R³ is independently a lipid moiety;
each R⁴ is independently a neutral spacer moiety or a lipid moiety;
each R⁵ is independently a cationic moiety;
each R⁶ is independently an ethylene glycol moiety of the formula —CH₂CH₂O(CH₂CH₂O),CH₃, and wherein each v is independently an integer from 1 to 200;
R⁷ is-H, alkyl, acyl, —OH, —OR⁷a, —NH₂, —NHR^{7a}, or a lipid moiety, wherein
R^{7a} is alkyl, acyl, or a lipid moiety; and
each R^{a} and R^{b} are independently —H, C1-C₄-alkyl, or a side chain moiety found on a naturally- or non-naturally-occurring amino acid.

31. The composition of claim 30, wherein
(i) when m is an integer from 0 to 3, each u is independently an integer from 30 to 50; and
when m is an integer from 4 to 15, each u is independently an integer from 1 to 10; and/or
(ii) when t is an integer from 0 to 3, each v is independently an integer from 30 to 50; and
when t is an integer from 4 to 15, each v is independently an integer from 1 to 10.

32.-33. (canceled)

34. The composition of claim 1, wherein the one or more polyanionic compounds comprises a nucleic acid.

35. The composition of claim 1, wherein the one or more polyanionic compounds comprises a nucleic acid and wherein the mass ratio of the one or more cationic peptide compounds to the nucleic acid is between 0.5:1 and 20:1.

36. The composition of claim 1, wherein the one or more polyanionic compounds comprises a nucleic acid and wherein the nucleic acid is an mRNA encoding a polypeptide and/or a protein.

37. (canceled)

38. The composition of claim 1, wherein the complexes further comprise: (i) one or more second agents, optionally wherein the one or more second agents are selected from the group consisting of Polyethylene glycol, a targeting element, and a combination thereof; and/or (ii) one or more small molecule active agents or drug substances.

39.-40. (canceled)

41. A method of delivering a polyanionic compound to a cell comprising contacting the cell with the composition of claim 1.

42.-45. (canceled)

46. A method of preparing a composition of claim 1, comprising contacting the one or more cationic peptide compounds with the one or more PEG compounds and the one or more polyanionic compounds.

47.-53. (canceled)

54. A method of delivering a polyanionic compound to lungs and/or spleen of a subject comprising administering to the subject the composition of claim 1, wherein the one or more cationic compounds are one or more tertiary amino lipidated and/or PEGylated cationic peptide compounds of formula (I), wherein the mass ratio of the one or more tertiary amino lipidated and/or PEGylated cationic peptide compounds of formula (I) to the polyanionic compound is between 1:1 and 10:1.

55.-59. (canceled)

60. The composition of claim 1, wherein the one or more cationic compounds are one or more tertiary amino lipidated and/or PEGylated cationic peptide compounds of formula (I) or salts thereof, wherein:
(a) n is 0; s is an integer from 1 to 5; r is an integer from 1 to 5; o is 0; q is 2; and p is 1, and formula (I) has a structure: 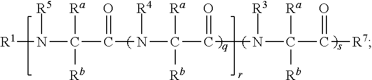
(b) n is 0; o is 0; p is 1; q is 0 or 1; r is an integer from 1 to 15; and s is an integer from 1 to 8; and formula (I) has a structure: 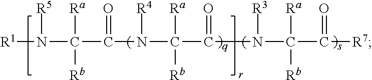
(c) n is 0; s is 3 or 4; r is 1; o is 0; q is 2; and p is 2, and formula (I) has a structure: 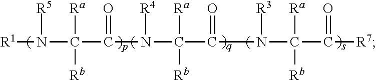
(d) n is 0; s is 3 or 4; r is an integer from 2 to 4; o is 0; q is 2; and p is 1, and formula (I) has a structure: 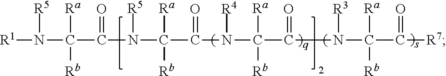
(e) n is 0; s is an integer from 1 to 5; r is an integer from 1 to 10; o is 0; q is 0; and p is 1, and formula (I) has a structure: 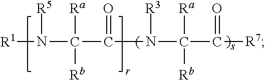
(f) R¹ is —COR^{1a}; R^{1a} is H; n is an integer from 0 to 4; o is 0; p is 1 or 2; q is 0; r is 1; s is an integer from 1 to 4; and formula (I) has a structure: 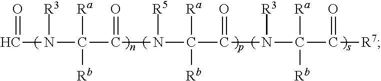
or
(g) combinations thereof.

61. The composition of claim 60, wherein
R¹ is H or alkyl;
each R³ independently is C₈-C₂₄alkyl;
each R⁴ is independently a neutral spacer moiety that is a C₁-C₄-alkyl substituted by cycloalkyl, heterocyclylalkyl, alkylaryl, arylalkyl, alkylheteroaryl, heteroarylalkyl, alkoxy, alkoxyalkyl, or hydroxyalkyl, wherein each cycloalkyl, heterocyclylalkyl, alkylaryl, arylalkyl, alkylheteroaryl, heteroarylalkyl, alkoxy, alkoxyalkyl, or hydroxyalkyl is optionally substituted with one or more substituents —OH, halo, or alkoxy;
each R⁵ is independently selected from the group consisting of aminoalkyl, alkylaminoalkyl, aminoalkylaminoakyl, guanidinoalkyl, N-heterocyclylalkyl, and N-heteroaryl, hydroxyalkyl, hydroxyether, alkoxyalkyl, and hydroxylheteroalkyl;
R⁷ is NH₂; and
each of R^{a} and R^{b} is H.

62. The composition of claim 60, wherein
each R³ independently is 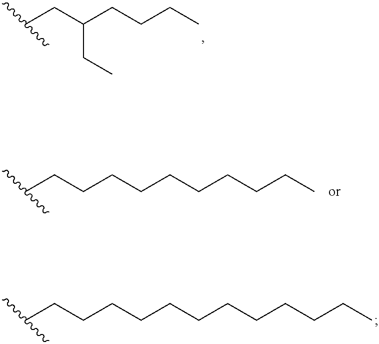
each R⁴ independently is selected from the group consisting of 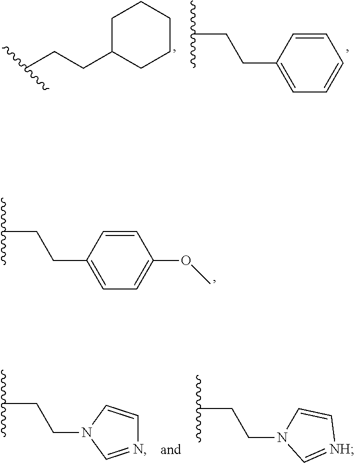
and
each R⁵ is independently selected from the group consisting of

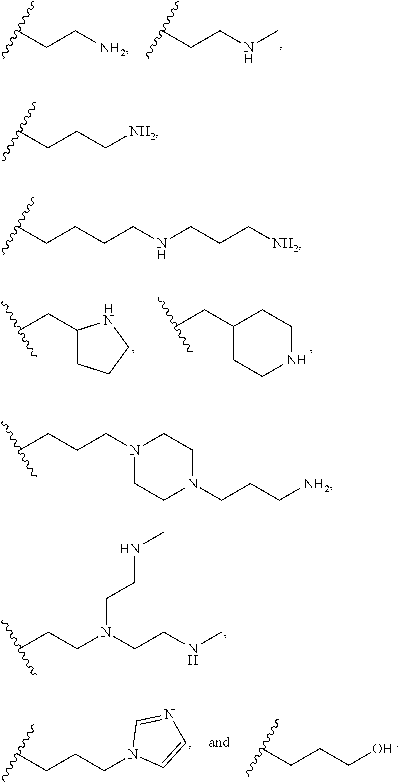

63. The composition of claim 30, wherein m is 1; n is 0; s is 3 or 4; t is 0; r is 1 or 2; o is 0; q is 2; and p is 1, and having a structure:

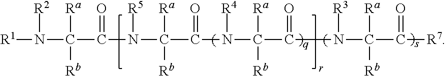

64. The composition of claim 63, wherein
R¹ is H;
R² is an ethylene glycol moiety of the formula —CH₂CH₂O(CH₂CH₂O)ᵤCH₃, and wherein each u is independently an integer from 2 to 50;
each R³ independently is C₈-C₂₄alkyl;
each R⁴ is independently a neutral spacer moiety that is a C₁-C₄-alkyl substituted by cycloalkyl, heterocyclylalkyl, alkylaryl, arylalkyl, alkylheteroaryl, heteroarylalkyl, alkoxy, alkoxyalkyl, or hydroxyalkyl, wherein each cycloalkyl, heterocyclylalkyl, alkylaryl, arylalkyl, alkylheteroaryl, heteroarylalkyl, alkoxy, alkoxyalkyl, or hydroxyalkyl is optionally substituted with one or more substituents —OH, halo, or alkoxy;
each R⁵ is independently selected from the group consisting of aminoalkyl, alkylaminoalkyl, aminoalkylaminoakyl, guanidinoalkyl, N-heterocyclylalkyl, and N-heteroaryl, hydroxyalkyl, hydroxyether, alkoxyalkyl, and hydroxylheteroalkyl;
R⁷ is NH₂; and
each of R^{a} and R^{b} is H.

65. The composition of claim 1, wherein the one or more tertiary amino lipidated and/or PEGylated cationic peptide compounds of formula (I) comprise more or more of a compound selected from compounds 1-36, 41-47, 49-55, 57-63, 65-71, 73-89, 91-103, and 111-118.
